(19) United States
(12) Patent Application Publication
(10) Pub. No.: US 2022/0022537 A1
(43) Pub. Date: Jan. 27, 2022

(54) SMOKING SUBSTITUTE APPARATUS

(71) Applicant: Nerudia Limited, Liverpool (GB)

(72) Inventors: Samantha MURRAY, Liverpool (GB); Chris LORD, Liverpool (GB); David JONES, Liverpool (GB); Jonathan MARCHBANK, Liverpool (GB); Tom SUDLOW, Liverpool (GB); Ian STUART, Liverpool (GB); Stephen JONES, Liverpool (GB)

(21) Appl. No.: 17/395,276

(22) Filed: Aug. 5, 2021

Related U.S. Application Data
(63) Continuation of application No. PCT/EP20/53119, filed on Feb. 7, 2020.
Application No. PCT/EP20/53119 is a continuation of application No. PCT/EP20/53193, filed on Feb. 7, 2020.
Application No. PCT/EP20/53193 is a continuation of application No. PCT/EP20/53195, filed on Feb. 7, 2020.
Application No. PCT/EP20/53195 is a continuation of application No. PCT/EP20/53112, filed on Feb. 7, 2020.
Application No. PCT/EP20/53112 is a continuation of application No. PCT/EP20/53186, filed on Feb. 7, 2020.
Application No. PCT/EP20/53186 is a continuation of application No. PCT/EP20/53116, filed on Feb. 7, 2020.
Application No. PCT/EP20/53116 is a continuation of application No. PCT/EP20/53194, filed on Feb. 7, 2020.
Application No. PCT/EP20/53194 is a continuation of application No. PCT/EP20/53189, filed on Feb. 7, 2020.
Application No. PCT/EP20/53189 is a continuation of application No. PCT/EP20/53120, filed on Feb. 7, 2020.
Application No. PCT/EP20/53120 is a continuation of application No. PCT/EP20/53115, filed on Feb. 7, 2020.
Application No. PCT/EP20/53115 is a continuation of application No. PCT/EP20/53130, filed on Feb. 7, 2020.
Application No. PCT/EP20/53130 is a continuation of application No. PCT/EP20/53139, filed on Feb. 7, 2020.
Application No. PCT/EP20/53139 is a continuation of application No. PCT/EP20/53141, filed on Feb. 7, 2020.
Application No. PCT/EP20/53141 is a continuation of application No. PCT/EP20/53191, filed on Feb. 7, 2020.
Application No. PCT/EP20/53191 is a continuation of application No. PCT/EP20/53110, filed on Feb. 7, 2020.
Application No. PCT/EP20/53110 is a continuation of application No. PCT/EP20/53200, filed on Feb. 7, 2020.
Application No. PCT/EP20/53200 is a continuation of application No. PCT/EP20/53106, filed on Feb. 7, 2020.
Application No. PCT/EP20/53106 is a continuation of application No. PCT/EP20/53190, filed on Feb. 7, 2020.
Application No. PCT/EP20/53190 is a continuation of application No. PCT/EP20/53176, filed on Feb. 7, 2020.

(30) Foreign Application Priority Data
Feb. 7, 2019 (EP) 19155858.4
Feb. 7, 2019 (EP) 19155861.8
Feb. 7, 2019 (EP) 19155862.6
Feb. 7, 2019 (EP) 19155864.2
Feb. 7, 2019 (EP) 19155866.7
Feb. 7, 2019 (EP) 19155871.7
Feb. 7, 2019 (EP) 19155873.3
Feb. 7, 2019 (EP) 19155875.8
Feb. 7, 2019 (EP) 19155877.4
Feb. 7, 2019 (EP) 19155878.2
Feb. 7, 2019 (EP) 19155879.0
Feb. 7, 2019 (EP) 19155887.3
Feb. 7, 2019 (EP) 19155894.9
Feb. 7, 2019 (EP) 19155903.8
Feb. 7, 2019 (EP) 19155908.7
Feb. 7, 2019 (EP) 19155929.3
Feb. 7, 2019 (EP) 19155933.5
Feb. 7, 2019 (EP) 19155934.3
Feb. 7, 2019 (EP) 19155936.8
Feb. 7, 2019 (EP) 19155945.9

(51) Int. Cl. A24F 40/42; A24F 40/485; A24F 40/70; A24F 40/10; A24B 15/167; A24B 15/28; A61M 15/00

(52) U.S. Cl. CPC A24F 40/42; A24F 40/485; A24F 40/70; A61M 2205/0238; A24B 15/167; A24B 15/285; A61M 15/009; A24F 40/10

(57) ABSTRACT

A substrate for a reservoir having an aerosol former stored therein, the substrate comprising: a property modifying agent disposed on the substrate, wherein the substrate is configured to be received in the reservoir to contact the aerosol former, so as to facilitate a release of the property modifying agent from the substrate to the aerosol former, and wherein the substrate is insoluble in the aerosol former.

### CROSS REFERENCE TO RELATED APPLICATIONS/INCORPORATION BY REFERENCE STATEMENT

This application is a non-provisional application claiming benefit to the international application no. PCT/EP2020/53119 filed on Feb. 7, 2020, which claims priority to EP 19155894.9 filed on Feb. 7, 2019. This application also claims benefit to the international application no. PCT/EP2020/53193 filed on Feb. 7, 2020, which claims priority to EP 19155934.3 filed on Feb. 7, 2019. This application also claims benefit to the international application no. PCT/EP2020/53195 filed on Feb. 7, 2020, which claims priority to EP 19155936.8 filed on Feb. 7, 2019. This application also claims benefit to the international application no. PCT/EP2020/53112 filed on Feb. 7, 2020, which claims priority to EP 19155866.7 filed on Feb. 7, 2019. This application also claims benefit to the international application no. PCT/EP2020/53186 filed on Feb. 7, 2020, which claims priority to EP 19155903.8 filed on Feb. 7, 2019. This application also claims benefit to the international application no. PCT/EP2020/53116 filed on Feb. 7, 2020, which claims priority to EP 19155871.7 filed on Feb. 7, 2019. This application also claims benefit to the international application no. PCT/EP2020/53194 filed on Feb. 7, 2020, which claims priority to EP 19155945.9 filed on Feb. 7, 2019. This application also claims benefit to the international application no. PCT/EP2020/53189 filed on Feb. 7, 2020, which claims priority to EP 19155908.7 filed on Feb. 7, 2019. This application also claims benefit to the international application no. PCT/EP2020/53120 filed on Feb. 7, 2020, which claims priority to EP 19155873.3 filed on Feb. 7, 2019. This application also claims benefit to the international application no. PCT/EP2020/53115 filed on Feb. 7, 2020, which claims priority to EP 19155861.8 filed on Feb. 7, 2019. This application also claims benefit to the international application no. PCT/EP2020/53130 filed on Feb. 7, 2020, which claims priority to EP 19155875.8 filed on Feb. 7, 2019. This application also claims benefit to the international application no. PCT/EP2020/53139 filed on Feb. 7, 2020, which claims priority to EP 19155877.4 filed on Feb. 7, 2019. This application also claims benefit to the international application no. PCT/EP2020/53141 filed on Feb. 7, 2020, which claims priority to EP 19155878.2 filed on Feb. 7, 2019. This application also claims benefit to the international application no. PCT/EP2020/53191 filed on Feb. 7, 2020, which claims priority to EP 19155929.3 filed on Feb. 7, 2019. This application also claims benefit to the international application no. PCT/EP2020/53110 filed on Feb. 7, 2020, which claims priority to EP 19155862.6 filed on Feb. 7, 2019. This application also claims benefit to the international application no. PCT/EP2020/53106 filed on Feb. 7, 2020, which claims priority to EP 19155864.2 filed on Feb. 7, 2019. This application also claims benefit to the international application no. PCT/EP2020/53190 filed on Feb. 7, 2020, which claims priority to EP 19155887.3 filed on Feb. 7, 2019. This application also claims benefit to the international application no. PCT/EP2020/53176 filed on Feb. 7, 2020, which claims priority to EP 19155879.0 filed on Feb. 7, 2019. This application also claims benefit to the international application no. PCT/EP2020/53200 filed on Feb. 7, 2020, which claims priority to EP 19155858.4 filed on Feb. 7, 2019 and to EP 19155933.5 filed on Feb. 7, 2019.

### FIELD OF THE DISCLOSURE

The present disclosure relates to a substrate for an aerosol former and, in particular, a substrate for releasing a property modifying agent to an aerosol former stored in the reservoir.

The present disclosure further relates to a smoking substitute apparatus and, in particular, a smoking substitute apparatus that is able to deliver flavor to a user by adding a flavorant to an aerosol former.

The present disclosure further relates to a flavor delivery article in the form of a flavored sticker for use with a smoking substitute apparatus, to deliver flavor or an additive to a user of the smoking substitute apparatus.

The present disclosure further relates to packaging for a smoking substitute apparatus and, in particular, to packaging which includes an aromatic additive.

The present disclosure further relates to a mouthpiece for a smoking substitute apparatus, and to a flavorant dispenser for applying flavorant to the mouthpiece.

The present disclosure further relates to a smoking substitute apparatus and, in particular, a smoking substitute apparatus able to deliver flavor to a user by adding a flavorant to an aerosol former.

The present disclosure further relates to a mouthpiece for a smoking substitute apparatus, where the mouthpiece has an electrode for stimulating a user's tongue to simulate flavor delivery.

The present disclosure further relates to a flavor delivery article for use with a smoking substitute apparatus and, in particular, a flavor delivery article that is able to deliver flavor to a user during the use of the smoking substitute apparatus.

The present disclosure further relates generally to smoking substitute apparatuses, and smoking substitute kits which are in some way configured to deliver a flavorant to a user without the need for flavored e-liquid, for example using printed flavorants, flavored or scented ink, and a kit including a smoking substitute apparatus and a flavorant applicator.

### BACKGROUND

The smoking of tobacco is generally considered to expose a smoker to potentially harmful substances. It is generally thought that a significant amount of the potentially harmful substances is generated through the heat caused by the burning and/or combustion of the tobacco and the constituents of the burnt tobacco in the tobacco smoke itself.

Combustion of organic material such as tobacco is known to produce tar and other potentially harmful by products. There have been proposed various smoking substitute systems in order to avoid the smoking of tobacco.

Such smoking substitute systems can form part of nicotine replacement therapies aimed at people who wish to stop smoking and overcome a dependence on nicotine.

Smoking substitute systems include electronic systems that permit a user to simulate the act of smoking by producing an aerosol (also referred to as a “vapor”) that is drawn into the lungs through the mouth (inhaled) and then exhaled. The inhaled aerosol typically bears nicotine and/or a flavorant without, or with fewer of, the odor and health risks associated with traditional smoking.

In general, smoking substitute systems are intended to provide a substitute for the rituals of smoking, whilst providing the user with a similar experience and satisfaction to those experienced with traditional smoking and with combustible tobacco products.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories. There are a number of different categories of smoking substitute systems, each utilizing a different smoking substitute approach.

One approach is the so-called “vaping” approach, in which a vaporizable liquid, typically referred to (and referred to herein) as “e-liquid”, is heated by a heating device (referred to herein as an electronic cigarette or “e-cigarette” device) to produce an aerosol vapor which is inhaled by a user. The e-liquid typically includes a base liquid as well as nicotine and/or a flavorant. The resulting vapor therefore also typically contains nicotine and/or a flavorant. The base liquid may include propylene glycol and/or vegetable glycerin.

A typical e-cigarette device includes a mouthpiece, a power source (typically a battery), a tank for containing e-liquid, as well as a heating device. In use, electrical energy is supplied from the power source to the heating device, which heats the e-liquid to produce an aerosol (or “vapor”) which is inhaled by a user through the mouthpiece.

E-cigarettes can be configured in a variety of ways. For example, there are “closed system” vaping smoking substitute systems, which typically have a sealed tank and heating element. The tank is pre-filled with e-liquid and is not intended to be refilled by an end user. One subset of closed system vaping smoking substitute systems includes a main body which includes the power source, wherein the main body is configured to be physically and electrically coupled to a consumable including the tank and the heating element. In this way, when the tank of a consumable has been emptied, that consumable is disposed of. The main body can be reused by connecting it to a new, replacement, consumable. Another subset of closed system vaping smoking substitute systems is completely disposable, and intended for one-use only.

There are also “open system” vaping smoking substitute systems which typically have a tank that is configured to be refilled by a user. In this way the entire device can be used multiple times.

An example vaping smoking substitute system is the myblu™ e-cigarette. The myblu™ e-cigarette is a closed system which includes a main body and a consumable. The main body and consumable are physically and electrically coupled together by pushing the consumable into the main body. The main body includes a rechargeable battery. The consumable includes a mouthpiece, a sealed tank which contains e-liquid, as well as a heater, which for this device is a heating filament coiled around a portion of a wick. The wick is partially immersed in the e-liquid, and conveys e-liquid from the tank to the heating filament. The device is activated when a microprocessor on board the main body detects a user inhaling through the mouthpiece. When the device is activated, electrical energy is supplied from the power source to the heating device, which heats e-liquid from the tank to produce a vapor which is inhaled by a user through the mouthpiece.

An alternative to the “vaping” approach is the so-called Heated Tobacco (“HT”) approach in which tobacco (rather than an e-liquid) is heated or warmed to release vapor. HT is also known as “heat not burn” (“HNB”). The tobacco may be leaf tobacco or reconstituted tobacco. In the HT approach the intention is that the tobacco is heated but not burned, i.e., the tobacco does not undergo combustion.

The heating, as opposed to burning, of the tobacco material is believed to cause fewer, or smaller quantities, of the more harmful compounds ordinarily produced during smoking. Consequently, the HT approach may reduce the odor and/or health risks that can arise through the burning, combustion and pyrolytic degradation of tobacco.

A typical HT smoking substitute system may include a device and a consumable. The consumable may include the tobacco material. The device and consumable may be configured to be physically coupled together. In use, heat may be imparted to the tobacco material by a heating element of the device, wherein airflow through the tobacco material causes components in the tobacco material to be released as vapor. A vapor may also be formed from a carrier in the tobacco material (this carrier may for example include propylene glycol and/or vegetable glycerin) and additionally volatile compounds released from the tobacco. The released vapor may be entrained in the airflow drawn through the tobacco.

As the vapor passes through the consumable (entrained in the airflow) from the location of vaporization to an outlet of the consumable (e.g., a mouthpiece), the vapor cools and condenses to form an aerosol for inhalation by the user. The aerosol may contain nicotine and/or flavor compounds.

For a smoking substitute device, it is desirable to deliver nicotine into the user's lungs, where it can be absorbed into the bloodstream. As explained above, in the so-called “vaping” approach, e-liquid is heated by a heating device to produce an aerosol vapor which is inhaled by a user. Many e-cigarettes also deliver flavor to the user to enhance the experience. In such e-cigarettes, e-liquid is often sold as a flavored product, e.g., a specific blend of flavor compounds is already homogeneously mixed with the e-liquid during the manufacturing process. As such, the user would have to purchase flavored consumables available on the market with limited opportunities to personalize the vaping experience according to their preferences. Further, when blended in the e-liquid, flavor components may interact with other constituent in the e-liquid during storage. In addition, it may put additional strain on the supply chain for distributing a large variety of consumables having different flavors.

Additionally, many e-cigarettes also deliver flavor to the user to enhance the experience. In such e-cigarettes, flavor compounds are contained in the e-liquid that is heated. However, in some cases, toxicology restrictions are placed on the amount of flavor that can be contained in the e-liquid, and this can result in some e-liquid flavors delivering a weak and underwhelming taste sensation to consumers in the pursuit of safety.

There may be a need for improved design of smoking substitute systems, in particular in regards to the delivery of flavor to a user.

The present disclosure has been devised in the light of the above considerations.

### SUMMARY OF THE DISCLOSURE

First Mode: A Substrate for Releasing a Property Modifying Agent to an Aerosol Former Stored in a Reservoir

At its most general, a first mode of the present disclosure relates to a substrate for releasing a property modifying agent to an aerosol former stored in a reservoir. This may allow the user to flavor the aerosol former at the point of use. Therefore, the substrate may enable the property modifying agent to be kept separate to the aerosol former, it may minimize the interaction between the two during transport and storage. Further, it may allow the user to specify the type and quantity of property modifying agent to be added to the aerosol former, thus it may enable the user to create an aerosol former with a specific flavor and/or color tailored to the user's needs.

According to a first aspect of the first mode there is provided a substrate for a reservoir having an aerosol former stored therein, the substrate comprising:
a property modifying agent disposed on the substrate;
wherein the substrate is configured to be received in the reservoir to contact an aerosol former stored therein, so as to facilitate a release of the property modifying agent from the substrate to the aerosol former; and
wherein the substrate is insoluble in the aerosol former.

The aerosol former may be an e-liquid. The e-liquid may, for example, comprise a base liquid and nicotine. The base liquid may include propylene glycol and/or vegetable glycerin. The e-liquid may be flavorless. That is, the e-liquid may not contain any flavorant and may consist solely of a base liquid of propylene glycol and/or vegetable glycerin and nicotine.

The property modifying agent may comprise a flavorant. The flavorant may be provided in solid, gel or liquid form. It may include menthol, licorice, chocolate, fruit flavor (including, e.g., citrus, cherry, etc.), vanilla, spice (e.g., ginger, cinnamon) and tobacco flavor. The flavorant may modify a flavor of the aerosol former upon contacting or mixing with the aerosol former.

The property modifying agent may comprise a colorant. The colorant may be provided in solid, gel or liquid form. The colorant may modify a color of the aerosol former upon contacting or mixing with the aerosol former. Advantageously, when the colorant is provided with the flavorant in the property modifying agent, the colorant may provide visual indication to a user for indicating a progress of agent release. For example, the concentration of the flavorant may correspond to a change in color of the aerosol former.

The substrate may allow the property modifying agent to be kept separately to the aerosol former stored in the reservoir during transportation and storage. The aerosol former and the property modifying agent may only come into contact once the substrate is received in the reservoir. This may advantageously eliminate or reduce the interaction between the aerosol former and the property modifying agent during transportation and storage. For example, the e-liquid may only be flavored with flavorant immediately before use.

The substrate may be configured to release the property modifying agent into the aerosol former when it is received in the reservoir. More specifically, the substrate may be configured to contact the aerosol former in the reservoir when it is received in the reservoir. For example, at least a part of the substrate may be immersed in the aerosol former when it is received in, or inserted into, the reservoir. Due to a difference in concentration, the property modifying agent absorbed or adsorbed in the substrate may therefore diffuse or transport out of the substrate into the aerosol former.

The substrate may be formed of an absorbent material configured to absorb or adsorb the property modifying agent. For example, the absorbent material may comprise a cellulose or fibrous material which absorbs or adsorbs the property modifying agent into the body of the material. The absorbent material may comprise a porous material, such as a foam, which absorbs the property modifying agent into the body of the material. Advantageously, the use of an absorbent material may allow a large quantity of property modifying agent to be absorbed or adsorbed therein. The absorbent material may function as a storage for storing the property modifying agent. Advantageously, the property modifying agent as stored within the body of absorbent material may gradually diffuse or transport towards its surface and thereby replenish property modifying agent released to the aerosol former. As a result, the concentration of property modifying agent at the surface of the substrate may be maintained.

Alternatively, the substrate may be formed of a non-absorbent material. For example, the substrate may comprise a non-absorbent surface for the property modifying agent material to attach or adhere onto.

Advantageously, the use of a non-absorbent material may prevent the aerosol former from being adsorbed into the substrate when said substrate is put in contact with the aerosol former, and thereby reduces the amount of aerosol former being retained on the substrate.

Optionally, the substrate is configured to be removably received in the reservoir. For example, the substrate may be retained in the reservoir for a predetermined period of time before it is removed, or withdrawn, from the reservoir. Doing so may allow the majority of the property modifying agent to be released into the aerosol former before the substrate is removed. Thereafter the substrate, exhausted of property modifying agent, may be removed and be disposed of. Advantageously, the removal of an exhausted substrate from the reservoir may avoid constricting the flow of aerosol former in the reservoir and as a result, the aerosol former may flow freely in the reservoir. Therefore, it may improve the efficiency of aerosol generation.

Optionally, the substrate is configured to be received in a reservoir of a container. That is, the reservoir forms a part of a container for storing aerosol former. The container is not configured to generate an aerosol. For example, the container may be a bottle or a canister. The container may be prefilled with unflavored and/or uncolored aerosol former. The container may comprise a container outlet for dosing or adding aerosol former to a corresponding reservoir of a smoking substitute apparatus or system.

Optionally, the substrate is configured to be received in a reservoir of a smoking substitute apparatus. That is, the reservoir may form a part of a smoking substitute apparatus. For example, the smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

The substrate may be fully received into the reservoir through an opening at the reservoir. The substrate may be retained in the reservoir without being removed. Alternatively, the substrate may be retained in the reservoir for a predetermined period of time. The substrate may be removed from the reservoir after a predetermined period of time with the use of a suitable removal mechanism. For example, the substrate may be removed by pulling on a part of the substrate by hand or a tweezer.

The substrate may be partially received into the reservoir through an opening at the reservoir. That is, a first end of the substrate may be received in the reservoir and in contact with the aerosol former, and a second end of the substrate extends, through the opening, outwardly from the reservoir. Advantageously, the second end of the substrate may form a tab for a user to pull onto when removing the substrate from the reservoir.

The first end of the substrate may form of an absorbent material whilst the second end of the substrate may form of a non-absorbent material, such that the aerosol former may not be transported, via capillary force, from the first end of the substrate towards the second end of the substrate when the first end is put in contact with the aerosol former. Advantageously, the second end of the substrate, or the tab, may be free of aerosol former and thereby allowing the user to grip onto the tab more easily.

The opening at the reservoir may be shaped correspond to a cross sectional profile of the substrate. For example, the opening may be a slit for receiving a planar substrate, e.g., a card or a stick, or it may be circular for receiving a cylindrical shaped substrate, e.g., a rod, or it may be rectangular for receiving a cuboid shaped substrate.

The opening may be closed by a closure for preventing the aerosol former stored in the reservoir from leaking out of the reservoir. The closure may be opened to provide passage for the substrate as it inserts and/or extends into the reservoir, and it may be closed and resealed once the substrate is received in, withdrawn from or removed from the reservoir. The closure may, for example, comprise a lid, a cap or a flap that covers the opening, or it may comprise a valve positioned across the opening to close said opening.

The closure may comprise a duck bill valve. For example, the valve may comprise a resilient member for forming a peripheral seal with the substrate as it extends through the opening. Said valve may prevent the aerosol former stored in the reservoir from leaking though the opening when the substrate is partially received in the reservoir. Advantageously, such arrangement may allow the smoking substitute apparatus to be kept in different orientations with the substrate partially inserted therein, yet preventing the aerosol former from leaking out of the reservoir through the opening.

Optionally, the property modifying agent is configured to be deposited on a surface of the substrate. For example, the property modifying agent may be coated onto the surface of the substrate. The property modifying agent may comprise a powder, or microcapsules, adhered to or partially embedded into the surface of the substrate. The property modifying agent may comprise a crystalline structure being formed onto the surface of the substrate by means of crystallization. The property modifying agent may comprise a liquid property modifying agent absorbed or adsorbed on to the surface of the substrate. Advantageously, the property modifying agent as deposited on the surface of the substrate may be directly and promptly released into the aerosol former once the substrate is received in the reservoir, as such reducing the amount of time require for releasing the property modifying agent.

Optionally, the substrate is coated in a soluble coating containing the property modifying agent. Optionally, the soluble coating is configured to at least partially dissolve in the aerosol former for releasing the property modifying agent. For example, the soluble coating may be a soluble gel or a soluble shell that dissolves in the aerosol former. The soluble coating may provide a means for the property modifying agent to adsorb or attach onto the surface of the substrate. The soluble coating may be formed using vacuum drying technique, for example a soluble shell may be formed by vacuum drying a gel coated on the surface of the substrate. The soluble coating may allow the property modifying agent to be incorporated therein and thus the rate of release of property modifying agent may correspond to the rate of dissolve of the soluble coating when the substrate is put in contact with the aerosol former. The concentration of the property modifying agent may be constant throughout the thickness of the coating, or it may have a varied concentration gradient. Advantageously, such arrangement allows the release of property modifying agent to be controlled more precisely.

The substrate may be formed of a material that does not dissolve in the aerosol former, for example a paper. Advantageously, the use of an insoluble substrate reduces or eliminate contamination of the aerosol former as it is being received in the reservoir. That is, an insoluble substrate only releases the property modifying substrate to the aerosol former and whilst maintaining its structural integrity.

Optionally, the substrate comprises an absorbent paper. For example, the substrate may comprise a card formed from blotting paper, bibulous paper, watercolor paper or any other absorbent paper known to the person skilled in the art. The absorbance of an absorbent paper, e.g., the amount of property modifying agent that can be adsorbed onto absorbent paper, may depend upon its thickness. Advantageously, the dosage and release profile of the property modifying agent may be controlled by varying the type or the thickness of the absorbent paper forming the substrate.

Furthermore, the use of absorbent paper allows the property modifying agent to be disposed on the substrate during its manufacturing process. For example, the property modifying agent may be applied to the pulp for producing the absorbent paper, or it may be applied to the paper prior to or after a drying process.

Optionally, at least a portion of substrate is soluble in the aerosol former. For example, the substrate may comprise rice paper which readily dissolves in the aerosol former. This may allow the property modifying agent to incorporate into the soluble portion of the substrate and thus the rate of release of property modifying agent may correspond to the rate of dissolve of soluble substrate when it is put in contact with the aerosol former. Advantageously, such arrangement allows the release of property modifying agent to be more precisely controlled.

Optionally, the substrate is configured to be removable received in the reservoir. That is, the substrate may be removed from the reservoir once the property modifying agent is released from the substrate to the aerosol former stored in the reservoir.

Optionally, the amount of property modifying agent being released to the aerosol former to a predetermined period of time, or a residence time, the substrate is received in the reservoir. More specifically, in the presence of aerosol former, the property modifying agent may be released from the substrate to the aerosol former at a given rate of release. Thus, the longer the residence time the more property modifying agent may be released to the aerosol former. The release of property modifying agent may continue until it reaches an equilibrium, e.g., the difference in concentration of property modifying agent between the aerosol former and substrate is insignificant.

Therefore, the concentration of property modifying agent, e.g., flavorant and/or colorant, in the aerosol former may be controlled by varying the residence time of the substrate. Advantageously, the flavor and/or color of the aerosol former may be tailored to a user's preference by removing the substrate from the reservoir after a predetermined period of time. For example, a user may opt to remove the substrate from the reservoir earlier if a less flavored and/or colored aerosol former is desired.

Optionally, the predetermined period of time, or the residence time, that the substrate is received in the reservoir, ranges from 1 minute to 2 hours. Optionally, said predetermined period of time is at least 5 minutes, or at least 60 minutes. Optionally, said predetermined period of time is any one of 1 minute, 5 minutes, 10 minutes, 15 minutes, 30 minutes, 60 minutes, 90 minutes and 120 minutes.

According to a second aspect of the first mode there is provided a reservoir for receiving the substrate, the reservoir is configured to store an aerosol therein, the reservoir comprising an opening for providing access to the substrate as it extends into the reservoir to contact the aerosol former. The reservoir may form a part of container or smoking substitute apparatus.

Optionally, the reservoir comprises a closure for closing the opening, wherein the closure is configured to open to provide access for the substrate. The opening may be closed by a closure for preventing the aerosol former stored in the reservoir from leaking out of the reservoir. The closure may be opened to provide passage for the substrate as it inserts and/or extends into the reservoir, and it may be closed and resealed once the substrate is received in, withdrawn from or removed from the reservoir.

Optionally, the closure comprises a lid, a cap or a flap for covering the opening, or it may comprise a valve positioned across the opening to said opening.

Optionally, the valve may comprise a resilient member for forming a peripheral seal with the substrate as it extends through the opening. The closure may comprise a duck bill valve. Said valve may prevent the aerosol former stored in the reservoir from leaking though the opening when the substrate is partially received in the reservoir. Advantageously, such arrangement may allow the smoking substitute apparatus to be kept in different orientations with the substrate partially inserted therein, yet preventing the aerosol former from leaking out of the reservoir through the opening.

Optionally, the resilient member may contact a surface of the substrate during its removal from the reservoir so as to remove at least some aerosol former from the surface of the substrate. Advantageously, such arrangement may reduce the amount of aerosol former being removed with the substrate.

Optionally, the reservoir comprises a wall, and wherein at least a portion of the wall is translucent or transparent such that the aerosol former stored in the reservoir is visible through said portion of the wall. Advantageously, this may allow a user to visually inspect the progress of mixing, for example, with the aid of a colorant in the property modifying agent.

Optionally, the closure comprises a valve positioned across the opening to close said opening. According to a third aspect of the first mode there is provided a smoking substitute apparatus comprising the reservoir. According to a fourth aspect of the first mode there is provided a smoking substitute kit comprising: the substrate and the reservoir or the smoking substitute apparatus.

According to a fifth aspect of the first mode there is provided a method of producing the substrate, comprising: preparing a pulp; applying a property modifying agent to the pulp; and drying the pulp to form the substrate.

Advantageously, applying the property modifying agent to the pulp may allow the property modifying agent to form together with the substrate, as such it may reduce the amount of property modifying agent that is released during transportation and storage.

Optionally, said drying comprises one or more of mechanical drying process, evaporative drying process and vacuum drying process.

Where the smoking substitute apparatus is in the form consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorant and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the aerosol-delivery device. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects of the first mode and preferred features described except where such a combination is clearly impermissible or expressly avoided.

The skilled person will appreciate that except where mutually exclusive, a feature or parameter described in relation to any one of the above aspects of the first mode may be applied to any other aspect of the first mode. Furthermore, except where mutually exclusive, any feature or parameter of the first mode described herein may be applied to any aspect and/or combined with any other feature or parameter of the first mode described herein.

Second Mode: A Flavored Sticker for Use with a Smoking Substitute Apparatus

At its most general, a second mode of the present disclosure relates to a flavored sticker for use with a smoking substitute apparatus. The flavored sticker may be applied to a mouthpiece of the smoking substitute apparatus to deliver flavor (e.g., a taste and/or scent) to a user of the smoking substitute apparatus.

In this manner, flavor may be delivered to a user through the sticker, rather than through a flavorant contained in an aerosol-former (e.g., e-liquid or tobacco material) of the of the smoking substitute apparatus. As a result, there may be no need to include any flavorants in the aerosol-former of the smoking substitute apparatus.

The sticker may be applied to the mouthpiece when the user wishes to experience flavor. The sticker may then be removed if the user no longer wants to receive flavor, or if they want to change the sticker. Stickers having different flavors may be provided, so that the user may apply a sticker with a desired flavor to the mouthpiece, without having to otherwise modify the smoking substitute apparatus. This may improve a versatility of the smoking substitute apparatus, as the user may experience a wide range of flavors simply by changing the sticker on the mouthpiece.

According to a first aspect of the second mode there is provided a sticker for use with a smoking substitute apparatus, the sticker comprising: an adhesive portion for adhering the sticker to a mouthpiece of the substitute apparatus; and a flavored portion, the flavored portion being arranged to cover an outlet of the mouthpiece when the sticker is adhered to the mouthpiece; wherein the flavored portion is permeable to an aerosol generated by the smoking substitute apparatus.

Thus, a user may adhere the sticker to the mouthpiece of a smoking substitute apparatus so that the flavored portion of the sticker covers the outlet of the mouthpiece. In this manner, an aerosol generated by a smoking substitute device and delivered to the user via the mouthpiece may pass through the flavored portion of the sticker and pick up flavor from the sticker. As a result, flavor may be delivered to the user, together with the aerosol. Additionally, due to the sticker's location on the mouthpiece, it may come into contact with the user's tongue so as to directly deliver flavor to the user's tongue. The smoking substitute device may be a vaping type device or a heated tobacco device.

The adhesive portion of the sticker may include an adhesive for adhering (sticking) the sticker to a surface of the mouthpiece. The adhesive may be configured to stick the sticker to mouthpiece when the adhesive portion is pressed against a surface of the mouthpiece. Preferably, the adhesive may be of a type that facilitates removal, so that the sticker may be easily removed from the mouthpiece after use. In some embodiments, the adhesive portion of the sticker may be disposed on a first side of the sticker, and the flavored portion may be disposed on a second, opposite side of the sticker. This may avoid bringing the adhesive portion into contact with the user's tongue.

The sticker may include a backing arranged to cover the adhesive portion before use. Then, to affix the sticker to the mouthpiece, the backing may be removed to expose the adhesive portion so that it can be applied to the mouthpiece.

The flavored portion is arranged to cover the outlet of the mouthpiece of the smoking substitute apparatus. For example, the flavored portion may have a shape that substantially matches a shape of the outlet of the mouthpiece, so that the flavored portion may be aligned with the outlet to cover the outlet. Alternatively, the flavored portion may be larger than the outlet, to facilitate placing it over the outlet. The shape and size of the sticker, and the layout of the adhesive and flavored portions may be adapted to the layout of the smoking substitute apparatus with which the sticker is intended to be used.

The flavored portion may include a flavorant for producing a flavor (e.g., taste and/or scent). The flavorant may be arranged to be entrained into the user's mouth by a flow of aerosol through the outlet of the mouthpiece when the sticker is adhered to the mouthpiece. Additionally, or alternatively, the flavorant may be arranged to produce a smell and/or taste that is perceived by the user when the user puts the mouthpiece having the sticker adhered thereto in their mouth. In this manner, flavor may be effectively delivered to the user.

The term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances.

The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations in the flavored portion of the sticker.

The flavored portion is permeable to an aerosol generated by the smoking substitute apparatus. This may mean that the flavored portion is capable of transmitting an aerosol generated by the smoking substitute apparatus. In this manner, aerosol generated by the smoking substitute apparatus may be delivered to the user through the flavored portion of the sticker.

The flavored portion may be heat-activated. For example, the flavored portion may be arranged to release flavorant in response to heat being applied to the flavored portion. For example, when the sticker is adhered to the mouthpiece of a smoking substitute apparatus, the mouthpiece may be inserted into a user's mouth. The user's mouth (e.g., lips and/or tongue) may heat the flavored portion of the sticker, which may cause the flavored portion to release flavorant to the user. In this manner, the sticker may only produce a flavor when in use. Prior to activation of the flavored portion (e.g., before being heated by a user's mouth), the sticker may be substantially odorless. As an example, the flavored portion may include flavorant that is contained within microcapsules (e.g., made of gelatin, or the like), the microcapsules being arranged to rupture upon application of heat (e.g., from the user's mouth). In this manner when heat is applied to the flavored portion, the microcapsules may rupture, causing release of flavorant.

The flavored portion may be friction-activated. For example, the flavored portion may be arranged to release flavorant in response to friction being applied to the flavored portion. For example, to activate the flavored portion, a user may rub the sticker to cause release of flavor. In this manner, prior to use of the sticker, no flavor may be emitted by the flavored portion, such that the sticker is substantially odorless. As an example, the flavored portion may include flavorant that is contained within microcapsules (e.g., made of gelatin, or the like), the microcapsules being arranged to rupture upon application of friction (e.g., from rubbing or scratching the sticker). In this manner when friction is applied to the flavored portion, the microcapsules may rupture, causing release of flavorant.

The flavored portion may be activated by the aerosol generated by the smoking substitute apparatus passing through the flavored portion. For example, the flavored portion may be arranged to release flavorant in response to an aerosol passing through the flavored portion. In this manner, the flavored portion may only deliver flavor when a user inhales an aerosol from the smoking substitute apparatus through the flavored portion of the sticker.

As an example, the flavored portion may include a barrier layer which encapsulates the flavorant. The barrier layer may be configured to rupture or disintegrate in the presence of aerosol, so that the flavorant is released. Similarly, the flavorant may be contained in microcapsules that are configured to rupture and release the flavorant when aerosol comes into contact with the microcapsules.

For example, the microcapsules may include a membrane made of a hydrogel composite containing Chitosan. Chitosan is substantially stable around pH 7.5. Thus, when a microcapsule enters the user's mouth, the Chitosan, and hence the microcapsule's membrane, may be stable and retain its integrity fora period of time. When the user inhales an aerosol having a more alkaline pH (e.g., an aerosol produced from an e-liquid having a pH around 7.8), the change in pH in the user's mouth may cause the Chitosan, and hence microcapsule's membrane, to rapidly break down and release the flavorant contained within.

The flavorant in the flavored portion may be a freeze-dried flavorant. In this manner, the flavorant may be activated by moisture in the user's mouth. Thus, the flavorant may not produce a flavor until it reaches the user's mouth.

The flavored portion may include a removable protective outer layer. The removable protective outer layer may serve to protect the flavored portion (e.g., from contamination) before use. The removable protective outer layer may then be removed to exposed the flavored portion for use. The removable protective outer layer may, for example, be a layer of material that covers the flavored portion and that can be removed from the flavored portion by peeling it off. In another example, the removable protective outer layer may be removed by rubbing and/or scratching the removable protective outer layer.

The flavored portion may include one or more apertures for transmitting the aerosol generated by the smoking substitute apparatus. The one or more apertures may be dimensioned to enable transmission of the aerosol, to render the flavored portion permeable to the aerosol. The one or more apertures may be an array of apertures that are distributed over an area of the flavored portion. In this manner, aerosol may be transmitted substantially uniformly by the flavored portion.

The flavored portion may include a porous material for transmitting the aerosol generated by the smoking substitute apparatus. This may render the flavored portion permeable to the aerosol. The porous material may, for example, be a mesh material, a cloth material, a fiber material, a filter material, or a paper material. Using a porous material for the flavored portion may enhance transmission of aerosol by the flavored portion, which may facilitate inhalation of the aerosol by the user.

The porous material may contain a flavorant. In this manner, when the aerosol passes through the porous material, it may entrain the flavorant contained therein to deliver flavor to the user. As an example, the porous material may be imbued with a liquid flavorant. As another example, the flavorant may be a powder which is contained within the porous material.

The adhesive portion may include a ring of adhesive arranged to surround the outlet of the mouthpiece when the sticker is adhered to the mouthpiece. Thus, when the sticker is adhered to the mouthpiece, the ring of adhesive may form a seal around the outlet of the mouthpiece, so that aerosol exiting the mouthpiece must pass through the flavored portion of the sticker. In other words, the ring of adhesive may prevent aerosol from escaping from the mouthpiece without passing through the flavored portion of the sticker. This may improve the quality of flavor delivery to the user. The ring of adhesive may be circular, or have any other suitable shape for surrounding the outlet of the mouthpiece.

The flavored portion may include a flavorant disposed on a surface of the sticker. In this manner, the flavorant may come in direct contact with the user's mouth (e.g., tongue) when the sticker is adhered to the mouthpiece and the mouthpiece is inserted into the user's mouth.

The flavored portion may include a flavorant that is embedded in a material forming the flavored portion. This may enable the flavorant to be entrained by an aerosol passing through the flavored portion. The flavorant may be embedded in the material forming the flavored portion during manufacture of the flavored portion.

According to a second aspect of the second mode of the disclosure, there is provided a smoking substitute apparatus comprising: a mouthpiece with an outlet for delivering an aerosol generated by the smoking substitute apparatus to a user; and a sticker according to the first aspect of the second mode of the disclosure, the sticker being adhered to the mouthpiece such that the flavored portion covers the outlet. In this manner, when the user inhales an aerosol through the mouthpiece of the smoking substitute apparatus, flavor from the sticker adhered to the mouthpiece may be delivered to the user.

The sticker may be disposed on an outside of the mouthpiece. Alternatively, the sticker may be disposed on an inside of the mouthpiece. In this case, it may be necessary to remove the mouthpiece from the smoking substitute apparatus in order to adhere the sticker to the inside of the mouthpiece. Accordingly, the mouthpiece may be removably mountable on the smoking substitute apparatus.

The mouthpiece of the smoking substitute apparatus may include a surface disposed around the outlet of the mouthpiece and arranged to receive the adhesive portion of the sticker. This may facilitate adhering the sticker to the mouthpiece, and ensure that the sticker is placed at a correct location on the mouthpiece. The surface may be substantially flat, to facilitate adhering of the sticker. The mouthpiece may further include a slot or indentation around the surface, to facilitate alignment of the sticker over the outlet.

The mouthpiece may include a tab arranged to hold the sticker in place over the mouthpiece. This may prevent the sticker from becoming unstuck and/or shifting position on the mouthpiece during use. The tab may, for example, be a flexible member which is arranged to press a surface of the sticker against the mouthpiece when the sticker is adhered to the mouthpiece.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

According to a third aspect of the second mode of the disclosure, there is provided a smoking substitute kit including: a smoking substitute apparatus, the smoking substitute apparatus having a mouthpiece with an outlet for delivering an aerosol generated by the smoking substitute apparatus to a user; and a sticker according to the first aspect of the second mode of the disclosure, wherein the flavored portion of the sticker is arranged to cover an outlet of the mouthpiece when the sticker is adhered to the mouthpiece.

In a similar manner to that discussed above, a user may stick the sticker to the mouthpiece so that the flavored portion of the sticker covers the outlet. In this manner, flavor may be delivered to the user when they use the smoking substitute apparatus.

The kit may include multiple stickers according to the first aspect of the second mode of the disclosure. Each sticker may have the same or different flavors. In this manner, the user may select a sticker to obtain a desired flavor.

The smoking substitute apparatus of the smoking substitute kit may be similar to the smoking substitute apparatus described above in relation to the second aspect of the second mode of the disclosure.

According to a fourth aspect of the second mode of the disclosure, there is provided a method of delivering flavor to a user of a smoking substitute apparatus, the smoking substitute apparatus having a mouthpiece with an outlet for conveying an aerosol generated by the smoking substitute system to the user, wherein the method includes: adhering a sticker according to the first aspect of the second mode of the disclosure to the mouthpiece so that the flavored portion of the sticker covers the outlet; generating, in the smoking substitute apparatus, an aerosol; and conveying the generated aerosol to the user via the outlet in the mouthpiece, so that the aerosol passes through the flavored portion of the sticker. In this manner, flavor may be delivered to the user by the sticker on the mouthpiece.

The method of the fourth aspect of the second mode of the disclosure may, for example, be performed using the smoking substitute kit of the third aspect of the second mode of the disclosure. Features of previous aspects of the second mode of the disclosure may therefore be shared with the fourth aspect of the second mode of the disclosure.

Where the flavored portion of the sticker is heat-activated, the method may further include the step of applying heat to the flavored portion to activate the flavored portion to deliver flavor to the user. For example, the flavored portion may be heated by the user pressing their lip or tongue against the sticker.

Where the flavored portion of the sticker is friction-activated, the method may further include the step of applying friction to the flavored portion to activate the flavored portion to deliver flavor to the user. For example, the flavored portion may be rubbed or scratched by the user to cause it to release flavorant.

Where the flavored portion of the sticker is activated by the aerosol generated by the smoking substitute apparatus passing through the flavored portion, the method may further include the step of activating, with the aerosol, the flavored portion of the sticker.

Where the flavored portion includes a removable protective outer layer, the method may include the step of removing the removable protective outer layer.

Where the adhesive portion includes a ring of adhesive arranged to surround the outlet of the mouthpiece when the sticker is adhered to the mouthpiece, the method may further include adhering the ring of adhesive around the outlet to form a seal around the outlet.

The disclosure includes the combination of the aspects of the second mode and preferred features described except where such a combination is clearly impermissible or expressly avoided.

The skilled person will appreciate that except where mutually exclusive, a feature or parameter described in relation to any one of the above aspects of the second mode may be applied to any other aspect of the second mode. Furthermore, except where mutually exclusive, any feature or parameter of the second mode described herein may be applied to any aspect and/or combined with any other feature or parameter of the second mode described herein.

Third Mode: Packaging for a Smoking Substitute Apparatus Including an Aromatic Additive

At its most general, a third mode of the present disclosure relates to packaging for a smoking substitute apparatus, the packaging including an aromatic additive. The aromatic additive may serve to produce an aroma (e.g., scent) when a user opens the packaging to use the smoking substitute apparatus, so that the user may perceive the aroma when using the smoking substitute apparatus. The aroma perceived by the user may result in a flavor sensation for the user, so that they may experience a flavor when using the smoking substitute apparatus. The packaging may also impart some of the aromatic additive to a smoking substitute apparatus contained therein. This may produce a flavor for the user when the user uses the smoking substitute apparatus, e.g., as the smoking substitute apparatus may be located near the user's nose and/or in the user's mouth.

Thus, the packaging of the disclosure may enable flavor to be delivered to a user of a smoking substitute apparatus. As a result, there may be no need to include any flavorants in the aerosol-former (e.g., e-liquid) of the smoking substitute apparatus.

According to a first aspect of the third mode of the disclosure, there is provided packaging for a smoking substitute apparatus, the packaging comprising a compartment for receiving a smoking substitute apparatus, and an aromatic additive. Thus, in use, a smoking substitute apparatus may be contained within the compartment of the packaging. For example, the packaging may be arranged to form an enclosure around the smoking substitute apparatus. The packaging may be any suitable packaging for displaying, storing, protecting and/or transporting the smoking substitute apparatus. The compartment for receiving the smoking substitute apparatus may be shaped to receive the smoking substitute apparatus.

In use, when a user opens the packaging to retrieve a smoking substitute apparatus contained therein, the aromatic additive may be perceived by the user (e.g., via their sense of smell), which may produce a flavor sensation when using the smoking substitute apparatus.

The aromatic additive may be incorporated into the packaging in any suitable manner. For example, the aromatic additive may be sprayed or coated onto a portion of the packaging (e.g., on an inner surface of the packaging). The aromatic additive may be incorporated into a material forming the packaging.

The term “aromatic additive” (also referred to as a “flavorant”) is used to describe a compound or combination of compounds that provide aroma and/or flavor. For example, the aromatic additive may be configured to interact with a sensory receptor of a user (such as an olfactory or taste receptor). The aromatic additive may include one or more volatile substances. Where a volatile substance is used, the volatile substance may diffuse within the compartment for receiving the smoking substitute apparatus, so that an aroma is produced upon opening the packaging.

The aromatic additive may be provided in solid or liquid form. The aromatic additive may be natural or synthetic. For example, the aromatic additive may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations throughout the packaging.

The packaging may be arranged to prevent release of the aromatic additive before the packaging is opened. In this manner, the packaging may only produce an aroma that is perceived by a user when the packaging is opened.

In some cases, the compartment of the packaging may be arranged to receive multiple smoking substitute apparatuses, so that multiple smoking substitute apparatuses may be provided in the same packaging. In some cases, the compartment of the packaging may be arranged to receive different component parts of a smoking substitute apparatus.

The packaging may include: an outer housing forming the compartment for receiving the smoking substitute apparatus; and an inner packaging component disposed within the outer housing, the inner packaging component being configured to hold the smoking substitute apparatus; wherein the inner packaging component comprises at least a part of the aromatic additive. In this manner, at least part of the aromatic additive may be disposed on a component of the packaging that is in contact with the smoking substitute apparatus when the smoking substitute apparatus is disposed in the packaging. The inner packaging component may be separate from the outer housing, e.g., the inner packaging component and outer housing may be formed as separate components. This may facilitate integrating the aromatic additive into the packaging, as only the fabrication process for the inner packaging component need be modified to incorporate the aromatic additive.

As the inner packaging component includes aromatic additive, some of the aromatic additive may be imparted to the smoking substitute apparatus while it is held in the inner packaging component. This may enhance flavor delivery to the user.

The outer housing may be a box or other suitable packaging container which defines a compartment in which the inner packaging component is received. For example, the outer housing may be a box made of cardboard or other suitable material. The box may have an openable lid for accessing the compartment.

The inner packaging component may be made of a same or different material as the outer housing. For example, the inner packaging component may be made of a plastic material. The inner packaging component may, for example, be formed using a thermoforming or vacuum forming technique.

In some cases, the inner packaging component may be arranged to hold multiple smoking substitute apparatuses, or different component parts of a smoking substitute apparatus.

The inner packaging component may include a recess for holding the smoking substitute apparatus, the recess having a shape that is complementary to a shape of the smoking substitute apparatus. In this manner, the smoking substitute apparatus may be placed in the recess in the inner packaging component. The recess may serve to hold the smoking substitute in place in the packaging when the smoking substitute apparatus is placed in the recess. This may ensure that the smoking substitute apparatus does not move around within the packaging, which may avoid the smoking substitute apparatus being damaged. For example, a cross-sectional shape of the recess may be complementary to a cross-sectional shape of the smoking substitute apparatus.

The recess may be arranged to form an interference fit with the smoking substitute apparatus when the smoking substitute apparatus is placed in the recess, so that the smoking substitute is securely held in place.

Where the inner packaging is arranged to hold multiple smoking substitute apparatuses, it may include multiple recesses, i.e., one recess for each smoking substitute apparatus, each recess having a shape that is complementary to a shape of the corresponding smoking substitute apparatus.

At least part of the aromatic additive comprised by the inner packaging component may be provided on a surface of the inner packaging component. For example, the aromatic additive may be provided as a coating on the surface of the inner packaging component. In some cases, the aromatic additive may be a volatile substance that is applied to the surface of the inner packaging component. Then when the inner packaging component is placed in the compartment in the outer housing, the aromatic additive may evaporate and diffuse throughout the compartment. In this manner, an aroma may be conveyed to a smoking substitute apparatus held in the inner packaging component as well as to the rest of the packaging.

The surface of the inner packaging component on which the aromatic additive may be provided may be arranged to contact the smoking substitute apparatus when the smoking substitute apparatus is held in the inner packaging component. In this manner, the smoking substitute apparatus may be in direct contact with the surface of the inner packaging component comprising the aromatic additive, so that aromatic additive may be transferred from the inner packaging component to the smoking substitute apparatus. Thus, after storage of the smoking substitute apparatus, some aromatic additive may be imparted to an outer surface of the smoking substitute apparatus. This may enhance flavor delivery to the user. For example, the aromatic additive may be a solid or a liquid (e.g., volatile substance) which is arranged to be transferred to the smoking substitute apparatus when the smoking substitute apparatus is placed in the inner packaging component.

At least part of the aromatic additive comprised by the inner packaging component may be integrated into a material forming the inner packaging component. In this manner, the aromatic additive may be directly incorporated into the material forming the inner packaging component. The aromatic additive may be incorporated into the material forming the inner packaging component during manufacture of the inner packaging component. For example, where the inner packaging component is made of plastic material, an aromatic additive may be added to a polymer solution which is used to make the plastic material.

In some embodiments, the compartment for receiving the smoking substitute apparatus may be a sealed enclosure containing an atmosphere comprising at least part of the aromatic additive. Then, when the sealed enclosure is opened, the atmosphere comprising aromatic additive may be released, which may produce an aroma perceived by a user. Some of the aromatic additive from the atmosphere in the sealed enclosure may settle on an inner surface of the packaging and on a smoking substitute apparatus contained within the sealed enclosure. In this manner, the user may perceive an aroma when they use the smoking substitute apparatus, e.g., due to aromatic additive which has settled on the smoking substitute apparatus.

As an example, the aromatic additive comprised in the atmosphere in the sealed enclosure may be in the form of a mist of ethanol and flavorant. For example, a mixture of ethanol and flavorant may be sprayed into the compartment using an atomizer. The mist may settle in the packaging and/or on the smoking substitute apparatus. Over time, the ethanol may evaporate, leaving the flavorant on the packaging and the smoking substitute apparatus. This may produce an aroma in the packaging and on the smoking substitute apparatus, and may also produce a taste or scent when a user uses the smoking substitute apparatus.

A smoking substitute apparatus may be introduced into the compartment of the packaging, prior to introducing the atmosphere comprising aromatic additive into the compartment and sealing the compartment to form the sealed enclosure. The atmosphere comprising aromatic additive may replace air in the sealed enclosure, to produce a strong aroma.

The sealed enclosure may be formed by a plastic pouch. The plastic pouch may be formed using a flow wrapping technique. The plastic pouch may be sealed following introduction of a smoking substitute apparatus and the atmosphere comprising aromatic additive into the plastic pouch.

In some embodiments, the packaging may further comprise a scented card disposed within the compartment for receiving the smoking substitute apparatus, wherein the scented card comprises at least part of the aromatic additive. This may facilitate introducing an aromatic additive into the packaging, as the scented card may be introduced into the packaging without having to otherwise modify a manufacturing process for the packaging. Thus, the scented card may be placed together with a smoking substitute apparatus into the compartment in the packaging. Aromatic additive from the scented card may diffuse throughout the packaging and/or be transferred from the scented card to the smoking substitute apparatus (e.g., where the scented card and smoking substitute apparatus are in contact), so that an aroma may be produced when the packaging is opened. The scented card may thus enable flavor delivery to a user of the smoking substitute apparatus.

The aromatic additive comprised by the scented card may be a non-tobacco scented aromatic additive. In other words, the aromatic additive may be arranged to produce an aroma that does not correspond to a tobacco aroma.

According to a second aspect of the third mode of the disclosure, there in provided a smoking substitute product comprising: packaging according the first aspect of the third mode of the disclosure; and a smoking substitute apparatus contained within the compartment for receiving a smoking substitute apparatus. In this manner, as discussed above in relation to the first aspect of the third mode of the disclosure, a user may perceive an aroma produced by the aromatic additive when opening the packaging to retrieve the smoking substitute apparatus. The user may also experience a flavor produced by the aromatic additive when using the smoking substitute apparatus, e.g., due to aromatic additive from the packaging which has been imparted to the smoking substitute apparatus. Thus, the smoking substitute produce may enable flavor to be experienced by the user when using the smoking substitute apparatus, without having to include a flavorant in an aerosol-former of the smoking substitute apparatus.

The smoking substitute apparatus may be in the form of a consumable for a smoking substitute system. By providing an aromatic additive in packaging containing a consumable for a smoking substitute system, an aroma or flavor may be delivered to the user each time the user uses a new consumable. For example, the consumable may be a cartridge (or “pod”) for a substitute smoking system.

The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

According to a third aspect of the third mode of the disclosure, there is provided a method of packaging a smoking substitute apparatus, the method comprising: forming a compartment for receiving the smoking substitute apparatus; including an aromatic additive in the compartment; and placing the smoking substitute apparatus in the compartment. The method of the third aspect of the third mode of the disclosure may be used, for example, to produce a smoking substitute produce according to the second aspect of the third mode of the disclosure, using packaging according to the first aspect of the third mode of the disclosure. Accordingly, features of the previous aspects of the third mode of the disclosure may be shared with the third aspect of the third mode of the disclosure and are not repeated.

The aromatic additive may be included in the compartment using any suitable method. For example, the aromatic additive may be coated (e.g., sprayed) onto a surface of the compartment, and/or incorporated into a material forming the packaging during manufacture of the packaging.

In some embodiments, the method may further comprise: forming an outer housing of the packaging to provide the compartment for receiving the smoking substitute apparatus; forming an inner packaging component for holding the smoking substitute apparatus, the inner packaging component comprising at least part of the aromatic additive; placing the smoking substitute apparatus in the inner packaging component, and placing the inner packaging component in the outer housing. The outer housing and inner packaging component may be similar to those discussed above in relation to the first aspect of the third mode of the disclosure.

The method may further include the steps of forming a recess in the inner packaging component, the recess having a shape complementary to a shape of the smoking substitute apparatus, and placing the smoking substitute apparatus in the recess.

Forming the inner packaging component may include making the inner packaging component out of plastic using a vacuum forming technique. This may facilitate manufacture of the inner packaging component. The aromatic additive may be incorporated directly into the plastic of the inner packaging component during manufacture. For example, the aromatic additive may be added to a polymer solution which is used to make the plastic.

In other embodiments, the method may further comprise: introducing into the compartment an atmosphere comprising at least part of the aromatic additive; and sealing the compartment to form a sealed enclosure around the smoking substitute apparatus containing the atmosphere. The atmosphere and sealed enclosure may be similar to those discussed above in relation to the first aspect of the third mode of the disclosure.

The compartment may be in the form of a sealable pouch, e.g., a plastic pouch. Thus, the method may include forming the compartment as a sealable pouch, introducing the smoking substitute apparatus and atmosphere comprising the aromatic additive into the sealable pouch, and sealing the sealable pouch to form a sealed enclosure around the smoking substitute apparatus containing the atmosphere.

Where the sealable pouch is made of plastic, the sealable pouch may be made using a flow wrapping technique.

In some embodiments, the method may comprise: placing a scented card in the compartment for receiving the smoking substitute apparatus, the scented card comprising at least part of the aromatic additive. The scented card may be similar to the scented card discussed above in relation to the first aspect of the third mode of the disclosure.

The aromatic additive comprised by the scented card may be a non-tobacco scented aromatic additive.

According to a fourth aspect of the third mode of the disclosure, there is provided a method of delivering flavor to a user of a smoking substitute apparatus, the method comprising: providing a smoking substitute apparatus in packaging, the packaging including an aromatic additive; opening the packaging to retrieve the smoking substitute apparatus; perceiving, by the user, the aromatic additive; and using, by the user, the smoking substitute apparatus. Thus, aroma and/or flavor may be delivered to the user via packaging of the smoking substitute apparatus, rather than through a flavorant in the smoking substitute apparatus (e.g., in an e-liquid of the apparatus).

The method of the fourth aspect of the third mode of the disclosure may, for example be performed using a smoking product according to the second aspect of the third mode of the disclosure. Providing smoking substitute apparatus in packaging may be performed using a method according to the third aspect of the third mode of the disclosure. Accordingly, features of the previous aspects of the third mode of the disclosure may be shared with the fourth aspect of the third mode of the disclosure, and are not repeated.

Using the smoking substitute apparatus may, for example, involve inhaling on the smoking substitute apparatus by the user to inhale an aerosol generated by the smoking substitute apparatus. When the user uses the smoking substitute apparatus, part of the smoking substitute may be disposed near the user's nose and/or in the user's mouth (e.g., a mouthpiece of the smoking substitute apparatus). In this manner, the user may smell and/or taste aromatic additive from the packaging that has been imparted to the smoking substitute apparatus. The user may also smell aromatic additive released by the packaging when the packaging is opened.

The skilled person will appreciate that except where mutually exclusive, a feature or parameter described in relation to any one of the above aspects of the third mode may be applied to any other aspect of the third mode. Furthermore, except where mutually exclusive, any feature or parameter of the third mode described herein may be applied to any aspect and/or combined with any other feature or parameter of the third mode described herein.

Fourth Mode: A Smoking Substitute Apparatus for Use with a Source of Pressurized Additive

At its most general, a fourth mode of the present disclosure relates a smoking substitute apparatus for use with a source of pressurized additive.

According to a first aspect of the fourth mode there provided a smoking substitute apparatus comprising: a passage extending between a fluid inlet and a fluid outlet, an aerosol generator for generating an aerosol in the passage downstream of the fluid inlet, and an additive inlet in fluid communication with the passage downstream of the aerosol generator, the additive inlet configured for connection with a pressurized additive source for supply of an additive from the additive source to the passage.

Providing an additive that is separate to the e-liquid may provide more versatility in how the additive can be delivered to the user. Further, the provision of a pressurized source of additive may allow the storage of a larger quantity of an additive in a smaller space. In this way, the apparatus (when used with the source of additive) may be smaller in overall size, so as to be more easily handled by a user.

The additive may be in, e.g., liquid or powder form. The additive may comprise a flavorant. The term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances.

The flavorant may be provided in solid (e.g., a powder) or liquid form. The flavorant may be natural or synthetic. For example, the flavorant may include menthol, licorice, chocolate, fruit flavor (including e.g., citrus, cherry etc.), vanilla, spice (e.g., ginger, cinnamon) and tobacco flavor. The flavorant may dispersed throughout, e.g., a base fluid. The flavorant may be evenly dispersed or may be provided in isolated locations and/or varying concentrations.

The outlet of the passage may be at a mouthpiece of the smoking substitute apparatus. In this respect, a user may draw fluid (e.g., air) into and through the passage by inhaling at the outlet (i.e., using the mouthpiece).

The smoking substitute apparatus may comprise a valve fluidly connected between the additive inlet and the passage for controlling the flow of additive from the inlet to the passage. The valve may be a one-way directional valve or a check valve. The valve may be movable between an open position and a closed position. In the open position additive may be able to flow from the inlet to the passage. In the closed position the flow of additive from the inlet to the passage may be substantially (or fully) prevented. That is, in the closed position the valve may substantially obstruct the inlet. The valve may be biased towards the closed position. This may prevent leakage of the additive from the source when the apparatus is not in use.

The valve may be configured to move from the closed position to the open position in response to fluid flow through the passage. The valve may be configured to move from the closed position to the open position in response to an inhalation from the outlet by a user. For example, the valve may be configured to move from the closed position to the open position in response to a pressure drop in the passage, which may be a result of an inhalation by a user at the outlet. In this respect, a force retaining the valve in a closed position may be the same (or slightly larger) than the force exerted on the valve from the pressurized contents of the receptacle such that an additional force (due to a pressure drop caused by inhalation) causes the valve to open. The valve may be configured to remain open during inhalation by the user.

In this way, additive (such as a flavorant) may be delivered from the additive source to the passage (and to the fluid outlet) during inhalation by a user. When a user is not inhaling at the outlet the valve may remain in the closed position. Thus, additive may only be delivered (or discharged) from the receptacle when required (i.e., during inhalation).

The valve may be controlled by other means (i.e., other than inhalation at the outlet). For example, the valve may be moved by an actuator controlled by a controller (e.g., forming part of the apparatus or in operative connection with the apparatus). The controller may cause the valve to open (move to the open position) upon receipt of a signal from a sensor. In one example, the sensor (e.g., a puff sensor) may detect inhalation at the outlet and the controller may open the valve upon receipt of a signal indicative of an inhalation at the outlet by a user. Such a sensor may be in the form of a pressure sensor or an acoustic sensor.

The valve may alternatively be controlled by a user input. For example, the apparatus may comprise a button that, when pressed by a user, may cause the valve to open. The button may be operatively connected to the valve (or an actuator of the valve) electrically (e.g., via a controller) or mechanically (e.g., by a mechanical linkage).

The smoking substitute apparatus may comprise a connector for releasable connection with a pressurized receptacle comprising the additive source. The connector may form part of, or be located at, the additive inlet. The connector may comprise a threaded connection, bayonet connection, snap-fit connection, bumps, detents, clips, etc. The connector may be configured such that, when connected with the receptacle, a seal is created between the receptacle and the additive inlet. The connector may comprise a sealing member (e.g., such as a sealing ring) to provide a seal between the receptacle and the inlet.

The connector may comprise a seal breaching portion for breaching a seal of the receptacle upon connection with the connector. For example, the seal breaching portion may comprise one or more protrusions for piercing a seal of the receptacle. The one or more protrusions may be pointed or may have a tapered cutting edge. The breaching portion may be arranged such that the seal is only breached once the receptacle and the additive inlet are connected (i.e., and a seal is formed between them). This may avoid additive leakage while connecting the receptacle to the additive inlet.

The smoking substitute apparatus may comprise a conduit extending between the passage and the additive inlet. The apparatus may comprise a housing through which the passage extends along a longitudinal axis. The conduit may extend laterally from the passage to the additive inlet (i.e., in a general lateral direction). The additive inlet may be at an outer surface of the housing. The connector may be integral with the housing or may be a separately formed component. The conduit may comprise a nozzle for dispersing additive into the passage.

The substitute smoking apparatus may comprise first and second portions. The first portion may comprise the aerosol generator and a first section of the passage. The second portion may comprise the additive inlet and a second section of the passage. The second portion may be removably mountable to the first portion such that the first and second sections of passage are fluidly connected, so as to define the passage.

The second portion may comprise the pressurized additive source. The pressurized additive source may be in the form of a cavity containing additive under pressure formed within the second portion.

The smoking substitute apparatus may be in the form of a consumable for, e.g., an e-cigarette device. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described below as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of a smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The passage may be at least partially defined by the tank. The tank may substantially (or fully) define the passage. In this respect, the tank may surround the passage.

The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage. The aerosol (or vapor) may mix with additive supplied via the additive inlet prior to being inhaled at the mouthpiece.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method. For example, and as is provided above, the controller may operate, e.g., an actuator, to move the valve so as to control additive flow into the passage.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). As provided above, the puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. The puff sensor may be operatively connected (e.g., via a controller) to the valve so as to move the valve to the open position when a puff is detected. When the smoking substitute apparatus is in the form of a consumable, the puff sensor may form part of the consumable or the main body.

In a second aspect of the fourth mode, there is provided a smoking substitute system comprising: a smoking substitute apparatus according to the first aspect of the fourth mode, and a receptacle comprising a cavity for containing a pressurized additive, the receptacle for connection to the smoking substitute apparatus such that the cavity is in fluid communication with the passage downstream of the aerosol generator.

As is set forth above, the smoking substitute apparatus of the second aspect of the fourth mode is as described above with respect to the first aspect of the fourth mode. Thus, for example, the smoking substitute apparatus of the second aspect of the fourth mode may comprise a valve, connector, housing, conduit, etc. as described above with respect to the first aspect of the fourth mode.

The receptacle may be integral with the smoking substitute apparatus (and, in this respect, may be considered a part of the smoking substitute apparatus). The receptacle may be mounted to the smoking substitute apparatus. The receptacle may be releasably mountable to the smoking substitute apparatus. The receptacle may be formed of a plastic or metal material. The receptacle is pressurized. That is, the contents are contained by the receptacle under a pressure that is greater than atmospheric pressure.

The receptacle may comprise a valve for controlling the flow of additive from the cavity. The valve may be the same as that described above with respect to the first aspect of the fourth mode (except that it forms part of the receptacle rather than part of the apparatus). Thus, the valve may be movable between an open position in which the additive is able to flow from the cavity, and a closed position in which the additive is prevented from flowing from the cavity. The valve may be biased towards the closed position. The valve may be configured to move from the closed position to the open position in response to fluid flow through the passage, when the receptacle is mounted to the smoking substitute apparatus. The valve may be configured to move from the closed position to the open position in response to an inhalation from the outlet by a user. The valve may be a check valve or a one-way valve.

The receptacle may comprise a connector for releasably connecting the receptacle to the smoking substitute apparatus. The connector may complement, or correspond to, a connector of the apparatus. The connector may comprise a threaded connection, bayonet connection, snap-fit connection, bumps, detents, clips, etc. The connector may be configured such that, when connected with the apparatus, a seal is created between the receptacle and the additive inlet of the apparatus. The connector may comprise a sealing member (e.g., such as a sealing ring) to provide a seal between the receptacle and the inlet. The connector may be configured to connect with the apparatus at or proximate to the additive inlet. Alternatively, the connector may be distal from the additive inlet (e.g., the connector may be in the form of a pair of arms wrapping around a housing of the apparatus or a further body (e.g., the main body) to which the apparatus is mounted).

The receptacle may comprise an opening for release of additive from the cavity and a seal for sealing the opening. The seal may be in the form of a removable seal. For example, the seal may comprise a barrier portion or covering the opening and a tab extending from the covering portion to facilitate removal of the seal by a user. Alternatively, the removable seal may be in the form of, e.g., a cap. The seal may alternatively be in the form of a frangible seal extending across the opening. The frangible portion may be a region of thinner and/or weaker material (e.g., a foil).

The additive may be in, e.g., liquid or powder form or a combination. The additive may comprise a flavorant. Alternatively, or additionally the additive may comprise a colorant or a coolant. As is set forth above, the flavorant may be provided in solid (e.g., a powder) or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may dispersed throughout, e.g., a base fluid. The flavorant may be evenly dispersed or may be provided in isolated locations and/or varying concentrations.

The additive may comprise a propellant. The propellant may be in the form of a gas pressurized within the receptacle. A flavorant (or other additive component) may be mixed with the propellant. Thus, the additive may be pressurized within the receptacle. For example, the cavity of the receptacle may have a pressure of between 50 to 750 kPa. The cavity of the receptacle may have a pressure of between 125 to 550 kPa. The cavity of the receptacle may have a pressure of between 300 to 500 kPa. The pressure in the cavity may be such that the additive (i.e., received through the outlet) is discharged at a velocity that is suitable for receipt in a user's mouth.

In a third aspect of the fourth mode there is provided an additive delivery article for supplying an additive to a smoking substitute apparatus, the additive delivery article comprising: a cavity for containing the additive under pressure, an outlet for releasing additive from the cavity, a connector portion for connecting the outlet to an additive inlet of the smoking substitute article, and a valve between the cavity and the outlet, the valve configured to move from a closed position in which flow of additive from the cavity is prevented and an open position in which additive is able to flow from the cavity to the outlet in response to a drop in pressure at the outlet.

The pressure drop may correspond to a user inhaling from a mouthpiece opening of the smoking substitute apparatus in fluid connection with the outlet of the flavor delivery article when connected thereto.

In a fourth aspect of the fourth mode there is provided a method of delivering an aerosol comprising an additive, the method comprising generating an aerosol flow, supplying an additive from a pressurized container to the aerosol flow to form a combined flow, and delivering the combined flow to a user.

The disclosure includes the combination of the aspects and preferred features of the fourth mode described except where such a combination is clearly impermissible or expressly avoided.

The skilled person will appreciate that except where mutually exclusive, a feature or parameter described in relation to any one of the above aspects of the fourth mode may be applied to any other aspect of the fourth mode. Furthermore, except where mutually exclusive, any feature or parameter of the fourth mode described herein may be applied to any aspect and/or combined with any other feature or parameter of the fourth mode described herein.

Fifth Mode: A Smoking Substitute Apparatus have Separate Storage for Aerosol Former and Property Modifying Agent

At its most general, a fifth mode of the present disclosure relates to a smoking substitute apparatus have separate storage for aerosol former and property modifying agent and means to release said property modifying agent to the aerosol former. This may allow the user to flavor the aerosol former at the point of use. Therefore, the smoking substitute apparatus may enable the property modifying agent to be kept separate to the aerosol former, it may minimize the interaction between the two during transport and storage. Further, it may allow the user to specify the quantity and type of property modifying agent to be added to the aerosol former, thus it may enable the user to create an aerosol former with a specific flavor and/or color tailored to the user's needs.

According to a first aspect of the fifth mode there is provided a smoking substitute apparatus, comprising:
a first compartment containing an aerosol former;
a second compartment in fluid communication with the first compartment, the second compartment is configured to receive one or more capsules each containing a property modifying agent;
wherein the second compartment is configured to release said property modifying agent from the capsules in the second compartment into the aerosol former in the first compartment.

The aerosol former may be an e-liquid. The e-liquid may, for example, comprise a base liquid and nicotine. The base liquid may include propylene glycol and/or vegetable glycerin. The e-liquid may be flavorless. That is, the e-liquid may not contain any flavorant and may consist solely of a base liquid of propylene glycol and/or vegetable glycerin and nicotine.

The property modifying agent may comprise a flavorant. The flavorant may be provided in solid, gel or liquid form. It may include menthol, licorice, chocolate, fruit flavor (including, e.g., citrus, cherry etc.), vanilla, spice (e.g., ginger, cinnamon) and tobacco flavor. The flavorant may modify a flavor of the aerosol former upon contacting or mixing with the aerosol former.

The property modifying agent may be a colorant. The colorant may be provided in solid, gel or liquid form.

The colorant may modify a color of the aerosol former upon contacting or mixing with the aerosol former. Advantageously, when the colorant is provided with the flavorant in the property modifying agent, the colorant may provide visual indication to a user for indicating a progress of release of property modifying agent. For example, the concentration of the flavorant may correspond to a change in color of the aerosol former.

The capsules may allow the property modifying agent to be stored therein, and therefore be kept separately to the aerosol former stored in the first compartment during transportation and storage. The aerosol former and the property modifying agent may only come into contact once the property modifying agent is released from the capsules, e.g., by a releasing mechanism. This may advantageously eliminate or reduce the interaction between the aerosol former and the property modifying agent during transportation and storage. For example, the e-liquid may be flavored with flavorant immediately before use.

The capsules may be configured to release the property modifying agent into the aerosol former using a releasing mechanism. More specifically, the capsule may have a capsule shell for containing or encapsulating the property modifying agent. By breaching or breaking the capsule shell, the property modifying agent as contained in the capsule may leak or flow out of the breached capsule shell and into the second compartment. Since the second compartment is in fluid communication with the first compartment, this allows the property modifying agent to flow into the first compartment to come into contact with the aerosol former stored in the first compartment. Alternatively, or in addition, the aerosol former in the first compartment may flow into the second compartment to come into contact with the property modifying agent released in the second compartment. Advantageously, this allows a user to control the release of the property modifying agent at the point of use by activating the releasing mechanism.

The capsules may comprise capsule shells that are formed from any one of hydrolyzed collagen, Hydroxy Propyl Methyl Cellulose, starch, Hydroxy Propyl Cellulose, PVA, Alginate or an extract from seaweed. More specifically, they are not soluble in the property modifying agent and therefore, they remain stable in storage. The capsule shells may be breached by rupturing the capsules. Alternatively, or in addition, the capsule shells are soluble in the aerosol former such that the capsule shells may be breached by putting the capsules in contact with the aerosol former so as to dissolve the capsule shells.

The first compartment may be separated from the second compartment by a partition wall, wherein an opening is provided in the partition wall for effecting said fluid communication. Alternatively, or in addition, the first compartment may be configured to be in fluid communication with the second compartment via a conduit. The opening and/or the conduit may be sized to allow passage of property modifying agent and the aerosol former, and it may be sized to prevent the capsules stored in the second compartment from entering the first compartment. Furthermore, the opening and/or the conduit may prevent the passage of breached capsule shells, i.e., the opening and/or conduit serves as a filter for the capsule shells. For example, the breached capsule shells may be retained in the second compartment. The first compartment may be adjacent to the second compartment or it may be spaced from the second compartment.

Optionally, the second compartment comprises one or more flexible walls, wherein moving the one or more flexible walls causes said one or more flexible walls to compress or ground the capsules stored in the second compartment. Said one or more flexible walls of the second compartment may be deformed when a force is applied thereon. For example, the flexible walls may form from elastic material that deforms elastically in reaction to a force. Or they may deform plastically such that the second compartment does not recover its original shape once the force is removed.

Optionally, said moving comprises biasing the one or more flexible walls towards a wall of the second compartment. More specifically, said moving may comprise biasing a plurality of flexible walls of the second compartment towards each other. For example, a user may squeeze or press on a pair of opposing flexible walls of the second compartment towards each other. Alternatively, or in addition, it may comprise biasing one or more flexible walls towards a rigid wall of the second compartment. For example, a user may press on a designated portion of the second compartment to move said flexible wall towards a corresponding rigid wall. Either way, the user may bias the one or more flexible walls towards a wall of the second compartment to reduce the spacing in the second compartment, and by doing so the walls may exert compressive stress on the capsule and thereby ruptures the capsule. Advantageously, such arrangement allows the user to actively release the property modifying agent in a timely manner.

Optionally, said moving comprises twisting the smoking substitute apparatus about its longitudinal axis. For example, the second compartment may comprise two opposing flexible walls and by twisting the smoking substitute apparatus about its longitudinal axis, the two opposing flexible walls may urge towards each other. Doing so the flexible walls exerts a compressive stress and/or shear stress on the capsules, and thereby ruptures the capsules stored in the second compartment.

Optionally, the one or more flexible walls, of the second compartment comprises protrusions, e.g., ribs or embossed patterns such as dots, that aids the rupturing of the capsules. More specially, the internal surfaces, e.g., inner surfaces of walls and flexible walls, of the second compartment comprises protrusions or projections. For example, said protrusions may allow pressure to concentrate locally at their tips and therefore the capsules may be breached with better efficiency.

Optionally, the second compartment comprises one or more movable elements. For example, the moveable elements may comprise a piston provided in the second compartment, wherein moving the piston towards the capsules causes the piston to compress and thereby ruptures the capsule, thereby releasing the property modifying agent stored therein. For example, the one or more moveable elements may comprise a needle provided in the second compartment, wherein moving the needle towards the capsules causes the needle to pierce through the capsule shells of the one or more capsules, thereby releasing the property modifying agent stored therein. Moreover, the moveable element may be actuated by an actuator that extends outwardly from the smoking substitute apparatus, such that a user may rupture the capsule using the actuator. The actuator may be a lever, a toggle or a button that are mechanically or electrically linked to the moveable element. Therefore, movable element may be configured to move by pulling on the lever, or by toggling the toggle, or by applying pressure on the button.

Optionally, the second compartment comprises means for contacting the one or more capsules stored in the second compartment with the aerosol former, wherein upon contacting the aerosol former a soluble shell of each of the capsules is configured to dissolve in the aerosol former, and thereby releasing the property modifying agent stored therein. For example, a user may put smoking substitute apparatus in a given orientation, e.g., turning the apparatus upside down or tilting it, such that the aerosol former in the first compartment may flow, through an opening on a partition wall separating the first compartment and the second compartment, into the second compartment. The soluble shell of the microcapsule may dissolve instantaneously upon contacting the aerosol former so as to promptly release the property modifying agent, or it may gradually dissolve.

The opening, or the fluid communication between the first and second compartments, may be sealed by a removable seal having a sealing portion and a tab portion. As such, the releasing mechanism may be a removable seal that blocks said fluid communication during storage and transportation and may be configured to be removed by the user. More specifically, the sealing portion may be configured to cover and seal the opening and the tab portion is arranged to extend outwardly from smoking substitute apparatus. In use, a user may pull on the tab portion to disengage the sealing portion from the opening and thereby allows the aerosol former to flow into the second compartment for contacting the soluble shell of the capsules. This may cause the soluble shell to dissolve in the aerosol former and thereby releases the property modifying agent stored therein.

Optionally, the property modifying agent may comprise a solid, a gel, liquid or a gas. Or in other words, the property modifying agent may be in the form of a solid, a gel, a liquid or a gas. For example, the property modifying agent may be provided as a free dried solid powder which may readily dissolve in the aerosol former. The property modifying agent may be in the form of a gel that may readily dissolve in the aerosol former. The property modifying agent may be a liquid that may be miscible or immiscible with the aerosol former. The property modifying agent may be a gas that may be readily absorbed into the aerosol former once the two are put into contact. The property modifying agent may be a solid/liquid suspension.

Optionally, the second compartment may comprise a compartment inlet for receiving the one or more capsules. Advantageously, this may allow the capsules to be supplied separately to the smoking substitute apparatus. More advantageously, a user may select to provide one or more capsules each containing the same property modifying agent, or a mix of different property modifying agents, in a desired quantity, to be added to the aerosol former according to the user's preference.

Optionally, the compartment inlet at the second compartment may be provided with a manually operated valve or a flap, for example a hinged or sprung door which may be opened for receiving the property modifying agent and closed to seal the second compartment.

Optionally, the compartment inlet may be configured to engage with a dispenser outlet of a dispenser for dispensing the property modifying agent from the dispenser into the second compartment. Advantageously, this may allow the property modifying agent to be dispensed into the second compartment with greater efficiency and accuracy. The dispenser may comprise a prefilled tube, a canister or a tank or any other suitable dispenser. Optionally, the compartment inlet may only open when it is engaged with the dispenser outlet. For example, the flap or check valve provided at the compartment inlet may only open upon engaging with a corresponding mechanism at the dispenser outlet. The compartment inlet may reseal upon disengaging the dispenser outlet therefrom. Advantageously, this may ensure the second compartment is assessable only when a corresponding dispenser is presented, as such it may prevent accidental discharge of the capsules, as well as the tampering of the smoking substitute apparatus.

Optionally, the quantity of property modifying agent releasable to the aerosol former relates to the number of capsules that are received the second compartment. More specifically, the user may adjust the flavor and/or color of the aerosol former by varying the number of capsules to be added to the second compartment, before releasing the property modifying agent from the capsules. For example, a larger quantity of capsules received in the second compartment would result in a larger quantity of property modifying agent to be released into the aerosol former. Advantageously, the flavor and/or color of the aerosol former may be tailored to the user's preference.

Optionally, the smoking substitute apparatus comprises an outer wall, and wherein at least a portion of the outer wall is translucent or transparent such that the capsules and/or aerosol former stored in the respective first and/or second compartment are visible through said portion of the outer wall. Advantageously, this allows a user to inspect the condition of capsules stored in the second compartment, e.g., whether the capsules have been ruptured or dissolved, as well as visualizing the progress of mixing the released property modifying agent and the aerosol former in the first compartment.

According to a second aspect of the fifth mode there is provided a dispenser for a smoking substitute apparatus, comprising;
a storage for storing one or more capsules; the one or more capsules each contains a property modifying agent; and
a dispenser outlet in communication with the storage;
wherein the dispenser outlet is configured to engage with a compartment inlet of the smoking substitute apparatus so as to facilitate dispensing of the one or more capsules from the storage to a second compartment of the smoking substitute apparatus.

The dispenser may be provided as a prefilled tube, a canister or a tank. Therefore, when the dispenser outlet is connected to the compartment inlet, it may allow a specified number of capsules to flow in to the second compartment. Advantageously, this may allow the capsules containing property modifying agent to be dispensed into the second compartment with greater efficiency and accuracy.

Optionally, the dispenser may comprise a dosing mechanism for controlling the number of capsules that are dispensed through the dispenser outlet, and thereby controlling the quantity of property modifying agent that is releasable to an aerosol former that is stored in a first compartment of the smoking substitute apparatus.

Optionally, the dispenser may comprise an activator configured to activate the dispenser in proximity of the aerosol forming device. The activator may, for example, comprise a docking mechanism or a key which only allows microcapsules to flow when it is connected or engaged to, or in proximity of the smoking substitute apparatus. For example, the dispenser may be activated by pushing on and/or twisting a portion of the dispenser to open a door, a flap, or a check valve, e.g., diaphragm valve, a ball valve, a duckbill valve or other suitable valves. This may enable the capsules to flow in to the second compartment. When the dispenser is disengaged or removed from the aerosol forming device, the valves on both the dispenser outlet and the compartment inlet may be closed.

Optionally, the activator at the dispenser is configured to recognize and identify markings or signals corresponding to a smoking substitute apparatus, so as to confirm the presence of said apparatus in its proximity. Upon confirming the presence of said apparatus, the activator activates the dispenser, e.g., it actuates the valve at the dispenser outlet. Advantageously, such mechanism may prevent accidental discharge of property modifying agent, as well as prohibiting a user from tampering with the dispenser. As such that the dispenser only dispenses the property modifying agent in the presence of the aerosol forming device. For example, the activator may be an optical scanner configured to scan and recognize an image (e.g., a barcode or QR code) printed on a surface the aerosol forming device to confirm its presence. Alternatively, the activator may be an RF scanner configured to read and recognize RF signals originated from an RFID on at the aerosol forming device. Alternatively, the activator may respond to a magnetic field originated from a magnet at the aerosol forming device.

According to a third aspect of the fifth mode of the present disclosure there is provided a smoking substitute kit, comprising: the smoking substitute apparatus; one or more capsules; and/or the dispenser for dispensing the one or more capsules to the smoking substitute apparatus.

According to a fourth aspect of the fifth mode of the present disclosure there is provided a method of adding property modifying agent to an aerosol former stored in a first compartment of a smoking substitute apparatus, comprising:
providing, in a second compartment of the smoking substitute apparatus in fluid communication with the first compartment, one or more capsules each containing the property modifying agent; releasing the property modifying agent from the capsules in the second compartment to the aerosol former in the first compartment.

Optionally, said releasing comprises biasing one or more flexible walls of the second compartment towards a wall of the compartment.

Optionally, said releasing comprises twisting the smoking substitute device around its longitudinal axis.

Optionally, said providing may comprise dispensing the capsules to the second compartment. Optionally, said providing may comprise dispensing the capsules to the second compartment using a dispenser.

Optionally, the method may comprise moving the aerosol forming device to facilitate mixing of the aerosol former and the property modifying agent once they are put into contact with each other.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorant and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the fifth mode described except where such a combination is clearly impermissible or expressly avoided.

The skilled person will appreciate that except where mutually exclusive, a feature or parameter described in relation to any one of the above aspects of the fifth mode may be applied to any other aspect of the fifth mode. Furthermore, except where mutually exclusive, any feature or parameter of the fifth mode described herein may be applied to any aspect and/or combined with any other feature or parameter of the fifth mode described herein.

Sixth Mode: A Smoking Substitute Apparatus Including Separate First and Second Airflows

At its most general, a sixth mode of the present disclosure relates to a smoking substitute apparatus including separate first and second airflows respectively directed past an aerosol generator and a flavor delivery portion.

According to a first aspect of the sixth mode there is provided a smoking substitute apparatus comprising: a first passage for flow of an aerosol; a second passage comprising a flavor delivery portion; and a mouthpiece in fluid communication with the first and second passages; wherein the first and second passages are arranged such that upon inhalation from the mouthpiece aerosol is drawn to the mouthpiece through the first passage, and upon exhalation into the mouthpiece air from the mouthpiece is directed through the second passage and past the flavor delivery portion.

The separation of the flavor delivery portion and the aerosol flow may allow, e.g., e-liquid (in aerosol form) and the flavorant to be delivered separately to a user. This may provide versatility in how the flavor is delivered to the user and, for example, what form it make take (both when delivered and pre-delivery).

The smoking substitute apparatus may be in the form of a consumable. The consumable may be configured for engagement with a main body (i.e., so as to form a closed substitute smoking system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, an aerosol former (e.g., e-liquid) may be replenished by replacing a used consumable with an unused consumable.

Alternatively, the smoking substitute apparatus may be a non-consumable device (e.g., that is in the form of an open substitute smoking system). In such embodiments an aerosol former (e.g., e-liquid) of the system may be replenished by re-filling the device with the aerosol former (rather than replacing a consumable component of the device).

The smoking substitute apparatus may comprise a reservoir configured to store an aerosol former, such as an e-liquid. The e-liquid may, for example, comprise a base liquid and nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The first passage may be at least partially defined by the tank. The tank may substantially (or fully) define the first passage. In this respect, the tank may surround the passage.

The first passage may comprise an aerosol generator. The aerosol generator may comprise a wick and a heater. The wick may comprise a porous material. A portion of the wick may be exposed to an airflow in the first passage. The wick may also comprise one or more portions in contact with liquid stored in the reservoir. For example, opposing ends of the wick may protrude into the reservoir and a central portion (between the ends) may extend across the first passage so as to be exposed to air flow in the first passage. Thus, air may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the first passage. This vapor may subsequently cool to form an aerosol in the passage.

In other embodiments the heater may be separate to the wick. For example, when the smoking substitute apparatus is a consumable, a main body (to which the consumable may be engaged) may comprise the heater. In such an embodiment, the heater may come into contact, or into proximity with, the wick when the consumable is engaged with the main body (i.e., so as to heat the wick during operation).

The smoking substitute apparatus may comprise a flow diverter. The flow diverter may be in the form of a valve. The flow diverter may be in the form of a directional control valve. The flow diverter may be movable to divert flow between the first and second passages. The flow diverter may be movable between a first position in which airflow between the aerosol generator and the mouthpiece is permitted, and a second position in which airflow between the aerosol generator and the mouthpiece is restricted. In the second position the flow diverter may prevent (or substantially prevent) airflow between the aerosol generator and the mouthpiece. In this respect, in the second position the flow diverter may substantially obstruct the first passage (i.e., at a location between the aerosol generator and the mouthpiece).

In the second position airflow between the mouthpiece and the flavor delivery portion may be permitted. In the second position the flow diverter may divert airflow received from the mouthpiece to the second passage (i.e., and past the flavor delivery portion).

In the first position the flow diverter may restrict airflow between the mouthpiece and the flavor delivery portion. In the first position the flow diverter may prevent (or substantially prevent) airflow between the mouthpiece and the flavor delivery portion. Similarly, in the first position the flow diverter may restrict, prevent (or substantially prevent) airflow between the aerosol generator and the flavor delivery portion (when the aerosol generator and flavor delivery portion are in fluid communication).

The flow diverter may be hingeably mounted. The fluid diverter may be mounted to a wall of the first or second passage. In the first position the fluid diverter may extend from its mounting towards the mouthpiece so as to obstruct the inlet of the second passage. The flow diverter may be biased towards the first position. The flow diverter may be biased by, for example, a spring. The flow diverter may alternatively be biased towards the second position.

The flow diverter may alternatively comprise a combination of one-way valves (e.g., duckbill valves). For example, one valve may be provided for allowing flow past the aerosol generator in a single direction (e.g., an inhale direction) and another may be provided for allowing flow past the flavor delivery portion in a single direction (e.g., an exhale direction).

The flow diverter may be configured to be moved between the first and second positions by exhalation and/or inhalation (i.e., by a user) at the mouthpiece.

The flow diverter may be configured to be moved from the first position to the second position by exhalation into the mouthpiece. That is, a pressure differential across at least a portion of the flow diverter (due to exhalation into the mouthpiece) may cause the flow diverter to move from the first position to the second position.

The flow diverter may be configured to be moved from the second position to the first position by an inhalation from the mouthpiece. That is, a pressure differential across at least a portion of the flow diverter (due to an inhalation from the mouthpiece) may cause movement of the flow diverter from the second position to the first position.

The flow diverter may comprise an actuator configured to move the flow diverter between the first and second positions. The actuator may be configured to move the flow diverter between the first and second positions in response to a control signal from a controller (e.g., comprising a microprocessor). The actuator may be configured to move the flow diverter from the first position to the second position in response to an exhalation into the mouthpiece. The actuator may be configured to move the flow diverter from the second position to the first position in response to an inhalation from the mouthpiece. For example, the actuator may be configured to move the flow diverter in response to the detection (e.g., by a sensor) of an inhalation or exhalation at the mouthpiece.

The first passage may extend between an inlet and an opposing mouthpiece opening of the mouthpiece (for inhalation/exhalation by a user). The first passage may be elongate and substantially linear so as to define a longitudinal axis. Where the smoking substitute apparatus is a consumable the first passage may extend from the mouthpiece at one end of the consumable to an opposing end of the consumable. This opposing end may be configured for engagement with a main body. When engaged in this way, the first passage may be in fluid communication with one or more further passages (e.g., defined between the consumable and the main body when engaged) so as to allow air to be drawn into the inlet of the first passage.

The second passage may comprise an outlet. The outlet may be arranged to discharge an airflow (i.e., a flavored airflow) towards a user's nose (i.e., nasal cavity) upon exhalation into the mouthpiece. The outlet may be arranged discharge an airflow in a direction that is at an angle with respect to the longitudinal axis of the first passage. The outlet may be spaced from the mouthpiece. That is, the outlet may not form part of the mouthpiece and, as such, may remain unobstructed when a user inhales and/or exhales at the mouthpiece. The outlet may be formed in an in use upper surface of the device.

The second passage may be connected to the first passage. The second passage may branch from the first passage. Thus, the second passage may comprise an inlet arranged to receive an airflow from the first passage. The inlet of the second passage may be located (axially) between the mouthpiece and the aerosol generator. The second passage may extend generally laterally from the first passage. The second passage may follow a generally curved profile. The fluid diverter may be located (axially) between the inlet of the second passage and the aerosol generator.

The flavor delivery portion may comprise a sintered polymer, a ceramic matrix, an open-cell foam and/or a cotton-based fibrous media. The flavorant may be deposited on one or more surfaces of the flavor delivery portion. Alternatively, or additionally, the flavor delivery portion may be impregnated with flavorant. The flavor delivery portion may comprise a channel (i.e., a pre-formed channel) through which air may flow. The channel may be the second passage, or may form part of the second passage.

The channel may follow, e.g., a linear, angular, helical or curved profile.

The flavorant may be in, e.g., liquid or powder form. The term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances.

The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may dispersed throughout, e.g., a base fluid. The flavorant may be evenly dispersed or may be provided in isolated locations and/or varying concentrations.

Where the smoking substitute apparatus is in the form consumable, the consumable may be configured to be physically coupled with a main body. For example, the consumable may be at least partially received in a recess of the main body, such that there is an interference fit between the main body and the consumable. Alternatively, the main body and the consumable may be physically coupled together by screwing one onto the other, or through a bayonet fitting.

The smoking substitute apparatus (or main body for engagement with the device) may comprise a power source. The power source may be electrically connected (or connectable) to a heater of the smoking substitute apparatus. The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may comprise a certain concentration of nicotine and the electrical interface may be used to identify this. The electrical interface may additionally or alternatively be used to identify when a consumable is connected to the main body.

The electrical interface may be used for identifying a flavor associated with the flavor delivery portion.

Again, where the smoking substitute apparatus is in the form of a consumable, the main body may comprise a further interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the electronic interface in the main body. Again, this interface may provide the main body with an indication of the flavor associated with the flavor delivery portion.

The controller (which may form part of the smoking substitute apparatus or a main body engaged with the smoking substitute apparatus) may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

An inhale/exhale sensor (or plurality of sensors) may be provided that is configured to detect an inhale or exhale is received at the mouthpiece. This sensor may be able to differentiate between an inhale and an exhale. The sensor may be operatively connected to the controller so as to provide a signal to the controller indicative of an inhale and/or exhale at the mouthpiece. The controller may control the fluid diverter in response to this signal. When the smoking substitute apparatus is in the form of a consumable, the puff sensor may form part of the consumable or the main body.

The smoking substitute apparatus may further comprise a user interface (UI). The UI may comprise a button, touchscreen, switch, etc. When the smoking substitute apparatus is in the form of a consumable, the UI may form part of the consumable or a main body for engagement with the consumable may comprise a UI.

The disclosure includes the combination of the aspects and preferred features described except where such a combination is clearly impermissible or expressly avoided.

The skilled person will appreciate that except where mutually exclusive, a feature or parameter described in relation to any one of the above aspects of the sixth mode may be applied to any other aspect of the sixth mode. Furthermore, except where mutually exclusive, any feature or parameter of the sixth mode described herein may be applied to any aspect and/or combined with any other feature or parameter of the sixth mode described herein.

Seventh Mode: A Mouthpiece for a Smoking Substitute Apparatus

At its most general, a seventh mode of the present disclosure relates to a mouthpiece for a smoking substitute apparatus that is arranged to receive a liquid flavorant applied thereto, and to distribute the flavorant over an area of the mouthpiece. In this manner, when a user puts the mouthpiece in their mouth to use the smoking substitute apparatus, flavorant located on the mouthpiece may produce a flavor for the user. In this manner, flavor may be delivered to the user via flavorant on the mouthpiece, rather than through a flavorant contained in an aerosol-former (e.g., e-liquid or tobacco material) of the of the smoking substitute apparatus. As a result, there may be no need to include any flavorants in the aerosol-former of the smoking substitute apparatus.

A flavorant may be applied to the mouthpiece when the user wishes to experience flavor. The user may also apply an amount of flavorant to the mouthpiece according to their taste, e.g., depending on whether they want to experience a strong flavor or not. Flavorants having different flavors may be applied to the mouthpiece, so that the user may experience different flavors, without having to otherwise modify the smoking substitute apparatus. This may improve a versatility of the smoking substitute apparatus.

According to a first aspect of the seventh mode there is provided a mouthpiece for a smoking substitute apparatus, the mouthpiece comprising: an outlet for conveying an aerosol generated by the smoking substitute apparatus to a user; and a textured outer surface configured to receive a liquid flavorant and distribute the liquid flavorant over the textured outer surface through capillary action of the liquid flavorant on the textured outer surface.

In this manner, due to the textured outer surface of the mouthpiece, liquid flavorant may be retained on the surface of the mouthpiece. The textured outer surface may further serve to distribute the liquid flavorant over an area of the mouthpiece via capillary action of the liquid flavorant on the textured outer surface, e.g., the liquid flavorant may be wicked across the textured outer surface. In this manner, after applying liquid flavorant to the mouthpiece, liquid flavorant may be present over an area corresponding to the textured outer surface. This may serve to provide a large contact area between the flavorant on the mouthpiece and the user's tongue when the user puts the mouthpiece in their mouth. As a result, flavor delivery to the user may be enhanced. The textured outer surface may also enable a larger amount of liquid flavorant to be carried by the mouthpiece than without any textured outer surface, which may enable flavorant to be delivered to the user for a longer period of time.

The term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances.

The liquid flavorant may be a flavorant that is provided in liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The textured outer surface may include a plurality of channels arranged to conduct the liquid flavorant through capillary action of the liquid flavorant in the channels. The channels may, for example, be in the form of grooves in the outer surface of the mouthpiece. Liquid flavorant may be retained in the channels due to a surface tension of the liquid flavorant in the channels. The plurality of channels may extend over an area of the mouthpiece, so that liquid flavorant is distributed over that area, via wicking (capillary action) of the flavorant through the channels. The channels may be referred to as capillary channels.

The textured outer surface may include a plurality of protrusions arranged to form the plurality of channels. In other words, the channels may be formed between adjacent protrusions on the surface of the mouthpiece. For example, the plurality of protrusions may include a set of identically shaped protrusions that are arranged in a regular array on the surface of the mouthpiece. This may result in a set of channels which are all oriented in the same direction. This may serve to conduct the liquid flavorant in a preferential direction. Alternatively, the protrusions may be arranged in a substantially random manner, which may result in random channel orientations.

The plurality of channels may include a first set of channels arranged in parallel along a first direction. In this manner, the first set of channels may conduct liquid flavorant along the first direction. This may serve to conduct liquid flavorant towards an area of the mouthpiece that is likely to come into contact with the user's tongue during use. This may also serve to provide an even distribution of the liquid flavorant over an area of the mouthpiece.

The first direction may be substantially perpendicular to a flow direction of aerosol through the outlet of the mouthpiece. Typically, aerosol may flow through the outlet in a direction that is parallel to a longitudinal axis of the smoking substitute apparatus. In use, the longitudinal axis of the smoking substitute apparatus may often be tilted downwards. By orienting the first set of channels so that they are substantially perpendicular to the flow direction of aerosol through the outlet, it may be possible to reduce the risk of liquid flavorant dripping down along the mouthpiece during use. In some cases, the first set of channels may be interconnected by a further channel, e.g., by a channel extending along the longitudinal axis.

The plurality of channels may include a second set of channels arranged in parallel along a second direction different from the first direction, the second set of channels intersecting the first set of channels. In this manner, liquid flavorant may be conducted along both the first and second directions, which may result in a more even distribution of the flavorant over the mouthpiece. For example, the first and second sets of channels may define a grid pattern on the surface of the mouthpiece. The second set of channels may be in fluid communication with the first set of channels, so that liquid flavorant present in the first and/or second set of channels may be wicked into the other set of channels.

The textured outer surface may include a fluid receiving portion for receiving the liquid flavorant, the fluid receiving portion being fluidly connected to the plurality of channels. In this manner, liquid flavorant deposited in the fluid receiving portion of the mouthpiece may be wicked through the plurality of channels so that it is distributed over an area of the mouthpiece. Thus, a user may only need to apply a liquid flavorant to a single area on the mouthpiece, i.e., to the fluid receiving portion. For example, the fluid receiving portion may be a reservoir (e.g., cavity or recess) defined in a surface of the mouthpiece, with one or more of the plurality of channels being arranged to conduct fluid (e.g., liquid flavorant) out of the reservoir.

The mouthpiece may include an absorbent material for absorbing liquid flavorant. In this manner, flavorant applied to the mouthpiece may be absorbed by the absorbent material on the mouthpiece. This may enable the mouthpiece to absorb a larger amount of flavorant, so that the mouthpiece may deliver flavorant to the user for a longer period of time. The absorbent material may define the textured outer surface of the mouthpiece, e.g., an outer surface of the absorbent material may correspond to the textured outer surface.

Any suitable absorbent material may be used. For example, the absorbent material may be a cloth material, or a sponge-like material.

The absorbent material may be a porous plastic. This may facilitate cleaning of the mouthpiece. This may also facilitate making of the mouthpiece, as the porous plastic may be formed into a mouthpiece using a molding technique. As an example, porous plastics manufactured by Porex corporation may be used.

The mouthpiece may further include an engagement portion arranged to engage a dispensing portion on a flavorant dispenser. The engagement portion may serve to align the flavorant dispenser over the textured surface of the mouthpiece, to facilitate dispensing of liquid flavorant onto the textured surface using the flavorant dispenser. For example, the engagement portion may include a protrusion on the mouthpiece which is arranged to engage the dispensing portion of the flavorant dispenser. Alternatively, or additionally, the engagement portion may include a recess or cavity in the surface of the mouthpiece, which is arranged to receive a protrusion on the dispensing portion of the flavorant dispenser.

The engagement portion may be in the form a key on the mouthpiece which is arranged to engage a corresponding interface in the dispensing portion of the flavorant dispenser. When the key is engaged in the interface, the dispensing portion may be configured to dispense flavorant onto the mouthpiece. This may serve to prevent dispensing of the liquid flavorant by the flavorant dispenser when a mouthpiece having the appropriate key is not present.

The engagement portion of the mouthpiece may include a wall that is arranged to define part of a receptacle on the mouthpiece for receiving liquid flavorant from the flavorant dispenser. For example, the wall may serve to define part of the fluid receiving portion of the textured outer surface. When the dispensing portion of the flavorant dispenser is engaged with the engagement portion of the mouthpiece, the wall of the engagement portion, together with a wall of the dispensing portion may form a receptacle for receiving liquid flavorant dispensed by the flavorant dispenser. In this manner, a predetermined volume of liquid flavorant may be dispensed into the receptacle. Then, when the dispensing portion is removed from the mouthpiece, liquid flavorant that was dispensed into the receptacle may exit the receptacle to be distributed across the textured surface.

According to a second aspect of the seventh mode of the disclosure, there is provided a smoking substitute apparatus including a mouthpiece according to the first aspect of the seventh mode of the disclosure. The mouthpiece may be removably mounted on the smoking substitute apparatus. Alternatively, the mouthpiece may be integrally formed as part of the smoking substitute apparatus.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

According to a third aspect of the seventh mode of the disclosure, there is provided a flavorant dispenser for dispensing liquid flavorant onto an outer surface of a mouthpiece of a smoking substitute apparatus, the flavorant dispenser comprising: a reservoir for holding a liquid flavorant; and a dispensing portion arranged to engage the outer surface of the mouthpiece and to dispense a predetermined volume of liquid flavorant from the reservoir onto the outer surface of the mouthpiece when the dispensing portion engages the outer surface of the mouthpiece.

In this manner, a user may engage the dispensing portion of the flavorant dispenser with the outer surface of the mouthpiece to dispense a predetermined volume of liquid flavorant thereon, so that flavorant may be delivered to the user when they use the mouthpiece. This may avoid the liquid flavorant being accidentally dispensed when the dispensing portion is not engaged with the mouthpiece.

Dispensing a predetermined volume of liquid flavorant may avoid an excessive amount of liquid flavorant being applied to the mouthpiece, which could result in liquid flavorant dripping off the mouthpiece. The predetermined volume may be set to a volume of liquid flavorant that the mouthpiece is capable of holding. For example, where the mouthpiece includes a textured outer surface for receiving liquid flavorant, the predetermined volume may correspond to an amount of liquid flavorant that the textured outer surface is capable of holding.

The reservoir may be any suitable container for receiving and holding liquid flavorant. The reservoir may be in fluid communication with the dispensing portion, so that liquid flavorant from the reservoir may be dispensed by the dispensing portion. The dispensing portion may include an outlet through which liquid flavorant may be dispensed.

In some embodiments, the dispensing portion may include a valve that is openable to dispense liquid flavorant from the reservoir; the dispensing portion may be arranged to engage an engagement portion of the outer surface of the mouthpiece; and the valve may be opened by engagement between the dispensing portion and the engagement portion on the outer surface of the mouthpiece. In this manner, when the dispensing portion is engaged with the outer surface of the mouthpiece, the engagement portion of the outer surface of the mouthpiece may engage the dispensing portion (e.g., a corresponding engagement portion of the dispensing portion), which causes the valve to open so that liquid flavorant may be dispensed onto the outer surface of the mouthpiece. Thus, liquid flavorant may be automatically dispensed onto the outer surface of the mouthpiece when the dispensing portion is engaged with the mouthpiece. This may facilitate applying liquid flavorant to the mouthpiece.

The valve may be movable between an open and a closed position. The valve may be disposed between the reservoir and the outlet of the dispensing portion. Thus, when the valve is in the open position, the reservoir may be in fluid communication with the outlet so that liquid flavorant from the reservoir may be dispensed; when the valve is in the closed position, the valve may block liquid flavorant from exiting the reservoir via the outlet of the dispensing portion.

The valve may be biased towards to closed position, to prevent liquid flavorant from leaking when the flavorant dispenser is not in use. The valve may be biased using any suitable mechanism, e.g., via a spring or the like.

The dispensing portion may include an interface which is configured to receive the engagement portion of the mouthpiece in the form of a key. When the key is engaged in the interface of the dispensing portion, the dispensing portion may be configured to dispense flavorant onto the mouthpiece. This may serve to prevent dispensing of the liquid flavorant by the flavorant dispenser when a mouthpiece having the appropriate key is not present.

The dispensing portion may include a wall arranged to form part of a receptacle for receiving liquid flavorant when the dispensing portion of the flavorant dispenser is engaged with the engagement portion of the mouthpiece. When the dispensing portion of the flavorant dispenser is engaged with the engagement portion of the mouthpiece, the wall of the dispensing portion, together with a wall of the engagement portion of the mouthpiece, may form a receptacle for receiving liquid flavorant dispensed by the flavorant dispenser. The receptacle may have a volume corresponding to the predetermined volume of liquid flavorant dispensed by the dispensing portion. Thus, when the dispensing portion engages the engagement portion of the outer surface of the mouthpiece, the valve is opened so that liquid flavorant is dispensed from the dispensing portion into the receptacle on the mouthpiece formed by the wall of the dispensing portion and a wall of the engagement portion of the mouthpiece. Then, when the dispensing portion is removed from the mouthpiece, liquid flavorant that was dispensed into the receptacle may exit the receptacle to be distributed across the surface of the mouthpiece, e.g., via a textured outer surface of the mouthpiece.

According to a fourth aspect of the seventh mode of the disclosure, there is provided a smoking substitute kit including a smoking substitute apparatus according to the second aspect of the seventh mode of the disclosure, and a flavorant dispenser according to the third aspect of the seventh mode of the disclosure. The dispensing portion of the flavorant dispenser may be arranged to engage the outer surface of the mouthpiece of the smoking substitute apparatus and to dispense a predetermined volume of liquid flavorant from the reservoir onto the outer surface of the mouthpiece when the dispensing portion engages the outer surface of the mouthpiece.

Where the dispensing portion includes a valve, and the outer surface of the mouthpiece includes an engagement portion: the dispensing portion may be arranged to engage an engagement portion of the outer surface of the mouthpiece; and the valve may be opened by engagement between the dispensing portion and the engagement portion on the outer surface of the mouthpiece.

The engagement portion of the mouthpiece may be in the form a key on the mouthpiece which is arranged to engage a corresponding interface in the dispensing portion of the flavorant dispenser. When the key is engaged in the interface, the dispensing portion may be configured to dispense flavorant onto the mouthpiece. This may serve to prevent dispensing of the liquid flavorant by the flavorant dispenser when a mouthpiece having the appropriate key is not present.

The engagement portion of the mouthpiece may include a first wall that is arranged to define a first part of a receptacle on the mouthpiece for receiving liquid flavorant from the flavorant dispenser, and the dispensing portion may include a second wall arranged to form a second part of the receptacle. In this manner, when the dispensing portion of the mouthpiece is engaged with the engagement portion, the first and second walls may together form the receptacle in which liquid flavorant is dispensed by the dispensing portion. A volume of the receptacle may correspond to the predetermined volume of liquid flavorant dispensed by the dispensing portion.

According to a fifth aspect of the seventh mode of the disclosure, there is provided a method of delivering flavor to a user of a smoking substitute apparatus, the method including: applying a liquid flavorant to an outer surface of a mouthpiece of the smoking substitute apparatus; using, by the user, the smoking substitute apparatus. In this manner, flavorant applied to the outer surface of the mouthpiece may be delivered to the user when they use the smoking substitute apparatus. Using the smoking substitute apparatus may include inhaling, by the user, an aerosol generated by the smoking substitute apparatus.

The method of the fifth aspect of the seventh mode of the disclosure may be used with any of the previous aspects of the seventh mode of the disclosure.

Applying the liquid flavorant to the outer surface of the mouthpiece may include using a flavorant dispenser of the third aspect of the seventh mode of the disclosure. For example, the dispensing portion of the flavorant dispenser may be engaged with the outer surface of the mouthpiece to dispense a predetermined volume of flavorant onto the outer surface of the mouthpiece. Where the dispensing portion includes a valve, and the outer surface of the mouthpiece includes an engagement portion, applying the liquid flavorant may include engaging the dispensing portion with the engagement portion of the outer surface of the mouthpiece, to open the valve and dispense liquid flavorant onto the outer surface of the mouthpiece.

Where the engagement portion of the mouthpiece includes a first wall that is arranged to define a first part of a receptacle on the mouthpiece for receiving liquid flavorant from the flavorant dispenser, and the dispensing portion includes a second wall arranged to form a second part of the receptacle, applying the liquid flavorant may include engaging the dispensing portion with the engagement portion of the outer surface of the mouthpiece to form, with the first wall and second wall, a receptacle for receiving liquid flavorant dispensed by the dispensing portion.

Where the mouthpiece is removably mountable on the smoking substitute apparatus, the method may further include mounting the mouthpiece on the smoking substitute apparatus (e.g., prior to applying the liquid flavorant).

The seventh mode of the disclosure includes the combination of the aspects and preferred features described except where such a combination is clearly impermissible or expressly avoided.

The skilled person will appreciate that except where mutually exclusive, a feature or parameter described in relation to any one of the above aspects of the seventh mode may be applied to any other aspect of the seventh mode. Furthermore, except where mutually exclusive, any feature or parameter of the seventh mode described herein may be applied to any aspect and/or combined with any other feature or parameter of the seventh mode described herein.

Eighth Mode: A Smoking Substitute Apparatus that Allows an Aerosol Former to be Stored Separately from a Property Modifying Agent

At its most general, an eighth mode of the present disclosure relates to a smoking substitute apparatus that allows an aerosol former to be stored separately from a property modifying agent, and only put into contact with each other prior to use. This may allow the user to flavor the aerosol former at the point of use. Therefore, the smoking substitute apparatus may enable the property modifying agent to be kept separate to the aerosol former, it may minimize the interaction between the two during transport and storage. Further, it may allow the user to specify the quantity and type of property modifying agent to be added to the aerosol former, thus it may enable the user to create an aerosol former with a specific flavor and/or color tailored to the user's needs.

According to a first aspect of the eighth mode there is provided a smoking substitute apparatus, comprising:
a first compartment for receiving an aerosol former;
a second compartment for receiving a property modifying agent configured to modify one or more properties of the aerosol former;
a barrier configured to prevent fluid communication between the first and second compartments;
wherein removing the barrier establishes said fluid communication and thereby allows the aerosol former and the property modifying agent to come into contact with each other.

The aerosol former may be an e-liquid. The e-liquid may, for example, comprise a base liquid and nicotine. The base liquid may include propylene glycol and/or vegetable glycerin. The e-liquid may be flavorless. That is, the e-liquid may not contain any flavorant and may consist solely of a base liquid of propylene glycol and/or vegetable glycerin and nicotine.

The property modifying agent may comprise a flavorant. The flavorant may be provided in solid, gel or liquid form. It may include menthol, licorice, chocolate, fruit flavor (including, e.g., citrus, cherry etc.), vanilla, spice (e.g., ginger, cinnamon) and tobacco flavor. The flavorant may modify a flavor of the aerosol former upon contacting or mixing with the aerosol former.

The property modifying agent comprise be a colorant. The colorant may be provided in solid, gel or liquid form. The colorant may modify a color of the aerosol former upon contacting or mixing with the aerosol former. Advantageously, when the colorant is provided with the flavorant in the property modifying agent, the colorant may provide visual indication to a user for indicating mixing progress. For example, the concentration of the flavorant may corresponds with a change in color of the aerosol former.

The smoking substitute apparatus may allow the aerosol former and the property modifying agent to be kept in respective compartments and separate to each other during transportation and storage. The aerosol former and the property modifying agent may only come into contact once a user removes the barrier. This advantageously reduce the interaction between the aerosol former and the property modifying agent during transportation and storage. For example, the e-liquid may only be flavored with flavorant prior to use.

Optionally, the first compartment is configured to be in fluid communication with the second compartment via a conduit. The first compartment may be adjacent to the second compartment or it may be spaced from the second compartment.

Optionally, the first compartment and the second compartment are separated by a partition wall. More specifically, the first compartment and second compartment may share the partition wall.

Optionally, the partition wall comprises an opening for effecting said fluid communication. As such, the opening provides a passage for the aerosol former in the first compartment to flow towards the second compartment, and for the flavor and/or colorant in the second compartment to enter the first compartment. Advantageously, the provision of an opening shortens the distance the aerosol former and the property modifying agent have to travel to come into contact with each other.

Optionally, the barrier is configured to close the opening prior to its removal. As such, the smoking substitute apparatus may be provided with the barrier blocking or sealing the opening. The barrier may be temporally or permanently be moved or removed from a sealing position that seals the opening, so as to establish said fluid communication.

Optionally, the barrier comprises a removable seal having a seal portion configured to seal the opening and a tab portion extending outwardly from the smoking substitute apparatus, whereby pulling the tab portion away from the smoking substitute apparatus detaches the seal portion from the opening and/or removes the removable seal from the smoking substitute apparatus. The seal portion may be formed together with the tab portion. The removable seal may seal the opening using induction sealing, adhesive sealing or any other sealing techniques known to the person skilled in the art. The tab portion may extend through the body of the smoking substitute apparatus such that the removable seal is accessible for a user. For example, the removable seal may extend through a slot extending from the opening towards an exterior wall of the aerosol forming device. By pulling on the tab portion, the seal portion may be forced to detach or break away from the opening and thereby establishes fluid communication between the first compartment and the second compartment. The removable seal may partially remain in the smoking substitute apparatus during use, or it may be completely withdrawn from the device.

Alternatively, the barrier comprises a valve for closing the opening, the valve is configured to be actuated by an actuator extending outwardly from the aerosol forming consumable; wherein actuating the actuator controls the flow of aerosol former and/or property modifying agent through said opening. The valve may be a check valve allowing the property modifying agent to flow from the second compartment towards the first compartment. Advantageously, the check valve may permit a one-way flow passage for the property modifying agent, as such a user may control the dose of flavorant and/or colorant to be added to the aerosol former in the first compartment, whilst preventing an ingress of aerosol former into the second compartment. Alternatively, the valve may be a two-way valve that permits free flow between the first and second compartments. Advantageously, the use of a two-way valve may allow the use of valves that are simpler in construction.

The actuator may be a lever, a button, a toggle or other actuator that is accessible by the user. The valve may advantageously allow a user to control the flow of aerosol former and the property modifying agent between the first and second compartments. For example, a user may dispense a desired amount of the property modifying agent from the first compartment into the second compartment. This may advantageously allow the user to personalize the flavor and/or color of the e-liquid by controlling the dosage of flavorant and/or colorant.

Alternatively, the barrier comprises a partition wall configured to separate the first compartment and the second compartment, wherein removing the partition wall merges the second compartment with the first compartment to form a combined compartment. For example, the partition wall may comprise a planar element movable along a pair of slots extending in between the first and second compartments. In use, a user may traverse the partition wall along the slot so as to remove the partition wall from the smoking substitute apparatus. This may enable the first and second compartments to merge into a single combined compartment, i.e., the aerosol former in the first compartment and the property modifying agent in the second compartment may mix in the combined compartment. Advantageously, such arrangement may allow the aerosol former and the property modifying agent to be mixed more promptly and efficiently in the combined compartment.

Optionally, the ratio of a volume of the first compartment to a volume of the second compartment is in the range of 1:1 to 9:1. More specifically, said ratio may be any one of 1:1, 2:1, 3:1, 4:1, 5:1, 6:1, 7:1, 8:1 and 9:1, or any one of 50:50, 60:40, 70:30, 80:20 and 90:10. Providing the first and second compartments with predetermined ratios may advantageously allow the proportion of the aerosol former and property modifying agent to be specified, and the dosage of the property modifying agent may be made repeatable.

Optionally, the second compartment comprises a compartment inlet, wherein the second compartment is configured to receive the property modifying agent through the compartment inlet. Advantageously, this may allow the property modifying agent to be supplied separately to the aerosol forming device. More advantageously, a user may select a property modifying agent, or a mix of different property modifying agents, in a desired quantity, to be added to the aerosol former according to his/her preference. The compartment inlet may be closed by a trap door or a check valve so as to prevent the property modifying agent stored in the second compartment from escaping or leaking out from there.

Optionally, the compartment inlet at the second compartment is provided with a manually operated valve or flap, for example a hinged or sprung door which may be opened for receiving the property modifying agent and closed to seal the second compartment. This arrangement may be particularly suitable for property modifying agent that is provided as a solid puck, a block or a pellet of compressed powder or a substrate coated with property modifying agent. When the manually operated valve is closed, the second compartment may be filled with said property modifying agent.

Optionally, the compartment inlet is configured to engage with a dispenser outlet of a dispenser for dispensing the property modifying agent from the dispenser into the second compartment. Advantageously, this may allow the property modifying agent to be dispensed into the second compartment with greater efficiency and accuracy. The dispenser may comprise a bottle, a syringe, a pressurized canister, a squeezable tube or any other suitable dispenser. Optionally, the compartment inlet may only open when it is engaged with the dispenser outlet. For example, the trap door or check valve provided at the compartment inlet may only open upon engaging with a corresponding mechanism at the dispenser outlet. The compartment inlet may reseal upon disengaging the dispenser outlet therefrom. Advantageously, this may ensure the second compartment is assessable only when a corresponding dispenser is presented, as such it may prevent accidental discharge of the property modifying agent, as well as the tampering of the aerosol forming device.

Optionally, the property modifying agent may be in the form of any one or more of a solid, a gel, a liquid or a gas. For example, the flavorant and/or colorant may be provided as a powder, a pellet, a block or granules, and may readily dissolve in the aerosol former. The flavorant and/or colorant may be a gel that may readily dissolve in the aerosol former. The flavorant and/or colorant may be a liquid that may be miscible or immiscible with the aerosol former. The flavorant and/or colorant may be a gas that may be absorbed into the aerosol former.

According to a second aspect of the eighth mode there is provided a dispenser for an aerosol forming device, comprising, a storage for storing the property modifying agent; and a dispenser outlet in communication with the storage; wherein the dispenser outlet is configured to engage with a compartment inlet of the aerosol forming device so as to facilitate dispensing of the property modifying agent from the storage to a second compartment of the aerosol forming device.

The dispenser may comprise a bottle, a syringe, a pressurized canister, a squeezable tube or any other suitable dispenser. Advantageously, this may allow the property modifying agent to be dispensed into the second compartment with greater efficiency.

Optionally, the dispenser comprises a dosing mechanism for controlling the quantity of property modifying agent that is dispensed through the dispenser outlet. Optionally, the dispenser comprises an activator configured to activate the dispenser in proximity of the aerosol forming device.

For example, the property modifying agent, in a liquid form, may be added to the second compartment in the aerosol forming device by the dispenser, e.g., a bottle, syringe, pressurized canister or squeezable tube. To prevent spilling, the dispenser may comprise an activator, for example a docking mechanism or a key which only allows the liquid property modifying agent to flow when it is connected or engaged to, or in proximity of the aerosol forming device. The compartment inlet and the dispenser outlet may each be sealed by a check valve, e.g., diaphragm valve, ball valve, duckbill valve that only opens when the two are engaged. This may provide one way flow passage for the liquid property modifying agent to flow from the dispenser storage to the second compartment of the aerosol forming device, but not in the other direction. When the dispenser is disengaged or removed from the aerosol forming device, the valves on both the dispenser outlet and the compartment inlet may be closed.

The property modifying agent may be provided in a powder or a granular form, e.g., freeze dried granulate. Accordingly, the dispenser may be provided as a prefilled tube, a canister or a tank. Therefore, when the dispenser outlet is connected to the compartment inlet, it may allow a specified amount of powder to flow in to the second compartment. Such dispenser may have an activator, for example a docking mechanism or a key which only allows powder or granulate to flow when it is connected or engaged to, or in proximity of the aerosol forming device. For example, the dispenser may be activated by pushing on and/or twisting a portion of the dispenser to open a door or a check valve, e.g., diaphragm valve, a ball valve, a duckbill valve or other suitable valves. This may enable the powder to flow in to the second compartment. When the dispenser is disengaged or removed from the aerosol forming device, the valves on both the dispenser outlet and the compartment inlet may be closed.

The property modifying agent may be provided as a gas. Thereby the property modifying agent may be added by a dispenser in the form of a pressurized canister. More specifically, the dispenser outlet may dock with the compartment inlet through means of a mechanical connection, e.g., a screw, a bayonet or a clip connection, so at to create a seal between the dispenser outlet and the compartment inlet. Advantageously, this may minimize or prevent the gaseous property modifying agent from escaping to the atmosphere. Upon engaging the dispenser outlet with the compartment inlet, the dispenser may disperse the gaseous property modifying agent in to the second compartment by means of a check valve, e.g., a diaphragm valve, a ball valve, a duckbill valve or other suitable valves, so as to provide one way flow passage for the gaseous property modifying agent to flow from the dispenser storage to the second compartment of the aerosol forming device, but not in the other direction. Upon removing the dispenser from the aerosol forming device, the valves on both the dispenser outlet and the compartment inlet may be closed.

Optionally, the activator of the dispenser is configured to recognize and identify markings or signals corresponding to an aerosol forming device, so as to confirm the presence of said device in its proximity. Upon confirming the presence of said device, the activator activates the dispenser, e.g., it actuates the valve at the dispenser outlet. Advantageously, such mechanism may prevent accidental discharge of property modifying agent, as well as prohibiting a user from tampering with the dispenser. As such that the dispenser only dispenses the property modifying agent in the presence of the aerosol forming device. For example, the activator may be an optical scanner configured to scan and recognize an image (e.g., a barcode or QR code) printed on a surface the aerosol forming device to confirm its presence.

Alternatively, the activator may be an RF scanner configured to read and recognize RF signals originated from an RFID at the aerosol forming device. Alternatively, the activator may respond to a magnetic field originated from a magnet at the aerosol forming device.

According to third aspect of the eighth mode of there is provided a smoking substitute kit, comprising:
the smoking substitute apparatus;
the property modifying agent; and/or
the dispenser.

According to a fourth aspect of the eighth mode there is provided a method of dispensing a property modifying agent to an aerosol former stored in a first compartment of an aerosol forming device, comprising:
providing the property modifying agent in a second compartment of the aerosol forming device;
removing a barrier that prevents fluid communication between the first compartment and the second compartment, thereby establishing said fluid communication to allow the aerosol former and the property modifying agent to come into contact with each other.

Optionally, said providing may comprise dispensing the property modifying agent in the second compartment.

Optionally, said providing may comprise dispensing the property modifying agent in the second compartment using a dispenser.

Optionally, the method may comprise moving the aerosol forming device to facilitate mixing of the aerosol former and the property modifying agent once they are put into contact with each other.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorant and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The eight mode of the disclosure includes the combination of the aspects and preferred features described except where such a combination is clearly impermissible or expressly avoided.

The skilled person will appreciate that except where mutually exclusive, a feature or parameter described in relation to any one of the above aspects of the eighth mode may be applied to any other aspect of the eighth mode. Furthermore, except where mutually exclusive, any feature or parameter of the eighth mode described herein may be applied to any aspect and/or combined with any other feature or parameter of the eighth mode described herein.

Ninth Mode: A Smoking Substitute Apparatus Comprising a User Controllable Valve

At its most general, a ninth mode of the present disclosure relates to a smoking substitute apparatus comprising a user controllable valve allowing a user to select a proportion of additive received through a mouthpiece of the apparatus.

According to a first aspect of the ninth mode there is provided a smoking substitute apparatus comprising: a first passage for receipt of an aerosol flow; a second passage arranged in parallel with the first passage and comprising an additive delivery portion for delivering an additive to an airflow in the second passage; a mouthpiece comprising an outlet in fluid communication with the first and second passages; and a valve for controlling aerosol flow through the first passage, the valve movable between: a first position in which an airflow drawn through the outlet is received substantially exclusively from first passage; and a second position in which an airflow drawn through the outlet is received from a combination of the first and second passages.

The smoking substitute apparatus may provide a convenient means for providing an additive separately to an aerosol (e.g., where the second passage is for receipt of an aerosol). Providing an additive that is separate to the e-liquid may provide more versatility in how flavor can be delivered to the user. The user controllable valve allows a user to control the amount of additive provided to the user via the outlet.

The phrase “substantially exclusively” means that minimal airflow is received (or no airflow is received) at the outlet from the second passage when the valve is in the first position. For example, if any airflow is received from the second passage, that airflow may only be incidental and may not contribute in any significant way to the characteristics of the airflow received at the outlet. For example, where the additive delivery portion delivers a flavorant to the airflow, that flavorant may not be detectable by a user when the valve is in the first position.

For example, when the valve is in the first position, the proportion of the airflow received at the outlet that is received from the first passage may be greater than 90%. The proportion of the airflow received at the outlet (when the valve is in the open position) that is received from the first passage may be greater than 95%. The proportion of the airflow received at the outlet that is received from the first passage may be 100%.

In the first position, the first passage may be substantially unobstructed by the valve. Thus, the valve may be in a retracted position.

In the first position the second passage may be substantially unobstructed by the valve. In the second position the second passage may be substantially unobstructed by the valve. In that respect, both the first and second passages may be considered open (i.e., to airflow) when the valve is in the first position. The second passage may have a greater resistance (or substantially greater resistance) to airflow than the first passage. In this way, a path of least resistance may be defined through the first passage. Thus, when the valve is in the first position (i.e., and both passages are open) air may only flow through the first passage and not the second passage due to the second passage having the greater resistance to airflow.

In the second position the first passage may at least partially obstructed by the valve. This obstruction may provide some resistance to airflow in the first passage. In other words, the resistance to airflow of the first passage may be greater when the valve is in the second position compared to when the valve is in the first position. This may mean that the resistance to airflow of the first and second passages are equal (or similar) such that a combination of airflows from the first and second passages is received when air is drawn from the outlet. That is, airflow through the second passage may indirectly controlled by movement of the valve of the first passage (i.e., due to that movement altering a resistance to airflow of the first passage).

The valve may be movable to a third position in which an airflow drawn through the outlet is received substantially exclusively from the second passage. In the third position, the valve substantially obstructs the first passage. This obstruction of the first passage may prevent (or substantially prevent) airflow from being drawn through the first passage.

Thus, a single valve may provide an airflow drawn through the outlet that is exclusively from the first passage, exclusively from the second passage, or from a combination of the first and second passages.

The second passage may comprise an airflow restrictor for restricting airflow through the second passage. The airflow restrictor may, for example, be in the form of a narrower section in the passage.

Alternatively, the airflow restrictor may comprise a channel that follows a tortuous path.

The additive delivery portion may be configured to restrict airflow through the second passage. The additive delivery portion may comprise a porous medium (e.g., a porous element). The porous medium may provide partial obstruction of the second passage so as to restrict airflow through the second passage. An additive may be deposited on the porous medium. Alternatively, the porous medium may be impregnated with the additive.

The additive delivery portion may be in the form of a porous wick. The porous wick may extend across at least a portion of the passage. A portion of the porous wick may extend into an additive reservoir (e.g., a tank containing an additive). In this way, the additive may be conveyed from the reservoir along the wick (i.e., so as to be exposed to an airflow in the passage).

The additive delivery portion may alternatively be in the form of a portion of the passage (e.g., of a wall of the passage) comprising an additive coating.

The additive delivery portion may be in the form of a plurality of particles packed in a container (e.g., a mesh container). In this respect, air may flow through gaps formed between the packed particles.

The valve may be biased into the first position. The valve may be biased by a spring. For example, the valve may be biased by a compression spring or a torsion spring.

Movement of the valve between the first and second positions may be in a direction generally transverse to a longitudinal axis of the first passage. Movement of the valve between the first and second positions may be substantially perpendicular to the longitudinal axis of the first passage. The valve may be slidably engaged with a wall of the passage (and/or a wall of a housing of the apparatus).

The valve may comprise a pushbutton for a user to move the valve between the first and second positions. The valve may comprise a barrier for obstructing the first passage. The barrier may be rigidly connected to the pushbutton. The pushbutton may define a first end of the valve and the barrier may define an opposing end of the valve. When the valve is in the first position the pushbutton may project beyond an outer surface of a housing of the apparatus. A compression spring may be disposed between the barrier and an opposing wall of the first passage so as to bias the valve into the first position (e.g., such that the barrier is retracted from the first passage). A sealing portion may be disposed between the valve and a wall of the passage so as to prevent leakage of an airflow (e.g., including an aerosol) from the first passage.

The valve may be configured to rotate between the first and second positions. The valve may hinge between the first and second positions. In that respect, the valve may be hingably mounted (e.g., to a wall of the first passage). In this respect, the valve may be in the form of a flap.

The valve may be configured to move from the first position to the second position in response to an airflow drawn from the outlet. The valve may be configured to move from the first position to the second position in response to a pressure drop in the first passage. The pressure drop may be a predetermined pressure drop. That is, the valve may only move from the first position to the second position in response to a predetermined pressure in the first passage. The predetermined pressure may be a pressure indicative of a user inhaling at the outlet. The valve may be configured to move in response to the magnitude of an inhale (e.g., flow rate drawn from outlet) by a user at the outlet. In this way, a proportion of airflow from the second passage (received through the outlet) may be controlled by the strength of an inhalation at the outlet. Thus, for example, where the valve is a flap, the valve may rotate (e.g., about a hinge) in the first passage driven by an airflow drawn from the outlet.

Alternatively, the valve may comprise an actuator for moving the valve. The actuator portion may be movable in response to a signal received from, e.g., a controller. The controller may control the actuator in response to a signal received from a sensor. For example, the apparatus may comprise a puff sensor for detecting inhalation at the outlet and the controller may be configured to control the actuator in response to a signal indicative of inhalation at the outlet. The apparatus may comprise, e.g., a pressure sensor for detecting a pressure in the first or second passage. The controller may control the actuator in response to a signal indicative of a pressure in the first or second passage. Thus, for example, the controller may move the valve from the first position to the second position in response to a pressure drop.

The additive delivery portion may be configured to deliver a flavorant to an airflow in the second passage. In that respect, the additive delivery portion may comprise a flavorant. The term “flavorant” may describe a compound or combination of compounds that provide flavor and/or aroma. For example, the flavorant may be configured to interact with a sensory receptor of a user (such as an olfactory or taste receptor). The flavorant may include one or more volatile substances.

The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not. The first passage may be at least partially defined by the tank. The tank may substantially (or fully) define the first passage.

In this respect, the tank may surround the first passage.

The first passage may comprise an aerosol generator for delivering an aerosol to an airflow in the first passage. The valve may be between the aerosol generator and the outlet. That is, the aerosol generator may be upstream of the valve. The aerosol generator may comprise a wick. The aerosol generator may further comprise a heater. The wick may comprise a porous material. A portion of the wick may be exposed to airflow in the first passage. The wick may also comprise one or more portions in contact with liquid stored in the reservoir. For example, opposing ends of the wick may protrude into the reservoir and a central portion (between the ends) may extend across the passage so as to be exposed to air flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in air flowing through the first passage. This vapor may subsequently cool to form an aerosol in the first passage.

The first and second passages may each comprise a respective inlet. When the apparatus is in the form of a consumable, each inlet may be formed in an end of the consumable for engagement with main body. Alternatively, the first and second passages may share an inlet. A mixing chamber may be fluidly connected between the passages and the outlet. When the valve is in the second position, the airflows from the first and second passages may mix in the mixing chamber prior to being drawn through the outlet. Alternatively, the first and second passages may extend to the outlet (e.g., such that mixing instead occurs in a user's mouth once inhaled).

The passages may be generally parallel to one another. The passages may be separated by a separator in the form of a wall extending between the passages. Each of the passages may have a generally continuous (i.e., constant cross-section) along its length.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method. As is provided above, the controller may be configured to control the valve (e.g., an actuator for moving the valve). Thus, the controller may control the valve to move between the first and second positions.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

As is provided above, a puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the ninth mode described except where such a combination is clearly impermissible or expressly avoided.

The skilled person will appreciate that except where mutually exclusive, a feature or parameter described in relation to any one of the above aspects of the ninth mode may be applied to any other aspect of the ninth mode. Furthermore, except where mutually exclusive, any feature or parameter of the ninth mode described herein may be applied to any aspect and/or combined with any other feature or parameter of the ninth mode described herein.

Tenth Mode: A Heater to Heat Fluid Passing Through a Smoking Substitute Device

At its most general, a tenth mode of the present disclosure relates to provision of a heater to heat fluid passing through a smoking substitute device and thereby cause a flavorant to be entrained in the fluid flow.

According to a first aspect of the tenth mode there is provided a smoking substitute device having a fluid inlet, a fluid outlet, and an aerosol generator arranged between the inlet and the outlet and in fluid communication with each, the aerosol generator operable to generate an aerosol from an aerosol precursor, wherein the smoking substitute device further comprises a heater arranged between the inlet and the aerosol generator, the heater being configured to heat fluid drawn through the inlet towards the aerosol generator, and a source of flavorant provided between the heater and the aerosol generator so as to be presented to a flow of fluid from said heater towards said aerosol generator, the flavorant comprising one or more substances that activate at least one of an olfactory receptor in a human nasal cavity and a taste receptor in a human oral cavity, and wherein a flow of fluid heated by the heater is effective to release flavorant from said source for entrainment in said fluid.

Optionally, the heater comprises one or more heating elements formed as a metal mesh.

Conveniently, the heater comprises one or more heater elements formed as coils.

Advantageously, the smoking substitute device is configured such that the aerosol generator and said heater are operable independently of one another.

Optionally, the aerosol generator is operable in combination with said heater.

Conveniently, the aerosol generator and said heater are operable in synchronism.

Advantageously, the smoking substitute device comprises a flavorant reservoir, wherein the flavorant is provided in the flavorant reservoir.

Optionally, the flavorant is provided as a liquid.

Conveniently, the source of flavorant is a flavored article comprising a substrate carrying said flavorant.

Advantageously, at least part of said substrate is impregnated with the flavorant.

Optionally, at least part of the substrate is coated with the flavorant.

Conveniently, the source of flavorant is a flavor pod comprising a container at least partially filled with the flavorant.

Advantageously, the source of flavorant is a removable flavor part of the smoking substitute device.

Optionally, the removable flavor part is a replaceable part of the smoking substitute device.

Conveniently, the removable flavor part is a consumable.

Advantageously, a part of the smoking substitute device is a reusable part comprising a power source for the heater and aerosol generator.

Optionally, the heater is provided within the reusable part.

Conveniently, the source of flavorant is provided within the reusable part.

Advantageously, a part of the smoking substitute device is a removable precursor part comprising a source of aerosol precursor for the aerosol generator.

Optionally, the removable precursor part is a replaceable part.

Conveniently, the removable precursor part is a consumable.

Advantageously, the source of flavorant is provided in the removable precursor part.

Optionally, the source of flavorant is attachable to said removable precursor part.

Conveniently, the aerosol precursor is substantially free of flavorant.

Advantageously, the aerosol precursor is a liquid.

Optionally, the aerosol precursor contains nicotine.

According to a second aspect of the tenth mode, a consumable may be provided comprising a source of flavorant for use with the smoking substitute device according to the first aspect of the tenth mode.

According to a third aspect of the tenth mode, a pack comprising a plurality of consumables according to the second aspect of the tenth mode may be provided.

The flavorant may include one or more volatile substances. The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol precursor (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol precursor (e.g., e-liquid) of the system may be replenished by re-filling, e.g., a precursor reservoir of the smoking substitute apparatus with the aerosol precursor (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a precursor reservoir configured to store an aerosol precursor, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The precursor reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The precursor reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise an aerosol generating heater. The wick may comprise a porous material. A portion of the wick may be exposed to fluid flow in the passage. The wick may also comprise one or more portions in contact with liquid stored in the precursor reservoir. For example, opposing ends of the wick may protrude into the precursor reservoir and a central portion (between the ends) may extend across the passage so as to be exposed to fluid flow in the passage. Thus, fluid may be drawn (e.g., by capillary action) along the wick, from the precursor reservoir to the exposed portion of the wick.

The aerosol generating heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to an aerosol generator of the smoking substitute apparatus (e.g., when engaged with the main body) and the heater of the smoking substitute apparatus. The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to an aerosol generator of the consumable and the heater of the smoking substitute apparatus.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the aerosol generator of the smoking substitute apparatus (e.g., via the electrical contacts) and the heater of the smoking substitute apparatus. A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the aerosol generator of the consumable and/or the heater of the smoking substitute apparatus in response to a puff detection by the sensor. The control may be in the form of activation of the aerosol generator and/or the heater of the smoking substitute apparatus in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the tenth mode described except where such a combination is clearly impermissible or expressly avoided.

The skilled person will appreciate that except where mutually exclusive, a feature or parameter described in relation to any one of the above aspects of the tenth mode may be applied to any other aspect of the tenth mode. Furthermore, except where mutually exclusive, any feature or parameter of the tenth mode described herein may be applied to any aspect and/or combined with any other feature or parameter of the tenth mode described herein.

Eleventh Mode: A Delivery of Flavor to a User by Olfactory Flavor Delivery

At its most general, an eleventh mode of the present disclosure relates to a delivery of flavor to a user by olfactory flavor delivery.

According to a first aspect of the eleventh mode, there is provided a flavor delivery article for use with a smoking substitute apparatus, wherein the flavor delivery article contains a fragranced fluid, and is configured to release a mist of the fragranced fluid upon actuation of the flavor delivery article for olfactory flavor delivery.

Advantageously, flavor can be sensed through the nose of a user compared to within the mouth of the user. Specifically, the effect of flavor can be provided through the sense of smell rather than the sense of taste. In this way, the delivery of flavor is separate from delivery of e-liquid vapor, which may or may not contain nicotine. This can affect and/or increase the amount and/or strength of flavor sensed by a user. Furthermore, as the mist (i.e., spray) of fragranced fluid is released upon actuation of the flavor delivery article, a user can choose whether they want flavor delivery during use of the smoking substitute device (i.e., during vaping).

Preferably, the flavor delivery article is configured to release a mist of the fragranced fluid upon actuation (i.e., activation) of the flavor delivery article by a user of the smoking substitute device.

Optionally, the flavor delivery article may be configured to release the mist of the fragranced fluid only when the flavor delivery article is actuated, and may be configured to not release the mist of the fragranced fluid when the flavor delivery article is not actuated.

In this way, a user can choose to activate flavor delivery or to deactivate flavor delivery during vaping depending on whether or not they would like to receive flavor during each use (i.e., each puff) of the smoking substitute device.

Conveniently, the flavor delivery article may be a compressible pocket. The compressible pocket may be biased into an uncompressed state, and configured to release the mist of the fragranced fluid upon compression of the compressible pocket.

The compressible pocket provides a user-friendly and simple way of actuating the flavor delivery article in order to deliver flavor. A user can easily compress the pocket like a blower to push a mist of the fragranced fluid, which is contained in the compressible pocket, into the surrounding ambient air. The mist of the fragranced fluid can then be received by the user by breathing in the mist of the fragranced fluid through the nose.

The compressible pocket may comprise a valve inlet for introducing ambient air into the flavor delivery article.

In this way, after compression of the pocket to release the mist of fragranced fluid, air can be reintroduced via the valve inlet into the compressible pocket to enable further and ongoing compressions of the pocket.

This valve inlet can help to prevent the formation of vacuum-like conditions in the pocket, which would prevent the pocket from being reusable. In other words, the valve inlet allows the pocket to refill with ambient air after each compression, such that the pocket can be actuated repeatedly. Thus, the release of the mist of fragranced fluid for olfactory flavor delivery can be repeated.

Optionally, the valve inlet may be a one-way valve which allows the flow of air through the valve inlet into the flavor delivery article, but prevents the flow of air through the valve inlet out of the flavor delivery article.

Accordingly, air can be allowed back into the pocket to enable ongoing compressions of the pocket by a user, and the air does not escape from the pocket via the valve inlet. This configuration helps to bias the pocket into an uncompressed state.

The flavor delivery article may comprise an outlet mister for releasing the mist of the fragranced fluid upon actuation of the flavor delivery article.

The outlet mister may release the mist of fragranced fluid in a specified particle size, such that the fragranced fluid can be easily and efficiently received by a user's nose for olfactory flavor delivery.

Optionally, the outlet mister may comprise a plurality of holes or slits for releasing the mist of the fragranced fluid.

The holes or slits may be sized to release a preferred and/or specified particle size of the mist of fragranced fluid.

Conveniently, the outlet mister may be a silicone valve.

When the flavor delivery apparatus comprises both an outlet mister and a valve inlet, the outlet mister and valve inlet may be integral with one another to form a single combined valve. Upon actuation of the flavor delivery apparatus, the single combined valve is configured to act as an outlet mister for releasing the mist of the fragranced fluid. When the flavor delivery apparatus is not actuated (e.g., after actuation of the flavor delivery apparatus), the single combined valve is configured to act as the valve inlet to allow the flow of air through the single combined valve into the pocket. This enables repeatable compressions of the pocket.

Advantageously, the fragranced fluid may be an oil-based liquid and/or a sugar-based liquid.

According to a second aspect of the eleventh mode, there is provided a smoking substitute apparatus for a smoking substitute device, the smoking substitute apparatus comprising a tank for containing a vaporizable e-liquid, a heating element for heating the vaporizable e-liquid, and the flavor delivery article according to the first aspect of the eleventh mode.

The smoking substitute apparatus may be a consumable.

Advantageously, flavor can be sensed through the nose rather than through the mouth of a user. The delivery of the vaporized e-liquid to the user is via the mouth, whereas the delivery of flavor to the user can be through the nose, via an olfactory flavor delivery mechanism. Therefore, the delivery of the e-liquid which may or may not contain nicotine is separate from the delivery of the flavor. This can affect and/or increase the amount and/or strength of flavor sensed by a user. Furthermore, as the mist of fragranced fluid is released upon actuation of the flavor delivery article, a user can choose whether they want flavor delivery in each use (i.e., each puff) of the smoking substitute device (i.e., during vaping) by actuating the flavor delivery article.

Optionally, the smoking substitute apparatus may further comprise an outer casing, and the flavor delivery article may be molded into the outer casing.

Accordingly, the flavor delivery article is positioned where a user may easily activate it (e.g., by pressing using a finger or otherwise the flavor delivery article), because the flavor delivery article is positioned on an outside of the smoking substitute device. Thus, the flavor delivery article can be easily operated to release a flavor during vaping, under the control of a user of the smoking substitute device.

One end of the smoking substitute apparatus may define a mouthpiece. When the flavor delivery article comprises an outlet mister for releasing the mist of the fragranced fluid upon actuation of the flavor delivery article, the outlet mister may be positioned so that it is closer to the end of the smoking substitute apparatus comprising the mouthpiece than it is to the other end of the smoking substitute apparatus.

In this way, the release of the mist of fragrance fluid occurs closer to or proximate the mouthpiece, and thus closer to a user of the smoking substitute device. The mist of fragranced fluid is thereby released in the region of a user's nose for olfactory flavor delivery.

The outlet mister may be configured to release the mist of the fragranced fluid generally towards the mouthpiece. Additionally, the outlet mister may be positioned, arranged and/or orientated such that, in use, the mist of fragranced fluid is released towards the nose of a user. For example, the outlet mister may be positioned such that in use of the smoking substitute device, the outlet mister is directly underneath or adjacent to a nose of a user.

By releasing the mist of the fragranced fluid towards the mouthpiece, the mist of fragranced fluid is directed towards a user's nose during vaping. In this way, the user can receive a stronger flavor as more of the released mist of fragranced fluid can be received by the user' nose.

Preferably, the flavor delivery article is separated from the tank for containing the e-liquid.

In this way, the fragranced liquid in the flavor delivery article is separate to the e-liquid and is therefore not in the primary air flow of the vaporized e-liquid through the mouthpiece. The fragranced fluid is thus not mixed or contaminated with the e-liquid during use, which may or may not contain nicotine.

According to a third aspect of the eleventh mode of the disclosure, there is provided an electronic cigarette device comprising the smoking substitute apparatus according to the second aspect of the eleventh mode of the disclosure, and a main body comprising a power source for providing power to the heating element of the smoking substitute apparatus.

The fragranced fluid may comprise a flavorant. The term “flavorant” is used herein to describe a compound or combination of compounds that provide flavor and/or aroma. For example, the flavorant may be configured to interact with a sensory receptor of a user (such as an olfactory or taste receptor).

The flavorant may include one or more volatile substances.

The flavorant may be provided in solid, gel or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the eleventh mode described except where such a combination is clearly impermissible or expressly avoided.

The skilled person will appreciate that except where mutually exclusive, a feature or parameter described in relation to any one of the above aspects of the eleventh mode may be applied to any other aspect of the eleventh mode. Furthermore, except where mutually exclusive, any feature or parameter of the eleventh mode described herein may be applied to any aspect and/or combined with any other feature or parameter of the eleventh mode described herein.

Twelfth Mode: A Flavor Delivery Article for a Smoking Substitute Device

At its most general, a twelfth mode of the present disclosure relates to a flavor delivery article for a smoking substitute device.

According to a first aspect of the twelfth mode, there is provided a flavor delivery article for a smoking substitute device, the flavor delivery article comprising a flavorant source comprising a fragranced fluid, and a flavor generator configured to agitate the fragranced fluid to thereby release the fragranced fluid from the flavor delivery article for olfactory flavor delivery.

The agitation (i.e., excitement) of the fragranced fluid leads to the release of the fragranced fluid from the flavor delivery article. The fragranced fluid may be released as a mist, aerosol or spray, for example.

Advantageously, a flavor can be sensed through the nose of a user rather than within the mouth of the user, by inhaling the fragranced fluid through the nose. In other words, the effect of flavor can be provided through the sense of smell rather than the sense of taste. In this way, the delivery of flavor is separate from delivery of e-liquid vapor in the smoking substitute device, which may or may not contain nicotine. This can affect and/or increase the amount, strength or intensity of flavor sensed by a user.

Preferably, the flavor generator is configured to agitate the fragranced fluid in response to a user input.

Accordingly, the delivery of flavor to a user depends on an action of a user. The flavor delivery article may only release the fragranced liquid when a user indicates that they require flavor. Accordingly, the flavor delivery article may not continuously release flavor. In this way, a user can choose whether they want flavor delivery during use of the smoking substitute device (i.e., during vaping).

The user input could be an action of a user, such as pushing a button or dial. Alternatively, the user input could be the act of drawing on (i.e., vaping) the smoking substitute device itself.

The flavor generator may be electrically powered.

Thus, the flavor generator may be powered by an electrical power source, such as a battery. The battery may be rechargeable. The user input may result in a change to the supply of power to the flavor generator to activate the flavor generator.

An amount of electrical power supplied to the flavor generator may be adjustable to modify the amount of fragranced fluid released from the flavor delivery article.

In this way, the strength, intensity and/or amount of flavor received by a user can be adjusted. The adjustment may be based on a user's preference.

Specifically, the amount of electrical power supplied to the flavor generator may be controllable by a user of the smoking substitute device to increase and/or decrease the amount of fragranced fluid released from the flavor delivery article, and thereby the amount of flavor received by a user. The user may be able to adjust the amount of electrical power supplied to the flavor generator by a button, dial or slider on the flavor delivery article and/or on the smoking substitute device, for example.

The flavor generator may be a piezoelectric motor.

A piezoelectric motor is an electromechanical device that uses the converse piezoelectric effect to convert electrical energy into mechanical energy. The piezoelectric motor is configured to receive an electrical input in the form of a high-frequency signal and convert this signal into vibrationary motion at the same frequency. This high-frequency oscillation agitates the fragranced fluid in the flavorant source, thereby releasing a mist of the fragranced fluid from the flavorant source.

The piezoelectric motor may comprise a piezoelectric element and a resonating surface, the resonating surface may be positioned adjacent to the flavorant source, and the piezoelectric element may be configured to vibrate the resonating surface upon receipt of an electrical signal, the resonating surface thereby providing a force on the flavorant source to agitate the fragranced fluid.

The piezoelectric element may comprise a piezoelectric material such as a ceramic material. The resonating surface may form a portion of the piezoelectric element, or may be formed from another element of the piezoelectric motor.

Optionally, the flavorant source may be an absorbable pad and the fragranced fluid may be a fragranced liquid absorbed into the pad.

In this way, the flavor generator (e.g., the piezoelectric motor) may agitate the fragranced liquid absorbed in the pad. This excitement of the fragranced liquid in the absorbable pad may result in the release of a mist, spray or aerosol of fragranced liquid from the pad, and therefore from the flavor delivery article.

The mist of fragranced liquid can then be inhaled through the nose of a user to deliver flavor.

Alternatively, the flavorant source may be a pocket, and the fragranced fluid may be a fragranced liquid contained in the pocket.

In this way, the flavor generator (e.g., the piezoelectric motor) may agitate the fragranced liquid contained in the pocket which in turn may release the fragranced liquid from the pocket, and therefore from the flavor delivery article. The fragranced liquid can then be inhaled through the nose of a user to deliver flavor.

The pocket may comprise an outlet mister, the outlet mister being configured to release a mist of the fragranced liquid upon agitation of the fragranced liquid by the flavor generator.

The outlet mister may release the mist of fragranced fluid in a specified particle size, such that the fragranced fluid can be easily and efficiently received by a user's nose for olfactory flavor delivery.

Accordingly, a mist, spray or aerosol of fragranced liquid may be released from the pocket, and therefore from the flavor delivery article. The mist of fragranced liquid can then be inhaled through the nose of a user to deliver flavor.

The flavor delivery article may further comprise attaching means for attaching the flavor delivery article to a smoking substitute device. The attaching means may be a clip, for example.

Accordingly, the flavor delivery article may be easily attachable and detachable from a smoking substitute device, such as an electronic cigarette. A user or the smoking substitute device can choose whether they would like the delivery of flavor by attaching or detaching the flavor delivery article to the smoking substitute device. The flavor delivery article may be attachable to a smoking substitute apparatus, such as a consumable, or a main body of the smoking substitute device.

Furthermore, a user of the smoking substitute device is able to change the flavor delivered simply by detaching a flavor delivery article having a first flavor (e.g., a fruit flavor) for a flavor delivery article having a second flavor (i.e., a spiced flavor). Further still, the flavor delivery article can be easily replaced once all of the fragranced fluid has been used.

Optionally, the flavorant source may be refillable with fragranced fluid in order to replenish diminished amounts of fragranced fluid.

According to a second aspect of the twelfth mode there is provided a smoking substitute apparatus comprising a tank for containing a vaporizable e-liquid, a heating element for heating the vaporizable e-liquid, and the flavor delivery article of the first aspect of the twelfth mode. The smoking substitute apparatus may be a consumable (i.e., a “pod”).

According to a third aspect of the twelfth mode there is provided a smoking substitute device comprising a tank for containing a vaporizable e-liquid, a heating element for heating the vaporizable e-liquid, an electrical power source for providing electrical power to the heating element, and the flavor delivery article of the first aspect of the twelfth mode. The electrical power source may be a battery, for example.

In this way, the smoking substitute device can supply vaporized e-liquid to the mouth of a user, and fragranced fluid to the nose of a user for olfactory flavor delivery. Accordingly, the delivery of flavor is separate from delivery of e-liquid vapor, which may or may not contain nicotine. This can affect and/or increase the amount and/or strength of flavor sensed by a user.

Preferably, the smoking substitute device may have opposing ends, wherein one end of the smoking substitute device defines a mouthpiece, and the flavor delivery article is configured to release the fragranced fluid generally towards the mouthpiece.

Additionally, the flavor delivery article may be positioned, arranged and/or orientated such that, in use, the fragranced fluid is released towards the nose of a user.

By releasing the fragranced fluid generally towards the mouthpiece, the fragranced fluid is directed towards a user's nose during vaping. In this way, the user can receive a stronger and/or more intense flavor as more of the released fragranced fluid can be received by the user's nose.

When the flavor generator is a piezoelectric motor, the electrical power source may be electrically connectable to the piezoelectric motor to provide electrical power to the piezoelectric motor. Thus, the electrical power source can power both the heating element and the piezoelectric motor. Therefore, whenever the heating element is operative to provide e-liquid vapor to a user, the flavor delivery article is also activated.

Alternatively, the piezoelectric motor may be electrically powered by a second power source which is different from the power source for providing power to the heating element.

The smoking substitute device may further comprise a controller for controlling the supply of electrical power to the heating element and/or the piezoelectric motor.

Accordingly, the supply of power to the piezoelectric motor can be started, stopped and/or varied, which in turn starts, stops and/or varies the amount of fragranced fluid released from the flavor delivery article for olfactory delivery to a user.

The smoking substitute device may further comprise a puff sensor operatively connectable to the controller so as to be able to provide a signal to the controller that is indicative of a detected puff by a user, wherein the controller may be configured to instruct the supply of electrical power to the heating element and/or the piezoelectric motor in response to the receipt of the signal.

In this way, the piezoelectric motor is automatically activated when a user inhales (i.e., draws on the smoking substitute device), so that flavor is automatically delivered when the user inhales. The pressure sensor may detect a drop in pressure when the user inhales which thereby activates the piezoelectric motor.

The fragranced fluid may comprise a flavorant. The term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances.

The flavorant may be provided in liquid, gel or gaseous form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless (i.e., not contain any flavorants) and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the twelfth mode described except where such a combination is clearly impermissible or expressly avoided.

The skilled person will appreciate that except where mutually exclusive, a feature or parameter described in relation to any one of the above aspects of the twelfth mode may be applied to any other aspect of the twelfth mode. Furthermore, except where mutually exclusive, any feature or parameter of the twelfth mode described herein may be applied to any aspect and/or combined with any other feature or parameter of the twelfth mode described herein.

Thirteenth Mode: A Smoking Substitute Apparatus Capable of Delivering Flavor to a User

At its most general, a thirteenth mode of the present disclosure relates to a smoking substitute apparatus capable of delivering flavor to a user.

According to a first aspect of the thirteenth mode there is provided a smoking substitute apparatus comprising a reservoir for containing a vaporizable e-liquid, an aerosol-generator configured to vaporize said e-liquid to produce an aerosol vapor, a flavor cavity containing a powder, the powder comprising a flavorant, a first flow passage for guiding said aerosol vapor from the aerosol-generator to an outlet of the first flow passage, and a second flow passage for guiding ambient air through the flavor cavity to an outlet of said second flow passage, wherein said outlet of the first flow passage and said outlet of the second flow passage are both located at a mouthpiece of the apparatus, and wherein flow of ambient air through said second flow passage is effective to entrain powder from said flavor cavity for inhalation by a user of the smoking substitute apparatus.

In this way, flavor can be delivered to a user of the smoking substitute device. The flavor is provided in a solid powder form, which requires less storage volume in the smoking substitute apparatus than other states of flavorant, such as flavorant in a liquid or gas state. Accordingly, the size of the flavor cavity can be reduced, thereby reducing the overall size of the smoking substitute apparatus.

The powder itself is entrained through the mouthpiece into the mouth of a user during each use of the smoking substitute apparatus (i.e., during each puff) to deliver flavor to the user. The delivery of the powder itself into a user's mouth can provide a strong and/or intense flavor. Furthermore, a flavored powder can travel deeper into the airways of a user than a flavored liquid (i.e., in the form of liquid droplets or otherwise), thereby providing a better taste experience for a user.

Optionally, the outlet of the first flow passage is distinct from the outlet of the second flow passage.

Powders may congeal if they come into contact with liquid, creating lumps and even blockages. By separating the first flow passage outlet from the second flow passage outlet, the entrained powder is separated from the vaporized e-liquid (i.e., the aerosol vapor) in the mouthpiece. Accordingly, the number and size of lumps and/or blockages of congealed powder in the mouthpiece can be reduced or even prevented. Thus, the possibility of a user breathing in an unpleasant lump of congealed powder is also reduced or eliminated. Additionally, the possibility of the mouthpiece becoming blocked (or partially blocked/obstructed) by congealed powder is reduced. This improves the experience for a user of the smoking substitute apparatus.

Preferably, the second flow passage may be fluidly isolated from the first flow passage.

Accordingly, the first and second flow passages are separate and distinct from each other along their entire length. As the entrained flavored powder does not come into contact with the aerosol vapor, the possibility of the formation of lumps or blockages of congealed powder is reduced or eliminated. Furthermore, as the flavorant is not mixed with the e-liquid, which may or may not contain nicotine, the strength and or taste of the flavor may be improved.

The second flow passage may extend from an inlet, through the flavor cavity, to the outlet of said second flow passage wherein said inlet is in an outer wall of the smoking substitute apparatus.

In this way, ambient air on the outside of (i.e., surrounding) the smoking substitute apparatus can be efficiently and effectively drawn through the inlet and into the second flow passage during each use (i.e., puff) of the smoking substitute apparatus. The ambient air can flow through the flavor cavity to entrain (i.e., draw in and transport in the flow of ambient air) flavored powder from the flavor cavity to the mouthpiece, and therefore into the mouth of the user.

Optionally, the inlet of the second flow passage may be in an outer side wall of the smoking substitute apparatus.

This provides a simple configuration in which ambient air can be drawn in through a side wall of the smoking substitute apparatus. This configuration of the second flow passage may also be simple to manufacture.

The second flow passage may be non-linear. For example, the second flow passage may be tortuous (i.e., follow a twisting and turning and/or winding path). The second flow passage may follow a serpentine, undulating or meandering path.

In this way, an enhanced amount of flavored powder may be entrained in the ambient air for delivery to a user. In other words, providing such a non-linear second flow passage may provide an improved dosage of flavored powder into a mouth of a user relative to the dosage of vaporized e-liquid. The nonlinear path of the second flow passage may also result in increased air turbulence in the second flow passage which may affect the amount of powder entrained to the mouthpiece, thereby affecting the flavor dosage and flavor strength received by a user.

Optionally, the second flow passage may be shaped as a spiral. The second flow passage may spiral around the first flow passage and the reservoir for containing the vaporizable e-liquid.

Such a configuration may further improve dosage of flavored powder into a mouth of a user, which in turn affects the strength and/or depth of flavor received by a user.

The width of the first flow passage may be larger than the width of the second flow passage. In other words, a cross-section of the first flow passage may be larger than a cross-section of the second flow passage.

In this way, the ratio of amount of flavored powder and the amount of vaporized e-liquid received by the user through the mouthpiece may be improved to provide an enhanced strength and/or depth of flavor.

The width of the second flow passage may vary along its length. In other words, the cross-section of the first flow passage may vary along the length of the second flow passage.

Accordingly, an improved dosage of flavored powder may be delivered to a user.

Alternatively, the first and section flow passages may each have a consistent cross-section along their respective lengths.

The first flow passage may be linear.

In this way, the vaporized e-liquid may be guided in a direct route from the aerosol generator to the mouthpiece for efficient delivery to a user of the smoking substitute apparatus.

The size and/or shape of the second flow passage inlet may be adjustable.

Alternatively, or additionally, the size and/or shape of the second flow passage outlet may be adjustable.

Adjusting the size and/or shape of the inlet or outlet of the second flow passage may result in a change in the amount of flavored powder entrained in the air flow during each use (i.e., each puff) of the smoking substitute apparatus.

Optionally, the smoking substitute apparatus may comprise a flow passage obstructer, which may be extendable across the second flow passage to block the second flow passage and prevent the entrainment of powder to the mouthpiece.

For example, the flow passage obstructer may be a cap or lid for positioning over the inlet of the second flow passage to prevent the entrainment of powder to the mouthpiece.

In this way, the transport of flavored powder from the flavor cavity to the mouthpiece, and therefore the mouth of a user, can be controlled. Specifically, if the flow passage obstructer is in an extended state across the second flow passage, the entrainment of powder to the mouthpiece is prevented, thus preventing flavor delivery to the mouth of a user. Accordingly, if the delivery of flavor is not required, flavor delivery can be prevented. If the delivery of flavor is subsequently required, the flow passage obstructer can be retracted into a retracted state in which the obstructer does not extend across the second flow passage. Flavor delivery can then be resumed.

The flow passage obstructer may also prevent powder from undesirably leaking from the smoking substitute apparatus.

Optionally, the flavor cavity may be refillable.

In this way, powders having different flavors can be used with a single smoking substitute apparatus. A user of the smoking substitute apparatus is able to change the flavor delivered by the smoking substitute apparatus.

A particle size of the flavored powder may also be chosen to alter and/or improve the impact or strength of flavor, and the amount of flavored powder entrained into the air flow through the second flow passage.

The aerosol-generator may be a heating element for heating the vaporizable e-liquid.

According to a second aspect of the thirteenth mode, there is provided a smoking substitute system comprising the smoking substitute apparatus according to the first aspect of the thirteenth mode, and a main body comprising a power source.

The powder comprises a flavorant. The term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances.

The flavorant may be provided in solid particle form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless (i.e., not contain any flavorants) and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a first flow passage for fluid flow therethrough. The first flow passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the thirteenth mode described except where such a combination is clearly impermissible or expressly avoided.

The skilled person will appreciate that except where mutually exclusive, a feature or parameter described in relation to any one of the above aspects of the thirteenth mode may be applied to any other aspect of the thirteenth mode. Furthermore, except where mutually exclusive, any feature or parameter of the thirteenth mode described herein may be applied to any aspect and/or combined with any other feature or parameter of the thirteenth mode described herein.

Fourteenth Mode: A Mouthpiece for a Smoking Substitute Apparatus Having One or More Electrode

At its most general, a fourteenth mode of the present disclosure relates to a mouthpiece for a smoking substitute apparatus, the mouthpiece having one or more electrodes disposed thereon to stimulate a user's tongue. Using the one or more electrodes, the user's tongue may be stimulated to simulate a flavor sensation. Simulation of flavor may be controlled, for example, by controlling electrical and/or thermal signals provided to the user via the one or more electrodes. In this manner, the mouthpiece of the present disclosure may enable a smoking substitute apparatus to deliver flavor to the user via stimulation of the user's tongue, rather than through a chemical flavorant delivered by the smoking substitute apparatus. This may be referred to as “digital” flavor simulation.

As a result, there may be no need to provide a chemical flavorant in the smoking substitute apparatus. For example, the smoking substitute apparatus may include an aerosol-former (e.g., e-liquid or tobacco material) which does not have any flavor additives therein.

The ability to deliver flavor without use of a chemical flavorant may have several benefits. First, it may avoid producing smells that are perceptible by people in the vicinity of a user of the smoking substitute apparatus. This may enable more discrete use of the smoking substitute apparatus. Second, it may avoid any potential health drawbacks associated with the use of chemical flavorants. Third, as no chemical flavorant is needed, it may be possible to reduce a size of the smoking substitute apparatus (e.g., because there is no need for a flavorant reservoir).

The simulated flavor may easily be changed by varying the stimulation provided to the user's tongue by the one or more electrodes. In this manner, a wide variety of flavor sensations may be achieved using a single mouthpiece, without having to use any flavorants. This may improve the versatility of the smoking substitute apparatus, as it may enable a user to easily change the flavor delivered by the apparatus on the fly (e.g., by controlling the simulated flavor through an application on a mobile device).

The present disclosure is based on the recent discovery that electrical and thermal signals applied to a user's tongue may be used to simulate flavor. (R. A. Nimesha Ranasinghe, “Digitally Simulating the Sensation of Taste Through Electrical and Thermal Stimulation”, Doctoral Thesis, National University of Singapore, 2012) explains how different electrical and thermal signals may be used to simulate different types of flavors.

By adjusting the electrical and/or thermal signals applied to the user's tongue, it may be possible to simulate sweet, sour, bitter and savory taste modalities. Accordingly, by adjusting the electrical and/or thermal signals applied to the user's tongue it may be possible to simulate a wide range of flavors, including menthol, licorice, chocolate, fruit flavor (including, e.g., citrus, cherry etc.), vanilla, spice (e.g., ginger, cinnamon) and tobacco flavor.

According to a first aspect of the fourteenth mode there is provided a mouthpiece for a smoking substitute apparatus, the mouthpiece comprising: an outlet for conveying an aerosol generated by the smoking substitute apparatus to a user; and one or more electrodes for stimulating the user's tongue. In this manner, the one or more electrodes may be used to stimulate the user's tongue when the user puts the mouthpiece in their mouth (e.g., to inhale an aerosol from the smoking substitute apparatus), in order to simulate a flavor sensation for the user. Thus, flavor delivery may be simulated when the user uses the smoking substitute apparatus, e.g., when the user inhales an aerosol from the smoking substitute apparatus.

The one or more electrodes may be disposed on an outer surface of the mouthpiece. The one or more electrodes may be arranged to contact the user's tongue when the mouthpiece is inserted into the user's mouth.

The outlet may serve to convey an aerosol from the smoking substitute apparatus to the user, when the mouthpiece is assembled with the smoking substitute apparatus. The outlet may be arranged such that, when the mouthpiece is assembled with the smoking substitute apparatus, the outlet is in fluid communication with an aerosol-conveying passage in the smoking substitute apparatus. In some embodiments, the mouthpiece may be a part or component of the smoking substitute apparatus, e.g., it may be an integral part of the smoking substitute apparatus. In other embodiments, the mouthpiece may be removably mountable on the smoking substitute apparatus or a component thereof.

Different electrode arrangements may be used, depending on the type of stimulation used. The one or more electrodes may include multiple electrodes arranged to contact different parts of the user's tongue, in order to stimulate different parts of the user's tongue. This may enable a flavor sensation to be produce over a larger area of the user's tongue.

The one or more electrodes may include one or more electrodes arranged to electrically stimulate the user's tongue. Electrical stimulation of the user's tongue may, for example, include application of a voltage and/or a current to the user's tongue. Electrical stimulation of the user's tongue may result in the user experiencing a flavor sensation on their tongue, and so may be used to simulate flavor. The flavor simulation may be controlled by controlling an electrical signal (e.g., voltage or current) delivered to the tongue via the one or more electrodes. Different electrical signals delivered to the user's tongue may result in different flavor sensations for the user. Properties of the electrical signal delivered to the user's tongue such as voltage, current level, frequency, etc. may be varied to simulate different flavors.

The one or more electrodes arranged to electrically stimulate the user's tongue may include a pair of electrodes. The pair of electrodes may be arranged to pass an electrical current through the user's tongue. In this manner, the user's tongue may be stimulated by passing a current through a part of the user's tongue located between the pair of electrodes. Preferably the current may be delivered to user's tongue in pulses. Parameters such as magnitude of the current, pulse duration, and/or pulse frequency may be controlled to simulate a desired flavor.

Additionally, or alternatively, the pair of electrodes may also enable two different parts of the user's tongue to be stimulated, e.g., by applying voltage pulses to the electrodes or by changing a temperature of the electrodes.

The pair of electrodes may include a first electrode and a second electrode arranged on opposite sides of the mouthpiece relative to the outlet. In other words, the first electrode and second electrode may be disposed on either side of the outlet. This may enable substantially uniform stimulation of the user's tongue around the outlet, which may enhance the favor sensation experienced by the user.

At least part of the one or more electrodes may be disposed on a forward-facing surface of the mouthpiece. This may facilitate bringing the one or more electrodes into contact with the user's tongue when the user inserts the mouthpiece into their mouth. The forward-facing surface may be a surface that, in use, is oriented towards the user. The outlet may be formed in the forward-facing surface.

At least part of the one or more electrodes may disposed on lateral-facing surface of the mouthpiece. In other words, part of the one or more electrodes may be disposed on a side surface of the mouthpiece. This may facilitate bringing the one or more electrodes into contact with the user's tongue when the user inserts the mouthpiece into their mouth, as the tongue is typically located near a side of the mouthpiece. The lateral-facing surface may be a surface of the mouthpiece that is substantially aligned with a longitudinal axis of the mouthpiece.

The one or more electrodes may include one or more electrodes arranged to thermally stimulate the user's tongue. Thermal stimulation of the user's tongue may involve varying a temperature of the one or more electrodes (e.g., heating or cooling an electrode) to cause a temperature change at a location on the user's tongue. A local temperature change on a user's tongue may result in a flavor sensation on the user's tongue. Flavor simulation may thus be controlled by controlling or varying a temperature of an electrode on the mouthpiece. The mouthpiece may include a temperature actuator for varying a temperature of the one or more electrodes arranged to thermally stimulate the user's tongue. The temperature actuator may be thermally coupled to the one or more electrodes arranged to thermally stimulate the user's tongue, so that a change in temperature of the temperature actuator may cause a change in temperature of the one or more electrodes. The temperature actuator may, for example, include a heater connected to the one or more electrodes arranged to thermally stimulate the user's tongue. Heating of the one or more electrodes may then be varied by controlling the heater (e.g., by controlling power supplied to the heater). As another example, a Peltier module may be used to vary the temperature of the one or more electrodes. A Peltier module may enable both heating and cooling of the electrodes, which may enable more rapid and accurate control of the temperature. In some cases, a temperature sensor may be connected to the one or more electrodes arranged to thermally stimulate the user's tongue, to monitor the temperature of the one or more electrodes. The temperature actuator may then be controlled based on an output from the temperature sensor (e.g., to bring the one or more electrodes to a desired temperature).

The mouthpiece may further include a connection interface for receiving a stimulation signal from the smoking substitute apparatus, to enable stimulation of the user's tongue based on the received stimulation signal. For example, the stimulation signal may be an electrical signal which may be applied to the user's tongue via the one or more electrodes. In this case, the connection interface may include an electrical connector for electrically connecting the one or more electrodes to a power supply and/or controller in a main body of the smoking substitute apparatus that generates the stimulation signal. In another example, the stimulation signal may be an electrical signal for activating or controlling the thermal actuator. In this case, the connection interface may include an electrical connector for electrically connecting the thermal actuator to a power supply and/or controller in the main body of the smoking substitute apparatus that generates the stimulation signal.

Where the mouthpiece is removably mountable in the smoking substitute apparatus, the connection interface may be configured to engage a corresponding connection interface in the smoking substitute apparatus when the mouthpiece is mounted in the smoking substitute apparatus. In this manner, the stimulation signal may be conveyed from a power supply and/or controller in the smoking substitute apparatus that generates the stimulation signal to the mouthpiece.

According to a second aspect of the fourteenth mode of the disclosure, there is provided a smoking substitute apparatus including a mouthpiece according to the first aspect of the fourteenth mode of the disclosure.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The mouthpiece may be removably mountable on the smoking substitute apparatus.

Where the mouthpiece is provided as part of a consumable that is engageable with a main body, the consumable may include separate sets of electrical contacts for connecting the one or more electrodes and the heater to the main body.

In some embodiments, the smoking substitute apparatus may be configured to generate a vaporless aerosol. This may avoid the risk of “passive vaping” by people in a vicinity of a user of the smoking substitute apparatus. Using an aerosol-former that produces a vaporless aerosol in combination with the mouthpiece of the disclosure may enable a user to use the smoking substitute apparatus without emitting any smells or vapors. Thus, use of the smoking substitute apparatus may be completely “invisible”, so that the apparatus may be used discretely and without affecting others.

For example, the smoking substitute apparatus may include a receptacle for receiving a nicotine-imbued porous substrate; and a heater arranged to heat an air flow passing through a nicotine-imbued porous substrate received in the receptacle. In this manner, the heater may heat an air flow passing through the nicotine-imbued porous substrate to produce a vaporless nicotine aerosol. As an example, the receptacle may be arranged such that the nicotine-imbued porous substrate is disposed in the passage in the apparatus, such that heated air flowing through the passage may pass through the nicotine-imbued substrate to form an aerosol. The heater may be located downstream of the substrate. The nicotine-imbued substrate may be flavorless (i.e., it does not contain any flavorant additive), such that flavor is delivered to the user solely through the one or more electrodes on the mouthpiece.

Where the smoking substitute apparatus includes a controller, the controller may be configured to control a stimulation signal provided to the one or more electrodes in the mouthpiece for stimulating the user's tongue. The controller may thus be used to control stimulation of the user's tongue via the one or more electrodes, to simulate a desired flavor. In this manner, flavor simulation may be controlled in real-time while a user is using the smoking substitute apparatus. Control of the stimulation signal by the controller may refer to generating and/or adjusting the signal. For example, the controller may adjust the stimulation signal, to vary the simulated flavor experience by the user.

The stimulation signal may be a signal (e.g., an electrical or thermal signal) that is provided to the one or more electrodes to stimulate the user's tongue to simulate a flavor. The stimulation signal may be generated by the controller itself, or via a separate component. For example, the controller may control a power supply of the smoking substitute apparatus to generate an electrical signal produced by the power supply.

The controller may be further configured to, in response to the user inhaling an aerosol from the smoking substitute apparatus, generate the stimulation signal provided to the one or more electrodes to stimulate the user's tongue. In this manner, the user's tongue may be automatically stimulated to simulate flavor when the user inhales an aerosol from the smoking substitute apparatus. For example, the smoking substitute apparatus may include a puff sensor for detecting when the user is inhaling through the mouthpiece (the puff sensor is discussed in more detail below). When such an event is detected, the controller may then generate the stimulation signal to stimulate the user's tongue. When no inhalation is detected, no stimulation signal may be generated, so that the user's tongue is not stimulated.

Where the one or more electrodes of the mouthpiece include one or more electrodes arranged to electrically stimulate the user's tongue; the stimulation signal may include an electrical current; and the controller may be configured to control one or more of a magnitude of the electrical current, a pulse duration of the electrical current, and a pulse frequency of the electrical current. In this manner, the user's tongue may be stimulated by passing an electrical current through the user's tongue. Properties of the current such as magnitude, pulse duration and pulse frequency may be varied to vary the simulated flavor experienced by the user. The controller may be configured to adjust these properties of the electrical current to predetermined values, in order to simulate a desired flavor. The controller may include a pulse generator for generating a pulsed current as the stimulation signal. A pulse width modulator may be used to control the frequency and width of the pulses. For example, the pulsed current may have a square-wave pulses.

Alternatively, or additionally, the stimulation signal may include a voltage signal. For example, pulsed voltages may be applied to the user's tongue to simulate a desired flavor.

Where the one or more electrodes of the mouthpiece include one or more electrodes arranged to thermally stimulate the user's tongue, the stimulation signal may be configured to cause a temperature change of the one or more electrodes that are arranged to thermally stimulate the user's tongue. In this manner, the user's tongue may be stimulated by varying a temperature of the one or more electrodes. For example, the stimulation signal may be arranged to vary a temperature of a temperature actuator (e.g., heater, Peltier module) which is thermally coupled to the one or more electrodes, thereby varying the temperature of the one or more electrodes. For example, the stimulation signal may be a current which is delivered to a heater to cause an increase in temperature of the heater.

The smoking substitute apparatus may include a temperature sensor arranged to monitor a temperature of the one or more electrodes. The controller may then be configured to adjust the stimulation signal delivered to the thermal actuator based on an output from the temperature sensor (e.g., to reach a desired temperature setpoint).

In some embodiments, the mouthpiece may include electrodes arranged to electrically stimulate the user's tongue and electrodes arranged to thermally stimulate the user's tongue. This may enable a combination of electrical and thermal stimulation of the user's tongue, which may provide enhanced flavor simulation. In such an embodiment, the controller may be configured to generate separate stimulation signals for electrical stimulation and thermal stimulation.

More generally the one or more electrodes of the mouthpiece may include multiple sets of electrodes arranged to stimulate different parts of the user's tongue. The controller may then be configured to provide separate stimulation signals for each of the multiple sets of electrodes, so that different stimulations may be provided to different parts of the user's tongue. This may enable a more complex and realistic flavor simulation.

In a fourth aspect of the fourteenth mode of the disclosure, the smoking substitute apparatus of the third aspect of the fourteenth mode of the disclosure may be part of a smoking substitute system. Accordingly, the fourth aspect of the fourteenth mode of the disclosure provides a smoking substitute system comprising: a smoking substitute apparatus according the third aspect of the fourteenth mode of the disclosure; and a mobile device, the mobile device being communicatively coupled to the controller of the smoking substitute apparatus; wherein the mobile device is configured to transmit a control signal to the controller; and wherein the controller is configured to control the stimulation signal based on the control signal received from the mobile device.

In this manner, a user may control the flavor simulation produced by the mouthpiece via the mobile device (e.g., via software installed on the mobile device). This may facilitate use of the smoking substitute apparatus, and enable easy and rapid change of the simulated flavor. This may also simplify construction of the smoking substitute apparatus, as it may avoid the need of including a selection mechanism for selecting a flavor directly on the smoking substitute apparatus.

The mobile device may be communicatively coupled to the controller via a wireless connection (e.g., Bluetooth, Wi-Fi) or via a wired connection (e.g., USB). The mobile device and controller being communicatively coupled may mean that the mobile device and controller are capable of exchanging data (e.g., transmitting data to, and receiving data from, one another).

The mobile device may be any suitable computing device, e.g., smartphone, tablet computer, laptop computer, etc. The mobile device may include software installed thereon for remotely controlling the smoking substitute apparatus. The mobile device may be configured to receive data from the smoking substitute apparatus (e.g., usage data), so that a user may monitor usage of their apparatus.

The control signal transmitted by the mobile device may include instructions which cause the controller to control the stimulation signal in a desired manner (e.g., to simulate a desired flavor). For example, the control signal may include an indication of parameters for the stimulation signal. Upon receipt of the control signal, the controller controls the stimulation signal based on the control signal. For example, the controller may generate a stimulation signal having parameters specified in the control signal received from the mobile device.

The mobile device may store sets of parameters of stimulation signals corresponding to various simulated flavors. To cause simulation of a desired flavor, the mobile device may then transmit a control signal include the parameters for the stimulation signal associated with the desired flavor. In this manner, parameters for the stimulation signals may be stored in the mobile device, rather than in the smoking substitute apparatus. In some cases, the mobile device may be configured to access an online library (e.g., in “the cloud”), which includes indications of stimulation signal parameters for various different flavors.

The mobile device may be further configured to generate a user interface enabling selection of a flavor; and, in response to a user selecting a flavor via the user interface, generate the control signal based on the flavor selected by the user. In this manner, a user may select the flavor simulated by the smoking substitute apparatus directly on the mobile device. This may enable the user to easily change the simulated flavor “on the fly”. The user interface generated by the mobile device may include a series of selectable flavor options. When the user selects one of the flavor options, the mobile device may transmit a corresponding control signal to the controller, so that a corresponding stimulation signal is generated to cause stimulation of the user's tongue via the one or more electrodes on the mouthpiece.

The mobile device may be further configured to perform a calibration procedure including: transmitting a test control signal to the controller to cause the controller to control the stimulation signal based on the test control signal; and receiving from a user, via a user interface on the mobile device, an indication of a flavor perceived by the user. Different users may respond differently to stimulation via the one or more electrodes. For instance, different users may experience different flavor sensations when a same stimulation signal is used, e.g., due to differences in how the different users perceive taste. Therefore, it may be beneficial to calibrate stimulation of a user's tongue for each user, to ensure accurate flavor simulation.

Thus, the mobile device may transmit a test control signal to the controller to stimulate the user's tongue. The test control signal may, for example, correspond to a test flavor. The user may then record, via the user interface on the mobile device, what flavor they experienced when their tongue was stimulated. The user interface may include multiple selectable options corresponding to possible flavors experienced by the user. The mobile device may record the user's response.

This procedure may be repeated for multiple different test control signals, with the user indicating each time via the user interface the flavor which they perceived. In this manner, it may be possible to build up a mapping between the different control signals and flavors experienced by the user. This mapping may then be used to when generating a control signal to simulate a desired flavor. As a result, flavors may be simulated more accurately for the user, as generation of the control signal may take into account how the user responded to previous simulations.

The mobile device may be further configured to adjust the test control signal transmitted to the controller, based on the indication received from the user. In this manner, the control signal may be adjusted in real-time based on the user's response. The control signal may be adjusted until the user indicates that a desired flavor is perceived by the user. This may enable fine-tuning of the flavor simulation, so that a desired flavor may be accurately simulated for that user.

According to a fifth aspect of the fourteenth mode of the disclosure, there is provided a method of simulating flavor delivery to a user of a smoking substitute apparatus, the smoking substitute apparatus having a mouthpiece with an outlet for conveying an aerosol generated by the smoking substitute apparatus to the user and one or more electrodes arranged to stimulate the user's tongue, the method comprising stimulating the user's tongue via the one or more electrodes to simulate a flavor. In this manner, flavor delivery may be simulated via the one or more electrodes when the user inhales an aerosol from the smoking substitute apparatus. The method of the fifth aspect of the fourteenth mode may be used with the smoking substitute apparatus, cartridge and/or mouthpiece of the previous aspects of the fourteenth mode of the disclosure. Features of the previous aspects of the fourteenth mode of the disclosure may be shared with the fifth aspect of the fourteenth mode of the disclosure, and are therefore not repeated.

The method may further comprise: receiving a flavor selection from the user of the smoking substitute system; controlling, based on the flavor selection, a stimulation signal provided to the one or more electrodes to simulate the selected flavor. In this manner, a user may select a flavor simulated by the smoking substitute system.

The method may further comprise performing a calibration procedure which includes the steps of: controlling the stimulation signal to simulate a test flavor; and receiving, from the user, an indication of a flavor perceived by the user. The calibration procedure may further include adjusting the stimulation signal based on the indication received from the user. The calibration procedure may be similar to the calibration procedure discussed above in relation to the smoking substitute system.

The method may further comprise steps described above in relation to the mobile device, the controller and/or the smoking substitute system.

The disclosure includes the combination of the aspects and preferred features of the fourteenth mode described except where such a combination is clearly impermissible or expressly avoided.

The skilled person will appreciate that except where mutually exclusive, a feature or parameter described in relation to any one of the above aspects of the fourteenth mode may be applied to any other aspect of the fourteenth mode. Furthermore, except where mutually exclusive, any feature or parameter of the fourteenth mode described herein may be applied to any aspect and/or combined with any other feature or parameter of the fourteenth mode described herein.

Fifteenth Mode: Presenting a Source of Flavorant to Fluid Flow Through a Smoking Substitute Device

At its most general, a fifteenth mode of the present disclosure relates to presenting a source of flavorant to fluid flow through a smoking substitute device to entrain flavorant therein.

Providing a flavorant in this way may provide more versatility in how flavor can be delivered to the user.

According to a first aspect of the fifteenth mode there is provided a smoking substitute system having a fluid inlet, a fluid outlet, and an aerosol generator operable to generate an aerosol from an aerosol precursor; the aerosol generator being arranged between the inlet and the outlet and in fluid communication with each such that fluid may be drawn from the inlet to the outlet via the aerosol generator; wherein the smoking substitute system further comprises a source of flavorant provided between the fluid inlet and the aerosol generator so as to be presented to a flow of fluid from said fluid inlet towards said aerosol generator, the source of flavorant being substantially thermally isolated from the aerosol generator, the flavorant comprising one or more substances that activate at least one of an olfactory receptor in a human nasal cavity; and a taste receptor in a human oral cavity, and wherein flow of fluid past or through said source of flavorant is effective to release flavorant from said source for entrainment in said fluid upstream of said aerosol generator.

Advantageously, the source of flavorant is thermally insulated.

Conveniently, the source of flavorant is thermally isolated from heat-generating components of the system. For example, the system may include a power source (such as a battery) for the aerosol generator, in which case the source of flavorant may be thermally isolated from the power source.

Optionally, the smoking substitute system comprises a flavorant reservoir wherein the flavorant is provided in the reservoir.

Conveniently, the flavorant is provided as a liquid.

Advantageously, said source of flavorant comprises a crystallized membrane.

Optionally, the source of flavorant is a flavored article comprising a substrate carrying said flavorant.

Conveniently, at least part of said substrate is impregnated with the flavorant.

Advantageously, at least part of the substrate is coated with the flavorant.

Optionally, the substrate is formed from a polymeric material.

Conveniently, the source of flavorant is a flavor pod comprising a container at least partially filled with the flavorant.

Advantageously, said source of flavorant is a removable flavor part of the smoking substitute device.

Optionally, said removable flavor part is a replaceable part of the smoking substitute device.

Conveniently, said removable flavor part is a consumable.

Advantageously, the smoking substitute system further comprises a source of aerosol precursor for said aerosol generator, the source of aerosol precursor being comprised within a removable precursor part of the smoking substitute system.

Optionally, said removable precursor part is a replaceable part.

Conveniently, said removable precursor part is a consumable.

Advantageously, said removable precursor part includes said source of flavorant.

Optionally, the source of flavorant is releasably attachable to said removable precursor part.

Conveniently, the aerosol precursor is substantially free of flavorant.

Advantageously, the aerosol precursor is a liquid.

Optionally, the aerosol precursor contains nicotine.

According to a second aspect of the fifteenth mode, a consumable may be provided comprising a source of flavorant for use with the smoking substitute device according to the first aspect of the fifteenth mode.

According to a third aspect of the fifteenth mode, a pack comprising a plurality of consumables according to the second aspect of the fifteenth mode may be provided.

The flavorant may include one or more volatile substances. The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed within the source of flavorant, or may be provided in isolated locations and/or varying concentrations.

The smoking substitute device may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol precursor (e.g., e-liquid) may be replenished by replacing a used consumable with an unused consumable.

Alternatively, the smoking substitute device may be a non-consumable apparatus (e.g., that is in the form of an open smoking substitute system). In such embodiments an aerosol precursor (e.g., e-liquid) of the system may be replenished by re-filling, e.g., a precursor reservoir of the smoking substitute apparatus with the aerosol precursor (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a precursor reservoir configured to store an aerosol precursor, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The precursor reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The precursor reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the precursor reservoir. For example, opposing ends of the wick may protrude into the precursor reservoir and a central portion (between the ends) may extend across the passage so as to be exposed to fluid flow in the passage. Thus, fluid may be drawn (e.g., by capillary action) along the wick, from the precursor reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the fifteenth mode described except where such a combination is clearly impermissible or expressly avoided.

The skilled person will appreciate that except where mutually exclusive, a feature or parameter described in relation to any one of the above aspects of the fifteenth mode may be applied to any other aspect of the fifteenth mode. Furthermore, except where mutually exclusive, any feature or parameter of the fifteenth mode described herein may be applied to any aspect and/or combined with any other feature or parameter of the fifteenth mode described herein.

Sixteenth Mode: A Flavor Delivery Article that Releases a Flavorant in the Presence of an Aerosol Generated from a Smoking Substitution Apparatus

At its most general, a sixteenth mode of the present disclosure relates to a flavor delivery article that releases a flavorant in the presence of an aerosol generated from a smoking substitution apparatus. This may allow the flavorant to be released when the user puffs on the smoking substitute apparatus. Therefore, this may allow a user to personalize and/or improve the vaping experience, without needing to add a flavorant to an aerosol former.

According to a first aspect of the sixteenth mode there is provided a flavor delivery article for use with a smoking substitute apparatus, comprising: a flavorant; wherein the flavor delivery article is configured to release flavorant upon contacting an aerosol generated from the smoking substitute apparatus.

The aerosol may be generated by heating an aerosol former, e.g., an e-liquid, at the smoking substitution apparatus. The e-liquid may, for example, comprise a base liquid and nicotine. The nicotine may comprise free base nicotine. The base liquid may include propylene glycol and/or vegetable glycerin. The e-liquid may be flavorless. That is, the e-liquid may not contain any flavorant and may consist solely of a base liquid of propylene glycol and/or vegetable glycerin and nicotine. Therefore, the aerosol may be flavorless.

The flavor delivery article may be applicable to a user's oral cavity, and wherein the flavor delivery article may be configured to release flavorant to the user's oral cavity when the user inhales the aerosol. For example, the flavor delivery article may be inserted and retained in the user's oral cavity when the user puffs on the smoking substitute apparatus.

The flavorant may be provided in solid, gel or liquid form. It may include menthol, licorice, chocolate, fruit flavor (including, e.g., citrus, cherry etc.), vanilla, spice (e.g., ginger, cinnamon) and tobacco flavor. The flavorant may modify a flavor of saliva and/or aerosol upon contacting or mixing with the saliva and/or aerosol in the user's oral cavity.

Optionally, the flavor delivery article comprises a barrier for enclosing the flavorant, wherein the barrier is configured to disintegrate upon contacting the aerosol thereby releases the flavorant enclosed therein. In use the flavor delivery article may be received in a user's oral cavity and may activate upon contacting the aerosol generated by the smoking substitute apparatus. In other words, the barrier may remain intact in the absence of the aerosol. That is, enclosing the flavorant the barrier may inhibit or at least reduce contact between the flavorant and saliva in the user's oral cavity. For example, the barrier may remain intact, e.g., it may not disintegrate or soften, or at least it does not immediately disintegrate, once the flavor delivery article is received in a user's oral cavity and in contact with saliva. In other words, the barrier may shield the flavorant from moisture. Such arrangement may prolong the longevity of flavor delivery article because the barrier is not susceptible to moisture during storage. Advantageously, the barrier may allow controlled release of the flavorant by interacting the flavor deliver article with the aerosol in the user's oral cavity and/or in the passage of a smoking substitute apparatus. For example, it may reduce the likelihood of flavorant consumption by an unauthorized person or minors. The flavorant may only be released when the user puffs on the smoking substitute apparatus to draw out an aerosol.

Optionally, the flavor delivery article is provided as part of a smoking substitute apparatus comprising; a mouthpiece comprising an outlet; a passage for conveying an aerosol generated by an aerosol generator to the outlet, the aerosol generator being positioned in the passage; wherein the flavor delivery article is positioned in the passage between the aerosol generator and the outlet.

Flavorants have commonly only ever been a constituent ingredient of the e-liquid in a smoking substitute apparatus. Heating the flavorants in the e-liquid is now thought to generate potentially harmful toxins. By removing the flavorants from the e-liquid and instead placing them such that they do not pass through the heater, then not only is there an advantage of better flavor delivery in the safer format of an aroma, but there is also increased stability and control in the absence of direct heater contact.

When the flavor delivery article is disposed in the passage, the flavor delivery article may be provided on a substrate in the passage. That is to say, a substrate that is not the passage is provided in the space defined by the passage and the flavor delivery article is on that substrate.

Optionally, the substrate may be held in the passage by an interference fit. For instance, it may be tapered in fit. This prevents the substrate from moving in or falling out of the passage as the smoking substitute apparatus is handled or used. The substrate may be, for example, about 30 mm long, about 10 mm wide and about 5 mm deep. The substrate may protrude from the passage and beyond the outlet such that, when in use, the substrate may directly contact the user's oral cavity and/or tongue.

Optionally, the substrate is removable and/or replaceable from its position in the passage. It may, for example, be removed by a user through the outlet in the mouthpiece. Where a depleted substrate is removed it may be possible to recharge the substrate with more flavor delivery articles. It may be that a tool is required for the user to remove the substrate from the passage so that the chances of accidental removal of the substrate are further minimized.

Optionally, the passage has one or more grooves or slots that correspond to the shape of the substrate or protrusions of the substrate to hold the substrate in position. For example, four linear grooves may be provided along the length of the passage to receive the tips of a substrate having an X-shaped cross section. The substrate may comprise an adhesive to adhere the substrate to the passage.

Optionally, the substrate may have a uniform cross-section along its length. Alternatively, the cross-section profile may be different along the length of the substrate, such as two or three different profiles. The cross-section profile may be open, such as flat, V-shaped, e-shaped. Alternatively, the cross-section profile may be closed, such as cylindrical, star shaped, square shaped of polygonal.

Optionally, the substrate material comprises one or more materials selected from paper, plastics, ceramics, metals, polymeric film and elastomeric material. For example, the substrate may comprise one or more of cellulose, hemicellulose, lignin, bagasse, paper foam, bamboo, rice paper, flax seed, pulp paper or cotton cellulose. Advantages of bagasse are that it is curbside recyclable, compostable, utilizes a waste stream and requires less chemical processing. Advantages of paper foam are that it is easily molded, has a low carbon footprint, required less water in production and is lightweight. Advantages of bamboo are that it is a renewable resource and recyclable. Advantages of rice paper are that it is fibrous and strong. Advantages of flax seed are that it is thin but strong and durable. Advantages of pulp paper are that it has a low raw material cost and low energy production cost. Advantages of cotton cellulose are that it is durable, often high in purity, and hollow-like.

Optionally, the substrate may be porous, corrugated and/or textured. This increases the surface area available for the vapor to pass over, release and be entrained with the flavorant.

Alternatively, when the flavor delivery article is disposed in the passage, the flavor delivery article may be provided directly on the surface of the passage. That is to say, the surface of the passage is the substrate on which the flavor delivery article is provided. Common passage materials include polymers (e.g., polypropylene or polyether ether ketone), ceramic (e.g., glass) or metallic (e.g., stainless steel) surface and so the flavor delivery article may be configured to specifically adhere to these materials.

When the flavor delivery article is provided directly on the surface of the passage, the surface of the passage may be textured. That is to say, the wall of the passage may not be smooth so that adherence of the flavor delivery article to the passage is improved, particularly when the device is being used.

When the flavor delivery article is provided directly on a substrate in, or on the surface of, the passage, a plurality of flavor delivery articles having different flavorants may be positioned thereon. The plurality of different flavor delivery articles may be positioned in a number of ‘bands’ or ‘zones’ on the substrate or surface of the passage. Alternatively, or simultaneously, multiple layers of different flavor delivery articles may be provided on top of each other on the substrate or surface of the passage. This allows for combinations and/or variation over time in the delivery of the flavor during the use of the smoking substitute apparatus.

Optionally, the flavor delivery article may be applied to the substrate or surface of the passage by methods such as spraying, rolling, brushing and dipping.

Optionally, the flavor delivery article comprises one or more of benzyl alcohol, cinnamyl alcohol, 3methyl benzyl alcohol and β-phenethyl alcohol. Using these compounds as flavorants, flavor enhancers and/or bacteriostatics is advantageous because they each have a boiling point greater than 205° C. such that they have a low volatility and survive long term storage before use. The flavor delivery article may comprise other preservatives, such as ethanol.

Optionally, the barrier encapsulates the flavorant. For example, the barrier may be a hydrogel coated onto a surface of the flavorant, or it may be a shell with substantial mechanical strength containing a body of flavorant. Either way, the barrier may fully contain the flavorant and may form a capsule, microcapsule or nanocapsule. The barrier may comprise a flexible and/or elastic wall or it may comprise a rigid wall. The barrier may be configured to disintegrate as it comes into contact with the aerosol. In other words, the barrier may break down in the presence of the aerosol. Advantageously, such arrangement may ensure the flavorant is completely enclosed in the barrier.

Optionally, at least a part of the flavorant is embedded in the barrier. For example, the flavorant may be mixed or blended into a barrier material, and therefore at least a portion of the flavorant may be embedded in the barrier. More specifically, a significant portion of the flavorant may be embedded and be enclosed in the barrier. On the other hand, a small part, or an insignificant part of the flavorant, may be deposited on a surface of the barrier. Advantageously, embedding the flavorant in the barrier may allow the flavorant to gradually release to the user's oral cavity when the barrier progressively disintegrates.

Optionally, the flavor delivery article comprises a porous structure. For example, the porous structure may form a sponge that provides fluid passages for the aerosol. Advantageously, the porous structure may increase a contacting area between the aerosol and the barrier, and thus it may improve the efficiency of flavorant release because the barrier may disintegrate in a more efficient manner.

The flavor delivery article may take any shape or form. For example, the flavor delivery article may be a capsule, or a microcapsule, having a spherical shell for encapsulating the flavorant. It may be a liposome, film former or adsorbent. Alternatively, the flavor delivery article may have a planar form, for example a sheet or a composite sheet, and may form from a planar flavorant sandwiched in between two planar barrier elements. The flavor delivery article may be a cylindrical or polygonal rod. The flavor delivery article may take the form of a block.

Once the barrier disintegrates in the user's oral cavity and exposing the flavorant to saliva, the flavorant may dissolve in the saliva. Therefore optionally, the flavorant comprises a water soluble flavorant. The flavorant may comprise a freeze-dried flavorant. For example, the flavorant may have a strong hydrophilic property. Advantageously, this may allow the flavorant to be released from the disintegrated barrier more readily, thus allowing the flavorant to be promptly delivered to taste receptors at the user's tongue.

Optionally, the barrier comprises one or more of chitosan, hyaluronic acid, dextran, poly-acrylamide, polyacrylic acid, guar gum succinate, kappa-carrageenan, poly (vinyl alcohol). Optionally, the barrier is formed from a hydrogel composite comprising chitosan. For example, the barrier may comprise chitosan with a degree of deacetylation (DDT %) ranging from 75% to 99%. The barrier may comprise chitosan with a degree of deacetylation (DDT %) of any one of 75%, 80%, 85%, 90%, 95% and 99%. Further, the barrier may comprise chitosan at a concentration between 0.1% w/w and 20% w/w. The barrier may comprise chitosan at a concentration of any one of 0.1% w/w, 0.5% w/w, 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 16% w/w, 17% w/w, 18% w/w, 19% w/w and 20% w/w. The barrier may comprise a polymer or a cyclic oligosaccharide.

Optionally, the flavor delivery article is disposed in the passage, the barrier is configured such that the flavor delivery article releases the flavorant for the use lifetime of a consumable smoking substitute apparatus, such as for a period of 24 hours, 48 hours, or 72 hours. This time may be determined by how long it takes the user to deplete the e-liquid in the reservoir. The barrier should remain intact after manufacture during shipping, storage and the shelf-life before consumer usage to retain all of the flavorant. The use lifetime may begin, for example, 6 to 9 months, or more, after the substrate was produced.

Optionally, the barrier is configured to disintegrate in response to a change in pH value induced by the aerosol. For example, chitosan in a hydrogel composite may swell or shrink depending on the pH value of its surroundings, e.g., the pH of saliva and/or aerosol in the user's oral cavity. Therefore advantageously, exposing the barrier to the aerosol may increase the alkalinity (e.g., increasing a pH value) of the barrier and thereby it may cause the barrier to disintegrate.

Optionally, the aerosol comprises a freebase nicotine for effecting the change in pH value. More specifically, an increase in pH value in the user's oral cavity may be caused by freebase nicotine contained in the aerosol. Advantageously, varying the pH with the use of the freebase nicotine may allow the change in pH to be precisely controlled. Advantageously, the freebase nicotine may be precisely dosed in the aerosol former, and therefore the nicotine content in each of the puff may be consistent.

Optionally, wherein the barrier is configured to disintegrate at a pH value at or above 7.5, or to disintegrate at a pH value at or above 7.8. More specifically, below a pH value of 7.5 the barrier may remain intact, e.g., it does not disintegrate instantaneously. Advantageously, this may allow the flavor delivery article to remain stable upon exposing to saliva in a user's oral cavity. This is because the pH value of saliva is usually at 7.4, and therefore exposing to the saliva is insufficient to cause the barrier to promptly disintegrate.

Optionally, the rate of barrier disintegration increases with pH value. Optionally, the barrier is configured to promptly disintegrate at a pH value at or above 7.8. For example, the aerosol containing freebase nicotine may have a pH value of 7.8, or above 7.8. Therefore, exposing the barrier to an aerosol that contains freebase nicotine causes the barrier to rapidly disintegrate. Advantageously, this may allow the flavorant to be promptly released as the user puffs on the smoking substitute apparatus.

Optionally, the barrier is configured to disintegrate in response to the presence of moisture or a predetermined chemical in the aerosol. By using a specific compound in the barrier that only disintegrates in response to a predetermined chemical in the aerosol it is possible to reduce the likelihood that the flavorant will be released under any other circumstances.

Optionally, the barrier is configured to disintegrate in response to a change in temperature induced by the aerosol. For example, the barrier may break up upon being cooled or heated by the aerosol. Optionally, is configured to disintegrate at a temperature above 37° C., or to disintegrate at a temperature above 40° C. That is, the flavor delivery article may remain stable, e.g., not disintegrating instantaneously, in a user's oral cavity until it is heated by a stream of aerosol having a temperature exceeding 37° C. For example, the aerosol generated by a heated tobacco apparatus, e.g., a heat not burn apparatus, may be exhausted at a temperature above 40° C., and therefore sufficient to cause the barrier to disintegrate rapidly. Advantageously, this may allow the flavor delivery article to remain stable at room temperature during storage. The barrier may comprise any one or more of poloxamers, cellulose and xyloglucan.

Optionally, the flavorant comprises a solid flavorant. Optionally, the flavorant comprises a freeze dried flavorant, wherein said freeze dried flavorant is configured to activate upon exposing to moisture. More specifically, when exposed to the saliva in the user's oral cavity, the freeze dried flavorant may rehydrate and thereby it may form a reconstituted flavorant. Advantageously, this may allow the flavorant to activate only in the presence of moisture, e.g., saliva in the user's oral cavity.

Optionally, the flavorant comprises a liquid flavorant. More specially, the liquid flavorant may be configured to flow or leak out of the disintegrated barrier. Advantageously, this may enable the flavorant to be delivered directly to the user's taste receptors without the need of rehydration.

Optionally, the flavorant comprises a gaseous flavorant. Optionally, said gaseous flavorant is configured to be absorbed into the saliva to form a reconstituted flavorant. Advantageously, the use of a gaseous flavorant allows the flavorant to be delivered olfactorily, in additional to the taste receptors.

Optionally, the flavorant comprises a mixture of any of solid flavorant, liquid flavorant, and gaseous flavorant. For example, the flavorant may comprise a solid/liquid suspension. Optionally, the solid flavorant, liquid flavorant, and/or gaseous flavorant may each be released at a different rate. Advantageously, this may allow the profile of flavorant release to be tailored to a user's needs. For example, the flavorant may comprise a mixture of solid flavorant and a liquid flavorant carrying different flavors. For example, upon barrier disintegration, the liquid flavorant may be promptly released, such that the user may immediately perceive a first flavor. Thereafter, the user may perceive a second flavor which is caused by a more gradual release of a solid flavorant. That is, such arrangement allows different flavorant to be released sequentially.

Optionally, the flavor delivery article is in the form of any one of a powder, a solid-liquid suspension, a paste and a coating on a substrate soluble or insoluble in saliva. For example, the flavor delivery article may be capsules or microcapsules. Optionally, the flavor delivery article may be a free-flowing powder.

This may allow the flavor delivery article to be dispensed with a dispenser.

Optionally, the flavor delivery article is coated or adhered onto a substrate. The substrate may be a card formed form an absorbent material such as blotting paper or a non-absorbent material such as plastic.

The substrate may be insoluble in saliva such that it does not interfere with the release of flavorant. Alternatively, the substrate may be soluble in saliva in that additional flavorant in the substrate may be released as the substrate dissolves. For example, the substrate may be a candy, such as hard-boiled sweets and lollipops, having the flavor delivery article coated on its surface or embedded therein. In use, the additional flavorant may be readily released as the substrate dissolves in the saliva, wherein the flavor delivery article may not activate until it is in contact with the aerosol.

Optionally, the flavor delivery article forms a compressed tablet. That is, flavor delivery article may comprise an aggregate of capsules or microcapsules. The compressed tablet may form from multiple layers of microcapsules and therefore such arrangement may promote sequential release of flavorant. More specifically, the core of compressed tablet may only come into contact with the aerosol and thereby releases the flavorant, once the peripheral layers of microcapsules have been disintegrated or eroded away. Advantageously, this may allow a prolonged release of flavorant, e.g., flavorant is released gradually and sequentially through different layers of the microcapsules. It may also allow sequential release of different flavors from different layers of microcapsules.

Optionally, the flavor delivery article comprises a solid-liquid suspension. More specifically, the flavor delivery article may comprise one or more flavor delivery articles, or microcapsules, that are suspended in a carrier liquid. The barrier of the flavor delivery article may be insoluble in the carrier liquid. The flavor delivery article may be neutrally buoyant in the carrier liquid. Advantageously, the solid-liquid suspension may allow the flavor delivery articles to be dosed more accurately and/or delivered more effectively into the user's oral cavity. For example, the suspension may be dispensed into a user's oral cavity using a spray pump such that each pump stroke dispenses a predetermined amount of suspension in the user's oral cavity. The suspension may also be dispensed using a dripper that dispenses a predetermined amount of suspension into a user's oral cavity.

The flavor delivery article may comprise a paste that comprises the one or more flavor delivery articles being suspended in a viscous carrier fluid. More specifically, the flavor delivery article may be in a paste form and does not flow under gravity in room temperature. In use, a user may apply or spread the paste onto a surface of the oral cavity. Advantageously, such arrangement allows the flavor delivery article to adhere onto a surface of the oral cavity, and thereby prolonging the release of flavorant.

The carrier liquid may comprise colorant. The carrier liquid may comprise a flavorant or it may be unflavored. The carrier liquid may comprise the same flavorant as that enclosed by the barrier, or it may comprise a flavorant that is different to that enclosed by the barrier.

According to a second aspect of the sixteenth mode there is provided a smoking substitute kit, comprising a flavor delivery article and a smoking substitute apparatus, wherein the smoking substitute apparatus is configured to generate an aerosol for contacting the flavor delivery article and thereby causes the release of a flavorant from the flavor delivery article.

According to a third aspect of the sixteenth mode there is provided a dispenser for dispensing the flavor delivery article, wherein the dispenser is configured to dispense the flavor delivery article to a user's oral cavity.

Optionally, the dispenser is configured to dispense a predetermined quantity of flavor delivery article.

According to a further aspect of the sixteenth mode there is provided a method of producing a flavor delivery article for a smoking substitute apparatus, comprising the steps of:

preparing a flavorant; and applying the flavorant to the flavor delivery article.

Optionally, the method comprises encapsulating or embedding the flavorant in a barrier.

For example, the method may comprise producing the flavorant and subsequently drying the flavorant to produce a solid flavorant, e.g., a flavor cake. The drying process may comprise a freeze-drying process. Advantageously, the use of freeze-drying process may reduce or minimize a high temperature the flavorant may otherwise expose to in other drying processes. The freeze-drying process may take place in molds in order to form the solid flavorant of a desired shape, or it can be carried out to produce a free-flowing powder flavorant.

The method may further comprise forming a barrier by mixing chitosan, along with other excipients, such as xanthan gum, water, and optionally propylene glycol and/or vegetable glycerin. The mixture may be dried to produce a barrier in the form of a barrier gel, or a hydrogel.

The method may further comprise inserting the flavorant into barrier gel in a mold, and thereby encapsulates the flavorant in the barrier. For example, the method may comprise: i) layering the gel in a mold; ii) inserting the flavorant; and iii) rolling the gel to form a sphere or a capsule.

The method may further comprise sandwiching the flavorant between layers of barrier gel in a mold, and thereby encapsulates the flavorant in the barrier. Advantageously, this may allow the flavor delivery article to be formed in a planar shape.

The method may further comprise injecting a flavorant in the form of a liquid, a gel, or a paste into the barrier of the flavor delivery article, and thereby encapsulates the flavorant.

The method may further comprise mixing or blending the flavorant with a barrier material, such as chitosan cross-linked with glutaraldehyde. The method may further comprise freeze-drying the mixture to from the flavor delivery article. Advantageously, this may form a flavor delivery article with a porous structure.

According to a further aspect of the sixteenth mode there is provided a method of delivering flavor to a user of a smoking substitute apparatus, the method comprising:

applying a flavor delivery article to a user's oral cavity; inhaling, by the user, an aerosol generated by a smoking substitute apparatus;

wherein the flavor delivery article is configured to release flavorant upon contacting the aerosol generated by the smoking substitute apparatus.

Alternatively, the application step may involve applying a flavor delivery article to the surface of the passage (e.g., on the surface of a wall of the passage) or on a substrate disposed in the passage.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Where the smoking substitute apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting.

Thus, the smoking substitute apparatus may comprise one or more engagement portions for engaging with a main body. In this way, one end of the smoking substitute apparatus may be coupled with the main body, whilst an opposing end of the smoking substitute apparatus may define a mouthpiece of the smoking substitute system.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorant and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage. The flavor delivery article may be disposed in the passage, for example on the surface of the passage (e.g., on the surface of a wall of the passage) or on a substrate that is disposed in the passage.

The smoking substitute apparatus may comprise an aerosol generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the sixteenth mode described except where such a combination is clearly impermissible or expressly avoided.

The skilled person will appreciate that except where mutually exclusive, a feature or parameter described in relation to any one of the above aspects of the sixteenth mode may be applied to any other aspect of the sixteenth mode. Furthermore, except where mutually exclusive, any feature or parameter of the sixteenth mode described herein may be applied to any aspect and/or combined with any other feature or parameter of the sixteenth mode described herein.

Seventeenth Mode: A Flavored Article which can be Placed Over at Least Part of the Fluid Inlet

At its most general, a seventeenth mode of the present disclosure relates to a flavored article which can be placed over at least part of the fluid inlet of a smoking substitute system such that a flavorant may be provided to a user.

Providing a flavorant in this way may allow for increased versatility in the provision of flavor to the user of a smoking substitute system.

According to a first aspect of the seventeenth mode of the present disclosure, there is provided a smoking substitute system comprising a smoking substitute device and a flavored article, the smoking substitute device having a fluid inlet, a fluid outlet, and an aerosol generator arranged between the inlet and the outlet and in fluid communication with both the inlet and the outlet, the aerosol generator being operable to generate an aerosol from an aerosol precursor; wherein the flavored article comprises a substrate carrying a flavorant, the flavored article being arranged externally of the device such that at least a part of the substrate extends at least partially across the inlet of the device such that fluid drawn into the device via the inlet passes through the flavored article to thereby release flavorant from the substrate for entrainment in fluid entering the device through the fluid inlet, the flavorant comprising one or more substances effective to activate at least one of an olfactory receptor in a human nasal cavity; and a taste receptor in a human oral cavity.

Optionally, the flavored article is substantially annular and extends around at least part of the smoking substitute device.

Advantageously, at least part of the substrate has a porous structure.

Conveniently, at least part of the substrate is formed from an air-permeable material.

Optionally, at least part of the substrate is formed from a foamed material.

Advantageously, at least part of the substrate is formed from polymeric material.

Conveniently, the polymeric material is silicone.

Optionally, at least part of the substrate is formed from a deformable material.

Advantageously, at least part of the substrate is formed from a resiliently deformable material.

Conveniently, the deformable material is a stretchable material.

Optionally, the deformable material is a viscoelastic material.

Advantageously, the deformable material is an elastomeric material.

Conveniently, at least part of the substrate is substantially rigid.

Optionally, at least part of the substrate is impregnated with the flavorant.

Advantageously, at least part of the substrate is coated with the flavorant.

Conveniently, the flavored article is a removable part of the smoking substitute system.

Optionally, the flavored article is a replaceable part of the smoking substitute system.

Advantageously, the flavored article is a consumable.

Conveniently, a part of the smoking substitute system comprising a source of aerosol precursor for the aerosol generator is a removable component of the smoking substitute system.

Optionally, said removable component is replaceable.

Advantageously, said replaceable component is a consumable.

Conveniently, the smoking substitute system includes a mouthpiece comprising said outlet.

Optionally, at least part of the flavored article is arranged proximate to said mouthpiece.

Advantageously, the aerosol precursor is substantially free of flavorant.

Conveniently, the aerosol precursor is a liquid.

Optionally, the aerosol precursor contains nicotine.

According to a second aspect of the seventeenth mode of the present disclosure, there is provided a flavored article which is configured for use with the smoking substitute system according to the first aspect of the seventeenth mode.

Optionally, a pack may be provided comprising a plurality of said flavored articles.

The flavorant may include one or more volatile substances. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed throughout the flavored article, or may be provided in isolated locations and/or varying concentrations.

The smoking substitute apparatus (smoking substitute device) may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol precursor (e.g., e-liquid) may be replenished by replacing a used consumable with an unused consumable.

Alternatively, the smoking substitute device may be a non-consumable apparatus (e.g., that is in the form of an open smoking substitute system). In such embodiments an aerosol precursor (e.g., e-liquid) of the system may be replenished by re-filling, e.g., a reservoir of the smoking substitute device with the aerosol precursor (rather than replacing a consumable component of the apparatus).

In light of this, it should be appreciated that some of the features described herein as being part of the smoking substitute device may alternatively form part of a main body for engagement with the smoking substitute device (i.e., when the smoking substitute device is in the form of a consumable).

Where the smoking substitute device is in the form of a consumable, the main body and the consumable may be configured to be physically coupled together. For example, the consumable may be at least partially received in a recess of the main body (or vice-versa), such that there is an interference fit between the main body and the consumable. Alternatively, the main body and the consumable may be physically coupled together by screwing one onto the other, or through a bayonet fitting.

Thus, the smoking substitute device may comprise one or more engagement portions for engaging with a main body. In this way, one end of the smoking substitute device may be coupled with the main body, whilst an opposing end of the smoking substitute device may define a mouthpiece of the smoking substitute system.

The smoking substitute device may comprise a reservoir configured to store an aerosol precursor, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute device may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute device may comprise a passage for fluid flow therethrough. The passage may extend through (at least a portion of) the smoking substitute device, between openings that define an inlet and an outlet of the passage. The outlet may be at a mouthpiece of the smoking substitute device. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute device may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute device (or main body engaged with the smoking substitute device) may comprise a power source. The power source may be electrically connected (or connectable) to a heater of the smoking substitute device (e.g., when engaged with the main body). The power source may be a battery (e.g., a rechargeable battery). A connector in the form of, e.g., a USB port may be provided for recharging this battery.

When the smoking substitute device is in the form of a consumable, the smoking substitute device may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable.

The electrical interface may also be used to identify the smoking substitute device (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute device is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute device or main body may comprise a controller, which may include a microprocessor. The controller may be configured to control the supply of power from the power source to the heater of the smoking substitute device (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute device may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute device may be configured to be activated when a puff is detected by the puff sensor. When the smoking substitute device is in the form of a consumable, the puff sensor may form part of the consumable or the main body.

The disclosure includes the combination of the aspects and preferred features of the seventeenth mode described except where such a combination is clearly impermissible or expressly avoided.

The skilled person will appreciate that except where mutually exclusive, a feature or parameter described in relation to any one of the above aspects of the seventeenth mode may be applied to any other aspect of the seventeenth mode. Furthermore, except where mutually exclusive, any feature or parameter of the seventeenth mode described herein may be applied to any aspect and/or combined with any other feature or parameter of the seventeenth mode described herein.

Eighteenth Mode: A Smoking Substitute Apparatus with a Flavorant on an Outside Surface

When there is no flavorant present in the e-liquid, or an amount of flavorant is present which delivers a weak and underwhelming taste sensation to consumers, it is desirable to supplement this with flavorant which is located elsewhere on or in the smoking substitute apparatus. Broadly speaking, an eighteenth mode of the present application relates to disclosures in which a smoking substitute apparatus is provided with a flavorant somewhere on its outside surface, such that when a consumer puffs on the smoking substitute device, they are also able to smell and/or taste the flavorant, to provide them with a more pleasurable “smoking” experience. This application also relates to kits of parts including components allowing a user to apply a flavorant of choice to the outside surface of their smoking substitute apparatus, in order to achieve the same effect.

A first aspect of the eighteenth mode of the present disclosure provides a smoking substitute apparatus having a flavorant-containing element printed onto an outer surface thereof. In some embodiments of the first aspect of the eighteenth mode of the disclosure the flavorant-containing element may be colorless.

However, in preferred embodiments of the first aspect of the eighteenth mode of the disclosure, the flavorant containing element may comprise a flavored and/or scented ink. In embodiments in which the flavorant-containing element contains ink, the ease with which a user may locate the flavorant-containing element is improved. The flavorant-containing element may be printed onto the outer surface of the smoking substitute apparatus using pad printing or tampo printing, a method in which a 2D image is transferred to a 3D surface.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable. Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Throughout this application, the term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances. The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations. The flavorant may be configured to release aroma or flavor in response to one or more of heat, pressure, friction and moisture.

Where the smoking substitute apparatus is in the form of a consumable, the consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

A second aspect of the eighteenth mode of the present disclosure provides a smoking substitute apparatus having a flavorant-containing element on its outer surface, the flavorant-containing element comprising a scented and/or flavored ink. Like the first aspect of the eighteenth mode of the present disclosure, the scented and/or flavored ink of the second aspect of the eighteenth mode of the disclosure may be applied to the smoking substitute apparatus by printing, e.g., by pad printing or tampo printing, as discussed above. Alternatively, the flavorant-containing element may be in the form of a sticker which is applied to the smoking substitute apparatus. The flavorant-containing element may be applied by spraying. The flavorant-containing element may be applied in a process wherein the regions of the smoking substitute apparatus to be coated are given a negative charge, and the ink is given a positive charge which is then attracted to the negatively charged regions of the smoking substitute apparatus to form the flavorant containing element. As will be appreciated, the charges in such an embodiment may be reversed. In an alternative embodiment, the flavorant-containing element may be applied to the inner surface of a mold which is used to form at least a portion of the smoking substitute apparatus by injection molding, such that when a heated precursor composition is injected into the mold, the flavorant-containing element is picked up by the heated precursor composition and is thereby affixed to an outer surface of that portion of the smoking substitute apparatus. The skilled person appreciates that there are several other different ways in which the flavorant-containing element may be applied to the surface of the smoking substitute apparatus.

For the purposes of the present application, the “top” side/surface of a smoking substitute apparatus is defined as the side/surface which faces upwards during normal use of the smoking substitute apparatus. Generally, when the smoking substitute apparatus is in the form of a consumable, when the consumable is engaged with a main body as described above, the top side of the consumable is aligned with the top side of the main body. In embodiments of the first aspect of the eighteenth mode of the disclosure in which the flavorant-containing element is or includes a scented element, it is preferred that the scented element be located on a top surface of the smoking substitute apparatus, enabling the user to smell the scented element as the same time as they are using the device. In embodiments in which the flavorant containing element is or includes a flavored element, it is preferred that the flavored element be located on a bottom surface of the smoking substitute apparatus, enabling the user's tongue to contact the flavored element during use, so that the taste of the flavored element is able to complement the taste of, e.g., the vaporized-liquid.

In some embodiments of the eighteenth mode, the flavorant-containing element may be both flavored and scented, and optionally the smoking substitute apparatus may include a plurality of such elements (i.e., a plurality of elements, each of which is both flavored and scented). In other embodiments, the smoking substitute apparatus may separately include a flavored element and a scented element, optionally located on the bottom surface and the top surface of the smoking substitute apparatus respectively.

In some embodiments of the eighteenth mode, the flavorant-containing element may include a first flavorant layer and a second flavorant layer. The first flavorant layer may be a different flavor from the second flavorant layer. In this way, as the user continues to lick the flavorant-containing element during vaping, they are able to taste a succession of different flavors. In some embodiments there may be more than two flavorant layers, each having different flavors.

In some embodiments of the eighteenth mode, the flavorant-containing element may be in the form of a visual graphic. In embodiments in which the flavorant-containing element is in the form of a visual graphic, it is preferably configured to indicate visually how much flavorant is remaining in the flavorant-containing element. In this way, a user of the smoking substitute apparatus is able to identify when the flavorant has run out, and needs, e.g., to be replaced. In one such embodiment, the flavorant-containing element may be configured to fade over time, wherein the extent to which the flavorant-containing element has faded provides the visual indication of the amount of flavorant is remaining in the flavorant-containing element. In other embodiments, the visual graphic may be configured to change color as the flavorant is volatilized or consumed by the user. For example, the graphic may include an ink which is configured to change color as the flavorant component of that ink is released.

Alternatively, the visual graphic may include a flavorant-containing layer and an ink layer, the flavorant-containing layer on top of the ink layer such that as the flavorant-containing layer evaporates or is consumed by the user, the ink layer is exposed.

In some embodiments of the eighteenth mode, the flavorant-containing element may comprise a thermochromic ink, or a smart polymer which is configured to change color in response to the application of heat.

Other Features of the Smoking Substitute Apparatus of the Eighteenth Mode

The above description relates primarily of features of the eighteenth mode of the present disclosure which relate in some manner to the delivery of flavorant to a user. The optional features set out below are applicable to all four of the aspects of the eighteenth mode of the disclosure which are discussed above.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage. This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable. The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the eighteenth mode described except where such a combination is clearly impermissible or expressly avoided.

The skilled person will appreciate that except where mutually exclusive, a feature or parameter described in relation to any one of the above aspects of the eighteenth mode may be applied to any other aspect of the eighteenth mode. Furthermore, except where mutually exclusive, any feature or parameter of the eighteenth mode described herein may be applied to any aspect and/or combined with any other feature or parameter of the eighteenth mode described herein.

Nineteenth Mode: A Smoking Substitute Apparatuses Configured in Some Way to Receive a Flavorant

When there is no flavorant present in the e-liquid, or an amount of flavorant is present which delivers a weak and underwhelming taste sensation to consumers, it is desirable to supplement this with flavorant which is located elsewhere on or in the smoking substitute apparatus. Broadly speaking, a nineteenth mode of the present disclosure relates to smoking substitute apparatuses, which are configured in some way to receive a flavorant, so that the user is able to experience flavor at the same time as inhaling the flavorless e-liquid, or vapor from the heated tobacco consumable. This disclosure also relates to kits of parts including components allowing a user to apply a flavorant of choice to the outside surface of their smoking substitute apparatus, in order to achieve the same effect.

A first aspect of the nineteenth mode of the disclosure, accordingly, provides a smoking substitute apparatus having a flavorant-receiving portion which is configured to receive a flavorant from a flavorant applicator. Herein, the term “flavorant-receiving portion” should be understood to mean a part of the smoking substitute apparatus which is adapted in some way to receive flavorant from the applicator. It will be appreciated by the skilled person that the specific features of the flavorant-receiving portion will differ depending on the type of flavorant-receiving portion. However, in preferred embodiments, the flavorant-receiving portion is located at a mouthpiece of the smoking substitute apparatus. In this way, when the flavorant is applied using a flavorant applicator, it will then be located on a part of the smoking substitute apparatus which is close to a user's nose and/or mouth, in use, allowing the most efficient delivery of the flavor. For the same reason, it is preferred that the flavorant-receiving portion is located on a top surface of the smoking substitute apparatus.

The flavorant-receiving portion may be configured to receive a liquid flavorant, such as a scented and/or flavored ink. In preferred embodiments, the flavorant-receiving portion may include a porous material which is configured to absorb a liquid flavorant. The porous material may include one or more of a porous polymer, activated carbon, and wood. The porous material may include a woven fabric or a non-woven fabric. Use of a porous material in this manner improves the capacity of the flavorant-receiving portion, since the liquid flavorant may be absorbed into it, rather than merely resting on the surface of the smoking substitute apparatus. In some embodiments, the liquid flavorant may be in the form of a semi-solid, a gel, a suspension, or a solid-liquid colloid. In some embodiments, the flavorant-receiving portion may be configured to receive a first flavorant layer and a second flavorant layer, wherein the second flavorant layer is located above the first flavorant layer. Preferably, in such embodiments, the first flavorant layer is less viscous than the second flavorant layer. For example, the first flavorant layer may be a liquid, and the second flavorant layer may be in the form of a semi-solid, a gel, a suspension, or a solid-liquid colloid. In this way, the more viscous second flavorant layer is able to contain the less viscous first flavorant layer until the user inhales on the smoking substitute apparatus, releasing the first flavorant layer. The first flavorant layer may be a different flavor from the second flavorant layer.

In some embodiments, the flavorant-receiving portion may be configured to cause the liquid flavorant applied thereto to spread out. In other words, the flavorant-receiving portion may be configured to promote wetting of the surface of the smoking substitute apparatus, in order to ensure coverage over a larger area of the smoking substitute apparatus. In this way, the flavorant is better able to stick of the surface of the smoking substitute apparatus, rather than settling in a large drop. As well as by using a porous material, this spreading out effect may be achieved by the flavorant-receiving portion including a plurality of microchannels or a textured surface, e.g., sandpaper. Spreading out of flavorant also increases the surface area, for improved volatilization. The flavorant-receiving portion may include linear grooves, striations or a coarse membrane.

The flavorant-receiving portion may include a recess which is configured to receive a droplet of flavorant. In preferred such embodiments, the recess is configured to retain the droplet therein against gravity, i.e., the recess is shaped in a manner which prevents the droplet from falling out. This may be achieved by providing a recess which is configured to retain the droplet by means of the surface tension of the liquid flavorant. In some embodiments, in order to better retain the liquid flavorant in the recess, a portion of an inner surface of the recess may be porous or absorbent. The recess is preferably cylindrical or substantially cylindrical. Alternatively, the recess may be in the form of a groove or elongate slot. In some embodiments, smoking substitute apparatus may include a plurality of flavorant-receiving portions, for example a plurality of recesses, each configured to retain a droplet of flavorant. The plurality of recesses may be arranged substantially equidistantly, for example in a polka dot patter. The smoking substitute apparatus may include a first flavorant-receiving portion on a first surface of the smoking substitute apparatus, and a second flavorant-receiving portion on a second surface of the smoking substitute apparatus, wherein the first surface may be opposite to the second surface.

In embodiments in which the flavorant-receiving portion includes a recess, the recess may have a volume between 0 and 2 ml, more preferably between 0.1 and 1 ml, and more preferably between 0.2 and 0.5 ml.

A second aspect of the nineteenth mode of the present disclosure provides a smoking substitute kit, including a smoking substitute apparatus of the third aspect of the nineteenth mode of the disclosure, and a flavorant applicator for applying a flavorant-containing element to the flavorant-receiving portion of the smoking substitute apparatus. By providing a user with a kit including the smoking substitute apparatus and the flavorant applicator, the user is given more control of both what flavorant is used, and how much is used. So, if the flavorant is running low, a user is easily able to add some more, in order to intensify the taste or aroma.

In some embodiments, the flavorant applicator is configured to apply a liquid flavorant to the flavorant-receiving portion of the smoking substitute apparatus, to form the flavorant-containing element. In some embodiments, the liquid flavorant may be colorless, but in other embodiments, the liquid flavorant may include a flavored or scented ink. As discussed earlier, the flavorant-containing element formed by the application of the liquid flavorant may be configured to indicate visually how much flavorant is remaining in the flavorant-containing element. In this way, a user of the smoking substitute apparatus is able to identify when the flavorant has run out, and needs, e.g., to be replaced. In one such embodiment, the flavorant-containing element may be configured to fade over time, wherein the extent to which the flavorant-containing element has faded provides the visual indication of the amount of flavorant is remaining in the flavorant-containing element.

In some embodiments, the flavorant applicator may be in the form of a felt-tip pen (such as a “dry wipe” marker, or a permanent marker), a rollerball pen, a gel pen, or a ball point pen. With such embodiments, the user may be able to prepare a smoking substitute apparatus such as is described with reference to the second aspect of the nineteenth mode of the present disclosure.

In other embodiments, the flavorant applicator is configured to apply a droplet of liquid flavorant to the flavorant-receiving portion of the smoking substitute apparatus. In order to do so, the flavorant applicator may be in the form of a dropper or a pipette. The dropper or pipette is preferably configured to dispense droplets having a volume between 0 and 2 ml, more preferably between 0.1 and 1 ml, and more preferably between 0.2 and 0.5 ml.

Other embodiments of the second aspect of the nineteenth mode of the disclosure may employ a “scratch and sniff” component. Specifically, the flavorant applicator may include flavorant which is encapsulated in a plurality of microcapsules. The flavorant may be encapsulated in an encapsulation material comprising one or more of: ethyl cellulose, polyvinyl alcohol, and sodium alginate. In order to deliver the flavorant to the flavorant-receiving portion of the smoking substitute apparatus, the microcapsules are preferably configured to rupture, disintegrate or break down under the application of pressure. For example, the microcapsules may be configured to burst when they are pressed against the flavorant-receiving portion of the smoking substitute apparatus, thus releasing the flavorant onto the flavorant-receiving portion. In other embodiments, the application of friction may be used to deliver the flavorant to the flavorant-receiving portion, specifically the microcapsules may be configured to rupture, disintegrate, or break down when the flavor applicator is rubbed against the flavorant-receiving portion of the smoking substitute apparatus thereby releasing the flavorant onto the flavorant-receiving portion of the smoking substitute apparatus.

The flavorant applicator may include a flavorant layer and a protective layer covering the flavorant layer, and the protective layer may be configured to rupture, disintegrate or break down when the flavorant applicator is pressed or rubbed against the flavorant-receiving portion of the smoking substitute apparatus, thereby releasing the flavorant onto the flavorant-receiving portion of the smoking substitute apparatus.

In other embodiments, the flavorant applicator may be configured to release the flavorant in response to the application of heat to its outer surface.

The flavorant applicator may come a wide variety of shapes, including spherical, substantially spherical, cylindrical, substantially cylindrical, cubic, substantially cubic, cuboidal, substantially cuboidal, prismatic, substantially prismatic, pyramidal, substantially pyramidal, conic, or substantially conic.

The following description relates to general features of, e.g., smoking substitute apparatuses, which may relate to the first or the second aspect of the nineteenth mode of the disclosure.

The smoking substitute apparatus may be in the form of a consumable. The consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable. Alternatively, the smoking substitute apparatus may be a non-consumable apparatus (e.g., that is in the form of an open smoking substitute system). In such embodiments an aerosol former (e.g., e-liquid) of the system may be replenished by re-filling, e.g., a reservoir of the smoking substitute apparatus with the aerosol former (rather than replacing a consumable component of the apparatus).

In light of this, it should be appreciated that some of the features described herein as being part of the smoking substitute apparatus may alternatively form part of a main body for engagement with the smoking substitute apparatus (i.e., when the smoking substitute apparatus is in the form of a consumable).

Throughout this application, the term “flavorant” is used to describe a compound or combination of compounds that provide flavor and/or aroma. For example, the flavorant may be configured to interact with a sensory receptor of a user (such as an olfactory or taste receptor). The flavorant may include one or more volatile substances. The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations. The flavorant may be configured to release aroma or flavor in response to one or more of heat, pressure, friction and moisture.

Where the smoking substitute apparatus is in the form of a consumable, the consumable may be configured for engagement with a main body (i.e., so as to form a closed smoking substitute system). For example, the consumable may comprise components of the system that are disposable, and the main body may comprise non-disposable or non-consumable components (e.g., power supply, controller, sensor, etc.) that facilitate the delivery of aerosol by the consumable. In such an embodiment, the aerosol former (e.g., e-liquid) may be replenished by replacing a used consumable with an unused consumable.

The smoking substitute apparatus may comprise a reservoir configured to store an aerosol former, such as an e-liquid. The e-liquid may, for example, comprise a base liquid and, e.g., nicotine. The base liquid may include propylene glycol and/or vegetable glycerin. The e-liquid may be flavorless. That is, the e-liquid may not contain any flavorants and may consist solely of a base liquid of propylene glycol and/or vegetable glycerin and nicotine.

The reservoir may be in the form of a tank. At least a portion of the tank may be translucent. For example, the tank may comprise a window to allow a user to visually assess the quantity of e-liquid in the tank. A housing of the smoking substitute apparatus may comprise a corresponding aperture (or slot) or window that may be aligned with a translucent portion (e.g., window) of the tank. The reservoir may be referred to as a “clearomizer” if it includes a window, or a “cartomizer” if it does not.

The smoking substitute apparatus may comprise a passage for fluid flow therethrough. The passage may extend through (at least a portion of) the smoking substitute apparatus, between openings that may define an inlet and an outlet of the passage. The outlet may be at a mouthpiece of the smoking substitute apparatus. In this respect, a user may draw fluid (e.g., air) into and through the passage by inhaling at the outlet (i.e., using the mouthpiece). The passage may be at least partially defined by the tank. The tank may substantially (or fully) define the passage. In this respect, the tank may surround the passage.

The smoking substitute apparatus may comprise an aerosol-generator. The aerosol generator may comprise a wick. The aerosol generator may further comprise a heater. The wick may comprise a porous material. A portion of the wick may be exposed to fluid flow in the passage. The wick may also comprise one or more portions in contact with liquid stored in the reservoir. For example, opposing ends of the wick may protrude into the reservoir and a central portion (between the ends) may extend across the passage so as to be exposed to fluid flow in the passage. Thus, fluid may be drawn (e.g., by capillary action) along the wick, from the reservoir to the exposed portion of the wick.

The heater may comprise a heating element, which may be in the form of a filament wound about the wick (e.g., the filament may extend helically about the wick). The filament may be wound about the exposed portion of the wick. The heating element may be electrically connected (or connectable) to a power source. Thus, in operation, the power source may supply electricity to (i.e., apply a voltage across) the heating element so as to heat the heating element. This may cause liquid stored in the wick (i.e., drawn from the tank) to be heated so as to form a vapor and become entrained in fluid flowing through the passage.

This vapor may subsequently cool to form an aerosol in the passage.

The smoking substitute apparatus (or main body engaged with the smoking substitute apparatus) may comprise a power source. The power source may be electrically connected (or connectable) to a heater of the smoking substitute apparatus (e.g., when engaged with the main body). The power source may be a battery (e.g., a rechargeable battery). A connector in the form of, e.g., a USB port may be provided for recharging this battery.

When the smoking substitute apparatus is in the form of a consumable, the smoking substitute apparatus may comprise an electrical interface for interfacing with a corresponding electrical interface of the main body. One or both of the electrical interfaces may include one or more electrical contacts. Thus, when the main body is engaged with the consumable, the electrical interface may be configured to transfer electrical power from the power source to a heater of the consumable. The electrical interface may also be used to identify the smoking substitute apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the smoking substitute apparatus is in the form of a consumable, the main body may comprise an interface, which may, for example, be in the form of an RFID reader, a barcode or QR code reader. This interface may be able to identify a characteristic (e.g., a type) of a consumable engaged with the main body. In this respect, the consumable may include any one or more of an RFID chip, a barcode or QR code, or memory within which is an identifier and which can be interrogated via the interface.

The smoking substitute apparatus or main body may comprise a controller, which may include a microprocessor. The controller may be configured to control the supply of power from the power source to the heater of the smoking substitute apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or smoking substitute apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., Wi-Fi®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater of the consumable in response to a puff detection by the sensor. The control may be in the form of activation of the heater in response to a detected puff. That is, the smoking substitute apparatus may be configured to be activated when a puff is detected by the puff sensor. When the smoking substitute apparatus is in the form of a consumable, the puff sensor may form part of the consumable or the main body.

The disclosure includes the combination of the aspects and preferred features of the nineteenth mode described except where such a combination is clearly impermissible or expressly avoided.

The skilled person will appreciate that except where mutually exclusive, a feature or parameter described in relation to any one of the above aspects of the nineteenth mode may be applied to any other aspect of the nineteenth mode. Furthermore, except where mutually exclusive, any feature or parameter of the nineteenth mode described herein may be applied to any aspect and/or combined with any other feature or parameter of the nineteenth mode described herein.

### SUMMARY OF THE FIGURES

So that the disclosure may be understood, and so that further aspects and features thereof may be appreciated, embodiments illustrating the principles of the disclosure will now be discussed in further detail with reference to the accompanying figures, in which:

FIG. 1A is a front view of a smoking substitute system, according to a first embodiment of the first mode, in an engaged position.

FIG. 1B is a front view of smoking substitute system of the first embodiment of the first mode in a disengaged position.

FIG. 1C is a section view of a smoking substitute apparatus of the first embodiment of the first mode.

FIG. 2A is a section view of a substrate and a smoking substitute apparatus according to a second embodiment of the first mode.

FIG. 2B is a section view of the substrate being received in smoking substitute apparatus of the second embodiment of the first mode.

FIGS. 3A and 3B are respectively section views of exemplary embodiments of the first mode of various substrates and their corresponding openings.

FIG. 4A is a section view of a substrate and a smoking substitute apparatus according to a third embodiment of the first mode.

FIG. 4B is a section view of the substrate being received in smoking substitute apparatus of the third embodiment of the first mode.

FIG. 5 is a sectional view of a substrate being received in a container of a fourth embodiment of the first mode.

FIGS. 6A and 6B illustrate the process of producing a substrate according to a fifth embodiment of the first mode.

FIG. 7A is a front view of a smoking substitute system according to an embodiment of the second mode of the disclosure, in an engaged position.

FIG. 7B is a front view of an exemplary embodiment of the second mode of the smoking substitute system of FIG. 7A in a disengaged position.

FIG. 7C is a section view of a consumable of the smoking substitute system of FIG. 7A, according to an embodiment of the second mode of the disclosure.

FIG. 7D is a front view of the consumable of FIG. 7C.

FIG. 8A is a cross-sectional side view of a sticker according to an embodiment of the second mode of the disclosure.

FIG. 8B is a top view of the sticker of FIG. 8A.

FIG. 9 is a schematic cross-sectional view a flavorant-containing microcapsule that may be included in in a sticker that is an embodiment of the second mode of the disclosure.

FIG. 10A is a cross-sectional schematic view of packaging according to an embodiment of the third mode of the disclosure.

FIG. 10B is an image of the packaging of FIG. 10A.

FIG. 11 is a perspective view of packaging according to another embodiment of the third mode of the disclosure.

FIG. 12 is an image of packaging according to another embodiment of the third mode of the disclosure.

FIG. 13A is a front view of a smoking substitute system that may be received in packaging of the third mode of the disclosure, the smoking substitute system being in an engaged position.

FIG. 13B is a front view of the smoking substitute system of FIG. 13A in a disengaged position.

FIG. 13C is a cross-sectional schematic view of a consumable of the smoking substitute system of FIGS. 13A and 13B.

FIG. 14A is a front view of a smoking substitute system, according to a first embodiment of the fourth mode, in an engaged position.

FIG. 14B is a front view of the smoking substitute system of the first embodiment of the fourth mode in a disengaged position.

FIG. 14C is a detailed section view of the smoking substitute system of the first embodiment of the fourth mode.

FIG. 14D is a further detailed section view of the smoking substitute apparatus of the first embodiment of the fourth mode during engagement with a receptacle.

FIG. 14E is a further detailed section view of a smoking substitute apparatus of the first embodiment of the fourth mode engaged with a receptacle.

FIG. 15 is section view of a smoking substitute apparatus according to a second embodiment of the fourth mode.

FIG. 16A is a front view of a smoking substitute system, according to a first embodiment of the fifth mode, in an engaged position.

FIG. 16B is a front view of smoking substitute system of the first embodiment of the fifth mode in a disengaged position.

FIG. 16C is a section view of a smoking substitute apparatus of the first embodiment of the fifth mode.

FIG. 17A is a section view of a capsule of a second embodiment of the fifth mode.

FIG. 17B is a front view of the capsule being received in a smoking substitute apparatus of the second embodiment of the fifth mode.

FIG. 17C illustrates a method of the fifth mode of releasing property modifying agent in the smoking substitute apparatus.

FIG. 17D illustrates another method of the fifth mode of releasing property modifying agent in the smoking substitute apparatus.

FIG. 18 illustrates a capsule being inserted into the smoking substitute apparatus of another embodiment of the fifth mode.

FIGS. 19A to 19C illustrates and exemplary embodiment of the fifth mode of the use of a dispenser for dispensing a capsule to a smoking substitute apparatus.

FIG. 20A is a front view of a smoking substitute system, according to a first embodiment of the sixth mode, in an engaged position.

FIG. 20B is a front view of smoking substitute system of the first embodiment of the sixth mode in a disengaged position.

FIG. 20C is a section view of a smoking substitute apparatus of the first embodiment of the sixth mode in a first position.

FIG. 20D is a section view of a smoking substitute apparatus of the first embodiment of the sixth mode in a second position.

FIG. 21A is a front view of a smoking substitute system, according to a first embodiment of the seventh mode of the disclosure, in an engaged position.

FIG. 21B is a front view of the smoking substitute system of the first embodiment of the seventh mode in a disengaged position.

FIG. 21C is a cross-sectional view of a smoking substitute apparatus of the first embodiment of the seventh mode.

FIG. 22 is a schematic side view of a smoking substitute apparatus according to an embodiment of the seventh mode of the disclosure.

FIG. 23 is an illustration of textured outer surfaces that may be included on a mouthpiece according to an embodiment of the seventh mode of the disclosure.

FIGS. 24A-24C show schematic cross-sectionals views of a flavorant dispenser according to an embodiment of the seventh mode of the disclosure, illustrating a process of dispensing a flavorant onto a mouthpiece according to an embodiment of the disclosure.

FIGS. 25A and 25B show top views of an engagement portion of a mouthpiece according to embodiments of the seventh mode of the disclosure.

FIG. 26A is a front view of a smoking substitute system, according to a first embodiment of the eighth mode, in an engaged position.

FIG. 26B is a front view of smoking substitute system of the first embodiment of the eighth mode in a disengaged position.

FIG. 26C is a section view of a smoking substitute apparatus of the smoking substitute system of FIGS. 26B and 26C.

FIG. 27 is a front view of the smoking substitute apparatus according to a second embodiment of the eighth mode.

FIG. 28 is a front view of the smoking substitute apparatus according to a third embodiment of the eighth mode.

FIGS. 29A, 29B and 29C show sectional views of a dispenser according to a fourth embodiment of the eighth mode.

FIG. 30A is a front view of a smoking substitute system, according to a first embodiment, in an engaged position.

FIG. 30B is a front view of smoking substitute system of the first embodiment of the ninth mode in a disengaged position.

FIG. 30C is a section view of an aerosol delivery device of the first embodiment of the ninth mode in a first position.

FIG. 30D is a section view of an aerosol delivery device of the first embodiment of the ninth mode in a second position.

FIG. 30E is a section view of an aerosol delivery device of the first embodiment of the ninth mode in a third position.

FIGS. 31A and 31B are schematics depicting a valve according to a second embodiment of the ninth mode.

FIGS. 32A and 32B are schematics depicting a valve according to a third embodiment of the ninth mode.

FIGS. 33A and 33B are schematics depicting a valve according a fourth embodiment of the ninth mode.

FIG. 34 is a front view of a smoking substitute device of the tenth mode and a main body in an engaged position.

FIG. 35 is a front view of a smoking substitute device of the tenth mode and a main body in a disengaged position.

FIG. 36 is a section view of a smoking substitute device of the tenth mode.

FIG. 37 is a section view of a main body of a smoking substitute system of the tenth mode comprising a heater.

FIG. 38 is a section view of an exemplary flavorant reservoir of the tenth mode having a wick extending into the fluid passage of a smoking substitute device.

FIG. 39 is a section view of an exemplary flavorant reservoir of the tenth mode having a fluid-permeable surface forming a peripheral wall of the fluid passage of a smoking substitute device.

FIG. 40 is an end-on view of an exemplary flavorant reservoir of the tenth mode having a plurality of bores extending therethrough.

FIG. 41 is an enlarged section view of a section of a flavored article of the tenth mode.

FIG. 42 is a section view of an exemplary flavor pod of the tenth mode having a porous wick.

FIG. 43 is a section view of an exemplary flavor pod of the tenth mode having a fluid-permeable surface.

FIG. 44 is an end-on view of an exemplary flavor pod of the tenth mode having a plurality of bores extending therethrough.

FIG. 45 is a front view of a smoking substitute device, main body, and flavor part of the tenth mode in an engaged position.

FIG. 46 is a front view of a smoking substitute device, main body and flavor part of the tenth mode in a disengaged position.

FIG. 47 is a section view of a smoking substitute device of the tenth mode comprising a heater and a flavorant reservoir.

FIG. 48A is a front view of a smoking substitute system of the eleventh mode in an engaged position.

FIG. 48B is a front view of the smoking substitute system of FIG. 48A in a disengaged position.

FIG. 48C is a section view of a smoking substitute apparatus of the eleventh mode.

FIG. 49 is a section view of a smoking substitute apparatus having a flavor delivery apparatus according to an embodiment of the eleventh mode.

FIG. 50 is an enlarged section view of a flavor delivery apparatus according to an embodiment of the eleventh mode.

FIG. 51A is a front view of a smoking substitute system of the twelfth mode in an engaged position.

FIG. 51B is a front view of the smoking substitute system of the twelfth mode in a disengaged position.

FIG. 51C is a section view of a smoking substitute apparatus of the twelfth mode.

FIG. 52 is a front view of a smoking substitute device of the twelfth mode having a flavor delivery article.

FIG. 53 is a partial side section view of a smoking substitute apparatus of the twelfth mode having a flavor delivery article.

FIG. 54A is a front view of a smoking substitute system of the thirteenth mode in an engaged position.

FIG. 54B is a front view of the smoking substitute system of FIG. 54A in a disengaged position.

FIG. 54C is a section view of a smoking substitute apparatus of the thirteenth mode.

FIG. 55 is a section view of a smoking substitute apparatus of an embodiment of the thirteenth mode.

FIG. 56 is an enlarged section view of a smoking substitute apparatus of an embodiment of the thirteenth mode.

FIG. 57A is a front view of a smoking substitute apparatus, according to a first embodiment of the fourteenth mode of the disclosure, in an engaged position.

FIG. 57B is a front view of smoking substitute apparatus of the first embodiment of the fourteenth mode in a disengaged position.

FIG. 57C is a section view of a cartridge according to an embodiment of the fourteenth mode.

FIG. 58 is schematic cross-sectional view of a smoking substitute apparatus according to an embodiment of the fourteenth mode of the disclosure.

FIG. 59 is a diagrammatic representation of a smoking substitute system according to an embodiment of the fourteenth mode of the disclosure.

FIG. 60 is a front view of a smoking substitute device of the fifteenth mode and a main body in an engaged position.

FIG. 61 is a front view of a smoking substitute device of the fifteenth mode and a main body in a disengaged position.

FIG. 62 is a section view of a smoking substitute device of the fifteenth mode.

FIG. 63 is a section view of a smoking substitute device of the fifteenth mode comprising a flavorant reservoir.

FIG. 64 is a section view of a main body of a smoking substitute system of the fifteenth mode comprising a fluid passage therethrough.

FIG. 65 is a section view of an exemplary flavorant reservoir of the fifteenth mode having a wick extending into the fluid passage of a smoking substitute device.

FIG. 66 is a section view of an exemplary flavorant reservoir of the fifteenth mode having a fluid-permeable surface forming a peripheral wall of the fluid passage of a smoking substitute device.

FIG. 67 is an end-on view of an exemplary flavorant reservoir of the fifteenth mode having a plurality of bores extending therethrough.

FIG. 68 is an enlarged section view of a section of a flavored article of the fifteenth mode.

FIG. 69 is a section view of an exemplary flavor pod of the fifteenth mode having a porous wick.

FIG. 70 is a section view of an exemplary flavor pod of the fifteenth mode having a fluid-permeable surface.

FIG. 71 is an end-on view of an exemplary flavor pod of the fifteenth mode having a plurality of bores extending therethrough.

FIG. 72 is a front view of a smoking substitute device, main body, and flavor part of the fifteenth mode in an engaged position.

FIG. 73 is a front view of a smoking substitute device, main body and flavor part of the fifteenth mode in a disengaged position.

FIG. 74A is a front view of a smoking substitute system, according to a first embodiment of the sixteenth mode, in an engaged position.

FIG. 74B is a front view of smoking substitute system of the first embodiment of the sixteenth mode in a disengaged position.

FIG. 74C is a section view of a smoking substitute apparatus of the first embodiment of the sixteenth mode.

FIG. 75A is a front view of a second embodiment of the sixteenth mode of a smoking substitute system with a consumable of the system engaged with a device of the system.

FIG. 75B is a front view of the second embodiment of the sixteenth mode of the smoking substitute system with the consumable of the system disengaged from the device.

FIG. 75C is a schematic of the consumable of the second embodiment of the sixteenth mode of the substitute smoking system.

FIG. 76A is a sectional view of a flavor delivery article, in the form of a capsule, according to an embodiment of the sixteenth mode of the present disclosure.

FIG. 76B is a sectional view of a flavor delivery article, in the form of a composite sheet, according to another embodiment of the sixteenth mode of the present disclosure.

FIG. 77 is a sectional view of a flavor delivery article, in the form of a tablet, according to another embodiment of the sixteenth mode of the present disclosure.

FIG. 78 is a sectional view of a flavor delivery article, in the form of a substrate product, according to another embodiment of the sixteenth mode of the present disclosure.

FIG. 79 is a sectional view of a flavor delivery article, in the form of a solid/liquid suspension, according to another embodiment of the sixteenth mode of the present disclosure.

FIG. 80 is a sectional view of a flavor delivery article, in the form of a porous flavor delivery article, according to another embodiment of the sixteenth mode of the present disclosure.

FIG. 81 shows cross-sectional views of possible flavor delivery article carrying substrate shape profiles of the sixteenth mode.

FIG. 82 is sectional view of a smoking substitute apparatus of the sixteenth mode having a thin, flat substrate (e.g., 1 mm thick) that is situated in the passage above the vaporizer engine.

FIGS. 83A to 83E are perspective views of five flavor delivery article of the sixteenth mode carrying substrate shapes.

FIG. 84 is a side view of a smoking substitute apparatus of the sixteenth mode having a substrate that protrudes from the passage of the smoking substitute apparatus (i.e., it is not fully contained within the smoking substitute apparatus) and through the outlet.

FIG. 85 is a sectional view of a smoking substitute apparatus of the sixteenth mode having a flavor delivery article on a cylindrical substrate in the passage.

FIG. 86 is a sectional view of a smoking substitute apparatus of the sixteenth mode having a flavor delivery article applied directly to the surface of the passage, such as at the positions indicated by the arrows and therebetween.

FIG. 87 is a front view of an exemplary smoking substitute device of the seventeenth mode and main body in an engaged position.

FIG. 88 is a front view of the smoking substitute device and main body of FIG. 87 in a disengaged position.

FIG. 89 is a section view of an exemplary smoking substitute device of the seventeenth mode.

FIG. 90 is a front view of the exemplary smoking substitute device and main body illustrated in FIG. 87 in combination with a flavored article according to the present disclosure, the smoking substitute device being in an engaged position with the main body.

FIG. 91 is a front view of the smoking substitute device and main body illustrated in FIG. 90 in a disengaged position.

FIG. 92 is a section view of the smoking substitute device illustrated in FIG. 89 in combination with a flavored article according to the present disclosure.

FIG. 93 is an end on view of an exemplary flavored article in accordance with the seventeenth mode of the present disclosure, the flavored article comprising a single flavorant.

FIG. 94 is a section view of a part of a flavored article in accordance with the seventeenth mode of the present disclosure, wherein the flavorant is contained within pores in the substrate.

FIG. 95 is a section view of a part of a flavored article in accordance with the seventeenth mode of the present disclosure, wherein the flavorant is coated on the substrate.

FIG. 96 is an end on view of an exemplary flavored article in accordance with the seventeenth mode of the present disclosure, the flavored article comprising two distinct flavorants.

FIG. 97 is a section view of an exemplary smoking substitute system according to the seventeenth mode of the present disclosure, and having a peripheral slot or groove for securing a flavored article in place.

FIG. 98 is a section view of the smoking substitute system illustrated in FIG. 95, with the flavored article in place.

FIG. 99 is a section view of an exemplary smoking substitute system according to the seventeenth mode of the present disclosure, and having a projecting lip or shoulder for securing a flavored article in place.

FIG. 100 is a section view of the smoking substitute system illustrated in FIG. 97, with the flavored article in place.

FIG. 101 is a section view of an exemplary smoking substitute system according to the seventeenth mode of the present disclosure, and having a clip or clasp for securing a flavored article in place.

FIG. 102 is a section view of the smoking substitute system illustrated in FIG. 99, with the flavored article in place.

FIG. 103 is a section view of an exemplary smoking substitute system according to the seventeenth mode of the present disclosure, and having a part of the outer casing extending across a peripheral groove or slot for securing a flavored article in place.

FIG. 104 is a section view of the smoking substitute system illustrated in FIG. 103, with the flavored article in place.

FIGS. 105A to 105C show a general smoking substitute system of the eighteenth mode configured to provide an e-liquid to a user.

FIG. 106A is a front view of a second embodiment of the eighteenth mode of a smoking substitute system with a consumable of the system engaged with a device of the system.

FIG. 106B is a front view of the second embodiment of the eighteenth mode of the smoking substitute system with the consumable of the system disengaged from the device.

FIG. 106C is a schematic of the consumable of the second embodiment of the eighteenth mode of the substitute smoking system.

FIG. 107 is a drawing of a smoking substitute apparatus of the eighteenth mode having a flavorant-containing element in the form of a visual graphic printed thereon.

FIGS. 108A to 108C show a general smoking substitute system of the nineteenth mode configured to provide an e-liquid to a user.

FIG. 109A is a front view of a second embodiment of the nineteenth mode of a smoking substitute system with a consumable of the system engaged with a device of the system.

FIG. 109B is a front view of the second embodiment of the nineteenth mode of the smoking substitute system with the consumable of the system disengaged from the device.

FIG. 109C is a schematic of the consumable of the second embodiment of the nineteenth mode of the substitute smoking system.

FIG. 110 shows a close-up view of a smoking substitute device of the nineteenth mode having a recess at its mouthpiece end.

FIG. 111 shows a smoking substitute kit of the nineteenth mode including a pen and a smoking substitute device.

FIG. 112 shows a smoking substitute kit of the nineteenth mode including a flavorant applicator and a smoking substitute device.

### DETAILED DESCRIPTION OF THE FIGURES

Aspects and embodiments of the various mode of the present disclosure will now be discussed with reference to the accompanying figures. Further aspects and embodiments of the various modes will be apparent to those skilled in the art. All documents mentioned in this text are incorporated herein by reference.

First Mode: A Substrate for Releasing a Property Modifying Agent to an Aerosol Former Stored in a Reservoir

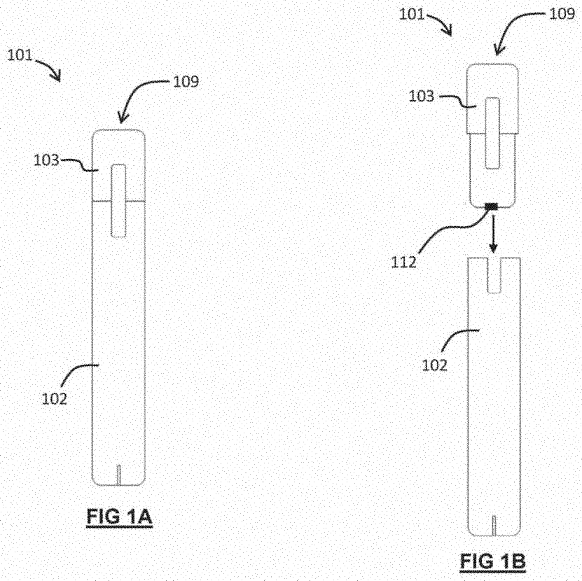

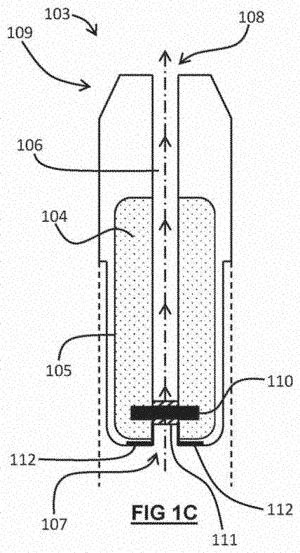

FIGS. 1A and 1B illustrate a smoking substitute system in the form of an e-cigarette system 101. The system 101 comprises an e-cigarette device defining a main body 102 of the system 101, and a smoking substitute apparatus in the form of an e-cigarette consumable (or “pod”) 103. In the illustrated embodiment the consumable 103 (smoking substitute apparatus) is removable from the main body (e-cigarette device), so as to be a replaceable component of the system 101. In other words, the e-cigarette system 101 is a closed system.

As is apparent from FIGS. 1A and 1B, the consumable 103 is configured to engage the main body 102. FIG. 1A shows the main body 102 and the consumable 103 in an engaged state, whilst FIG. 1B shows the main body 102 and the consumable 103 in a disengaged state. When engaged, a portion of the consumable 103 is received in a cavity of the main body 102 and is retained in the engaged position by way of a snap-engagement mechanism. In other embodiments, the main body 102 and consumable 103 may be engaged by screwing one into (or onto) the other, through a bayonet fitting, or by way of an interference fit.

The system 101 is configured to vaporize an aerosol-former, which in the illustrated embodiment, is in the form of a nicotine-based e-liquid 104. The e-liquid 104 comprises nicotine and a base liquid including propylene glycol and/or vegetable glycerin. In the present embodiment, the e-liquid 104 is flavorless (and does not include any added flavorant). That is, if the e-liquid 104 were to be inhaled (i.e., in aerosol form) by a user, it would not have a particularly perceptible flavor or taste.

As is more apparent from FIG. 1C, this e-liquid 104 is stored within a reservoir in the form of a tank 105 that forms part of the consumable 103. In the illustrated embodiment, the consumable 103 is a “single-use” consumable 103. That is, upon exhausting the e-liquid 104 in the tank 105, the intention is that the user disposes of the entire consumable 103. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105 surrounds, and thus defines a portion of, a passage 106 that extends between an inlet 107 and an outlet 108 at opposing ends of the consumable 103. In this respect, the passage comprises an upstream end at the end of the consumable 103 that engages with the main body 102, and a downstream end at an opposing end of the consumable 103 that comprises a mouthpiece 109 of the system 101. When the consumable 103 is engaged with the main body 102, a user can inhale (i.e., take a puff) via the mouthpiece 109 so as to draw air through the passage 106, and so as to form an airflow (indicated by arrows) in a direction from the inlet 107 to the outlet 108 of the passage 106. Although not illustrated, the passage 106 may be partially defined by a tube (e.g., a metal tube) extending through the consumable 103. The passage 106 is in fluid communication with a gap defined between the consumable 103 and the main body 102 (when engaged) such that air outside of the system 101 is drawn into the passage 106 (during an inhale).

The smoking substitute system 101 is configured to vaporize the e-liquid 104 for inhalation by a user. To provide this, the consumable 103 comprises a heater having of a porous wick 110 and a resistive heating element in the form of a heating filament 111 that is helically wound around a portion of the porous wick 110. The porous wick 110 extends across the passage 106 (i.e., transverse to a longitudinal axis of the passage 106) and opposing ends of the wick 110 extend into the tank 105 (so as to be submerged in the e-liquid 104). In this way, e-liquid 104 contained in the tank 105 is conveyed from the opposing ends of the porous wick 110 to a central portion of the porous wick 110 so as to be exposed to the airflow in the passage 106 (i.e., caused by a user inhaling).

The helical filament 111 is wound about this exposed central portion of the porous wick 110 and is electrically connected to an electrical interface in the form of electrical contacts 112 mounted at the end of the consumable that is proximate the main body 102 (when engaged). When the consumable 103 is engaged with the main body 102, the electrical contacts 112 contact corresponding electrical contacts (not shown) of the main body 102. The main body electrical contacts are electrically connected to a power source (not shown) of the main body 102, such that (in the engaged position) the filament 111 is electrically connected to the power source. In this way, power can be supplied by the main body 102 to the filament 111 in order to heat the filament 111. This heat is transferred from the filament 111 to the porous wick 110 which causes e-liquid 104 conveyed by the porous wick 110 to increase in temperature to a point at which it vaporizes. The vaporized e-liquid becomes entrained in the airflow and, between the vaporization point at the filament 111 and the outlet 108 of the passage 106, condenses to form an aerosol. This aerosol is then inhaled, via the mouthpiece 109, by a user of the system 101.

The power source of the main body 102 may be in the form of a battery (e.g., a rechargeable battery). The main body 102 may comprise a connector in the form of, e.g., a USB port for recharging this battery. The main body 102 may also comprise a controller that controls the supply of power from the power source to the main body electrical contacts (and thus to the filament 111). That, is the controller may be configured to control a voltage applied across the main body electrical contacts, and thus the voltage applied across the filament 111. In this way, the filament 111 may only be heated under certain conditions (e.g., during a puff and/or only when the system is in an active state). In this respect, the main body 102 may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102 and consumable 103 may comprise a further interface which may, for example, be in the form of an RFID reader, a barcode or QR code reader. This interface may be able to identify a characteristic (e.g., a type) of a consumable 103 engaged with the main body 102. In this respect, the consumable 103 may include any one or more of an RFID chip, a barcode or QR code, or memory within which is an identifier and which can be interrogated via the interface.

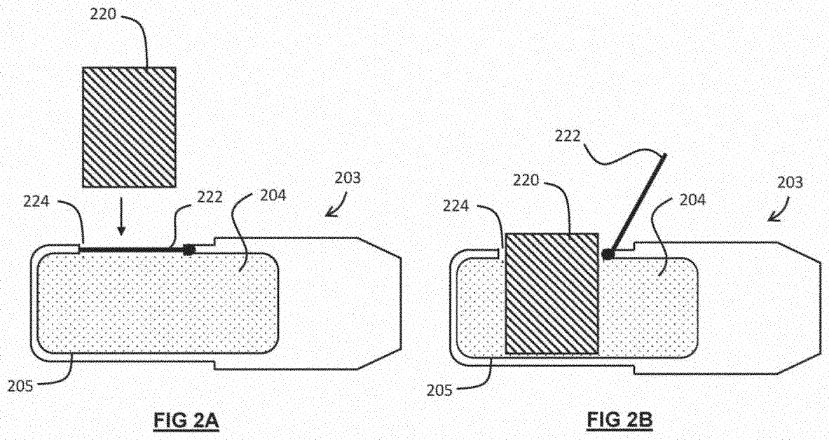

FIGS. 2A and 2B show a smoking substitute apparatus, or consumable 203, and a substrate 220 for the consumable 203 according to an embodiment of the present disclosure. FIG. 2A shows the substrate 220 being separated from the consumable 203, and FIG. 2B shows the substrate is partially received in a reservoir 205 of the consumable 203. For the purpose of illustration, some details of the consumable 203 are omitted from drawing but they are otherwise presented, for example the air flow passage and the heater. The consumable 203 in this embodiment is similar, structurally and functionally, to the consumable 103 as shown in FIGS. 1A, 1B and 1C. However, the consumable 203 differs to the consumable 103 in that it is configured to receive the substrate 220 into the tank, or reservoir 205 of the consumable 203.

In the illustrated embodiment, the reservoir 205 comprises a supply of aerosol former, or e-liquid 204. The aerosol former is in a liquid form. The aerosol former comprises nicotine and a base liquid, e.g., propylene glycol. The aerosol former does not contain any flavorant and therefore the aerosol former is not flavored. Additionally, the aerosol former in this embodiment does not contain any colorant. That is, the aerosol former is in the natural color of the propylene glycol and the nicotine, and therefore it appears as a translucent or transparent liquid.

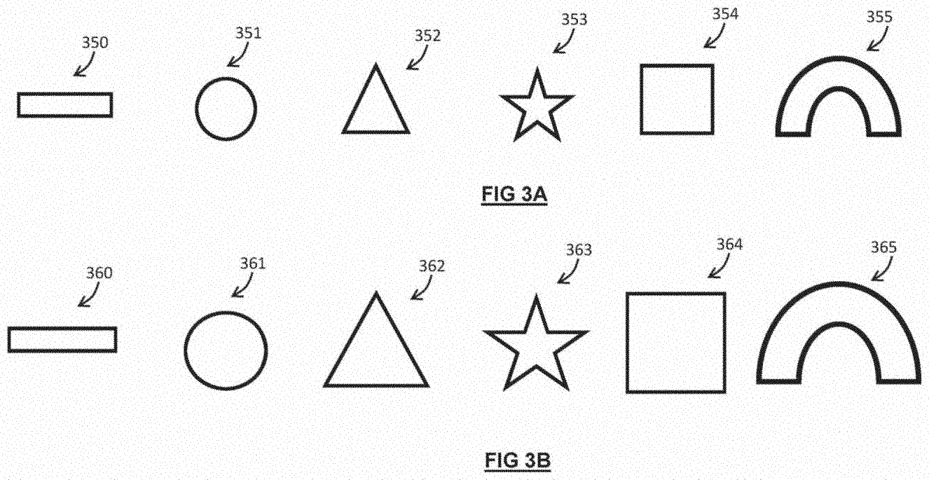

The consumable 203 comprises an opening 224, which is has a shape corresponding to a cross-sectional profile of the substrate 220. In the illustrated embodiment, the substrate 220 is a card formed from an absorbent paper material, e.g., a bibulous paper. Sectional views of the planar substrate 220 and its corresponding opening 224 are respectively shown in FIGS. 3A and 3B as planar substrate 350 and planar opening 360. The substrate 220 has a planar form and therefore the opening 224 is a slit that extends longitudinally along a side wall of the reservoir 205. More specifically, the planar substrate 220 is configured to be inserted into the reservoir through the slit 225.

FIGS. 3A and 3B respectively illustrates various substrates 351, 352, 353, 354, 355 and corresponding openings 361, 362, 363, 364, 365 as featured in other embodiments. For example, the substrate may have a cross sectional profile of a circle 351, polygon such as a triangle 352 and a square 354, a star 353, a crescent 355 or other profiles, and configured to extend through an opening with corresponding shape, e.g., a circle 351, polygon such as a triangle 352 and a square 354, a star 353, a crescent 355 or other profiles. Furthermore, in other embodiments, the cross-sectional profile of the substrate may vary along its longitudinal axis. For example, one end of the substrate can be thinner than an opposing end. In addition, the substrate 220 may be curved in the longitudinal direction, e.g., the substrate 220 may have a curved or angled axis.

The substrate 220 comprises a property modifying agent disposed thereon. That is, upon contacting the aerosol former 204 stored in the reservoir 205, the property modifying agent is configured to be released from the substrate 220 and to be diffused into the aerosol former 204. In the illustrated embodiment, the property modifying agent is a power deposited on the substrate 220. The property modifying agent comprises a flavorant and a colorant respectively configured to modify the flavor and color of the aerosol form 204 as stored in the reservoir 205. In other embodiments, the property modifying agent may be one or more microcapsules, a gel, a liquid or a gas, either coated onto, embedded, absorbed or adsorbed in the substrate 220. The property modifying agent may comprise only a flavorant without the presence of a colorant or it may comprise only a colorant without the presence of a flavorant.

To prevent leakage of the aerosol former through the opening 224, e.g., at least during transportation and storage, the opening is provided a closure 222 for sealing the opening 224. In the illustrated embodiment, a flap 222 is hingedly attached to a sidewall of the reservoir 205 at a position adjacent to the opening 224. As such the flap is pivotable from a closed position where the flap covers the opening 224 to an opened position where the flap moves away from the opening 224. More specifically, in the closed position the flap 222 seals said opening 224 and in the opened position the opening 224 is free of obstruction.

The substrate 220 is configured to be partially inserted into the reservoir 205 through the opening 224 when the flap 222 is put into the opened position. In the illustrated embodiment, at least 90% of the length of substrate 220, towards an immersed end, is immersed in the aerosol former 204 so as to allow a significant amount of the property modifying agent to release from the substrate 220. The un-immersed portion of substrate 220, towards a tab end, forms a tab that allows the user to pull onto in order to withdraw the substrate 220 from the reservoir.

The amount of property modifying agent that is releasable to the aerosol former 204 is directly related to the contacting area between the substrate 220 and the aerosol former 204, a user may control the concentration of, or rate of release of, property modifying agent in the aerosol former by specifying the portion of substrate to submerge into the aerosol former 204. That is, an increased portion of the substrate 220 that remains emerged above the liquid surface of the aerosol former 204 means any property modifying agent in said emerged portion will not, or less likely to, be released into the aerosol former 204. For example, a user may only submerge 50%, or less, of the length of the substrate 220 in the aerosol former 204 so as to limit the amount, or rate, of release of property modifying agent into the aerosol former 204.

In the illustrated embodiment, the substrate 220 may be retained in the reservoir 205 and in contact with the aerosol former 204 for a predetermined period of time. This allow the property modifying agent to be released into the aerosol former 204. Said release approaches an equilibrium as the concentration of property modifying agent in the aerosol former increases throughout the predetermined period of time. Thereafter, the user may opt to withdraw the substrate 220 by pulling on its tab. Since said predetermined period of time is directly related to the amount of property modifying agent that is releasable to the aerosol former, a user may control the concentration of property modifying agent in the aerosol former by specifying the predetermined period of time before the substrate 220 is withdrawn from the reservoir 205. The predetermined period of time is the given example is less than or up to 60 minutes. In other embodiments, in the case of fast releasing property modifying agent, e.g., one with strong hydrophilic properties, the predetermined period of time can be less than a minute. In other embodiments, in the case of slow releasing property modifying agent, the predetermined period of time can be more than 60 minutes.

In the illustrated embodiment, the flap 222 is fully opened when the substrate 220 is partially received in the reservoir 205. As such, the consumable 203 lies on its side when receiving the substrate 220, with the opening 224 facing upwardly, in order to prevent aerosol former from flowing or leaking out of the opening 224 under gravity.

The user may remove the substrate 220 from the reservoir 205 after the lapse of said predetermined period of time, i.e., the aerosol former 204 has already received the required amount of property modifying agent from the substrate 220. The flap 222 may pivot toward the closed position in order to seal the opening 224 before the consumable 203 is assembled on to the main body.

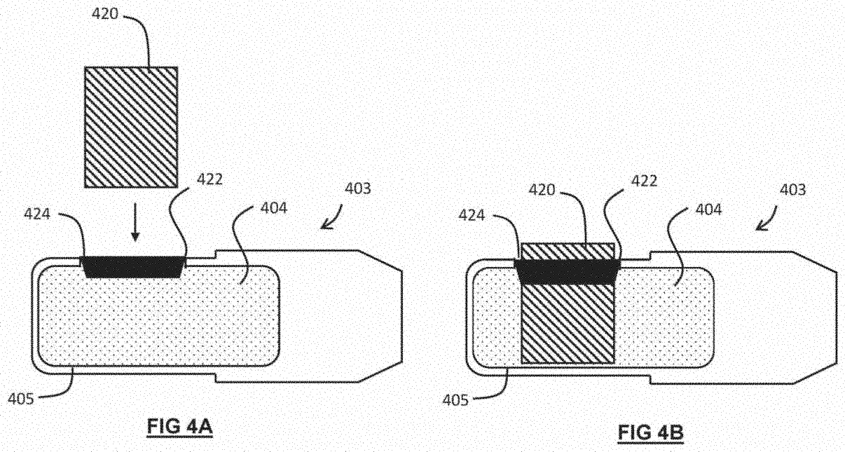

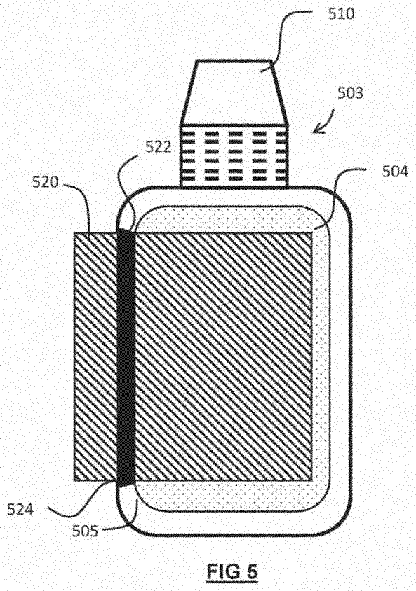

FIG. 4 shows a substrate 420 and a consumable 403 according to an embodiment according to the present disclosure. FIG. 4A shows the substrate 420 separated from the consumable 403, and FIG. 4B shows the substrate is partially received in a reservoir 405 of the consumable 403. The consumable 403 is structurally and functionally similar to the consumable 403. The consumable 403 differs to consumable 203 in that the consumable 403 comprises a valve 422 positioned across the opening 424 for sealing said opening. The valve 422 is a duck bill valve that comprises a resilient member, e.g., rubber, for forming a seal around the peripheral of the substrate 420 when it is partially received in the reservoir 405. As such, when the substrate 420 is partially received in the reservoir, the opening 424 remains sealed so as to prevent aerosol former 404 from leaking out of the reservoir. Therefore, in the illustrated embodiment, the consumable 403 may be rested in any orientation whilst the aerosol former 404 is prevented from leaking out of the opening 424.

The user may remove the substrate 420 from the reservoir 405 after the lapse of said predetermined period of time, i.e., the aerosol former 404 has already received the required amount of property modifying agent from the substrate 420. As the user withdraws the substrate 420 through the duck bill valve 422, the resilient member of the duck bill valve scrapes along the surface of the substrate and thereby removes excessive aerosol former retained thereon. Once the substrate is fully withdrawn, the resilient member seals the opening 424, thereby allowing the consumable 403 to be assembled on to the main body.

FIG. 5 shows a substrate 520 and a container 503 according to an embodiment according to the present disclosure, showing the substrate is partially received in a reservoir 505 of the container 503. The container 503 comprises an outlet 510 for dosing or adding aerosol former to a corresponding consumable. The container 503 is in the form of a bottle and stored with a supply of aerosol former 504. The container 503 comprises a valve 522 positioned across an opening 524 for sealing said opening. The valve 522 is a duck bill valve that comprises a resilient member, e.g., rubber, for forming a seal around the peripheral of the substrate 520 when it is partially received in the reservoir 505. As such, when the substrate 520 is partially received in the reservoir, the opening 524 remains sealed so as to prevent aerosol former 504 from leaking out of the reservoir. Therefore, in the illustrated embodiment, the consumable 503 may be rested in any orientation whilst the aerosol former 504 is prevented from leaking out of the opening 524.

The user may remove the substrate 520 from the reservoir 505 after the lapse of said predetermined period of time, i.e., the aerosol former 504 has already received the required amount of property modifying agent from the substrate 520. As the user withdraws the substrate 520 through the duck bill valve 522, the resilient member of the duck bill valve scrapes along the surface of the substrate and thereby removes excessive aerosol former retained thereon. Once the substrate 520 is fully withdrawn, the resilient member seals the opening 524. The user may then add the property modified aerosol former 504 to a smoking substitute apparatus via the container outlet 510.

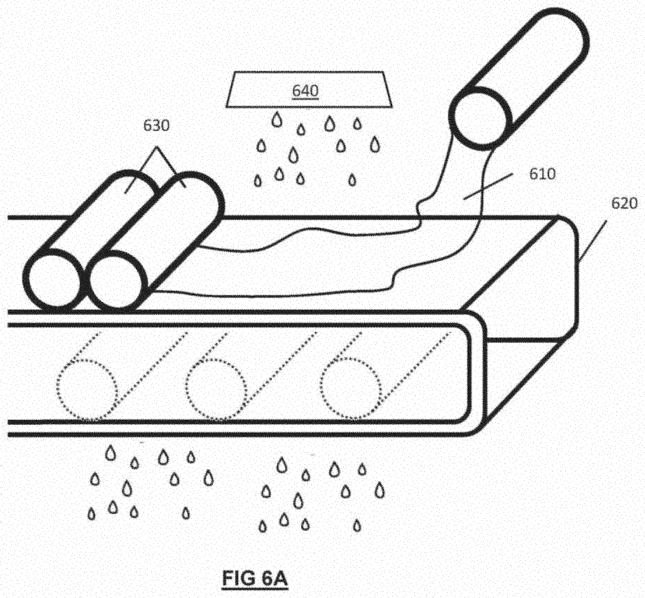

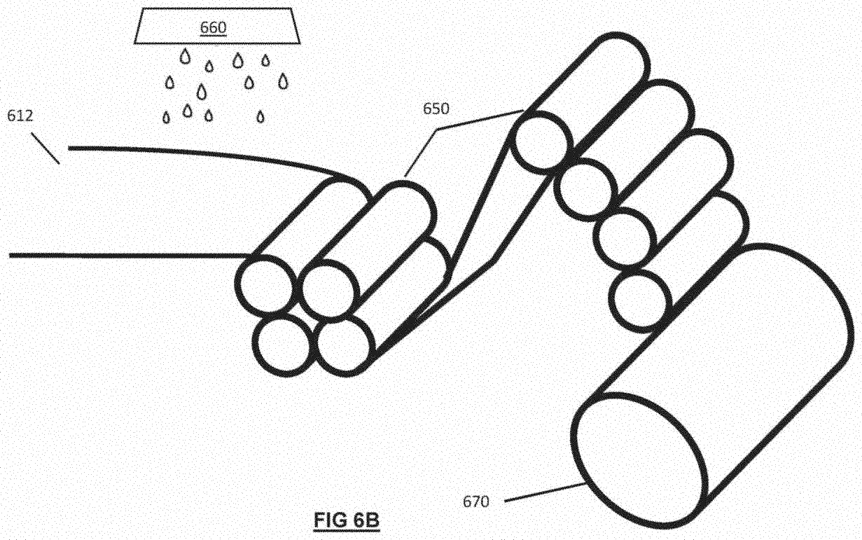

FIGS. 6A and 6B illustrate a manufacturing process for the substrate. As shown in FIG. 6A, a flow of beaten paper pulp 610 is fed onto a moving conveyer belt having fine mesh screening 620. As the paper pulp is being conveyed along the conveyor belt 620 excessive water in the pulp drips through the fine mesh screening and thereby the pulp's water content reduces as it progresses along the conveyor belt 620. That is, the fine mesh screening is water permeable mesh. As the paper pulp is being conveyed along the conveyor belt 620, property modifying agent, e.g., flavorant and/or colorant, is applied to the paper pulp. In this illustrated embodiment, the property modifying agent is applied using a sprayer 640 such that a fine mist of property modifying agent may be readily absorbed or adsorbed to the paper pulp. This may allow property modifying agent to be added to a core portion of the substrate.

The paper pulp, added with property modifying agent, may then continue to be conveyed by the conveyor belt 620 to be mechanically pressed using a set of rollers 630. This further reduces water content in the pulp and thereby forms a continuous sheet of substrate.

The continuous sheet of substrate 612 then passes over a series of steam-heated cylinders 650 to remove any remaining moisture in the substrate 612. In the illustrated embodiment, further property modifying agent in the form of an aerosol is sprayed on using sprayer 660 to the substrate 612 before it is dried by the steam-heated cylinders 650 and wound onto a set of large reels 670. This may allow the property modifying agent to be added to the surface of the substrate 612. The reel of substrate 670 may then be cut to size to create the finished substrate for use with the container 503 and/or the consumable 103, 203, 403.

The substrate may be added with further property modifying agent with a secondary addition process. This may allow more property modifying agent to be added to the substrate. For example, further property modifying agent may be added by spraying an aerosol of property modifying agent to the surface of substrate, or by dipping the substrate into a storage of property modifying agent.

Other aspects of the first mode of the present disclosure are set out in the following numbered clauses.

1. A substrate for a reservoir having an aerosol former stored therein, the substrate comprising: a property modifying agent disposed on the substrate;
wherein the substrate is configured to be received in the reservoir to contact the aerosol former, so as to facilitate a release of the property modifying agent from the substrate to the aerosol former, and
wherein the substrate is insoluble in the aerosol former.

2. The substrate of clause 1;
wherein the property modifying agent is configured to be deposited on a surface of the substrate.

3. The substrate of clause 1;
wherein the substrate is coated in a soluble coating containing the property modifying agent.

4. The substrate of any one of the preceding clauses, wherein the substrate comprises an absorbent paper.

5. The substrate of any one of the preceding clauses, wherein the substrate is configured to be removably received in the reservoir.

6. The substrate of any one of the preceding clauses, wherein the amount of property modifying agent releasable to the aerosol former corresponds to a predetermined period of time the substrate is received in the reservoir.

7. The substrate of clause 6, wherein the predetermined period of time ranges from 1 minute to 2 hours.

8. The substrate of any one of the preceding clauses, wherein the substrate is configured to be received in a reservoir of a smoking substitute apparatus.

9. The substrate of any one of the preceding clauses, wherein the property modifying agent comprises flavorant and/or colorant.

10. A reservoir for receiving the substrate of clauses 1 to 9, the reservoir is configured to store an aerosol former therein, the reservoir comprising an opening for providing access to the substrate as it extends into the reservoir to contact the aerosol former.

11. The reservoir of clause 10, wherein the reservoir comprises a closure for closing the opening, wherein the closure is configured to open to provide access for the substrate.

12. The reservoir of clause 11, wherein the closure comprises a valve having a resilient member for forming a peripheral seal with the substrate as it extends through the opening.

13. A smoking substitute apparatus comprising the reservoir of clauses 11 to 12.

14. A smoking substitute kit comprising:
the substrate of any one of the clauses 1 to 9; and
the reservoir of any one of clauses 10 to 12 or the smoking substitute apparatus of clause 13.

15. A method of producing a substrate, comprising:
preparing a pulp;
applying a property modifying agent to the pulp; and
drying the pulp to form the substrate.

Second Mode: A Flavored Sticker for Use with a Smoking Substitute Apparatus

Aspects and embodiments of the second mode of the present disclosure will now be discussed with reference to the accompanying figures. Further aspects of the second mode and embodiments will be apparent to those skilled in the art. All documents mentioned in this text are incorporated herein by reference.

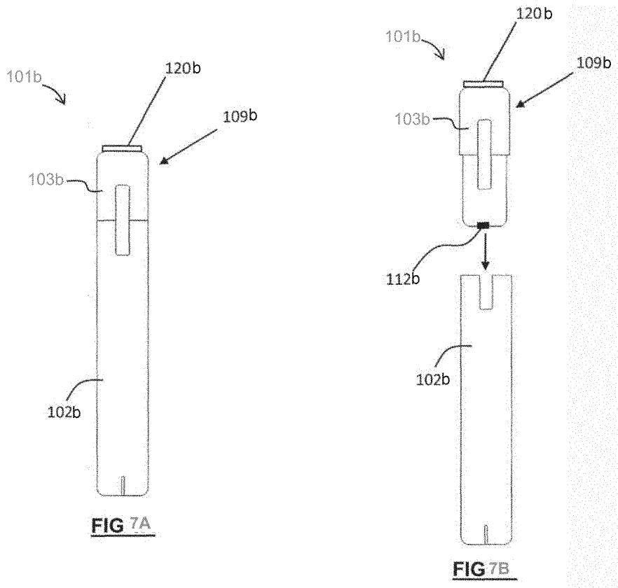

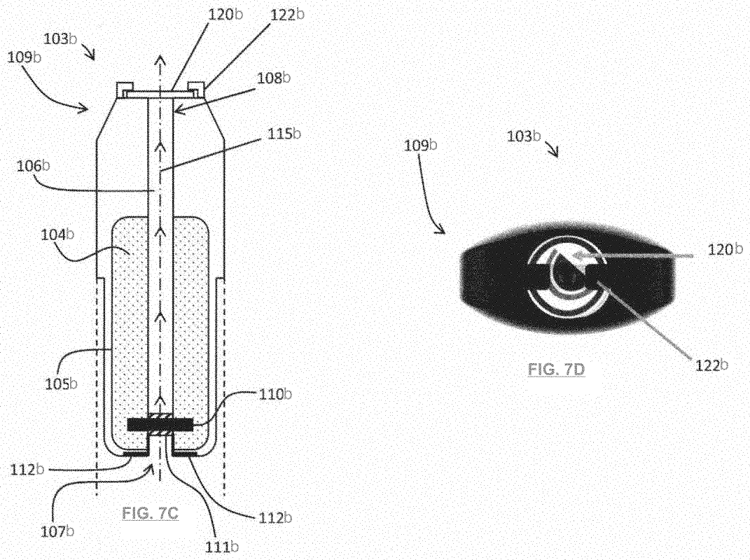

FIGS. 7A and 7B illustrate a smoking substitute system in the form of an e-cigarette system 101b. The system 101b comprises an e-cigarette device defining a main body 102b of the system 101b, and a smoking substitute apparatus in the form of an e-cigarette consumable (or “pod”) 103b. In the illustrated embodiment the consumable 103b (smoking substitute apparatus) is removable from the main body (e-cigarette device), so as to be a replaceable component of the system 101b. In other words, the e-cigarette system 101b is a closed system. An expanded schematic cross-sectional view of the consumable 103b is shown in FIG. 7C; a front view of the consumable 103b is shown in FIG. 7D.

As is apparent from FIGS. 7A and 7B, the consumable 103b is configured to engage the main body 102b. FIG. 7A shows the main body 102b and the consumable 103b in an engaged state, whilst FIG. 7B shows the main body 102b and the consumable 103b in a disengaged state. When engaged, a portion of the consumable 103b is received in a cavity of the main body 102b and is retained in the engaged position by way of a snap-engagement mechanism. In other embodiments, the main body 102b and consumable 103b may be engaged by screwing one into (or onto) the other, through a bayonet fitting, or by way of an interference fit.

The system 101b is configured to vaporize an aerosol-former, which in the illustrated embodiment, is in the form of a nicotine-based e-liquid 104b. The e-liquid 104b comprises nicotine and a base liquid including propylene glycol and/or vegetable glycerin. In the present embodiment, the e-liquid 104b is flavorless (and does not include any added flavorant). That is, if the e-liquid 104b were to be inhaled (i.e., in aerosol form) by a user, it would not have a particularly perceptible flavor or taste.

As is more apparent from FIG. 7C, this e-liquid 104b is stored within a reservoir in the form of a tank 105b that forms part of the consumable 103b. In the illustrated embodiment, the consumable 103b is a “single-use” consumable 103b. That is, upon exhausting the e-liquid 104b in the tank 105b, the intention is that the user disposes of the entire consumable 103b. In other embodiments, the e-liquid 104b (i.e., aerosol former) may be the only part of the system that is truly “single-use”. That is, the tank 105b may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105b surrounds, and thus defines a portion of, a passage 106b that extends between an inlet 107b and an outlet 108b at opposing ends of the consumable 103b. In this respect, the passage comprises an upstream end at the end of the consumable 103b that engages with the main body 102b, and a downstream end at an opposing end of the consumable 103b that comprises a mouthpiece 109b of the system 101b. When the consumable 103b is engaged with the main body 102b, a user can inhale (i.e., take a puff) via the mouthpiece 109b so as to draw air through the passage 106b, and so as to form an airflow (indicated by arrows) in a direction from the inlet 107b to the outlet 108b of the passage 106b. Although not illustrated, the passage 106b may be partially defined by a tube (e.g., a metal tube) extending through the consumable 103b. The passage 106b is in fluid communication with a gap defined between the consumable 103b and the main body 102b (when engaged) such that air outside of the system 101b is drawn into the passage 106b (during an inhale).

The smoking substitute system 101b is configured to vaporize the e-liquid 104b for inhalation by a user. To provide this, the consumable 103b comprises a heater having of a porous wick 110b and a resistive heating element in the form of a heating filament 111b that is helically wound around a portion of the porous wick 110b. The porous wick 110b extends across the passage 106b (i.e., transverse to a longitudinal axis of the passage 106b) and opposing ends of the wick 110b extend into the tank 105b (so as to be submerged in the e-liquid 104b). In this way, e-liquid 104b contained in the tank 105b is conveyed from the opposing ends of the porous wick 110b to a central portion of the porous wick 110b so as to be exposed to the airflow in the passage 106b (i.e., caused by a user inhaling).

The helical filament 111b is wound about this exposed central portion of the porous wick 110b and is electrically connected to an electrical interface in the form of electrical contacts 112b mounted at the end of the consumable that is proximate the main body 102b (when engaged). When the consumable 103b is engaged with the main body 102b, the electrical contacts 112b contact corresponding electrical contacts (not shown) of the main body 102b. The main body electrical contacts are electrically connected to a power source (not shown) of the main body 102b, such that (in the engaged position) the filament 111b is electrically connected to the power source. In this way, power can be supplied by the main body 102b to the filament 111b in order to heat the filament 111b. This heat is transferred from the filament 111b to the porous wick 110b which causes e-liquid 104b conveyed by the porous wick 110b to increase in temperature to a point at which it vaporizes. The vaporized e-liquid becomes entrained in the airflow and, between the vaporization point at the filament 111b and the outlet 108b of the passage 106b, condenses to form an aerosol. This aerosol is then inhaled, via the mouthpiece 109b, by a user of the system 101b.

The power source of the main body 102b may be in the form of a battery (e.g., a rechargeable battery).

The main body 102b may comprise a connector in the form of, e.g., a USB port for recharging this battery. The main body 102b may also comprise a controller that controls the supply of power from the power source to the main body electrical contacts (and thus to the filament 111b). That, is the controller may be configured to control a voltage applied across the main body electrical contacts, and thus the voltage applied across the filament 111b. In this way, the filament 111b may only be heated under certain conditions (e.g., during a puff and/or only when the system is in an active state). In this respect, the main body 102b may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102b and consumable 103b may comprise a further interface which may, for example, be in the form of an RFID reader, a barcode or QR code reader. This interface may be able to identify a characteristic (e.g., a type) of a consumable 103b engaged with the main body 102b. In this respect, the consumable 103b may include any one or more of an RFID chip, a barcode or QR code, or memory within which is an identifier and which can be interrogated via the interface.

The controller may further comprise a communication interface for communicating with an external device. For example, the controller may include a wireless interface for wireless communication (e.g., Bluetooth, Wi-Fi) or a wired interface for wired communication (e.g., USB).

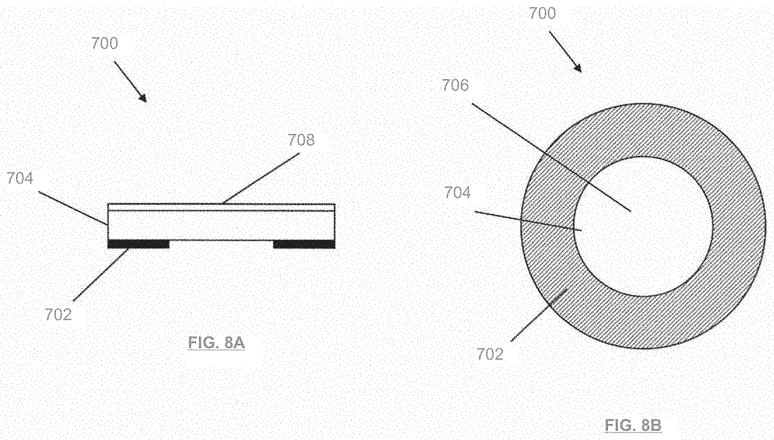

A sticker 120b is affixed to the mouthpiece 109b of the consumable 103b, such that the sticker 120b covers the outlet 108b of the mouthpiece 109b (see FIG. 7C). The sticker 120b includes an adhesive portion which serves to adhere the sticker 120b to the mouthpiece 109b, and a flavored portion which is arranged to cover the outlet 108b of the mouthpiece 109b. The sticker is described in more detail below in relation to FIGS. 8A and 8B. The flavored portion of the sticker 120b is permeable to the aerosol generated in the apparatus 101b, such that the aerosol may pass through the flavored portion of the sticker 120b when a user inhales the aerosol through the mouthpiece 109b.

The flavored portion of the sticker 120b includes a flavorant which may be entrained by the aerosol when the aerosol passes through the flavored portion of the sticker 120b, to provide flavor to the user. The flavorant may also produce a taste on the user's tongue when the user puts the mouthpiece 109b in their mouth. In this manner, flavor may be delivered to the user without having to include any flavorant in the e-liquid.

The mouthpiece 109b includes a pair of flexible tabs 122b disposed adjacent to the outlet 108b and arranged to hold the sticker 120b in place on the mouthpiece 109b. The flexible tabs 122b are arranged to press the sticker 120b against an outer surface of the mouthpiece 109b, to prevent the sticker from moving or becoming unstuck during use. Together the flexible tabs 122b define a groove in which the sticker 120b is received. This may serve to ensure correct placement of the sticker 120b, to ensure that it completely covers the outlet 108b of the mouthpiece 109b. The sticker 120b may be affixed to the mouthpiece 109b by bending one or both of the flexible tabs 122b to enable the sticker 120b to be positioned over the outlet 108b.

For illustration purposes, the flexible tabs are not depicted in FIGS. 7A and 7B.

FIG. 7D illustrates a front view of the consumable 103b, i.e., a view of the consumable looking towards the outlet 108b along an axis defined by arrows 115 in FIG. 7C.

The consumable 103b and the sticker 120b may be provided separately (e.g., as parts of a kit for a smoking substitute apparatus). Then, before using the consumable 103b, the user may affix the sticker 120b to the mouthpiece 109b, so that they receive flavor when inhaling an aerosol through the mouthpiece 109b. When the sticker 120b is used up (e.g., when it no longer has any flavorant), the user may remove the sticker 120b and replace it with a new sticker 120b. The sticker 120b may be configured to last for a certain amount of time, e.g., for a predetermined number of puffs, or for a predetermined amount of time in the user's mouth.

Different stickers 120b may be provided, each having a different flavor. In this manner, a user may select a desired flavor by affixing a sticker having the desired flavor to the mouthpiece 109b.

FIG. 8A shows as cross-sectional side view of a sticker 700 that is and embodiment of the disclosure, the sticker being intended for use with a smoking substitute apparatus (e.g., apparatus 101b). FIG. 8B shows a top view of sticker 700. For example, sticker 700 may correspond to sticker 120b described above.

The sticker 700 has a generally circular shape and includes an adhesive portion 702 for adhering the sticker to a mouthpiece of a smoking substitute apparatus (e.g., mouthpiece 109b). The adhesive portion 702 is formed by a ring of adhesive material which is disposed on a first surface of a substrate 704 of the sticker 700. FIG. 8B shows the first surface of the substrate 704 with the ring of adhesive forming the adhesive portion 702. The ring of adhesive material is arranged to encircle an outlet of the mouthpiece (e.g., outlet 108b) when the sticker 700 is adhered to the mouthpiece 109b. A central portion 706 of the substrate may thus cover the outlet of the mouthpiece when the sticker 700 is adhered to the mouthpiece. In this manner, when the sticker is adhered to the mouthpiece, the adhesive portion 702 may form a seal around the outlet, so that aerosol exiting the outlet is forced through the central portion 706 of the substrate 704.

The adhesive material forming the adhesive portion 702 may be any suitable pressure-activated adhesive. In this manner, when the sticker 700 is pressed against an outer surface of the mouthpiece, the adhesive material causes the sticker 700 to adhere to the mouthpiece. A backing layer (not shown) may be provided to protect the adhesive portion 702 prior to use. Then, when a user wishes to apply the sticker to a mouthpiece, the user may remove the backing layer to expose the adhesive portion 702, so that the sticker may be adhered to the mouthpiece. The backing layer may, for example be made of paper, vinyl, or some other suitable material.

The substrate 704 may be made of a material that is permeable to an aerosol generated by the smoking substitute system, so that the aerosol may pass through the central portion 706 of the substrate 704 when the sticker 700 is adhered to a mouthpiece of the smoking substitute system. Preferably, the substrate 704 may be made of a porous material, to facilitate transmission of the aerosol. The porous material may, for example, be a mesh material, a cloth material, a fiber material, a filter material, or a paper material. In some cases, only the central portion 706 of the substrate 704 may be made of a porous material, and the remainder of the substrate 704 may be made of a non-porous material. In some embodiments, one or more apertures (not shown) may be formed in the central portion 706 of the substrate 704, to further facilitate transmission of the aerosol through the substrate 704.

In the example shown, the sticker 700 further includes a flavorant layer 708 disposed on a second surface of the substrate 704. The second surface of the substrate 704 is on an opposite side of the substrate 704 compared to the first surface of the substrate 704 on which the adhesive ring forming the adhesive portion 702 is disposed. Thus, when the sticker 700 is adhered to a mouthpiece, the flavorant layer 708 may face towards a user, so that it may come in direct contact with a user's mouth (e.g., tongue), when the user puts the mouthpiece in their mouth.

The flavorant of the flavorant layer 708 may be arranged to produce a taste sensation when the user's tongue comes into contact with the flavorant layer. Additionally, or alternatively, the flavorant may be arranged to be entrained by a flow of aerosol from the mouthpiece into the user's mouth, to deliver flavor to the user. For example, the flavorant may include a volatile liquid and/or flavorant particles which may be entrained by a flow of aerosol through the central portion 706 of the substrate 704.

The flavorant may be provided in solid or liquid form. The flavorant may be natural or synthetic. For example, the flavorant may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations in the flavored portion of the sticker.

In some embodiments, various mechanisms may be used to “activate” delivery of the flavorant to the user. This may ensure that flavorant is retained in the flavorant layer 708 and is not released before use. For example, the flavorant layer 708 may be heat- and/or friction-activated. In such a case, the flavorant may be contained in an array of microcapsules arranged to form the flavorant layer 708. The microcapsules may then be arranged to rupture to release flavorant upon heating (e.g., when the sticker comes into contact with the user's mouth) and/or when friction is applied to the flavorant layer 708 (e.g., by rubbing or scratching the flavorant layer 708). In some cases, the flavorant layer 708 may be activated by the aerosol itself. For example, a chemical in the aerosol may cause the microcapsules to rupture and release flavorant when aerosol passes through the central portion 706 of the substrate 704.

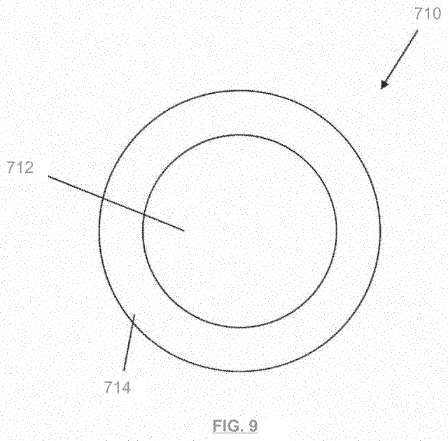

Such a scenario is discussed below in relation to FIG. 9.

Additionally, or alternatively, the sticker 700 may include a removable protective outer layer arranged to cover and protect the flavorant layer 708 prior to use. The removable protective outer layer may for example be a sticker arranged to cover the flavorant layer 708 which can be removed (e.g., peeled off) to expose the flavorant layer. The removable protective layer may be made of any suitable material, e.g., paper or vinyl.

In an alternative embodiment, flavorant may be directly incorporated into the substrate 704, instead of providing a separate flavorant layer 708. For example, the substrate 704 may be made of a porous material that is imbued with a flavorant liquid.

FIG. 9 shows a schematic cross-sectional view of a microcapsule 710 containing a flavorant 712. The microcapsule 710 is arranged to disintegrate in the presence of an aerosol generated by a smoking substitute system (e.g., system 101b), to release the flavorant 712 contained therein. A plurality of such microcapsules 710 containing flavorant may disposed in the flavorant layer 708 of the sticker 700.

The microcapsule 710 includes an outer membrane 714 made of a hydrogel composite which contains Chitosan. Chitosan is substantially stable around pH 7.5. Thus, when the microcapsule 710 is placed in a user's mouth, the Chitosan, and hence the outer membrane 714, may be stable and retain its integrity for a period of time. In this manner, the flavorant 712 contained in the microcapsule 710 is not released and so does not produce a flavor in the user's mouth.

A nicotine-containing e-liquid may produce an aerosol which has a pH around 7.8. Thus, when a user inhales such an aerosol produced with such an e-liquid, a pH in the user's mouth may increase above pH 7.5, which may cause rapid breakdown of the Chitosan in the outer membrane 714, resulting in disintegration of the microcapsule's outer membrane 714 and release of the flavorant 712.

The flavorant 712 in the microcapsule 710 may be in solid or liquid form. Preferably, the flavorant 712 may be a freeze-dried flavorant. In other words, during manufacture the flavorant 712 may have been freeze-dried to remove moisture from the flavorant 712. This may reduce the risk of flavorant 712 escaping from the microcapsule 710 prior to use. When the flavorant 712 is released from the microcapsule 710 due to disintegration of the outer membrane 714 in the user's mouth, the flavorant 712 may be activated by moisture in the user's mouth (e.g., the flavorant 712 may dissolve in the user's saliva), to produce a flavor in the user's mouth.

The outer membrane 714 may include Chitosan having a Deacetylation (DDT) of 75%-99%. The hydrogel composite forming the outer membrane may, for example, include 0.1% w/w up to 20% w/w of Chitosan. The hydrogel composite may further include excipients such as Xanthan Gum, water, propylene glycol and/or vegetable glycerin.

Any of the following compounds may also break down upon contacting the aerosol, and so may be included in the hydrogel composite (instead of, or in addition to, Chitosan): Hyaluronic Acid, Dextran, Poly-Acrylamide, Polyacrylic Acid, Guar Gum Succinate, Kappa-Carrageenan, Poly(vinyl Alcohol).

The microcapsule 710 may be manufactured by first freeze-drying a flavorant to remove any moisture from the flavorant and produce a flavorant cake. The flavorant cake may be molded into a desired shape (e.g., a ball) using a mold. Chitosan, together with excipients forming the hydrogel may be then be mixed together to produce the hydrogel composite. The hydrogel composite may be layered in a mold, into which the flavorant cake is inserted. The hydrogel composite may then be rolled to form an outer membrane around the flavorant cake.

The flavorant layer 708 may include a plurality of microcapsules 710. In use, the sticker 700 may be adhered to a mouthpiece of a smoking substitute apparatus and inserted into a user's mouth. In this manner, the microcapsules 710 may be located in the user's mouth. Then, when the user inhales an aerosol from the smoking substitute apparatus, the aerosol may pass through the central portion 706 of the sticker and thus come into contact with the microcapsules 710 in the flavorant layer 708. As a result, the outer membrane 714 of the microcapsules 710 may disintegrate due to the change in pH caused by the aerosol. This may cause release the flavorant 712 into the user's mouth.

Third Mode: Packaging for a Smoking Substitute Apparatus Including an Aromatic Additive

Aspects and embodiments of the third mode of the present disclosure will now be discussed with reference to the accompanying figures. Further aspects and embodiments of the third mode will be apparent to those skilled in the art. All documents mentioned in this text are incorporated herein by reference.

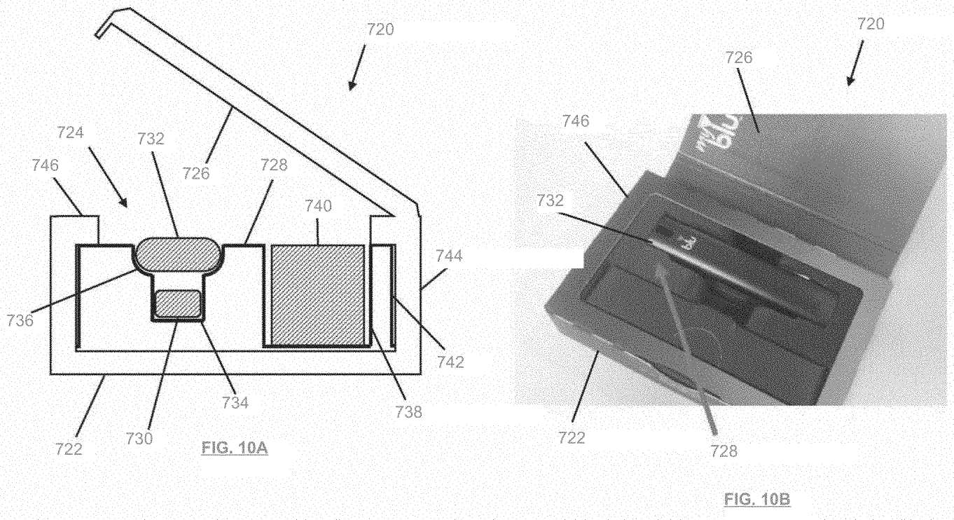

FIGS. 10A and 10B illustrate packaging 720 for a smoking substitute apparatus according to an embodiment of the disclosure. FIG. 10A shows a schematic cross-sectional view of packaging 720, whilst FIG. 10B shows an image of packaging 720. Packaging 720 includes an outer housing 722 in the form of a box made of cardboard which defines a compartment 724 in which a smoking substitute apparatus may be received. The outer housing 722 includes a lid 726 arranged to cover an opening of the outer housing 722. The lid 726 may be opened to access the compartment 724 in the outer housing 722, as shown in FIGS. 10A and 10B.

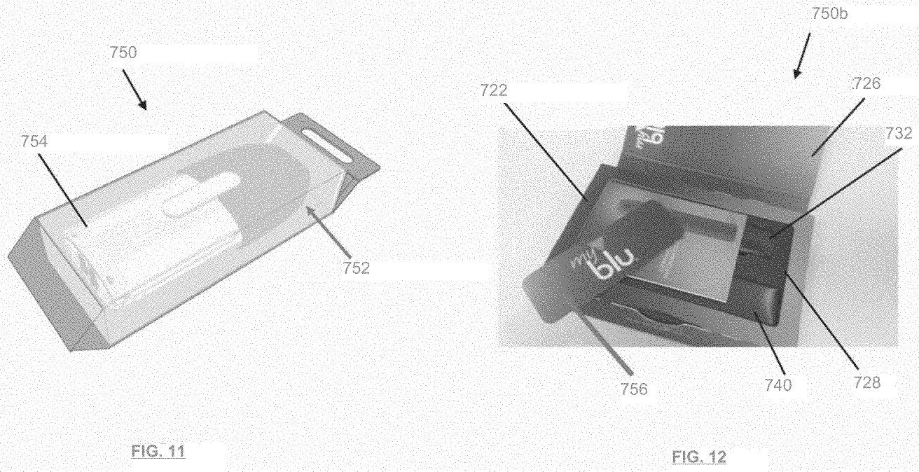

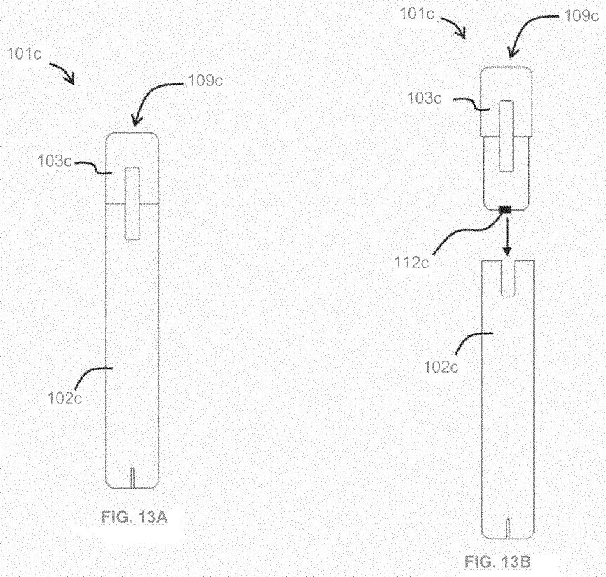

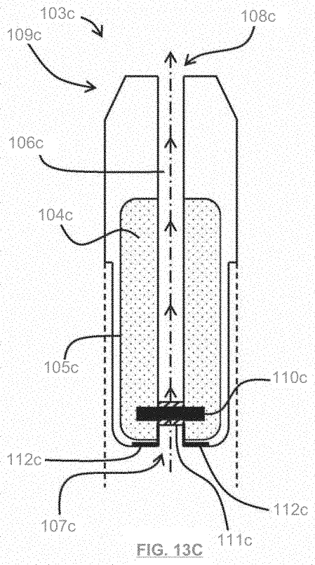

An inner packaging component 728 is disposed within the inner compartment 724 in the outer housing 722. The inner packaging component 728 is arranged to hold a smoking substitute apparatus in the form of a consumable 730 (e.g., cartridge) for a smoking substitute system, and a main body 732 for the smoking substitute system. Examples of the consumable 730 and main body 732 for a smoking substitute system are described in more detail below in relation to FIGS. 13A-13C.

The inner packaging component 728 includes a first recess 734 defined therein which is arranged to receive the consumable 730, and a second recess 736 defined therein which is arranged to receive the main body 732. In the example shown, the first recess 734 is located below the second recess 736 and accessed via the second recess 736. This may serve to make the packaging 720 more compact. However, in other examples, the first and second recesses 734, 736 may be disposed adjacent to one another. A shape of the first recess 734 is complementary to a shape of the consumable 730. As can be seen in FIG. 10A, a cross-sectional shape of the first recess 734 substantially matches a cross-sectional shape of the consumable 730. In this manner, when the consumable 730 is inserted into the first recess 734, it may be securely held in place in the first recess 734. This may prevent the consumable 730 from moving around within the packaging 720, to avoid the consumable being damaged. Similarly, a shape of the second recess 736 is complementary to a shape of the main body 732, so that the main body may be securely held in place when it is inserted into the second recess 736.

The inner packaging component 728 further includes a third recess 738 arranged to receive a box 740. The box 740 may, for example, contain one or more components for the smoking substitute system. For example, the box 740 may contain a power cable for charging a battery in the main body 732.

The inner packaging component 728 is arranged such that a sidewall 742 of the inner packaging component 728 is in contact with a sidewall 744 of the outer housing 722, to prevent the inner packaging component from moving around within the compartment 724 in the outer housing 722. Additionally, the inner packaging component 728 is prevented from falling out of the outer housing 722 by a retaining lip 746 disposed on the sidewall 744 of the outer housing 722.

The inner packaging component 728 may be made of plastic and may, for example, be manufactured using a thermoforming (e.g., vacuum forming) technique. Surfaces of the inner packaging component 728 in the first recess 734 and second recess 736 may include a textured surface comprising a soft material (e.g., a textile material or the like). This may reduce the risk of scratching a surface of the consumable 730 or the main body 732 when they are inserted into their respective recesses.

An aromatic additive is included in the inner packaging component 728. The aromatic additive may be provided in various forms. In one embodiment, the aromatic additive is provided as a coating on a surface of the inner packaging component 728. For example, the aromatic additive may be sprayed or otherwise applied to the surface of the inner packaging component 728 during manufacture of the inner packaging component 728. Preferably, the aromatic additive may be provided on a surface of the inner packaging component 728 located in the first recess 734 and the second recess 736. In this manner, when the consumable 730 and main body 732 are held in the inner packaging component 728, they may come in direct contact with portion of the inner packaging component 728 comprising the aromatic additive. In this manner, aromatic additive may be transferred from the inner packaging component 728 to the consumable 730 and main body 732 when they are received in the packaging 720. Then, when the user uses the consumable 730 and/or main body 732, they may perceive an aroma produced by the aromatic additive located on the consumable 730 and/or main body 732.

Additionally, or alternatively, the aromatic additive may be directly incorporated into the material forming the inner packaging component 728. For example, where the inner packaging component 728 is made of plastic, the aromatic additive may be mixed into a polymer solution used to make the plastic for making the inner packaging component. This may produce an inner packaging component 728 which is itself scented, and which may release an aroma.

The aromatic additive may also be arranged to produce an aroma when the packaging 720 is opened by the user. For example, prior to use, the lid 726 of the outer housing 722 may be closed, to prevent the aromatic additive from diffusing outside the outer housing 722. The aromatic additive in and/or on the inner packaging component 728 may diffuse inside the compartment 724 in the outer housing. Then, when the user opens the lid 726, the aromatic additive may be released, producing an aroma perceived by the user.

The packaging 720 containing the consumable 730 and main body 732, as illustrated in FIG. 10A, may be considered as a smoking substitute product. In other examples, the packaging may be arranged to hold a different number of components, and/or different types of components for a smoking substitute system.

FIG. 11 shows a perspective view of packaging 750 for a smoking substitute apparatus according to another embodiment of the disclosure. The packaging 750 includes a sealed plastic pouch 752 which defines a sealed enclosure in which a consumable 754 for a smoking substitute system is received. The sealed plastic pouch 752 is made of a transparent plastic to enable a user to see the consumable 754 within the pouch 752. The sealed plastic pouch 752 may, for example, be made using a flow wrapping technique. In the example shown, the consumable 754 is a cartridge for a smoking substitute system.

The sealed plastic pouch 752 contains an atmosphere comprising an aromatic additive. The atmosphere comprising the aromatic atmosphere is sealed inside the pouch 752 together with the consumable 754. In this manner, when the sealed plastic pouch 752 is opened by a user, the atmosphere comprising the aromatic additive may be released and produce an aroma that is perceived by the user. Additionally, some of the aromatic additive in the atmosphere in the sealed plastic pouch 752 may settle on a surface of the consumable 754, which may produce a scent and/or taste for the user when the user uses the consumable 754. As an example, the atmosphere in the sealed plastic pouch 752 may include a mist that is a mixture of ethanol and a flavorant.

To produce the packaging 750, first the plastic pouch 752 may be manufactured, e.g., using a flow wrapping technique. The consumable 754 may then be placed inside the pouch 752, and an atmosphere comprising an aromatic additive may be introduced into the plastic pouch 752. For example, a mixture of ethanol and flavorant may be atomized and sprayed into the plastic pouch 752. Subsequently, the plastic pouch 752 may be sealed, to form a sealed enclosure around the consumable 754 containing the atomized mixture of ethanol and flavorant. The atomized mixture of ethanol and flavorant may then settle on an inner surface of the pouch 752 and/or on the consumable. Over time, the ethanol may evaporate, leaving the flavorant on the inner surface of the pouch 752 and the consumable 754. This may produce an aroma in the packaging 750 and on the consumable 754, and may also produce a taste or scent when a user uses the consumable 754.

Together, the packaging 750 and consumable 754 may be considered as a smoking substitute product. Providing an individually packaged (wrapped) consumable in this manner may be beneficial, as it may enable the delivery of an aroma (via the aromatic additive in the packaging 750) for each consumable. In this manner, a user may experience a “fresh” aroma each time they open the packaging for a new consumable.

In other examples, other components of a smoking substitute system may be contained within the sealed plastic pouch 752, e.g., a main body of a smoking substitute system, or even a whole smoking substitute system.

FIG. 12 shows an image of packaging 750b for a smoking substitute apparatus that is another embodiment of the third mode of the disclosure. The packaging 750b is similar to packaging 750 described above in relation to FIGS. 10A and 10B. Accordingly, components of packaging 750b which correspond to components of packaging 750 described above are given the same reference numerals in FIG. 12 as the corresponding components in FIGS. 10A and 10B, and are not described again.

The packaging 750b includes a scented card 756, which may be placed inside the inner compartment 724 of the outer housing 722. The scented card 756 may be made of a paper or cardboard material, and includes an aromatic additive for producing an aroma. The aromatic additive comprised by the scented card 756 is a non-tobacco scented aromatic additive. For example, the aromatic additive may include menthol, licorice, chocolate, fruit flavor (including, e.g., citrus, cherry etc.), vanilla, spice (e.g., ginger, cinnamon). The aromatic additive may be provided as a coating on an outer surface of the scented card 756 (e.g., it may be sprayed onto the scented card 756), or it may be incorporated into a material forming the scented card 756.

The scented card 756 may be placed in the inner compartment 724 of the outer housing 722 together with a smoking substitute apparatus (e.g., consumable 730 and/or main body 732). When the lid 726 of the outer housing 722 is closed, aromatic additive from the scented card 756 may diffuse within the inner compartment 724. This may produce an aroma that is perceived by a user when the user opens the lid 726. Some of the aromatic additive may be settle on a surface of the smoking substitute apparatus, which may produce a smell or taste when the user uses the smoking substitute apparatus. Additionally, some of the aromatic additive may transferred from the scented card 756 to the smoking substitute apparatus (e.g., where the scented card and smoking substitute apparatus are in contact). In this manner, the scented card 756 may enable flavor delivery to a user of the smoking substitute apparatus.

We now describe, with relation to FIGS. 13A, 13B and 13C a smoking substitute system that may be contained in packaging of the disclosure, e.g., to form a smoking substitute product. FIGS. 13A and 13B illustrate a smoking substitute system in the form of an e-cigarette system 101c. The system 101c comprises an e-cigarette device defining a main body 102c of the system 101c, and a smoking substitute apparatus in the form of an e-cigarette consumable (or “pod”) 403. In the illustrated embodiment the consumable 103c (smoking substitute apparatus) is removable from the main body (e-cigarette device), so as to be a replaceable component of the system 101c. In other words, the e-cigarette system 101c is a closed system.

As is apparent from FIGS. 13A and 13B, the consumable 103c is configured to engage the main body 102c. FIG. 13A shows the main body 102c and the consumable 103c in an engaged state, whilst FIG. 13B shows the main body 102c and the consumable 103c in a disengaged state. When engaged, a portion of the consumable 103c is received in a cavity of the main body 102c and is retained in the engaged position by way of a snap-engagement mechanism. In other embodiments, the main body 102c and consumable 103c may be engaged by screwing one into (or onto) the other, through a bayonet fitting, or by way of an interference fit.

The system 101c is configured to vaporize an aerosol-former, which in the illustrated embodiment, is in the form of a nicotine-based e-liquid 104c. The e-liquid 104c comprises nicotine and a base liquid including propylene glycol and/or vegetable glycerin. In the present embodiment, the e-liquid 104c is flavorless (and does not include any added flavorant). That is, if the e-liquid 104c were to be inhaled (i.e., in aerosol form) by a user, it would not have a particularly perceptible flavor or taste.

As is more apparent from FIG. 13C, this e-liquid 104c is stored within a reservoir in the form of a tank 105c that forms part of the consumable 103c. In the illustrated embodiment, the consumable 103c is a “single-use” consumable 103c. That is, upon exhausting the e-liquid 104c in the tank 105c, the intention is that the user disposes of the entire consumable 103c. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105c surrounds, and thus defines a portion of, a passage 106c that extends between an inlet 107c and an outlet 108c at opposing ends of the consumable 103c. In this respect, the passage comprises an upstream end at the end of the consumable 103c that engages with the main body 102c, and a downstream end at an opposing end of the consumable 103c that comprises a mouthpiece 109c of the system 101c. When the consumable 103c is engaged with the main body 102c, a user can inhale (i.e., take a puff) via the mouthpiece 109c so as to draw air through the passage 106c, and so as to form an airflow (indicated by arrows) in a direction from the inlet 107c to the outlet 108c of the passage 106c. Although not illustrated, the passage 106c may be partially defined by a tube (e.g., a metal tube) extending through the consumable 103c. The passage 106c is in fluid communication with a gap defined between the consumable 103c and the main body 102c (when engaged) such that air outside of the system 101c is drawn into the passage 106c (during an inhale).

The smoking substitute system 101c is configured to vaporize the e-liquid 104c for inhalation by a user. To provide this, the consumable 103c comprises a heater having of a porous wick 110c and a resistive heating element in the form of a heating filament 111c that is helically wound around a portion of the porous wick 110c. The porous wick 110c extends across the passage 106c (i.e., transverse to a longitudinal axis of the passage 406) and opposing ends of the wick 110c extend into the tank 105c (so as to be submerged in the e-liquid 104c). In this way, e-liquid 104c contained in the tank 105c is conveyed from the opposing ends of the porous wick 110c to a central portion of the porous wick 110c so as to be exposed to the airflow in the passage 106c (i.e., caused by a user inhaling). In other embodiments the heating filament 111c and/or wick 110c may form part of the main body (but, may engage the tank 105c during engagement of the main body 102c and the consumable 103c).

The helical filament 111c is wound about this exposed central portion of the porous wick 110c and is electrically connected to an electrical interface in the form of electrical contacts 112c mounted at the end of the consumable that is proximate the main body 102c (when engaged). When the consumable 103c is engaged with the main body 102c, the electrical contacts 112c contact corresponding electrical contacts (not shown) of the main body 102c. The main body electrical contacts are electrically connected to a power source (not shown) of the main body 102c, such that (in the engaged position) the filament 111c is electrically connected to the power source. In this way, power can be supplied by the main body 102c to the filament 111c in order to heat the filament 111c. This heat is transferred from the filament 111c to the porous wick 110c which causes e-liquid 104c conveyed by the porous wick 110c to increase in temperature to a point at which it vaporizes. The vaporized e-liquid becomes entrained in the airflow and, between the vaporization point at the filament 111c and the outlet 108c of the passage 106c, condenses to form an aerosol. This aerosol is then inhaled, via the mouthpiece 109c, by a user of the system 101c.

The power source of the main body 102c may be in the form of a battery (e.g., a rechargeable battery).

The main body 102c may comprise a connector in the form of, e.g., a USB port for recharging this battery. The main body 102c may also comprise a controller that controls the supply of power from the power source to the main body electrical contacts (and thus to the filament 111c). That, is the controller may be configured to control a voltage applied across the main body electrical contacts, and thus the voltage applied across the filament 111c. In this way, the filament 111c may only be heated under certain conditions (e.g., during a puff and/or only when the system is in an active state). In this respect, the main body 102c may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102c and consumable 103c may comprise a further interface which may, for example, be in the form of an RFID reader, a barcode or QR code reader. This interface may be able to identify a characteristic (e.g., a type) of a consumable 103c engaged with the main body 102c. In this respect, the consumable 103c may include any one or more of an RFID chip, a barcode or QR code, or memory within which is an identifier and which can be interrogated via the interface.

In some cases, all or part of the smoking substitute system 101c may be received in packaging of the disclosure. For example, only the consumable 103c may be provided in packaging of the disclosure (e.g., in sealed plastic pouch 752), with the main body 102c being provided in separate packaging. In other cases, both the main body 102c and the consumable 103c may be provided in the same packaging (e.g., packaging 720 or 300).

The disclosure may be as described in the following clauses:

1. Packaging for a smoking substitute apparatus, the packaging comprising a compartment for receiving a smoking substitute apparatus, and an aromatic additive.

2. Packaging according to clause 1, wherein the packaging includes:
an outer housing forming the compartment for receiving the smoking substitute apparatus; and
an inner packaging component disposed within the outer housing, the inner packaging component being configured to hold the smoking substitute apparatus;
wherein the inner packaging component comprises at least a part of the aromatic additive.

3. Packaging according to clause 2, wherein the inner packaging component includes a recess for holding the smoking substitute apparatus, the recess having a shape that is complementary to a shape of the smoking substitute apparatus.

4. Packaging according to clause 2 or 3, wherein at least part of the aromatic additive comprised by the inner packaging component is provided on a surface of the inner packaging component.

5. Packaging according to clause 4, wherein the surface of the inner packaging component is arranged to contact the smoking substitute apparatus when the smoking substitute apparatus is held in the inner packaging component.

6. Packaging according to one of clauses 2 to 5, wherein at least part of the aromatic additive comprised by the inner packaging component is integrated into a material forming the inner packaging component.

7. Packaging according to clause 1, wherein the compartment for receiving the smoking substitute apparatus is a sealed enclosure containing an atmosphere comprising at least part of the aromatic additive.

8. Packaging according to clause 7, wherein the sealed compartment is formed by a plastic pouch.

9. Packaging according to any preceding clause, further comprising a scented card disposed within the compartment for receiving the smoking substitute apparatus, wherein the scented card comprises at least part of the aromatic additive.

10. Packaging according to clause 9, wherein the aromatic additive comprised by the scented card is a non-tobacco scented aromatic additive.

11. A smoking substitute product comprising:
packaging according to any preceding clause; and
a smoking substitute apparatus contained within the compartment for receiving a smoking substitute apparatus.

12. A smoking substitute produce according to clause 11, wherein the smoking substitute apparatus is a consumable for a smoking substitute system.

13. A method of packaging a smoking substitute apparatus, the method comprising:
forming a compartment for receiving the smoking substitute apparatus;
including an aromatic additive in the compartment; and
placing the smoking substitute apparatus in the compartment.

14. A method according to clause 13, further comprising:
forming an outer housing of the packaging to provide the compartment for receiving the smoking substitute apparatus;
forming an inner packaging component for holding the smoking substitute apparatus, the inner packaging component comprising at least part of the aromatic additive;
placing the smoking substitute apparatus in the inner packaging component, and
placing the inner packaging component in the outer housing.

15. A method according to clause 13, further comprising:
introducing into the compartment an atmosphere comprising at least part of the aromatic additive; and
sealing the compartment to form a sealed enclosure around the smoking substitute apparatus containing the atmosphere.

16. A method according to any one of clauses 13 to 15, further comprising: placing a scented card in the compartment for receiving the smoking substitute apparatus, the scented card comprising at least part of the aromatic additive.

17. A method of delivering flavor to a user of a smoking substitute apparatus, the method comprising:
providing a smoking substitute apparatus in packaging, the packaging including an aromatic additive;
opening the packaging to retrieve the smoking substitute apparatus; perceiving, by the user, the aromatic additive; and
using, by the user, the smoking substitute apparatus.

Fourth Mode: A Smoking Substitute Apparatus for Use with a Source of Pressurized Additive

Aspects and embodiments of the fourth mode of the present disclosure will now be discussed with reference to the accompanying figures. Further aspects and embodiments of the fourth mode will be apparent to those skilled in the art. All documents mentioned in this text are incorporated herein by reference.

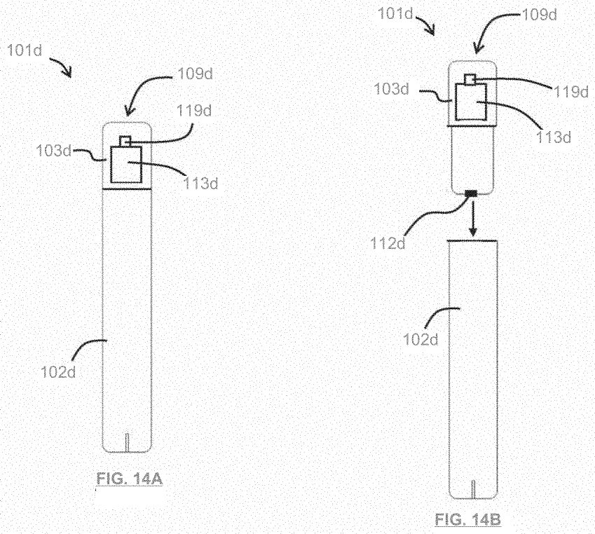

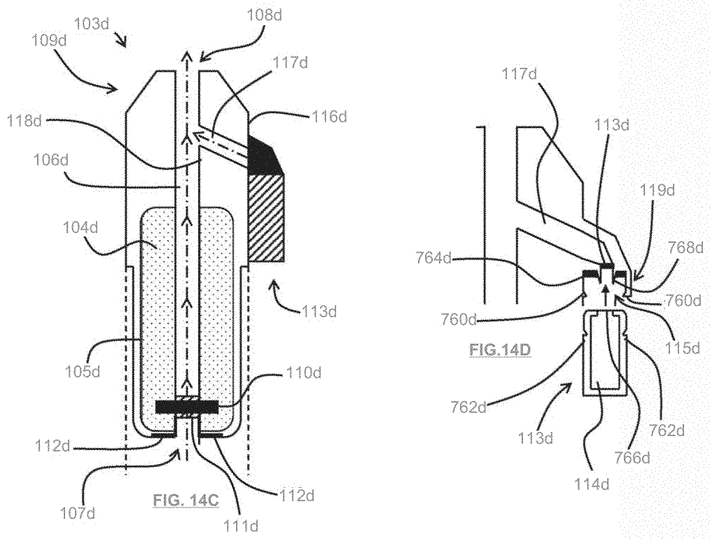

FIGS. 14A and 14B illustrate a smoking substitute system in the form of an e-cigarette system 101d. The system 101d comprises an e-cigarette device defining a main body 102d of the system 101d, and a smoking substitute apparatus in the form of an e-cigarette consumable (or “pod”) 103d. In the illustrated embodiment the consumable 103d (smoking substitute apparatus) is removable from the main body (e-cigarette device), so as to be a replaceable component of the system 101d. In other words, the e-cigarette system 101d is a closed system.

As is apparent from FIGS. 14A and 14B, the consumable 103d is configured to engage the main body 102d. FIG. 14A shows the main body 102d and the consumable 103d in an engaged state, whilst FIG. 14B shows the main body 102d and the consumable 103d in a disengaged state. When engaged, a portion of the consumable 103d is received in a cavity of the main body 102d and is retained in the engaged position by way of a snap-engagement mechanism. In other embodiments, the main body 102d and consumable 103d may be engaged by screwing one into (or onto) the other, through a bayonet fitting, or by way of an interference fit.

The system 101d is configured to vaporize an aerosol-former, which in the illustrated embodiment, is in the form of a nicotine-based e-liquid 104d. The e-liquid 104d comprises nicotine and a base liquid including propylene glycol and/or vegetable glycerin. In the present embodiment, the e-liquid 104d is flavorless (and does not include any added flavorant). That is, if the e-liquid 104d were to be inhaled (i.e., in aerosol form) by a user, it would not have a particularly perceptible flavor or taste.

As is more apparent from FIG. 14C, this e-liquid 104d is stored within a reservoir in the form of a tank 105d that forms part of the consumable 103d. In the illustrated embodiment, the consumable 103d is a “single-use” consumable 103d. That is, upon exhausting the e-liquid 104d in the tank 105d, the intention is that the user disposes of the entire consumable 103d. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105d surrounds, and thus defines a portion of, a passage 106d that extends between an inlet 107d and an outlet 108d at opposing ends of the consumable 103d. In this respect, the passage comprises an upstream end at the end of the consumable 103d that engages with the main body 102d, and a downstream end at an opposing end of the consumable 103d that comprises a mouthpiece 109d of the system 101d.

When the consumable 103d is engaged with the main body 102d, a user can inhale (i.e., take a puff) via the mouthpiece 109d so as to draw air through the passage 106d, and so as to form an airflow (indicated by arrows) in a direction from the inlet 107d to the outlet 108d of the passage 106d. Although not illustrated, the passage 106d may be partially defined by a tube (e.g., a metal tube) extending through the consumable 103d. The passage 106d is in fluid communication with a gap defined between the consumable 103d and the main body 102d (when engaged) such that air outside of the system 101d is drawn into the passage 106d (during an inhale).

The smoking substitute system 101d is configured to vaporize the e-liquid 104d for inhalation by a user. To provide this, the consumable 103d comprises a heater having of a porous wick 110d and a resistive heating element in the form of a heating filament 111d that is helically wound around a portion of the porous wick 110d. The porous wick 110d extends across the passage 106d (i.e., transverse to a longitudinal axis of the passage 106d) and opposing ends of the wick 110d extend into the tank 105d (so as to be submerged in the e-liquid 104d). In this way, e-liquid 104d contained in the tank 105d is conveyed from the opposing ends of the porous wick 110d to a central portion of the porous wick 110d so as to be exposed to the airflow in the passage 106d (i.e., caused by a user inhaling).

The helical filament 111d is wound about this exposed central portion of the porous wick 110d and is electrically connected to an electrical interface in the form of electrical contacts 112d mounted at the end of the consumable that is proximate the main body 102d (when engaged). When the consumable 103d is engaged with the main body 102d, the electrical contacts 112d contact corresponding electrical contacts (not shown) of the main body 102d. The main body electrical contacts are electrically connected to a power source (not shown) of the main body 102d, such that (in the engaged position) the filament 111d is electrically connected to the power source. In this way, power can be supplied by the main body 102d to the filament 111d in order to heat the filament 111d. This heat is transferred from the filament 111d to the porous wick 110d which causes e-liquid 104d conveyed by the porous wick 110d to increase in temperature to a point at which it vaporizes. The vaporized e-liquid becomes entrained in the airflow and, between the vaporization point at the filament 111d and the outlet 108d of the passage 106d, condenses to form an aerosol. This aerosol is then inhaled, via the mouthpiece 109d, by a user of the system 101d.

The power source of the main body 102d may be in the form of a battery (e.g., a rechargeable battery).

The main body 102d may comprise a connector in the form of, e.g., a USB port for recharging this battery. The main body 102d may also comprise a controller that controls the supply of power from the power source to the main body electrical contacts (and thus to the filament 111d). That, is the controller may be configured to control a voltage applied across the main body electrical contacts, and thus the voltage applied across the filament 111d. In this way, the filament 111d may only be heated under certain conditions (e.g., during a puff and/or only when the system is in an active state). In this respect, the main body 102d may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102d and consumable 103d may comprise a further interface which may, for example, be in the form of an RFID reader, a barcode or QR code reader. This interface may be able to identify a characteristic (e.g., a type) of a consumable 103d engaged with the main body 102d. In this respect, the consumable 103d may include any one or more of an RFID chip, a barcode or QR code, or memory within which is an identifier and which can be interrogated via the interface.

As is apparent from FIGS. 14C and 14D, the smoking substitute system 101d further comprises an additive source in the form of a receptacle 113d (formed of, e.g., metal or plastic) comprising a cavity 114d for containing a pressurized additive (i.e., contained above atmospheric pressure) which, in the present embodiment, comprises a flavorant (for delivering flavor to a user).

In order to deliver the additive to a user, the consumable 103d comprises an additive inlet 115d in fluid communication with the passage 106d downstream of the wick 110d. The additive inlet 115d is formed in an outer surface of a housing 116d of the consumable 103d and is fluidly connected to the passage 106d by a conduit 117d. This conduit 117d extends generally laterally with respect to the passage 106d, but is not perpendicular to the passage 106d, so as to provide for a smooth flow of additive from the conduit 117d to the passage 106d. As will be described further below, the inlet 115d is configured for connection with the receptacle 113d for supply of the additive from the cavity 114d of the receptacle 113d.

To control the flow of additive, the consumable 103d comprises a valve 118d between the additive inlet 115d and the passage 106d (i.e., in the conduit 117d). The valve 118d may be a one-way directional valve or a check valve. The valve 118d is movable between an open position (FIG. 14E) and a closed position (FIG. 14D). In the open position additive is able to flow from the inlet 115d to the passage 106d. In the closed position the valve 118d obstructs the inlet 115d and flow of additive from the inlet 115d to the passage 106d is prevented. The valve 118d is biased towards the closed position.

Although not immediately apparent from the figures, the valve 118d is configured to move from the closed position to the open position in response to an inhalation from the outlet 108d by a user. In other words, inhalation at the outlet 108d causes a pressure drop in the conduit 117d. The differential pressure across the valve 118d causes the valve 118d to move from the closed position to the open position. Thus, when a user is inhaling the valve 118d is in the open position and additive is combined with the aerosol flow in the passage 106d. Conversely, when a user is not inhaling, the valve 118d remains in the closed position (as shown in FIG. 14D).

For releasable connection (i.e., mounting) of the receptacle 113d to the consumable 103d, the consumable comprises a connector 119d. In the illustrated embodiment the connector 119d operates by way of a snap-fit mechanism. In particular, the connector 119d comprises bump features 760d that are configured to engage with corresponding grooves 762d formed in an outer surface of the receptacle 113d. As the receptacle 113d is moved into connection with the connector 119d (as indicated by the arrow of FIG. 14D) the outermost bump feature 760d is deflected by an outer surface of the receptacle 113d, until it reaches the corresponding groove 762d and engages with the groove 760d.

The connector 119d comprises a sealing member in the form of an elastomeric sealing ring 764d. This sealing ring 764d is compressed as the receptacle 113d engages with the connector 119d so as to form a fluid seal between the receptacle 113d and the connector 119d. This prevents leakage of pressurized additive between the receptacle 113d and the connector 119d.

To prevent leakage of the pressurized additive prior to connection of the receptacle 113d with the connector 119d, the receptacle 113d comprises a frangible seal 766d (or membrane) that extends across an opening of the receptacle 113d. The frangible seal 766d is a thinner piece of material (e.g., foil) that may be integrally formed with the receptacle 113d or attached in another manner. In order to release additive from the cavity 114d (upon connection of the receptacle 113d to the connector 119d), the connector 119d comprises a seal breaching portion in the form of a tapered ring 768d extending about an opening to the connector 119d (in fluid connection with the inlet 115d). The ring 768d is tapered from its base to a distal end so as to be pointed. Thus, the ring is able to pierce the frangible seal 766d to release pressurized additive from the cavity 114d of the receptacle 113d. As is shown by the dashed arrows of FIG. 14E, this allows the additive to flow from the receptacle, through the inlet 115d and to the passage 106d (via the conduit 117d). The additive then mixes with aerosol (generated at the wick 110d) and may be inhaled via the outlet 108d by a user. In this way, the additive may change a property (e.g., flavor) of the aerosol inhaled by the user.

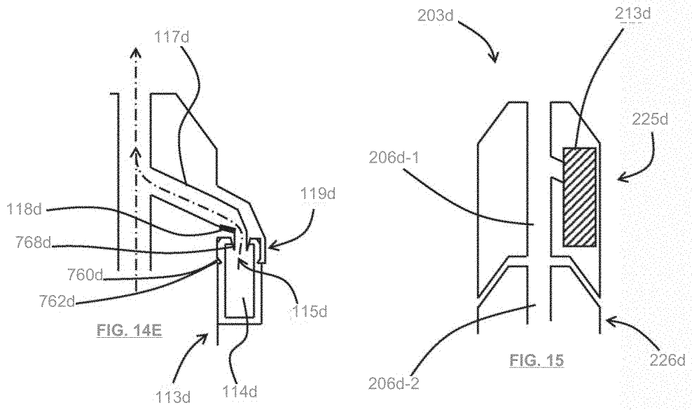

FIG. 15 shows a further embodiment of a smoking substitute apparatus, again in the form of a consumable 203d. This consumable 203d differs from the previously described consumable 103d, in that it comprises first 225d and second 226d portions that are releasably mountable to one another (e.g., by a screw connection, bayonet connection, snap engagement, etc.). The first portion 225d comprises the receptacle 113d and a first passage section 206d-1. The second portion 226d comprises the remaining components of the consumable 103d (e.g., generator, wick, heater, etc. (not shown)), and a second passage section 206d-2. When the first 225 and second 226 portions are mounted together, the first 206d-1 and second 206d-2 passage sections define the passage of the consumable 103d.

Fifth Mode: A Smoking Substitute Apparatus have Separate Storage for Aerosol Former and Property Modifying Agent

Aspects and embodiments of the fifth mode of the present disclosure will now be discussed with reference to the accompanying figures. Further aspects and embodiments of the fifth mode will be apparent to those skilled in the art. All documents mentioned in this text are incorporated herein by reference.

FIGS. 1A and 1B illustrate a smoking substitute system in the form of an e-cigarette system 101e. The system 101e comprises an e-cigarette device defining a main body 102e of the system 101e, and a smoking substitute apparatus in the form of an e-cigarette consumable (or “pod”) 103e. The smoking substitute apparatus is a smoking substitute apparatus. In the illustrated embodiment the consumable 103e (smoking substitute apparatus) is removable from the main body (e-cigarette device), so as to be a replaceable component of the system 101e. In other words, the e-cigarette system 101e is a closed system.

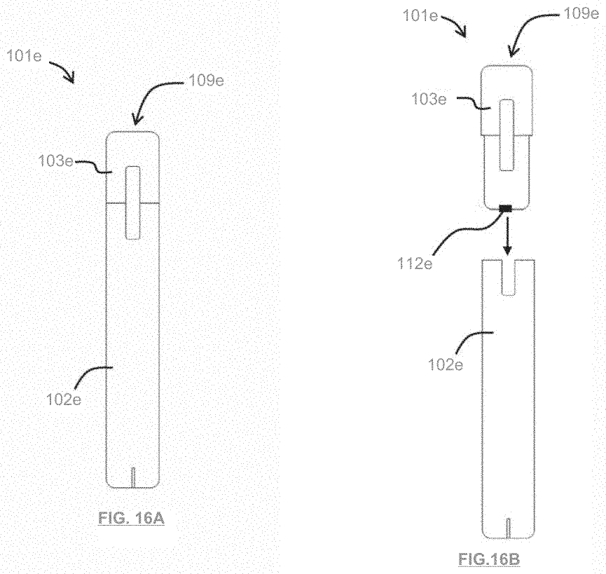

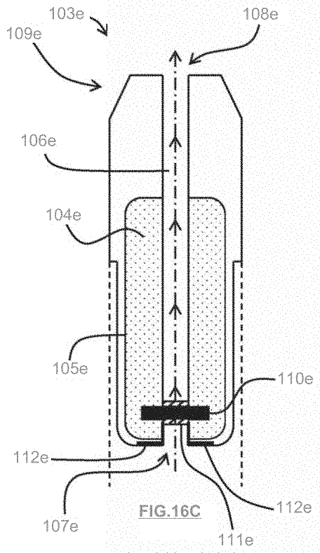

As is apparent from FIGS. 16A and 16B, the consumable 103e is configured to engage the main body 102e. FIG. 16A shows the main body 102e and the consumable 103e in an engaged state, whilst FIG. 16B shows the main body 102e and the consumable 103e in a disengaged state. When engaged, a portion of the consumable 103e is received in a cavity of the main body 102e and is retained in the engaged position by way of a snap-engagement mechanism. In other embodiments, the main body 102e and consumable 103e may be engaged by screwing one into (or onto) the other, through a bayonet fitting, or by way of an interference fit.

The system 101e is configured to vaporize an aerosol-former, which in the illustrated embodiment, is in the form of a nicotine-based e-liquid 104e. The e-liquid 104e comprises nicotine and a base liquid including propylene glycol and/or vegetable glycerin. In the present embodiment, the e-liquid 104e is flavorless (and does not include any added flavorant). That is, if the e-liquid 104e were to be inhaled (i.e., in aerosol form) by a user, it would not have a particularly perceptible flavor or taste.

As is more apparent from FIG. 16C, this e-liquid 104e is stored within a reservoir in the form of a tank 105e that forms part of the consumable 103e. In the illustrated embodiment, the consumable 103e is a “single-use” consumable 103e. That is, upon exhausting the e-liquid 104e in the tank 105e, the intention is that the user disposes of the entire consumable 103e. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105e surrounds, and thus defines a portion of, a passage 106e that extends between an inlet 107e and an outlet 108e at opposing ends of the consumable 103e. In this respect, the passage comprises an upstream end at the end of the consumable 103e that engages with the main body 102e, and a downstream end at an opposing end of the consumable 103e that comprises a mouthpiece 109e of the system 101e. When the consumable 103e is engaged with the main body 102e, a user can inhale (i.e., take a puff) via the mouthpiece 109e so as to draw air through the passage 106e, and so as to form an airflow (indicated by arrows) in a direction from the inlet 107e to the outlet 108e of the passage 106e. Although not illustrated, the passage 106e may be partially defined by a tube (e.g., a metal tube) extending through the consumable 103e. The passage 106e is in fluid communication with a gap defined between the consumable 103e and the main body 102e (when engaged) such that air outside of the system 101e is drawn into the passage 106e (during an inhale).

The smoking substitute system 101e is configured to vaporize the e-liquid 104e for inhalation by a user. To provide this, the consumable 103e comprises a heater having of a porous wick 110e and a resistive heating element in the form of a heating filament 111e that is helically wound around a portion of the porous wick 110e. The porous wick 110e extends across the passage 106e (i.e., transverse to a longitudinal axis of the passage 106e) and opposing ends of the wick 110e extend into the tank 105e (so as to be submerged in the e-liquid 104e). In this way, e-liquid 104e contained in the tank 105e is conveyed from the opposing ends of the porous wick 110e to a central portion of the porous wick 110e so as to be exposed to the airflow in the passage 106e (i.e., caused by a user inhaling).

The helical filament 111e is wound about this exposed central portion of the porous wick 110e and is electrically connected to an electrical interface in the form of electrical contacts 112e mounted at the end of the consumable that is proximate the main body 102e (when engaged). When the consumable 103e is engaged with the main body 102e, the electrical contacts 112e contact corresponding electrical contacts (not shown) of the main body 102e. The main body electrical contacts are electrically connected to a power source (not shown) of the main body 102e, such that (in the engaged position) the filament 111e is electrically connected to the power source. In this way, power can be supplied by the main body 102e to the filament 111e in order to heat the filament 111e. This heat is transferred from the filament 111e to the porous wick 110e which causes e-liquid 104e conveyed by the porous wick 110e to increase in temperature to a point at which it vaporizes. The vaporized e-liquid becomes entrained in the airflow and, between the vaporization point at the filament 111e and the outlet 108e of the passage 106e, condenses to form an aerosol. This aerosol is then inhaled, via the mouthpiece 109e, by a user of the system 101e.

The power source of the main body 102e may be in the form of a battery (e.g., a rechargeable battery). The main body 102e may comprise a connector in the form of, e.g., a USB port for recharging this battery. The main body 102e may also comprise a controller that controls the supply of power from the power source to the main body electrical contacts (and thus to the filament 111e). That, is the controller may be configured to control a voltage applied across the main body electrical contacts, and thus the voltage applied across the filament 111e. In this way, the filament 111e may only be heated under certain conditions (e.g., during a puff and/or only when the system is in an active state). In this respect, the main body 102e may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102e and consumable 103e may comprise a further interface which may, for example, be in the form of an RFID reader, a barcode or QR code reader. This interface may be able to identify a characteristic (e.g., a type) of a consumable 103e engaged with the main body 102e. In this respect, the consumable 103e may include any one or more of an RFID chip, a barcode or QR code, or memory within which is an identifier and which can be interrogated via the interface.

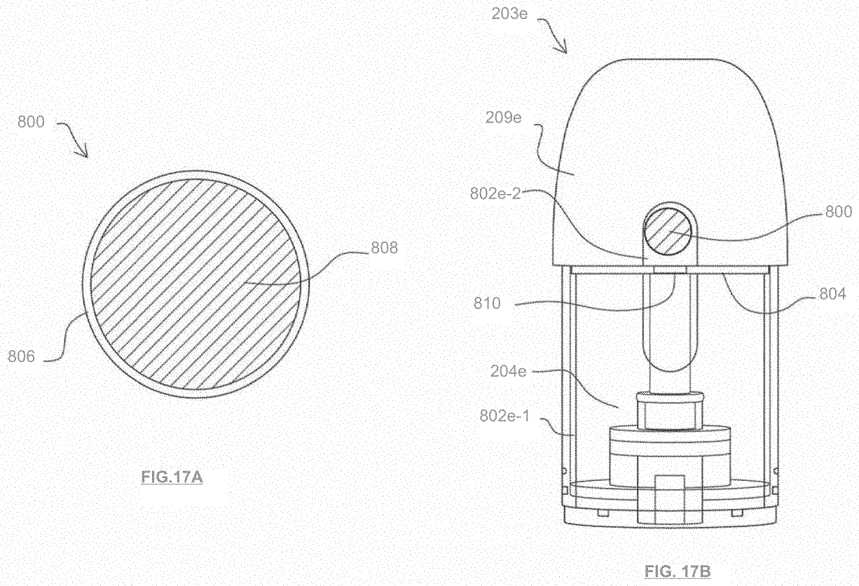

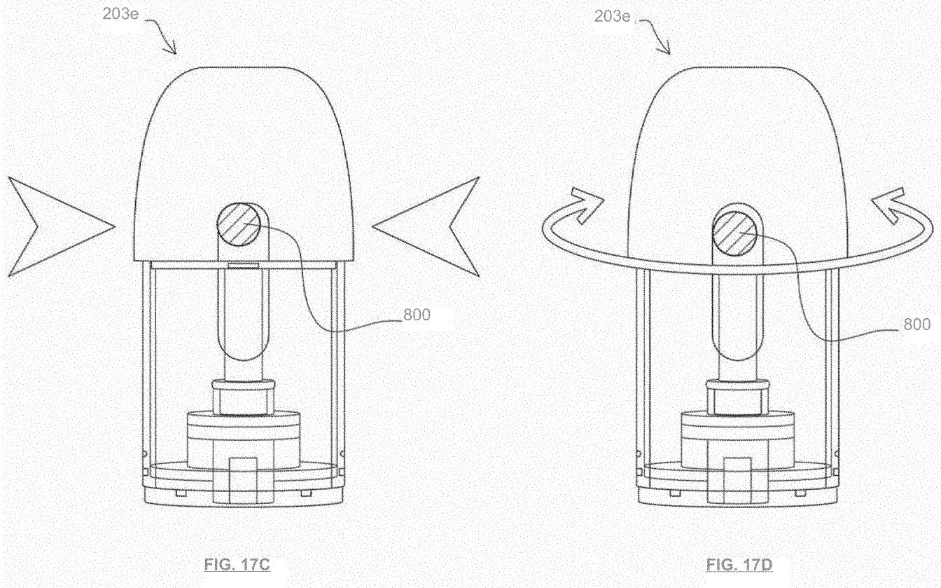

FIGS. 17A and 17B respectively shows a capsule 800 and a smoking substitute apparatus, or a consumable 203e for receiving the capsule 800 according to an embodiment of the present disclosure. The consumable 203e comprises a tank that is divided into a first compartment 802e-1 and a second compartment 802e-2 by a partition wall 804. In other words, the first compartment 802e-1 and the second compartment 802e-2 shares the partition wall 804. The partition wall 804 extends across the tank, e.g., from a sidewall of the tank towards an opposing sidewall of the tank, as such a base portion of the second compartment 802e-2 is adjacent to an upper portion of the first compartment 802e-1.

The capsule 800 comprises a shell 806 encapsulating a body of property modifying agent 808. In the illustrated embodiment the shell 806 is formed from a hydrolyzed collagen, such as a gelatin. The shell is insoluble in the property modifying agent, as such it remains stable during storage and transportation. The property modifying agent 808 in the illustrated embodiment is a liquid property modifying agent, and comprises a flavorant and a colorant respectively configured to modify the flavor and color of the aerosol former 204e as stored in the first compartment 802e-1. In other embodiments, the property modifying agent may be a solid, a gel, a liquid or a gas, and may comprise only a flavorant without the presence of a colorant or may comprise a colorant without the presence of a flavorant. For example, the property modifying agent 808 may alternatively be a powder encapsulated in the shell 806.

In the illustrated embodiment, the first compartment 802e-1 contains a supply of aerosol former, or e-liquid 204e, and the second compartment 802e-2 receives the capsule 800. That is, in this example, the consumable is provided with a capsule 800 contained in the second compartment 802e-2. The aerosol former 204e is in a liquid form. The aerosol former 204e comprises nicotine and a base liquid, e.g., propylene glycol. The aerosol former 204e does not contain any flavorant and therefore the aerosol former is not flavored. The aerosol former 204e in this embodiment does not contain any colorant. That is, the aerosol former 204e is in the natural color of the propylene glycol and the nicotine, and therefore it appears as a translucent or transparent liquid.

The partition wall 804 as shown in FIG. 17B comprises an opening 810. The opening 810 effects fluid communication between the first compartment 802e-1 and the second compartment 802e-2. However, the opening 810 is sized such that the capsule 800 as received in the first compartment 802e-1 is retained therein, e.g., the opening 810 is sized smaller than the cross-sectional diameter of the capsule 800. The opening 810 is unobstructed in the illustrated embodiment as shown in FIG. 17B. That is, the consumable 203e in this example is supplied with the aerosol former 204e freely flowable between the first compartment 802e-1 and second compartment 802e-2, e.g., the aerosol former 204e may be in contact with the capsule 800 and is separated from the property modifying agent 808 encapsulated in the capsule shell 806.

The second compartment 802e-2 of the consumable 203e in the illustrated embodiment comprises flexible sidewalls that deforms upon applying a force thereat. In some other embodiments, the consumable is formed from an elastic material and therefore both the first compartment and the second compartment comprise flexible sidewalls. Additionally, the mouthpiece 209e that surrounds the second compartment 802e-2 is also formed from an elastic material such that the second compartment 802e-2 may be configured to deform by compressing or twisting the mouthpiece 209e.

To release the property modifying agent 808 from the capsule 800, a user may compress, e.g., squeeze on, the mouthpiece 209e so as to bias the flexible sidewalls against each other, as shown in FIG. 17C.

Doing so urges the flexible sidewalls towards the capsule 800 and thereby compresses the capsule 800. The compressive stress acting on the capsule 800 causes the capsule shell 806 to rupture, and thereby releases the body of property modifying agent 808 to the cavity of the second compartment 802e-2. The property modifying agent 808 then flows or drips, under gravity, through the opening 810 and into the aerosol former 204e stored in the first compartment 802e-1. In the illustrated embodiment, the ruptured capsule shell 806 may be retained in the second compartment 802e-2 as its size precludes it form passing through the opening 810.

Alternatively, a user may twist the consumable 203e about its longitudinal axis, as shown in FIG. 17D. Doing so reduces the spacing between the flexible sidewalls and thereby urges the flexible sidewalls towards the capsule 800 to compress it. The compressive stress acting on the capsule 800 causes the capsule shell 806 to rupture, and thereby releases the body of property modifying agent 808 to the cavity of the second compartment 802e-2. The property modifying agent 808 then flows or drips, under gravity, through the opening 810 and into the aerosol former 204e stored in the first compartment 802e-1. In the illustrated embodiment, the ruptured capsule shell 806 may be retained in the second compartment 802e-2 as its size precludes it form passing through the opening 810. That is, the flexible sidewalls are configured to compress the capsule 800 stored in the second compartment 802e-2 either by compressing the flexible sidewalls or by twisting the consumable 203e.

In some other embodiments, the flexible sidewalls of the second compartment may comprise protrusion, in the form of ribs or projections for rupturing the capsules stored in the second compartment. The protrusions may allow pressure to build up at their tips, thereby allowing the capsules to be ruptured more efficiently.

In some other embodiments, the second compartment may comprise a moveable element for rupturing the capsule. The movable element may comprise an actuator that extends outwardly from the consumable that allows a user to move the moveable element and thereby to rupture the capsule. The movable element may be a movable wall positioned at an upper end of the second compartment away from the first compartment, and may configure to travel along the longitudinal axis of the consumable. In use, said movable wall may move towards the partition wall and thereby ruptures the capsules stored in the second compartment. That is, the moveable wall may function as a piston for rupturing the capsules. Alternatively, or in addition, the movable element may comprise one or more needles in the second compartment that is configured to travel along the longitudinal axis of the consumable. In use, said needles may move towards the capsules to pierce through the capsule shell, and thereby releasing the property modifying agent to the second compartment.

In some other embodiments, the capsules may comprise a capsule shell that is soluble in the aerosol former. In such cases, the opening may be sealed by a removable seal in order to prevent the aerosol former from coming into contact with the capsule shell, and thereby prevents the capsule shell from dissolving in the aerosol former prior to seal removal. More specifically, a seal portion of the removable seal may cover the opening in a sealing position and thereby hermetically seals the opening. Therefore, in the sealing position, the seal portion of the removable seal prevents fluid communication between the first compartment and the second compartment. The removal seal is configured to be removed from the sealing position and thereby detaches the seal portion form the opening, and thereby establishes fluid communication between the first compartment and second compartment.

The removable seal may additionally comprise a tab portion in connection with the seal portion. That is, the seal portion may be formed together with the tab portion to form the removable seal. Said tab portion may extends, along a slot in the consumable and outwardly from the base of the consumable through a check valve, such that at least a part of the tab portion is accessible to a user. The tab portion comprises an enlarged portion external to the consumable for a user to grip onto.

Prior to use or in use, the user may pull on the tab portion away from the consumable to detach the seal portion from the opening. The removable seal may be completely removed from the consumable to be disposed of. Doing so establishes fluid communication between the first compartment and second compartment, thereby allowing the aerosol former in the first compartment to come into contact with the capsule in the second compartment. More specifically, the capsule shell of the capsules may dissolve in the aerosol former once they come into contact with each other. As such, the body of the property modifying agent may then be released into the aerosol former.

FIG. 18 illustrates a consumable 814 according to another embodiment of the present disclosure. The consumable 814 is similar, functionally and structurally to the consumable 203e as shown in FIG. 17B. The consumable 814 differs to the consumable 203e in that the consumable 814 comprises a compartment inlet 816 at the second compartment 802e-3 for providing access to the second compartment 802e-3. More specifically, the compartment inlet 816 is sized to allow capsules 800 to be inserted through there and into the second compartment 802e-3. The compartment inlet 816 is closed by a flap 818 that is hingedly attached to the consumable 814 at a location adjacent to the compartment inlet 816. Therefore, the flap 818 is pivotable between an opened position for providing access to the second compartment 802e-3 and a closed position where the second compartment 802e-3 is sealed off. When the flap 818 in the opened position, a user may add capsules 800, either manually or with the use of a dispenser, to the second compartment 802e-2.

The quantity of property modifying agent releasable to the aerosol former relates to the number of capsules that is being added to the second compartment 802e-3. For example, a user may add a predetermined number of capsules to the second compartment 802e-3 in order to achieve the desired flavor and/or color. The user may add a mix of capsules each having a different flavorant (e.g., blueberry flavorant in a first capsule and cherry flavorant in a second capsule) so as to achieve a mixed flavor in the aerosol former.

In some embodiments, the volume of the second compartment 802e-3 is limited so as to limit the maximum amount of property modifying agent a user can add to the aerosol former. This prevents the user form overdosing the property modifying agent in the aerosol former.

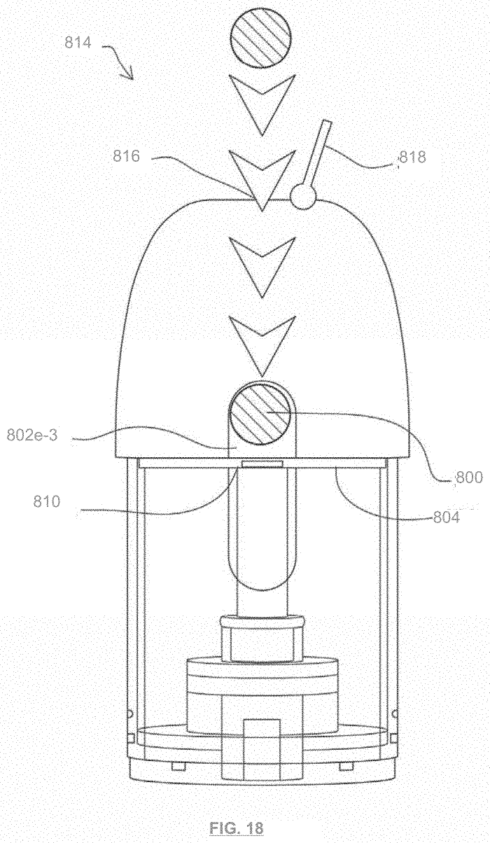

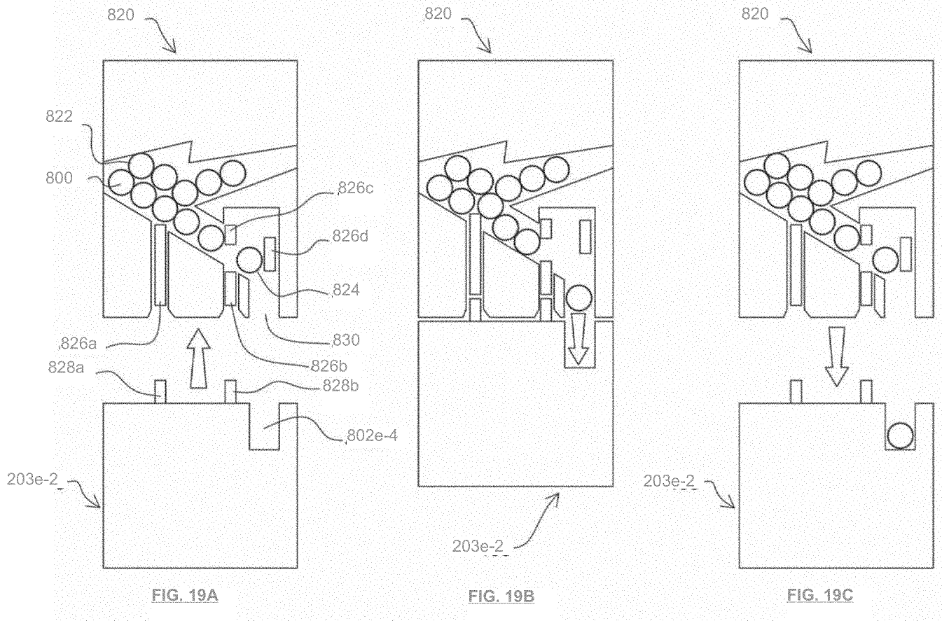

FIGS. 19a, 19b and 19c sequentially show a dispenser 820 dispensing a predetermined number of capsules 800 to a second compartment 802e-4 of a consumable 203e-2. FIG. 19a and FIG. 19c show the dispenser 820 being separated from the consumable 203e-2 and FIG. 19b shows the dispenser 820 is engaged with the consumable 203e-2. The dispenser 820 comprises a storage 822 for storing a supply of capsules 800 and a dispensing chamber 824 for storing a predetermined number of capsules to be dispensed. For example, the dispensing chamber 824 is sized to receive the required number of capsules to be dispensed, in this case a single capsule. However, the dispensing chamber may be sized to receive a plurality of capsules such that said plurality of capsules may be dispensed at once. When the dispenser is separated from the consumable, as shown in FIG. 19a, a dispensing element 826d prevents the capsule from being dispensed from the dispensing chamber 824.

The dispenser comprises a dispensing mechanism, which is formed of dispensing elements 826a, 826b, 826c and 826d, and is configured to be activated by a corresponding activating mechanism 828a and 828b at the consumable 203e-2. More specifically, the activating mechanism 828a and 828b are protrusions that, when the dispenser 820 is engaged with the consumable 203e-2 as shown in FIG. 19B, push onto the dispensing element 826a and 826b and thereby urges the dispensing mechanism towards an upward direction. This causes the dispensing element 826d to move and thereby allows a capsule to dispense through a dispenser outlet 830 into the second compartment 802e-4. Furthermore, when the dispensing mechanism is activated as shown in FIG. 19B, a slot formed between the dispensing element 826b and 826c also moves in line with a capsule 440 stored in the storage 822 and thereby allowing the capsule in the storage 822 to drop into the dispensing chamber 824. That is, activating the dispensing mechanism causes a capsule to be dispensed from the dispensing chamber 824, as well as allowing a capsule form the storage 822 to replenish the dispensing chamber 824.

Sixth Mode: A Smoking Substitute Apparatus Including Separate First and Second Airflows

Aspects and embodiments of the sixth mode of the present disclosure will now be discussed with reference to the accompanying figures. Further aspects and embodiments of the sixth mode will be apparent to those skilled in the art. All documents mentioned in this text are incorporated herein by reference.

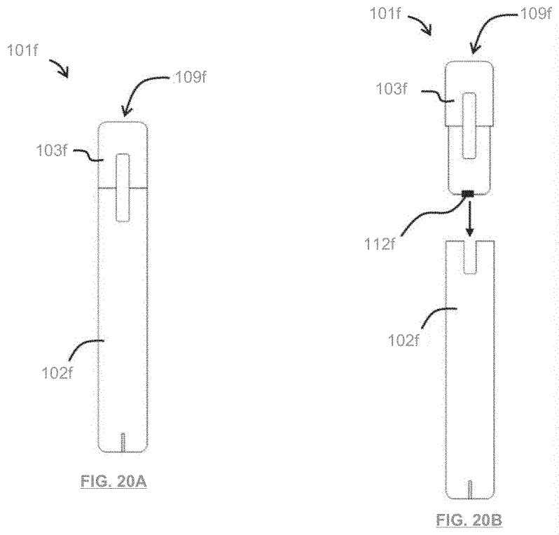

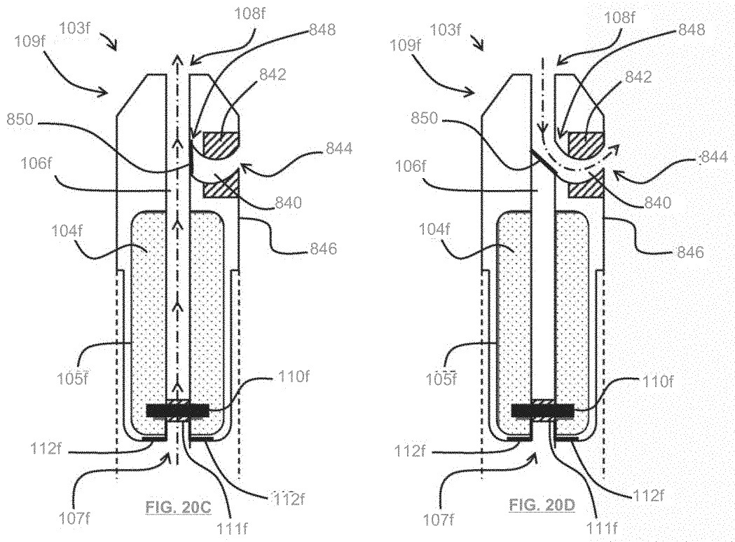

FIGS. 20A and 20B illustrate a smoking substitute system in the form of an e-cigarette system 101f. The system 101f comprises a main body 102f (i.e., an e-cigarette device) of the system 101f, and a smoking substitute apparatus in the form of an e-cigarette consumable (or “pod”) 103. In the illustrated embodiment the consumable 103f (smoking substitute apparatus) is removable from the main body (e-cigarette), so as to be a replaceable component of the system 101f. In other words, the e-cigarette system 101f system 101f is a closed system.

As is apparent from FIGS. 20A and 20B, the consumable 103f is configured to engage the main body 102f. FIG. 20A shows the main body 102f and the consumable 103f in an engaged state, whilst FIG. 20B shows the main body 102f and the consumable 103f in a disengaged state. When engaged, a portion of the consumable 103f is received in a cavity of the main body 102f and is retained in the engaged position by way of a snap-engagement mechanism. In other embodiments, the main body 102f and consumable 103f may be engaged by screwing one into (or onto) the other, through a bayonet fitting, or by way of an interference fit.

The system 101f is configured to vaporize an aerosol-former, which in the illustrated embodiment, is in the form of a nicotine-based e-liquid 104f. The e-liquid 104f comprises nicotine and a base liquid including propylene glycol and/or vegetable glycerin. In the present embodiment, the e-liquid 104f is flavorless (and does not include any added flavorant). That is, if the e-liquid 104f were to be inhaled (i.e., in aerosol form) by a user, it would not have a particularly perceptible flavor or taste.

As is more apparent from FIGS. 20C and 20D, this e-liquid 104f is stored within a reservoir in the form of a tank 105f that forms part of the consumable 103f. In the illustrated embodiment, the consumable 103f is a “single-use” consumable 103f. That is, upon exhausting the e-liquid 104f in the tank 105f, the intention is that the user disposes of the consumable 103f. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the device or stored in another component that is itself not single-use (e.g., a refillable cartomizer).

The tank 105f surrounds, and thus defines a portion of, a first passage 106f that extends between an inlet 107f and a mouthpiece opening 108f at opposing ends of the consumable 103f. In this respect, the first passage 106f comprises an upstream end at the end of the consumable that engages with the main body 102f, and a downstream end at an opposing end of the consumable 103f, which defines a mouthpiece 109f (comprising the mouthpiece opening 108f) of the system 101f. When the consumable 103f is engaged with the main body 102f, a user can inhale (i.e., take a puff) via the mouthpiece 109f so as to draw air through the first passage 106f, and so as to form an airflow (indicated by arrows in FIG. 20C) in a direction from the inlet 107f to the mouthpiece opening 108f of the first passage 106f. Although not illustrated, the first passage 106f may be at least partly defined by a tube (e.g., a metal tube) extending through the consumable 103f.

The smoking substitute system 101f is configured to vaporize the e-liquid 104f (and subsequently aerosolize the e-liquid) for inhalation by a user. To provide this, the consumable 103f comprises a heater having of a porous wick 110f and a resistive heating element in the form of a heating filament 111f that is helically wound around a portion of the porous wick 110f. The porous wick 110f extends across the first passage 106f (i.e., transverse to a longitudinal axis of the first passage 106f) and opposing ends of the wick 110f extend into the tank 105f (so as to be submerged in the e-liquid 104f). In this way, e-liquid 104f contained in the tank 105f is conveyed from the opposing ends of the porous wick 110f to a central portion of the porous wick 110f so as to be exposed to the airflow in the first passage 106f (i.e., caused by a user inhaling).

The helical filament 111f is wound about this exposed central portion of the porous wick 110f and is electrically connected to an electrical interface in the form of electrical contacts 112f mounted at the end of the consumable that is proximate the main body 102f (when engaged). When the consumable 103f is engaged with the main body 102f, the electrical contacts 112f contact corresponding electrical contacts (not shown) of the main body 102f. The main body electrical contacts are electrically connected to a power source (not shown) of the main body 102f, such that (in the engaged position) the filament 111f is electrically connected to the power source. In this way, power can be supplied by the main body 102f to the filament 111f in order to heat the filament 111f. This heat is transferred from the filament 111f to the porous wick 110f which causes e-liquid 104f conveyed by the porous wick 110f to increase in temperature to a point at which it vaporizes. The vaporized e-liquid becomes entrained in the airflow and, between the vaporization point at the filament 111f and the mouthpiece opening 108f of the first passage 106f, condenses to form an aerosol. This aerosol is then inhaled, via the mouthpiece 109f, by a user of the system 101f.

The power source of the main body 102f may be in the form of a battery (e.g., a rechargeable battery). The main body 102f may comprise a connector in the form of, e.g., a USB port for recharging this battery. The main body 102f may also comprise a controller that controls supply of power from the power source to the main body electrical contacts (and thus to the filament 111f). That, is the controller may be configured to control a voltage applied across the main body electrical contacts, and thus the voltage applied across the filament 111f. In this way, the filament 111f may only be heated under certain conditions (e.g., during a puff and/or only when the system is in an active state). In this respect, the main body 102f may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing).

The consumable 103f further comprises a second passage 840. The second passage comprises a flavor delivery portion 842 in fluid communication (i.e., via the second passage 840) with the mouthpiece 109f and, in particular, with the mouthpiece opening 108f of the mouthpiece 109f. The flavor delivery portion 842 may comprise a sintered polymer, a ceramic matrix, an open-cell foam and/or a cotton-based fibrous media. Flavorant may be deposited on one or more surfaces of the flavor delivery portion 110f. Alternatively, or additionally, the flavor delivery portion 110f may be impregnated with flavorant. In the illustrated embodiment the flavor delivery portion 110f comprises a channel that forms part of the second passage 840.

The flavorant may be provided in solid or liquid form. It may include menthol, licorice, chocolate, fruit flavor (including, e.g., citrus, cherry etc.), vanilla, spice (e.g., ginger, cinnamon) and tobacco flavor. The flavorant may be evenly dispersed or may be provided in isolated locations and/or varying concentrations.

The second passage 840 comprises an outlet 844 that, as will be described further below, is arranged to discharge an airflow (i.e., a flavored airflow) towards a user's nose (i.e., nasal cavity) upon exhalation into the mouthpiece 109f. In particular, the outlet 844 is formed in an in use upper surface 846 of the consumable 103f and is arranged so as to discharge an airflow (as depicted with arrows in FIG. 20D) in a direction that is at an angle with respect to a longitudinal axis of the first passage 106f. The outlet 844 is spaced from the mouthpiece 109f such that it does not form part of the mouthpiece 109f and, as such, may remain unobstructed when a user inhales or exhales at the mouthpiece 109f.

The second passage 840 is connected to, and branches from, the first passage 106f. Thus, the second passage 840 comprises an inlet 848 arranged to receive an airflow from the first passage 106f (as is shown in FIG. 20D) and that is located axially between the mouthpiece 109f and the wick 110f. The second passage 840 extends generally laterally from the first passage 106 and follows a generally curved profile. In particular, this curved profile is such that an airflow entering the mouthpiece opening 108f and passing into the second passage 840 is gradually redirected from flowing in an axial direction (i.e., parallel to the longitudinal axis of the first passage 106) to a lateral direction (i.e., perpendicular to the axial direction).

As is apparent from a review of FIGS. 20C and 20D, the first passage 106f and the second passage 840 are arranged such that upon inhalation from the mouthpiece 109f (i.e., through the mouthpiece opening 108f) air flows past the wick 110f to the mouthpiece opening 108f. Further, upon exhalation into the mouthpiece opening 108f air flows from the mouthpiece 1091 and past the flavor delivery portion 842. In this way, flavorant of the flavor delivery portion 842 may be entrained in the air flowing past the flavor delivery portion 842. This flavored airflow may then be discharged from the outlet 844 of the second passage 840 towards a user's nasal cavity. Thus, exhalation by a user leads to the user smelling the flavored (or scented) air. If an exhalation is performed prior to an inhalation, the flavor or aroma of the flavored air may be smelled by a user prior to and during the inhalation. This may alter a user's overall experience during the inhalation.

In order to provide different inhalation and exhalation flow paths, the illustrated embodiment comprises a flow diverter 118. The flow diverter is movable between a first position (shown in FIG. 20C) and a second position (shown in FIG. 20D). In the first position airflow from the wick 110f to the mouthpiece 109f is permitted (i.e., such that air can be inhaled through the mouthpiece opening 108f by a user), whilst airflow from the mouthpiece 109f to the flavor delivery portion 842 is prevented. In the second position airflow between the wick 110f and the mouthpiece 109f is prevented, whilst airflow from the mouthpiece 109f to the flavor delivery portion 842 is permitted.

The flow diverter 850 is hingably mounted to a wall of the first passage 106f at the side of the second passage inlet 848 that is closer to the wick 110f. In this way, in the first position the flow diverter 850 extends from its hingable mounting towards the mouthpiece opening 108f so as to extend across (and obstruct) the second passage inlet 848. In the second position, the flow diverter 850 extends diagonally across the first passage 106 so as to obstruct the first passage 106. The angle of the flow diverter 850 with respect to the longitudinal axis of the first passage 106f facilitates flow of air from the first passage 106f into the second passage 840 (through the second passage inlet 848). Whilst not shown, the flow diverter 850 may be biased to the first or second position.

The flow diverter 850 may be configured to be moved between the first and second positions by exhalation and/or inhalation (i.e., by a user) at the mouthpiece. For example, the flow diverter 850 may be configured to be moved from the first position to the second position by exhalation into the mouthpiece opening 108f of the mouthpiece 109f and/or may be configured to be moved from the second position to the first position by an inhalation from the mouthpiece 109f.

The flow diverter 850 may alternatively or additionally comprise an actuator configured to move the flow diverter 850 between the first and second positions. The actuator may be configured to move the flow diverter 850 between the first and second positions in response to a control signal from the controller. For example, the actuator may be configured to move the flow diverter 850 in response to the detection (e.g., by a sensor) of an inhalation or exhalation at the mouthpiece 109f.

Although not shown, the main body 102f and consumable 103f may comprise a further interface which may, for example, be in the form of an RFID reader, a barcode or QR code reader. This interface may be able to identify a characteristic (e.g., a type) of a consumable 103f engaged with the main body 102f. In this respect, the consumable 103f may include any one or more of an RFID chip, a barcode or QR code, or memory within which is an identifier and which can be interrogated via the interface. For example, the interface may allow the detection of the flavor associated with the flavor delivery portion 842. The controller may control, e.g., the power supply in response to the detected flavor.

Seventh Mode: A Mouthpiece for a Smoking Substitute Apparatus

Aspects and embodiments of the seventh mode of the present disclosure will now be discussed with reference to the accompanying figures. Further aspects and embodiments of the seventh mode will be apparent to those skilled in the art. All documents mentioned in this text are incorporated herein by reference.

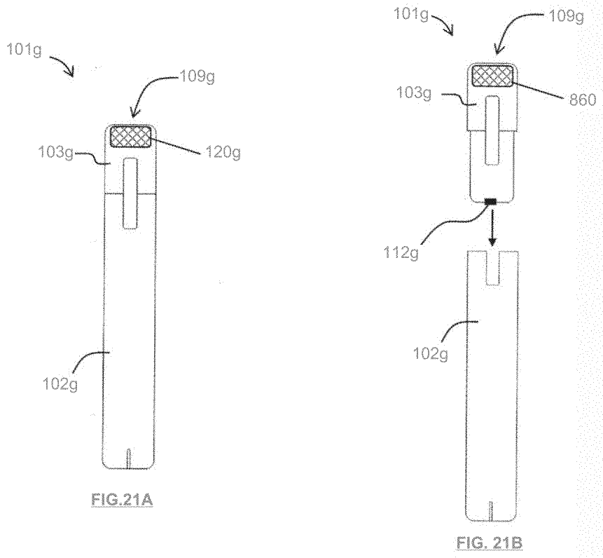

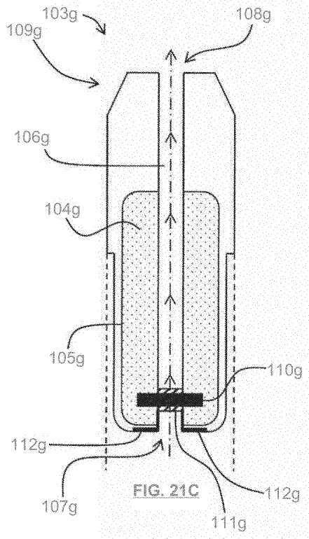

FIGS. 21A and 21B illustrate a smoking substitute system in the form of an e-cigarette system 101g. The system 101g comprises an e-cigarette device defining a main body 102g of the system 101g, and a smoking substitute apparatus in the form of an e-cigarette consumable (or “pod”) 103g. In the illustrated embodiment the consumable 103g (smoking substitute apparatus) is removable from the main body (e-cigarette device), so as to be a replaceable component of the system 101g. In other words, the e-cigarette system 101g is a closed system.

As is apparent from FIGS. 21A and 21B, the consumable 103g is configured to engage the main body 102g. FIG. 21A shows the main body 102g and the consumable 103g in an engaged state, whilst FIG. 21B shows the main body 102g and the consumable 103g in a disengaged state. When engaged, a portion of the consumable 103g is received in a cavity of the main body 102g and is retained in the engaged position by way of a snap-engagement mechanism. In other embodiments, the main body 102g and consumable 103g may be engaged by screwing one into (or onto) the other, through a bayonet fitting, or by way of an interference fit.

The system 101g is configured to vaporize an aerosol-former, which in the illustrated embodiment, is in the form of a nicotine-based e-liquid 104g. The e-liquid 104g comprises nicotine and a base liquid including propylene glycol and/or vegetable glycerin. In the present embodiment, the e-liquid 104g is flavorless (and does not include any added flavorant). That is, if the e-liquid 104g were to be inhaled (i.e., in aerosol form) by a user, it would not have a particularly perceptible flavor or taste.

As is more apparent from FIG. 21C, this e-liquid 104g is stored within a reservoir in the form of a tank 105g that forms part of the consumable 103g. In the illustrated embodiment, the consumable 103g is a “single-use” consumable 103g. That is, upon exhausting the e-liquid 104g in the tank 105g, the intention is that the user disposes of the entire consumable 103g. In other embodiments, the e-liquid (i.e., aerosol former) may be the only part of the system that is truly “single-use”. That is, the tank 105g may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105g surrounds, and thus defines a portion of, a passage 106g that extends between an inlet 107g and an outlet 108g at opposing ends of the consumable 103g. In this respect, the passage comprises an upstream end at the end of the consumable 103g that engages with the main body 102g, and a downstream end at an opposing end of the consumable 103g that comprises a mouthpiece 109g of the system 101g. When the consumable 103g is engaged with the main body 102g, a user can inhale (i.e., take a puff) via the mouthpiece 109g so as to draw air through the passage 106g, and so as to form an airflow (indicated by arrows) in a direction from the inlet 107g to the outlet 108g of the passage 106g. Although not illustrated, the passage 106g may be partially defined by a tube (e.g., a metal tube) extending through the consumable 103g. The passage 106g is in fluid communication with a gap defined between the consumable 103g and the main body 102g (when engaged) such that air outside of the system 101g is drawn into the passage 106g (during an inhale).

The smoking substitute system 101g is configured to vaporize the e-liquid 104g for inhalation by a user. To provide this, the consumable 103g comprises a heater having of a porous wick 110g and a resistive heating element in the form of a heating filament 111g that is helically wound around a portion of the porous wick 110g. The porous wick 110g extends across the passage 106g (i.e., transverse to a longitudinal axis of the passage 106g) and opposing ends of the wick 110g extend into the tank 105g (so as to be submerged in the e-liquid 104g). In this way, e-liquid 104g contained in the tank 105g is conveyed from the opposing ends of the porous wick 110g to a central portion of the porous wick 110g so as to be exposed to the airflow in the passage 106g (i.e., caused by a user inhaling).

The helical filament 111g is wound about this exposed central portion of the porous wick 110g and is electrically connected to an electrical interface in the form of electrical contacts 112g mounted at the end of the consumable that is proximate the main body 102g (when engaged). When the consumable 103g is engaged with the main body 102g, the electrical contacts 112g contact corresponding electrical contacts (not shown) of the main body 102g. The main body electrical contacts are electrically connected to a power source (not shown) of the main body 102g, such that (in the engaged position) the filament 111g is electrically connected to the power source. In this way, power can be supplied by the main body 102g to the filament 111g in order to heat the filament 111g. This heat is transferred from the filament 111g to the porous wick 110g which causes e-liquid 104g conveyed by the porous wick 110g to increase in temperature to a point at which it vaporizes. The vaporized e-liquid becomes entrained in the airflow and, between the vaporization point at the filament 111g and the outlet 108g of the passage 106g, condenses to form an aerosol. This aerosol is then inhaled, via the mouthpiece 109g, by a user of the system 101g.

The power source of the main body 102g may be in the form of a battery (e.g., a rechargeable battery).

The main body 102g may comprise a connector in the form of, e.g., a USB port for recharging this battery. The main body 102g may also comprise a controller that controls the supply of power from the power source to the main body electrical contacts (and thus to the filament 111g). That, is the controller may be configured to control a voltage applied across the main body electrical contacts, and thus the voltage applied across the filament 111g. In this way, the filament 111g may only be heated under certain conditions (e.g., during a puff and/or only when the system is in an active state). In this respect, the main body 102g may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102g and consumable 103g may comprise a further interface which may, for example, be in the form of an RFID reader, a barcode or QR code reader. This interface may be able to identify a characteristic (e.g., a type) of a consumable 103g engaged with the main body 102g. In this respect, the consumable 103g may include any one or more of an RFID chip, a barcode or QR code, or memory within which is an identifier and which can be interrogated via the interface.

The mouthpiece 109g of the consumable 103g includes a textured outer surface 860 which is arranged to receive a liquid flavorant and distribute the liquid flavorant across the textured outer surface 860. In the example shown, the textured outer surface 860 includes a plurality of crisscrossing channels. The channels are formed as fine grooves in the outer surface of the mouthpiece 109g. Liquid flavorant applied to textured outer surface 860 may be retained in the channels due to surface tension of the liquid flavorant in the channels, and may be distributed across the textured outer surface 860 via capillary action of the liquid flavorant in the channels. In this manner, when a drop of liquid flavorant is applied to the textured outer surface 860, the liquid flavorant may be distributed over an area corresponding to the textured outer surface 860. The textured outer surface 860 is disposed adjacent to the outlet 108g of the mouthpiece, so that it may come into contact with the user's tongue in use. In this manner, liquid flavorant held on the textured outer surface 860 may produce a flavor in the user's mouth.

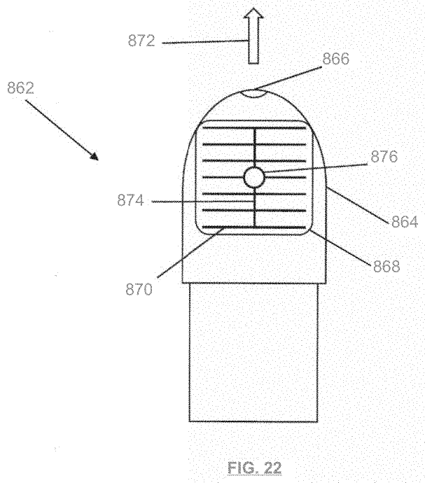

FIG. 22 shows a schematic side view of a smoking substitute apparatus, in the form of consumable 862 according to an embodiment of the disclosure. The consumable 862 may be similar in configuration to the mouthpiece 103 described above, e.g., it may be configured for engagement with a main body to form a smoking substitute system. The consumable 862 includes a mouthpiece 864 having an outlet 866 for conveying an aerosol generated by the smoking substitute system to a user. Adjacent to the outlet 866 is a textured outer surface 868 for receiving a liquid flavorant.

The textured outer surface 868 includes a plurality of channels formed as fine grooves in the outer surface of the mouthpiece 864. The plurality of channels includes a first set of channels 870 which are arranged in parallel and oriented along a first direction that is substantially perpendicular to a flow direction of aerosol through the outlet 866 of the mouthpiece 864. The direction of flow of aerosol through the outlet 866 is illustrated by arrow 872, and corresponds to a longitudinal direction of the consumable 862. The first set of channels 870 are interconnected by a longitudinal channel 874 which extends in the longitudinal direction of the consumable 862, i.e., the first set of channels 870 are in fluid communication via the longitudinal channel 874. The textured outer surface 868 further includes a fluid receiving portion 876 which is arranged at an intersection between the longitudinal channel 874 and a first one of the first set of channels 870. The fluid receiving portion 876 may be a shallow cavity or recess formed in the outer surface of the mouthpiece 864. The fluid receiving portion 876 is in fluid communication with the longitudinal channel 874 and the first one of the first set of channels 870.

The longitudinal channel 874 and the channels in the first set of channels 870 may be similar to the channels described above in relation to the textured outer surface 860. In particular, they are arranged to receive a liquid flavorant, and to wick liquid flavorant along their length through capillary action. Liquid flavorant deposited in the fluid receiving portion 876 may be wicked out of the fluid receiving portion 876 and into the plurality of channels. The longitudinal channel 874 may serve to convey liquid flavorant from the fluid receiving portion to each of the first set of channels 870. Thus, liquid flavorant may be substantially evenly distributed throughout the plurality of channels. In this manner, liquid flavorant deposited in the flavorant receiving portion 876 may be distributed throughout the plurality of channels (including the longitudinal channel 874 and the first set of channels). Thus, a user may only need apply liquid flavorant to the fluid receiving portion 876. The textured outer surface 868 may provide a large area containing flavorant which may come into contact with the user's mouth (e.g., tongue), which may enable effective delivery of flavorant to the user.

In use, the consumable may typically be held so that the longitudinal direction of the consumable 862 (i.e., the direction indicated by arrow 872) is tilted along a downwards angle. As the first set of channels 870 is substantially perpendicular to the longitudinal direction of the consumable 862, this may serve to prevent liquid flavorant in the first set of channels 870 from dripping downwards, which could cause liquid flavorant to drip off the mouthpiece 864. This may avoid liquid flavorant coming into contact with electrical components in the smoking substitute apparatus. As only one longitudinal channel 870 is provided, a comparatively small amount of liquid flavorant may be held in the longitudinal channel 870 relative to the first set of channels 870. This may reduce the risk of flavorant liquid dripping out of the longitudinal channel 870.

FIG. 23 illustrates a series of different textured outer surfaces that may be included in a mouthpiece 880 according to the disclosure. The mouthpiece 880 forms part of a consumable 882, which may be similar in configuration to the consumable 103g described above.

As illustrated in window 884 of FIG. 23, the mouthpiece 880 may have a textured outer surface comprising a plurality of randomly arranged protrusions. The randomly arranged protrusions form a series of channels (gaps) between them, the channels being dimensioned to wick liquid flavorant deposited on the surface of the mouthpiece 880. In this manner, liquid flavorant may be substantially evenly distributed across a surface of the mouthpiece 880.

Alternatively, as illustrated in window 886 of FIG. 23, the mouthpiece 880 may have a textured outer surface comprising a regular array of protrusions. In the example shown, the protrusions are substantially cylindrical in shape. The array of protrusions forms a series of channels between the protrusions, the channels being dimensioned to wick liquid flavorant deposited on the surface of the mouthpiece 880. In this manner, liquid flavorant may be substantially evenly distributed across a surface of the mouthpiece 880.

As another example, as shown in window 888 of FIG. 23, the mouthpiece 880 may include a series of crisscrossing channels which are formed as grooves in the outer surface of the mouthpiece 880. The crisscrossing channels are formed by a first set of parallel channels oriented along a first direction, and a second set of parallel channels oriented along a second direction perpendicular to the first direction. In this manner, the crisscrossing channels form a grid on the outer surface of the mouthpiece 880. In this manner, liquid flavorant may be wicked along the channels in the first and second directions, to provide a substantially even distribution of liquid flavorant over an area of the mouthpiece.

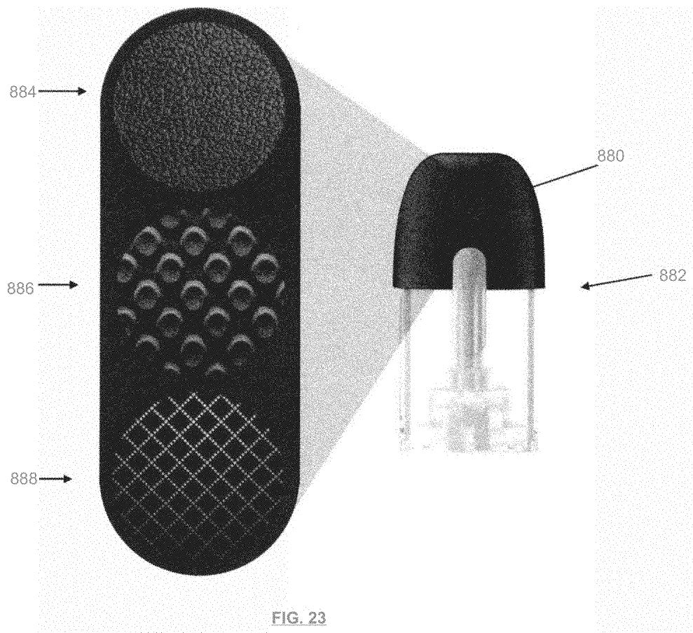

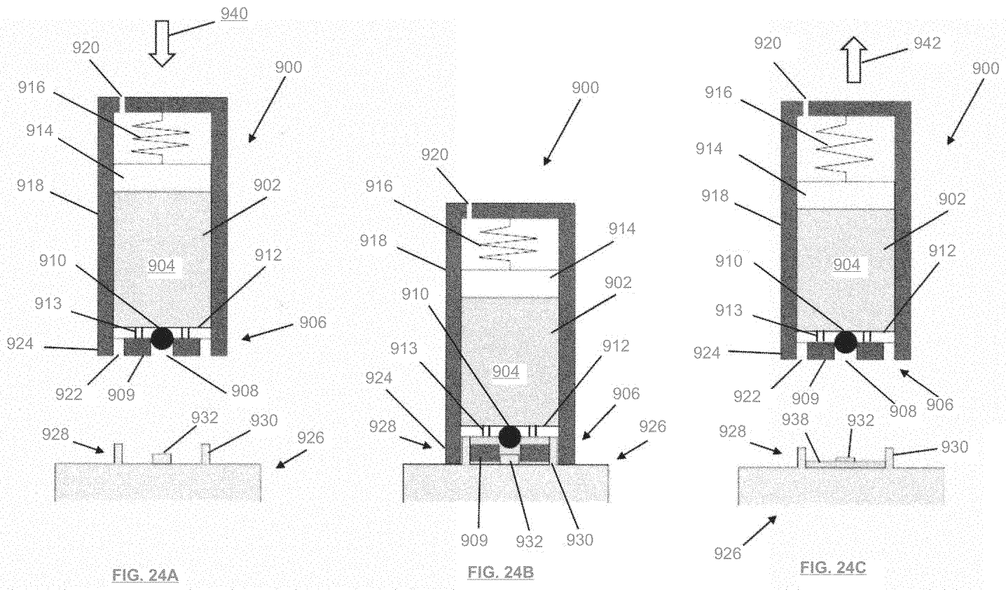

FIGS. 24A-24C show schematic cross-sectional views of a flavorant dispenser 900 according to an embodiment of the disclosure. The flavorant dispenser 900 may be used to dispense flavorant onto a mouthpiece of a smoking substitute apparatus, for flavor delivery to a user.

The flavorant dispenser 900 includes a reservoir 902 for holding a liquid flavorant 904. The reservoir 902 is disposed in an outer housing 918 of the flavorant dispenser 900, the outer housing 918 forming a sidewall of the reservoir 902. The flavorant dispenser further includes a dispensing portion 906 for dispensing liquid flavorant from the reservoir 902. The dispensing portion 906 includes an outlet 908 through which liquid flavorant 904 may be dispensed, and a ball valve 910. The ball valve 910 is movable between a closed position in which the ball valve 910 blocks the outlet 908 (FIGS. 24A, 24C), and an open position where the ball valve 910 does not block the outlet 908 (FIG. 24B), so that liquid flavorant 904 from the reservoir can exit via the outlet 908. The outlet 908 is defined by a ring 909, in which the ball valve 910 is held when it is in the closed position.

The ball valve 910 is mounted on a plate 912 disposed in the outer housing 918, the plate 912 forming a first end of the reservoir 902 adjacent the dispensing portion 906. The plate 912 is movable with the ball valve 910 between the open and closed positions. The plate includes a series of apertures 913. When the ball valve 910 is in the closed position, the plate 912 abuts against a surface of the ring 909, so that the apertures 913 are blocked. When the ball valve 910 is in the open position, the plate 912 is spaced from the ring 909, so that the apertures 913 are no longer blocked, and liquid flavorant 904 from the reservoir can exit the flavorant dispenser via the apertures 913 and the outlet 908.

The flavorant dispenser 900 further includes a piston 914 mounted in the outer housing 918 of the flavorant dispenser 900, such that the piston 914 forms a second end of the reservoir 902, opposite the first end of the reservoir 902. The piston 914 is movable within the reservoir 902 along a longitudinal direction of the reservoir 902. A spring 916 is mounted between an outer housing 918 of the flavorant dispenser 900 and the piston 914, and arranged to push the piston 914 towards the outlet 908. In this manner, liquid flavorant 904 in the reservoir is urged towards the outlet 908 by the piston 914, under a biasing force of the spring 916. This causes the plate 912 to be pressed against the surface of the ring 909, so that the ball valve 910 blocks the outlet 908 and the apertures 913 are blocked by the surface of the ring 909. Thus, the spring 916 biases the ball valve 908 towards the closed position. This may prevent liquid flavorant 904 from leaking out of the flavorant dispenser 900 when the dispenser is not in use. The outer housing 918 further includes an aperture 920 arranged to allow pressure equalization inside the housing 918 due to movement of the piston 914.

The flavorant dispenser 900 is designed for use with a mouthpiece 926. In particular, the flavorant dispenser 900 is configured to dispense flavorant 904 onto the mouthpiece 926 when the mouthpiece 928 is brought into engagement with the flavorant dispenser 900, as described below.

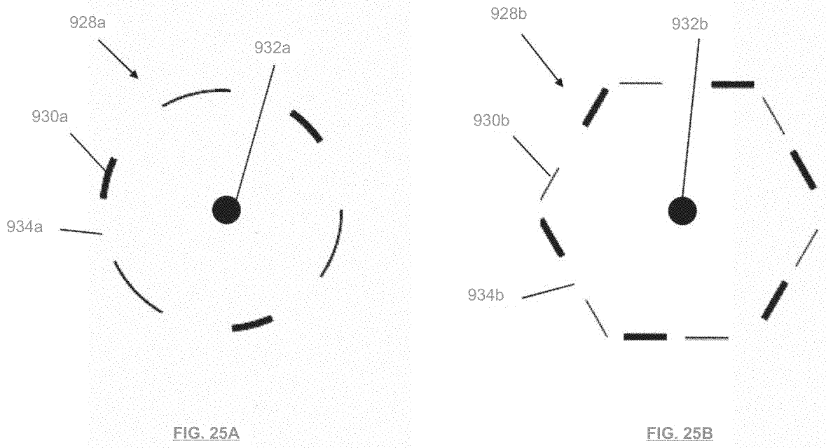

The mouthpiece 926 may, for example, be similar to mouthpiece 109g described above. For illustration purposes, only a portion of the mouthpiece 926 is shown in FIGS. 24A-24C. The mouthpiece 926 may be mounted on, or form part of, a smoking substitute apparatus. The mouthpiece 926 includes an engagement portion 928 which includes a plurality of sidewalls 930 arranged in a ring around a central protrusion 932. FIGS. 25A and 25B show top views of possible configurations of the engagement portion 928. FIG. 25A shows a top view of an engagement portion 928a including a plurality of sidewalls 930a arranged in a circular ring around central protrusion 932a. As can be seen, some of the sidewalls 930a have different thicknesses. Gaps 934a are formed between adjacent sidewalls 930a. FIG. 25B shows a top view of an engagement portion 928b including a plurality of sidewalls 930b arranged in a hexagonal ring around central protrusion 932b. As can be seen, some of the sidewalls 930b have different thicknesses. Gaps 934b are formed between adjacent sidewalls 930b.

The dispensing portion 906 of the flavorant dispenser 900 includes a set of recesses 922 defined between the ring 909 and a sidewall 924 of the outer housing 918. The recesses 922 are arranged to receive the engagement portion 928 of the mouthpiece 926. In particular, the recesses 922 are arranged to receive the plurality of sidewalls 930 of the engagement portion 928 of the mouthpiece 926. The set of recesses 922 may be shaped and arranged to receive a specific engagement portion 928, e.g., engagement portion 928a or 928b. Thus, for instance, the recesses 922 may be arranged in a circular or a hexagonal ring, and the set of recesses 922 may include recesses having different widths to accommodate sidewalls 930 having different widths. The ring 909 is configured to receive the central protrusion 932 when the sidewalls 930 are engaged in the recesses 922.

The dispensing portion 906 is arranged such that, when the sidewalls 930 are received in the recesses 922, the sidewalls 930 abut against the plate 912 to press the plate 912 away from the ring 909. This enables flavorant to flow through the apertures 913 in the plate 912 and through the outlet 908, so that flavorant may be dispensed. In this manner, engagement between the dispensing portion 906 and the engagement portion 928 on the mouthpiece 926 causes flavorant 904 to be dispensed from the flavorant dispenser 900.

As a result of the arrangement of the set of recesses 922, the dispensing portion 926 is configured for engagement with a mouthpiece having a specifically arranged engagement portion. In some cases, the dispensing portion 926 and engagement portion of the mouthpiece may be arranged so that the dispensing portion 926 and the engagement portion can only be engaged when they are in a specific orientation. In this manner, the engagement portion of the mouthpiece may be considered as a key which is arranged for engagement with the dispensing portion.

We will now describe a process of dispensing flavorant onto the mouthpiece 926 using flavorant dispenser 900, with reference to FIGS. 24A-24C.

In FIG. 24A, the ball valve 910 is in the closed position and the reservoir 902 contains liquid flavorant 904. The ball valve 910 is maintained in the closed position, via the piston 914 which presses the liquid flavorant 904 against the plate 912, under action of the spring 916. The flavorant dispenser 900 is approached towards the engagement portion 928 of the mouthpiece 926, as indicated by arrow 940.

In FIG. 24B, the dispensing portion 906 of the flavorant dispenser 900 is engaged with the engagement portion 928 of the mouthpiece 926. In particular, the sidewalls 930 of the engagement portion 928 are received in the recesses 922 of the dispensing portion 906, and the central protrusion 932 is received in the ring 909. In this configuration, the sidewalls 930 abut against the plate 912 so that the plate 912 is spaced apart from the ring 909. As a result, the ball valve 910 is lifted out of the ring 909 so that the outlet 908 is no longer blocked, and the apertures 913 in the plate 912 are no longer blocked by the surface of the ring 909. Accordingly, liquid flavorant 904 may flow out of the reservoir 902 via the apertures 913 and the outlet 908.

When the dispensing portion 906 is engaged with the engagement portion 928 of the mouthpiece 926, the sidewalls 930 of the engagement portion 928 and the sidewall 924 of the outer housing 918 together define a receptacle (cavity) into which liquid flavorant 904 dispensed by the dispensing portion 906 is received. In particular, the sidewall 924 of the outer housing 918 may serve to prevent liquid flavorant 904 from escaping via gaps 934a, 934b between the sidewalls 930 of the engagement portion. Thus, whilst the dispensing portion 906 is engaged with the engagement portion 928, dispensed liquid flavorant may be contained within the sidewalls 930. The receptacle defined by the sidewalls 930 and sidewall 924 may have a predetermined volume, so that only a predetermined volume of liquid flavorant 904 may be dispensed when the dispensing portion 906 is engaged with the engagement portion 928.

After liquid flavorant 904 has been dispensed by the dispensing portion 906, the dispensing portion 906 may be disengaged from the mouthpiece, as shown in FIG. 24C and indicated by arrow 942. In this configuration, the ball valve 910 once again returns to the closed position due to the pressure exerted by the spring 916. A volume 938 of liquid flavorant 904 corresponding to the predetermined volume dispensed by the dispensing portion 906 is left on the mouthpiece 926. As the sidewall 924 of the outer housing 918 no longer blocks the gaps 934a, 934b between the sidewalls 930 of the engagement portion 928, the volume 938 of liquid flavorant may spread over a surface of the mouthpiece 926. For example, if the mouthpiece includes a textured outer surface, the textured outer surface may serve to distribute the volume 938 of liquid flavorant over an area of the mouthpiece 926.

Accordingly, the flavorant dispenser 900 may serve to dispense a predetermined volume of liquid flavorant 904 onto the surface of the mouthpiece 926 in a controlled manner. Specificity between the dispensing portion 906 and the engagement portion 928 of the mouthpiece 926 may ensure that the flavorant dispenser 900 is only used to dispense liquid flavorant onto the mouthpiece 926. This may prevent accidental dispensing of liquid flavorant, or dispensing of liquid flavorant on devices which are not intended for use with the flavorant dispenser 900.

In some embodiments, the mouthpiece 926 and flavorant dispenser 900 may be provided as a kit.

Eighth Mode: A Smoking Substitute Apparatus that Allows an Aerosol Former to be Stored Separately from a Property Modifying Agent

Aspects and embodiments of the eighth mode of the present disclosure will now be discussed with reference to the accompanying figures. Further aspects and embodiments of the eighth mode will be apparent to those skilled in the art. All documents mentioned in this text are incorporated herein by reference.

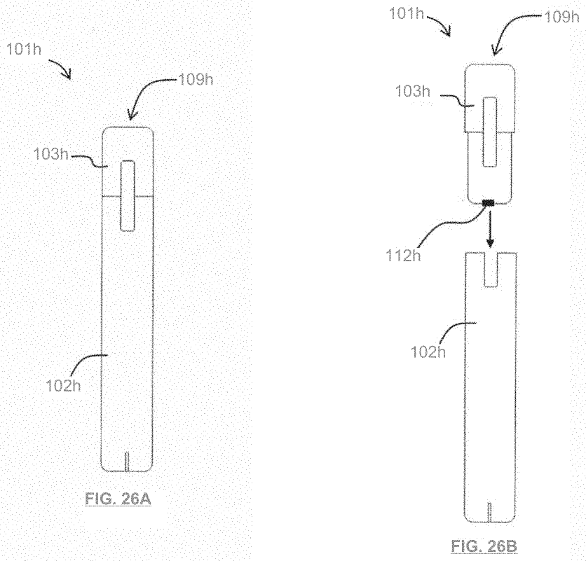

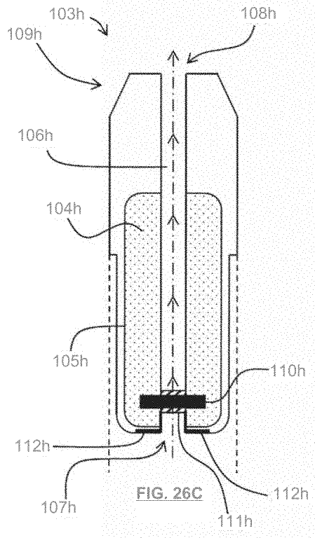

FIGS. 26A and 26B illustrate a smoking substitute system in the form of an e-cigarette system 101h. The system 101h comprises an e-cigarette device defining a main body 102h of the system 101h, and a smoking substitute apparatus in the form of an e-cigarette consumable (or “pod”) 103h. In the illustrated embodiment the consumable 103h (smoking substitute apparatus) is removable from the main body (e-cigarette device), so as to be a replaceable component of the system 101h. In other words, the e-cigarette system 101h is a closed system.

As is apparent from FIGS. 26A and 26B, the consumable 103h is configured to engage the main body 102h. FIG. 26A shows the main body 102h and the consumable 103h in an engaged state, whilst FIG. 26B shows the main body 102h and the consumable 103h in a disengaged state. When engaged, a portion of the consumable 103h is received in a cavity of the main body 102h and is retained in the engaged position by way of a snap-engagement mechanism. In other embodiments, the main body 102h and consumable 103h may be engaged by screwing one into (or onto) the other, through a bayonet fitting, or by way of an interference fit.

The system 101h is configured to vaporize an aerosol-former, which in the illustrated embodiment, is in the form of a nicotine-based e-liquid 104h. The e-liquid 104h comprises nicotine and a base liquid including propylene glycol and/or vegetable glycerin. In the present embodiment, the e-liquid 104h is flavorless (and does not include any added flavorant). That is, if the e-liquid 104h were to be inhaled (i.e., in aerosol form) by a user, it would not have a particularly perceptible flavor or taste.

As is more apparent from FIG. 26C, this e-liquid 104h is stored within a reservoir in the form of a tank 105h that forms part of the consumable 103h. In the illustrated embodiment, the consumable 103h is a “single-use” consumable 103h. That is, upon exhausting the e-liquid 104h in the tank 105h, the intention is that the user disposes of the entire consumable 103h. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105h surrounds, and thus defines a portion of, a passage 106h that extends between an inlet 107h and an outlet 108h at opposing ends of the consumable 103h. In this respect, the passage comprises an upstream end at the end of the consumable 103h that engages with the main body 102h, and a downstream end at an opposing end of the consumable 103h that comprises a mouthpiece 109h of the system 101h. When the consumable 103h is engaged with the main body 102h, a user can inhale (i.e., take a puff) via the mouthpiece 109h so as to draw air through the passage 106h, and so as to form an airflow (indicated by arrows) in a direction from the inlet 107h to the outlet 108h of the passage 106h. Although not illustrated, the passage 106h may be partially defined by a tube (e.g., a metal tube) extending through the consumable 103h. The passage 106h is in fluid communication with a gap defined between the consumable 103h and the main body 102h (when engaged) such that air outside of the system 101h is drawn into the passage 106h (during an inhale).

The smoking substitute system 101h is configured to vaporize the e-liquid 104h for inhalation by a user. To provide this, the consumable 103h comprises a heater having of a porous wick 110h and a resistive heating element in the form of a heating filament 111h that is helically wound around a portion of the porous wick 110h. The porous wick 110h extends across the passage 106h (i.e., transverse to a longitudinal axis of the passage 106h) and opposing ends of the wick 110h extend into the tank 105h (so as to be submerged in the e-liquid 104h). In this way, e-liquid 104h contained in the tank 105h is conveyed from the opposing ends of the porous wick 110h to a central portion of the porous wick 110h so as to be exposed to the airflow in the passage 106h (i.e., caused by a user inhaling).

The helical filament 111h is wound about this exposed central portion of the porous wick 110h and is electrically connected to an electrical interface in the form of electrical contacts 112h mounted at the end of the consumable that is proximate the main body 102h (when engaged). When the consumable 103h is engaged with the main body 102h, the electrical contacts 112h contact corresponding electrical contacts (not shown) of the main body 102h. The main body electrical contacts are electrically connected to a power source (not shown) of the main body 102h, such that (in the engaged position) the filament 111h is electrically connected to the power source. In this way, power can be supplied by the main body 102h to the filament 111h in order to heat the filament 111h. This heat is transferred from the filament 111h to the porous wick 110h which causes e-liquid 104h conveyed by the porous wick 110h to increase in temperature to a point at which it vaporizes. The vaporized e-liquid becomes entrained in the airflow and, between the vaporization point at the filament 111h and the outlet 108h of the passage 106h, condenses to form an aerosol. This aerosol is then inhaled, via the mouthpiece 109h, by a user of the system 101h.

The power source of the main body 102h may be in the form of a battery (e.g., a rechargeable battery).

The main body 102h may comprise a connector in the form of, e.g., a USB port for recharging this battery. The main body 102h may also comprise a controller that controls the supply of power from the power source to the main body electrical contacts (and thus to the filament 111h). That, is the controller may be configured to control a voltage applied across the main body electrical contacts, and thus the voltage applied across the filament 111h. In this way, the filament 111h may only be heated under certain conditions (e.g., during a puff and/or only when the system is in an active state). In this respect, the main body 102h may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102h and consumable 103h may comprise a further interface which may, for example, be in the form of an RFID reader, a barcode or QR code reader. This interface may be able to identify a characteristic (e.g., a type) of a consumable 103h engaged with the main body 102h. In this respect, the consumable 103h may include any one or more of an RFID chip, a barcode or QR code, or memory within which is an identifier and which can be interrogated via the interface.

FIG. 27 illustrates an aerosol forming device, or a consumable 203h, according to an embodiment of the present disclosure. The consumable 203h comprises a tank 205h that is equally divided into a first compartment 205h-1 and a second compartment 205h-2 by a partition wall 950. In other words, the first compartment 205h-1 and the second compartment 205h-2 shares the partition wall 950. The partition wall 950 extends from a base of the consumable along the length of the first compartment 205h-1 and second compartment 205h-2. In the illustrated embodiment the first compartment 205h-1 and the second compartment 205h-2 are of equal volume, e.g., 1:1. In other embodiments, the ratio of the volume of the first compartment to the volume of the second compartment may be in the range of 1:1 to 9:1.

In the illustrated embodiment, the first compartment 205h-1 contains a supply of aerosol former, or e-liquid 204h, and the second compartment 205h-2 contains a property modifying agent 204h-2, or an additive. The aerosol former is in a liquid form. The aerosol former comprises nicotine and a base liquid, e.g., propylene glycol. The aerosol former does not contain any flavorant and therefore the aerosol former is not flavored. The aerosol former in this embodiment does not contain any colorant. That is, the aerosol former is in the natural color of the propylene glycol and the nicotine, and therefore it appears as a translucent or transparent liquid.

The property modifying agent 204h-2 in the illustrated embodiment is a liquid property modifying agent, comprises a flavorant and a colorant respectively configured to modify the flavor and color of the e-liquid 204h-1 as stored in the first compartment 205h-1. In other embodiments, the property modifying agent may be a solid, a gel, a liquid or a gas, and may comprise only a flavorant without the presence of a colorant or may comprise a colorant without the presence of a flavorant.

The partition wall 950 as shown in FIG. 27 comprises an opening 952. The opening 952 effects fluid communication between the first compartment 205h-1 and the second compartment 205h-2. The opening 952 is sealed by a barrier or removable seal 954 prior to its removal. More specifically, a seal portion 956 of the removable seal 954 covers the opening 952 in a sealing position and thereby hermetically seals the opening 952. Therefore, in the sealing position, the seal portion 956 of the removable seal 954 prevents fluid communication between the first compartment 205h-1 and the second compartment 205h-2. The removable seal 954 is configured to be removed from the sealing position and thereby detaches the seal portion 956 form the opening 952, and thereby establishes fluid communication between the first compartment 205h-1 and second compartment 205h-2.

The removable seal 954 additionally comprises a tab portion 958 in connection with the seal portion 956. That is the seal portion 956 is formed together with the tab portion 958 to form the removable seal. Said tab portion 958 extends, along a slot in the consumable 203h and outwardly from the base of the consumable 203h through a check valve 960, such that at least a part of the tab portion 958 is accessible to a user. The tab portion 958 comprises an enlarged portion 962 external to the consumable 203h for a user to grip onto.

Prior to use or in use, the user may pull on the tab portion 958 away from the consumable 203h to detach the seal portion 956 from the opening 952. The removable seal 954 is then completely removed from the consumable 203h to be disposed of. Doing so establishes fluid communication between the first compartment 205h-1 and second compartment 205h-2, thereby allowing the aerosol former 204a in the first compartment 205h-1 to come into contact with the property modifying agent 204h-2 in the second compartment 205h-2. More specifically, the property modifying agent 204h-2 may diffuse into or mix with the aerosol former 204a to form a modified aerosol former once they come into contact with each other. Furthermore, a user may shake the consumable 203h to promote mixing of the aerosol former 204a and the property modifying agent 204h-2.

In the illustrated embodiment, the property modifying agent 204a comprises a mixture of flavorant and colorant. The colorant may serve as a visual indicator indicating the degree of homogeneity in the modified aerosol former. More specifically, the user may visually inspect the modified aerosol former through a translucent wall of the consumable 203h. For example, a uniform distribution of color in the modified aerosol former across the first and second compartment indicates that the flavorant and colorant are homogenously mixed with the aerosol former. On the other hand, localized fluctuation in color signals uneven blending of the aerosol former 204a and the property modifying agent 204h-2 and further mixing is required.

In another embodiment, the opening is closed by a valve that is actuated by an actuator extending outwardly from the consumable. The actuator is a lever that is mechanically linked to the valve to actuate the valve from a closed position which prevents fluid communication to an opened position which establishes fluid commutation between the first and second compartments.

In such embodiment, the valve is a check valve 960 which allows the property modifying agent to flow from the second compartment towards the first compartment. This allows the flavorant and colorant to be dosed to the aerosol former in the first compartment, whilst preventing the aerosol former form entering the second compartment. In use, a user may actuate the actuator to control the flow of property modifying agent through said opening, and therefore dispense a desired amount of the property modifying agent from the first compartment into the second compartment. This advantageously allows the user to personalize the flavor and/or color of the e-liquid by controlling the dosage of flavorant and/or colorant.

In another embodiment, the partition wall 950 does not comprise an opening. Instead, the partition wall is removable for merging the second compartment with the first compartment to form a combined compartment. In this case, the partition wall comprises a planar element movable along a pair of slots extending in between the first and second compartments. In use, a user may pull the removable partition wall along the slot so as to remove said wall from the smoking substitute apparatus. The first and second compartments are then merge into a single combined compartment, i.e., the aerosol former in the first compartment and the property modifying agent in the second compartment may mix in the combined compartment.

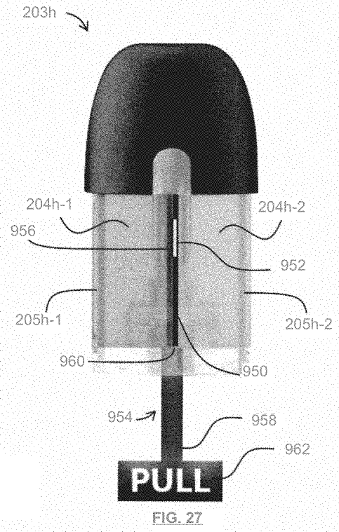

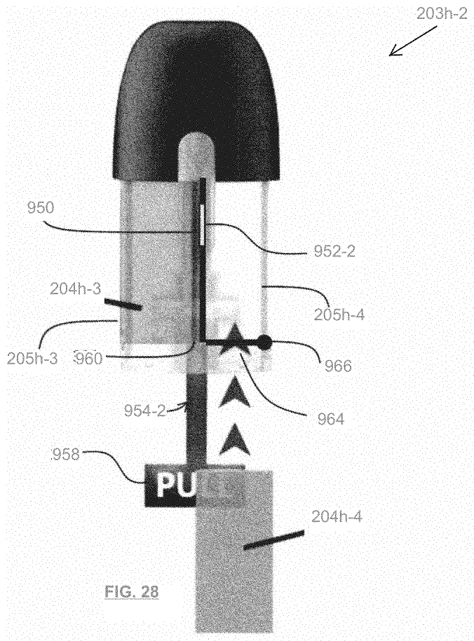

FIG. 28 illustrates an aerosol forming device, or a consumable 203h-2, according to another embodiment of the present disclosure. The consumable 203h-2 is the similar in construction to the consumable 203h as shown in FIG. 27. For example, the consumable 203h-2 also comprises a partition wall 950 separating a first compartment 205h-3 and second compartment 205h-4. A removable seal 954-2 is provided for sealing an opening 952-2 at the partition wall 950. Removing the removable seal 954-2 establishes fluid communication between the first compartment 205h-3 and the second compartment 205h-4.

The consumable 203h-2 differs to the consumable 203h of FIG. 27 in that the consumable 203h-2 additionally comprises a compartment inlet 964 at the base of the second compartment 205h-4. The compartment inlet 964 is closed by a cover 966 hingedly connected to the body of the consumable 203h-2. The cover 966 is pivotable between a closed position that hermetically seals the second compartment 205h-4, and an open position that allows a property modifying agent 204h-2 to be added to the second compartment 205h-4. More specifically, the consumable 203h-2 is supplied with only an aerosol former 204h-1 provided in the first compartment 205h-3. The second compartment 205h-4 is empty having a void. Such arrangement allows the user to add a desired amount, and/or variety, of property modifying agent to the second compartment before it is mixed with the aerosol former. For example, a user may opt to add different flavorants (e.g., tobacco and blueberry flavor) into the second compartment 205h-4 so as to create a personalized vaping experience.

The property modifying agent 204h-2 in the illustrated example is a solid property modifying agent 204h-2 comprising a mixture of flavorant and colorant. In use, a user may pivot the cover 966 from the close position towards the open position, before insert the solid property modifying agent 204h-2 into the second compartment 205h-4. The user may then put the cover 966 into the close position to seal the second compartment 205h-4 from the atmosphere.

Similar to the consumable 203h of FIG. 27, upon removing the removable seal 954-2 from the consumable 203h-2 fluid communication is established, via the opening 952-2, between the first compartment 205h-3 and second compartment 205h-4. This allows the aerosol former 204h-1 to flow through the opening 952-2 and contacts the solid property modifying agent 204h-2 in the second compartment 205h-4. In the presence of aerosol former 204h-1, the solid property modifying agent 204h-2 dissolves and thereby releases the flavorant and colorant into the aerosol former 204h-1.

The solid property modifying agent 204h-2 may promptly dissolve in the aerosol former 204h-1, as such a modified aerosol former may be produced instantaneously. Alternatively, the solid property modifying agent 204h-2 may dissolve gradually and therefore the concentration of flavorant and colorant in the modified aerosol former may vary with time. This allows the user to control the strength of flavor and color in the generated aerosol according to his/her preference.

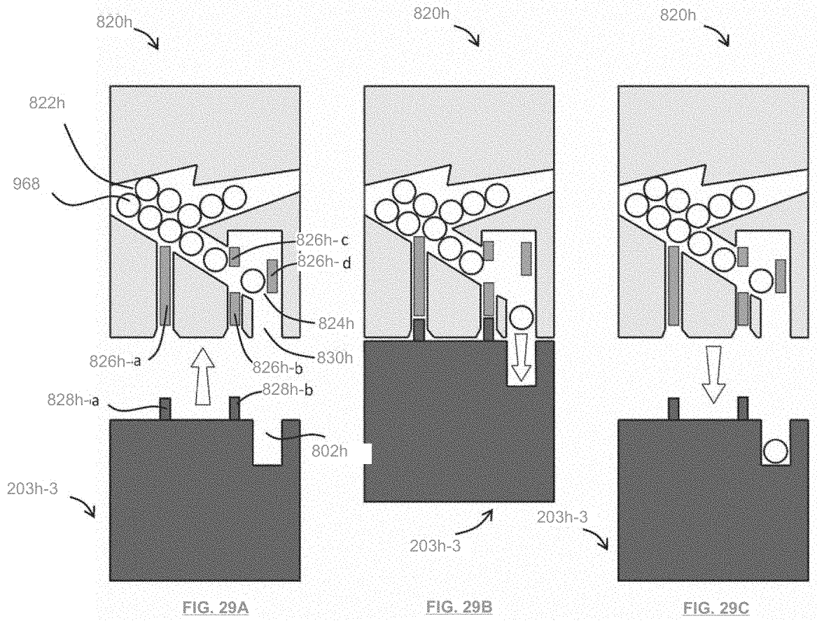

FIGS. 29A, 29B and 29C sequentially show a dispenser 820h dispensing a property modifying agent 968 to a second compartment 802h of a consumable 203h-3. The property modifying agent 968 in this case is solid property modifying agent in the form of pellets 968, comprising flavorant and colorant. FIG. 29A and FIG. 29C show the dispenser 820h being separated from the consumable 203h-3 and FIG. 29B shows the dispenser 820h is engaged with the consumable 203h-3. The dispenser 820h comprises a storage 822h for storing a supply of pellets 968 and a dispensing chamber 824h for storing a predetermined number of pellets to be dispensed. For example, the dispensing chamber 824h is sized to receive the required number of pellets to be dispensed, in this case a single pellet. However, the dispensing chamber 824h may be sized to receive a plurality of pellets such that said plurality of pellets may be dispensed at once. When the dispenser is separated from the consumable, as shown in FIG. 29A, a dispensing element 826h-d prevents the pellet from being dispensed out of the dispensing chamber 824h.

The dispenser comprises a dispensing mechanism, which is formed of dispensing elements 826h-a, 826h-b, 826h-c and 826h-d, and is configured to be activated by a corresponding activating mechanism 828h-a and 828h-b at or near a compartment inlet of the consumable 203h-3. More specifically, the activating mechanism 828h-a and 828h-b are protrusions that, when the dispenser 820h is engaged with the consumable 203h-3 as shown in FIG. 29B, push onto the dispensing element 826h-a and 826h-b and thereby urges the dispensing mechanism towards an upward direction. This causes the dispensing element 826h-d to move and thereby allows the pellet 968 in the dispensing chamber 824h to dispense through a dispenser outlet 830h into the second compartment 802h. Furthermore, when the dispensing mechanism is activated as shown in FIG. 29B, a slot formed between the dispensing element 826h-b and 826h-c also moves in line with a pellet 968 stored in the storage 822h and thereby allowing the pellet 968 in the storage 822h to drop into the dispensing chamber 824h. That is, activating the dispensing mechanism causes a pellet to be dispensed out of the dispensing chamber 824h, as well as allowing a pellet from the storage 822h to replenish the dispensing chamber.

Ninth Mode: A Smoking Substitute Apparatus Comprising a User Controllable Valve

Aspects and embodiments of the ninth mode of the present disclosure will now be discussed with reference to the accompanying figures. Further aspects and embodiments of the ninth mode will be apparent to those skilled in the art. All documents mentioned in this text are incorporated herein by reference.

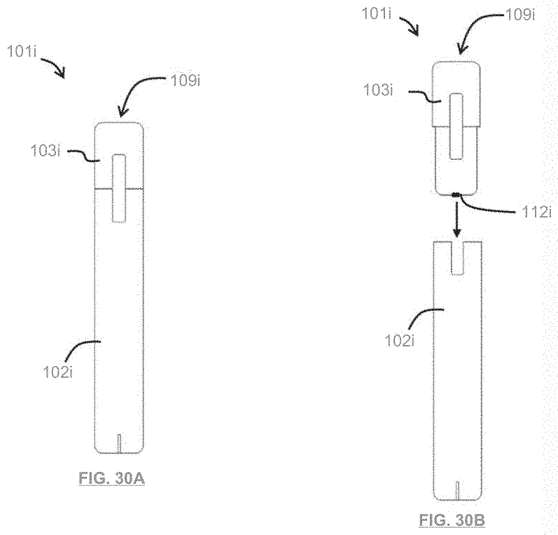

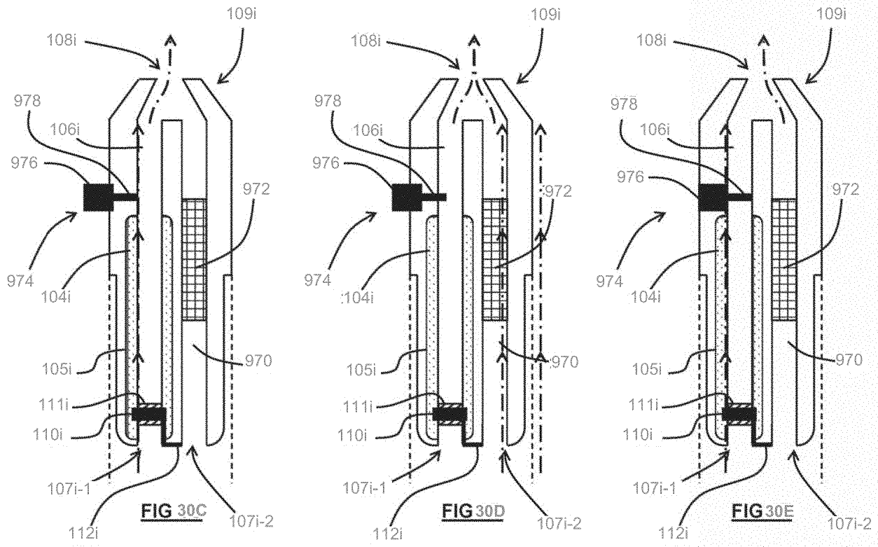

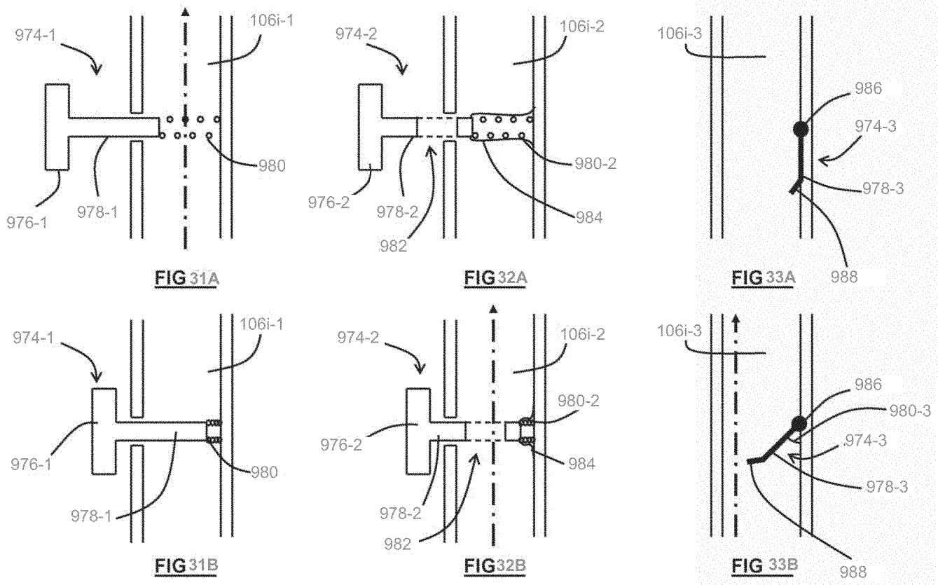

FIGS. 30A and 30B illustrate a smoking substitute system in the form of an e-cigarette system 101i. The system 101i comprises an e-cigarette device defining a main body 102i of the system 101i, and an aerosol delivery device in the form of an e-cigarette consumable (or “pod”) 103i. In the illustrated embodiment the consumable 103i (aerosol delivery device) is removable from the main body (e-cigarette device), so as to be a replaceable component of the system 101i. In other words, the e-cigarette system 101i is a closed system.

As is apparent from FIGS. 30A and 30B, the consumable 103i is configured to engage the main body 102i. FIG. 30A shows the main body 102i and the consumable 103i in an engaged state, whilst FIG. 30B shows the main body 102i and the consumable 103i in a disengaged state. When engaged, a portion of the consumable 103i is received in a cavity of the main body 102i and is retained in the engaged position by way of a snap-engagement mechanism. In other embodiments, the main body 102i and consumable 103i may be engaged by screwing one into (or onto) the other, through a bayonet fitting, or by way of an interference fit.

The system 101i is configured to vaporize an aerosol-former, which in the illustrated embodiment, is in the form of a nicotine-based e-liquid 104i. The e-liquid 104i comprises nicotine and a base liquid including propylene glycol and/or vegetable glycerin. In the present embodiment, the e-liquid 104i is flavorless (and does not include any added flavorant). That is, if the e-liquid 104i were to be inhaled (i.e., in aerosol form) by a user, it would not have a particularly perceptible flavor or taste.

As is more apparent from FIGS. 30C to 30E, this e-liquid 104i is stored within a reservoir in the form of a tank 105i that forms part of the consumable 103i. In the illustrated embodiment, the consumable 103i is a “single-use” consumable 103i. That is, upon exhausting the e-liquid 104i in the tank 105i, the intention is that the user disposes of the entire consumable 103i. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105i surrounds, and thus defines a portion of, a first passage 106i that extends between an inlet 107i-1 and an outlet 108i at opposing ends of the consumable 103i. In this respect, the passage 106i comprises an upstream end at the end of the consumable 103i that engages with the main body 102i, and a downstream end at an opposing end of the consumable 103i that comprises a mouthpiece 109i of the system 101i. When the consumable 103i is engaged with the main body 102i, a user can inhale (i.e., take a puff) via the mouthpiece 109i so as to draw air through the passage 106i, and so as to form an airflow (indicated by arrows in FIGS. 30C and 30E) in a direction from the inlet 107i-1 to the outlet 108i. The first passage 106i is in fluid communication with a gap defined between the consumable 103i and the main body 102i (when engaged) such that air outside of the system 101i is drawn into the first passage 106i (during an inhale).

The smoking substitute system 101i is configured to vaporize the e-liquid 104i for inhalation by a user. To provide this, the consumable 103i comprises a heater having a porous wick 110i and a resistive heating element in the form of a heating filament 111i that is helically wound around a portion of the porous wick 110i. The porous wick 110i extends across the first passage 106i (i.e., transverse to a longitudinal axis of the passage 106i) and opposing ends of the wick 110i extend into the tank 105i (so as to be submerged in the e-liquid 104i). In this way, e-liquid 104i contained in the tank 105i is conveyed from the opposing ends of the porous wick 110i to a central portion of the porous wick 110i so as to be exposed to the airflow in the first passage 106i (i.e., caused by a user inhaling). In other embodiments the heating filament 111i and/or wick 110i may form part of the main body (but may engage the tank 105i during engagement of the main body 102i and the consumable 103i).

The helical filament 111i is wound about this exposed central portion of the porous wick 110i and is electrically connected to an electrical interface in the form of electrical contacts 112i mounted at the end of the consumable that is proximate the main body 102i (when engaged). When the consumable 103i is engaged with the main body 102i, the electrical contacts 112i contact corresponding electrical contacts (not shown) of the main body 102i. The main body electrical contacts are electrically connected to a power source (not shown) of the main body 102i, such that (in the engaged position) the filament 111i is electrically connected to the power source. In this way, power can be supplied by the main body 102i to the filament 111i in order to heat the filament 111i. This heat is transferred from the filament 111i to the porous wick 110i which causes e-liquid 104i conveyed by the porous wick 110i to increase in temperature to a point at which it vaporizes. The vaporized e-liquid becomes entrained in the airflow in the first passage 106i and, between the vaporization point at the filament 111i and the outlet 108i, condenses to form an aerosol. This aerosol is then inhaled, via the mouthpiece 109i, by a user of the system 101i.

The power source of the main body 102i may be in the form of a battery (e.g., a rechargeable battery).

The main body 102i may comprise a connector in the form of, e.g., a USB port for recharging this battery. The main body 102i may also comprise a controller that controls the supply of power from the power source to the main body electrical contacts (and thus to the filament 111i). That, is the controller may be configured to control a voltage applied across the main body electrical contacts, and thus the voltage applied across the filament 111i. In this way, the filament 111i may only be heated under certain conditions (e.g., during a puff and/or only when the system is in an active state). In this respect, the main body 102i may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102i and consumable 103i may comprise a further interface which may, for example, be in the form of an RFID reader, a barcode or QR code reader. This interface may be able to identify a characteristic (e.g., a type) of a consumable 103i engaged with the main body 102i. In this respect, the consumable 103i may include any one or more of an RFID chip, a barcode or QR code, or memory within which is an identifier and which can be interrogated via the interface.

The consumable 103i additionally comprises a second passage 970 that is arranged in parallel with the first passage 106i and (like the first passage 106i) is in fluid communication with the outlet 108i. Thus, the second passage 970 extends between an inlet 107i-2 and the outlet 108i. The second passage 970 comprises an additive delivery portion, in the form of a porous element 972 impregnated with a flavorant. The porous element 972 is located in the second passage 970 between the inlet 107i-2 and the outlet 108i so as to partially obstruct the passage 970. This porous element 972 delivers a flavorant to an airflow as it flows through the filter 972. Thus, when air is drawn through the outlet 108i and flows from the second passage 970 flavorant is carried from the porous element 972 to the outlet 108i.

Because the porous element 972 partially obstructs the passage 970, In addition to delivering a flavorant, the porous element 972 acts as an airflow restrictor. In other words, the porous element 972 increases the resistance to airflow of the second passage 970, such that the resistance to airflow of the second passage 970 is substantially greater than the resistance to airflow of the first passage 106i. As will be described further below, this allows the airflow through the second passage 970 to be controlled by way of a valve 974 forming part of the first passage 106i.

The valve 974 is movable transversely across the first passage 106i between first (FIG. 30C), second (FIG. 30D) and third (FIG. 30E) positions. That is, the valve 974 is movable progressively from a position in which the first passage 106i is unobstructed by the valve (i.e., the first position) to a position in which is obstructs the first passage 106i (i.e., the third position). In the illustrated embodiment, the valve is slidably engaged with a wall of the first passage 106i and comprises a barrier 978 and a pushbutton 976 rigidly connected to the barrier 978. Thus, a user can press the pushbutton 976 to move the barrier 978 transversely across the first passage 106i.

In the first position (FIG. 30C) the barrier 978 is retracted so as not to extend into the first passage 106i. In this position, the second passage 970 has a substantially greater resistance to airflow than the first passage 106i. Hence, the first passage 106i defines a path of least resistance an airflow drawn through the outlet 108i (as shown by the arrows) will flow through the first passage 106i and not the second passage 970. That is, the airflow received at the outlet 108i is exclusively formed of aerosol flowing from the first passage 106i.

In the second position (FIG. 30D), the barrier 978 extends partway across the first passage 106i. In this position the barrier 978 increases the resistance to airflow of the first passage 106i such that air (drawn through the outlet 108i) flows through both the first 106i and second 970 passages. Thus, the airflow received at the outlet 108i is a combination of aerosol (flowing through the first passage 106i) and flavored air (flowing from the second passage 970).

In the third position (FIG. 30E), the barrier 978 extends across the first passage 106i so as to fully obstruct the first passage 106i. Thus, air is forced to flow through the second passage 970 and the air received at the outlet 108i is exclusively formed of flavored air received from the second passage 970.

As should be apparent, the valve 974 allows a user to control (or select) the amount of additive (in this case flavorant) received through the outlet 108i.

FIGS. 31A to 33B illustrate three exemplary valves that may, for example, form part of the substitute smoking system described above with reference to FIGS. 30A to 30E.

In FIGS. 31A and 31B, the valve 974-1 is similar to that described above, except that it further comprises a biasing means in the form of a compression spring 980. The compression spring 980 is disposed between a distal end of the barrier 978-1 and an opposing wall of the first passage 106i-1, and is configured to urge the valve 974-1 towards the first position (i.e., retracted from the first passage 106i-1). Thus, in order to move the valve 974-1 from the first position (FIG. 31A) to the third position (FIG. 31B), a user must push the valve 974-1 against the biasing force of the spring 980.

The valve 974-2 of FIGS. 32A and 32B operates in a slightly different manner. In this embodiment the barrier 978-2 of the valve 974-2 comprises an aperture 982 that allows an airflow to pass through the barrier 978-2. Thus, in this embodiment, in the first position (FIG. 32B), the valve 974-2 extends across the first passage 106i-2 and an airflow passes through the aperture 982. In the third position (FIG. 32A) the valve 978-2 is retracted from the passage 106i-2, but the airflow is blocked by a shroud 984 of the biasing member (i.e., a compression spring 980-2). Thus, in this embodiment the valve 974-2 is biased by the compression spring 980-2 into the third position.

In the embodiment of FIGS. 33A and 33B, the valve 974-3 is hingeably mounted, by way of a hinge 986, to a wall of the first passage 106i-3. Thus, the valve 974-3 is configured to rotate (about the hinge 986) between the first (FIG. 33A) and second (FIG. 33B) positions. In particular, the valve 974-3 rotates in response to an airflow in the first passage 106i-3. This rotation is facilitated by lip 988 at a distal end of the barrier 978-3. As is shown in FIG. 33B, rotation of the valve 974-3 causes the barrier 978-3 to partially obstruct the first passage 106i-3. In this embodiment the valve 974-3 is biased towards the first position by a biasing member 980-3 extending between the valve 974-3 and the wall of the first passage 106i-3 to which the valve 974-3 is mounted.

Tenth Mode: A Heater to Heat Fluid Passing Through a Smoking Substitute Device

Aspects and embodiments of the tenth mode of the present disclosure will now be discussed with reference to the accompanying figures. Further aspects and embodiments will be apparent to those skilled in the art. All documents mentioned in this text are incorporated herein by reference.

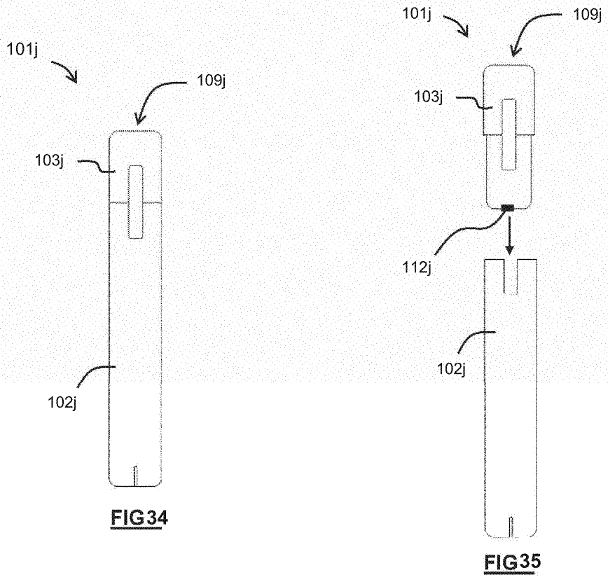

FIGS. 34 and 35 illustrate a smoking substitute system in the form of an e-cigarette system 101j. The system 101j comprises an e-cigarette device defining a reusable main body 102j of the system 101j, and a smoking substitute device in the form of an e-cigarette consumable (or “pod”) 103j, which may also be referred to as a precursor part. In the illustrated embodiment the consumable 103j (smoking substitute device) is removable from the main body (e-cigarette device), so as to be a replaceable component of the system 101j. In other words, the e-cigarette system 101j is a closed system.

As is apparent from FIGS. 34 and 35, the consumable 103j is configured to engage the main body 102j. FIG. 34 shows the main body 102j and the consumable 103j in an engaged state, whilst FIG. 35 shows the main body 102j and the consumable 103j in a disengaged state. When engaged, a portion of the consumable 103j is received in a cavity of the main body 102j and is retained in the engaged position by way of a snap-engagement mechanism. In other embodiments, the main body 102j and consumable 103j may be engaged by screwing one into (or onto) the other, through a bayonet fitting, or by way of an interference fit.

The system 101j is configured to vaporize an aerosol-former or aerosol precursor, which, in the illustrated embodiment, is in the form of a nicotine-based e-liquid 104j. The e-liquid 104j comprises nicotine and a base liquid including propylene glycol and/or vegetable glycerin. In the present embodiment, the e-liquid 104j is flavorless (and does not include any added flavorant). That is, if the e-liquid 104j were to be inhaled (i.e., in aerosol form) by a user, it would not have a particularly perceptible flavor or taste. It is to be appreciated, however, that in other embodiments the e-liquid 104j may comprise an inherent flavorant. In the present specification, the term flavorant may be understood as referring to one or more substances effective to activate at least one of an olfactory receptor in a human nasal cavity; and a taste receptor in a human oral cavity.

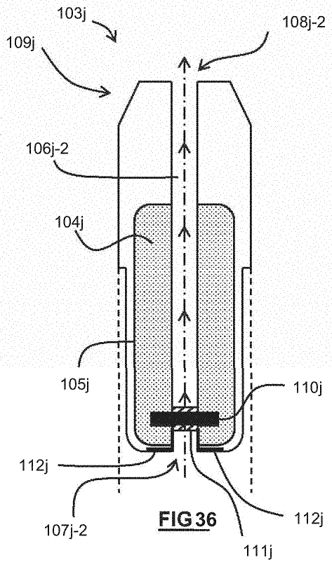

As is more apparent from FIG. 36, the e-liquid 104j is stored within a precursor reservoir in the form of a tank 105j that forms part of the consumable 103j. In the illustrated embodiment, the consumable 103j is a “single-use” consumable 103j. That is, upon exhausting the e-liquid 104j in the tank 105j, the intention is that the user disposes of the entire consumable 103j. In other embodiments, the e-liquid (i.e., aerosol precursor) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105j surrounds, and thus defines a portion of, a fluid flow passage 106j-2 that extends between an inlet 107j-2 and an outlet 108j-2 at opposing ends of the consumable 103j. In this respect, the passage 106j-2 comprises an upstream end at the end of the consumable 103j that engages with the main body 102j, and a downstream end at an opposing end of the consumable 103j that comprises a mouthpiece 109j of the system 101j. The fluid flow passage 106j-2 comprises an inlet 107j-2 at its upstream end, and an outlet at its downstream end, the outlet 108j-2 thus forming part of, or being located at, the mouthpiece 109j. When the consumable 103j is engaged with the main body 102j, a user can inhale (i.e., take a puff) via the mouthpiece 109j so as to draw air through the passage 106j-2, and so as to form an airflow (indicated by arrows) in a direction from the inlet 107j-2 to the outlet 108j-2 of the passage 106j-2.

The passage 106j-2 may be partially defined by a tube (e.g., a metal tube or a tube formed from plastic material) extending through the consumable 103j. At its upstream inlet end, the passage 106j-2 may be in fluid communication with a gap defined between the consumable 103j and the main body 102j when the consumable 103j and the main body 102j are interengaged, such that when a user draws on the outlet 108j-2 at the mouthpiece 109j, ambient air from outside of the system 101j is drawn into the passage 106j-2 via the inlet 107j-2.

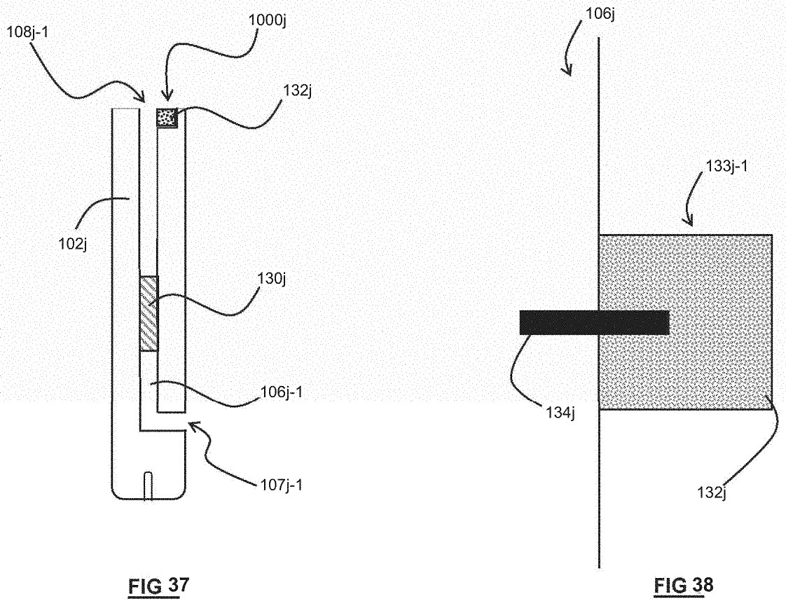

Alternatively, the main body 102j may additionally comprise a fluid passage 106j-1, extending from a main body inlet 107j-1 to a main body outlet 108j-1 as illustrated, for example, in FIG. 37. In such an arrangement, the main body outlet 108j-1 may be arranged such that when the main body 102j and consumable 103j are interengaged, a substantially fluid-tight seal is formed at the connection point between main body outlet 108j-1 and fluid inlet 107j-2, meaning that main body fluid passage 106j-1 and consumable passage 106j-2 become interconnected to form a single passage 106j extending through the smoking substitute system 101j and fluidly connecting main body inlet 107j-1 and consumable outlet 108j-2. Furthermore, the main body fluid passage 106j-1 or the consumable fluid passage 106j-2 may be shaped to provide a recess or receptacle 1000 into which a source of flavorant 132j may be received, as will be described in more detail hereinafter.

The smoking substitute system 101j is configured to vaporize the e-liquid 104j for inhalation by a user. To provide this function, the consumable 103j is provided with an aerosol generator comprising a porous wick 110j and a resistive heating element in the form of a precursor heating filament 111j that is helically wound around a portion of the porous wick 110j. The aerosol generator is arranged between the inlet 107j-1 and the outlet 108j-2 of the smoking substitute system 101j, and is in fluid communication with both the inlet 107j-1 and the outlet 108j-2. The porous wick 110j extends across the passage 106j (e.g., transverse to a longitudinal axis of the passage 106j), and opposing ends of the wick 110j extend into the tank 105j so as to be submerged in the e-liquid 104j so as to draw e-liquid from the tank 105j via wicking or capillary action. In this way, e-liquid 104j contained in the tank 105j is conveyed from the opposing ends of the porous wick 110j to a central portion of the porous wick 110j so as to be exposed to the airflow in the passage 106j caused by a user inhaling or drawing on the mouthpiece 109j. The helical filament 111j is wound about the exposed central portion of the porous wick 110j and is electrically connected to an electrical interface in the form of electrical contacts 112j mounted at the end of the consumable that is proximate the main body 102j (when engaged).

When the consumable 103j is engaged with the main body 102j, the electrical contacts 112j make contact with corresponding electrical contacts (not shown) of the main body 102j. The main body electrical contacts are electrically connected to a power source (not shown) of the main body 102j, such that the filament 111j is electrically connected to the power source when the consumable 103j and the main body 102j are interengaged. In this way, power can be supplied by the main body 102j to the filament 111j in order to heat the filament 111j. This heat is transferred from the filament 111j to the porous wick 110j which causes e-liquid 104j conveyed by the porous wick 110j to increase in temperature to a point at which it vaporizes. The vaporized e-liquid becomes entrained in the airflow past the wick 110j and, between the vaporization point at the filament 111j and the outlet 108j-2 of the passage 106j, condenses to form an aerosol. This aerosol is then inhaled, via the mouthpiece 109j, by a user of the system 101j.

The power source of the main body 102j may be in the form of a battery (e.g., a rechargeable battery). The main body 102j may comprise a connector in the form of, e.g., a USB port for recharging this battery. The main body 102j may also comprise a controller that controls the supply of power from the power source to the main body electrical contacts (and thus to the filament 111j). That, is the controller may be configured to control a voltage applied across the main body electrical contacts, and thus the voltage applied across the filament 111j. In this way, the filament 111j may only be heated under certain conditions (e.g., during a puff and/or only when the system is in an active state). In this respect, the main body 102j may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102j and consumable 103j may comprise a further interface which may, for example, be in the form of an RFID reader, a barcode or QR code reader. This interface may be able to identify a characteristic (e.g., a type) of a consumable 103j engaged with the main body 102j. In this respect, the consumable 103j may include any one or more of an RFID chip, a barcode or QR code, or memory within which is an identifier and which can be interrogated via the interface.

In the illustrated smoking substitute system 101j, an additional heater 130j (i.e., separate from and in addition to the heater of the aerosol generator) is provided upstream of the aerosol generator along a fluid passage 106j connecting the fluid inlet 107j-1 or 107j-2 and the aerosol generator. Further, a source of flavorant 132j is provided along the fluid passage 106j, between the additional heater 130j and the aerosol generator, such that the source of flavorant 132j is presented to a flow of fluid from the additional heater 130j towards the aerosol generator. The arrangement is thus configured such that said flow of fluid is heated by the additional heater 130j to release flavorant 132j from the source for entrainment in said fluid upstream of the aerosol generator.

The flavorant 132j may be formulated such that heated fluid is required to release the flavorant 132j from the source. In other words, a fluid flow (for example from inhalation) without operation of the additional heater 130j would cause substantially no flavorant 132j to be entrained therein. This would allow, for example, for a user to select whether or not to use the flavorant during a vaping session. Further, this type of configuration may allow for control of intensity of the flavor, for example by varying the temperature of the additional heater 130j.

Using the additional heater 130j to heat a fluid flow, said fluid flow then being flowed past the source of flavorant to heat it indirectly, rather than heating the source of flavorant 132j directly, may allow for a greater range of flavorants to be used in smoking substitute system 101j. For example, it may allow the use of thermally sensitive flavorants 132j which could be degraded by the direct application of heat from a heater. Further, heating the fluid rather than heating the flavorant 132j directly may prolong the life of the additional heater 130j by preventing it from becoming fouled with residual flavorant 132j.

The additional heater 130j may be operable in combination (for example in synchronism or with a predetermined operating time offset) with the heating filament 111j of the aerosol generator. Additionally, or alternatively, the additional heater 130j and the heating filament 111j of the aerosol generator may be operable independently of one another.

In embodiments in which the e-liquid 104j contains an inherent flavorant, it will be appreciated that the release of flavorant 132j from the source in the manner noted above will serve to supplement, and optionally blend with, the flavorant of the e-liquid in use. For example, the flavorant 132j of the source may have the same flavor as that in the e-liquid 104j in order to supplement the flavor provided within the e-liquid and thereby provide the user with an intensified flavor sensation. Alternatively, the flavorant 132j of the source may be complementary to that in the e-liquid 104j (i.e., the flavors/aromas of the two flavorants, when mixed, provide a pleasing sensory combination to a user). Providing the flavorant 132j separately to the aerosol precursor 104j affords the user an opportunity to change between different flavorants 132j without necessitating a change of e-liquid, for example during a vaping session.

The additional heater 130j may comprise one or more heating elements such as resistive heating elements in the form of a mesh or a coil, around or through which fluid may flow. Such heating elements may extend partially or substantially completely across the passage 106j to maximize heat transfer from the or each element to the fluid. Alternatively, or additionally, the or each heating element may form a part of the peripheral wall of passage 106j.

Various arrangements within the smoking substitute device 101j, and specific configurations of both the additional heater 130j and the source of flavorant 132j are possible, some examples of which are described below.

For example, the additional heater 130j may be located in the main body 102j, as illustrated in FIG. 37. The configuration of the passage 106j-1 illustrated in FIG. 37 is for illustrative purposes only, and is not intended to be limiting. For example, the fluid inlet 107j-1 might alternatively be located closer to or further away from the end of main body 102j which is configured to engage with the smoking substitute device 103j than illustrated, or may even be located on a different face of the main body 102j. The recess 1000 maybe omitted, or may be located at a different position along the fluid passage 106j-1. Other components of the main body 102j (e.g., the power source) are not illustrated in FIG. 37 for clarity, but may be arranged to be adjacent to or surround all or part of the fluid passage 106j-1 as required.

In the case of embodiments in which the additional heater 130j is provided within the main body 102j of the system, the source of flavorant 132j may take the form of a flavorant reservoir 133j located either in the main body 102j, or as part of the consumable 103j. The flavorant reservoir 133j may surround, and thus define, at least part of the passage 106j. Alternatively, the flavorant reservoir 133j may be located adjacent to, but not surround the passage 106j. The flavorant provided in such a flavorant reservoir 133j may be provided, for example, as a flavored liquid. A reservoir 133j that is provided within the main body 102j may be user-refillable, and may thus be located proximate to the interface between main body 102j and the consumable pod 103j to allow convenient user-access for refilling. The flavorant reservoir may be at least partially filled during manufacture of the consumable 103j such that the user is not required to fill the reservoir with flavorant before using the consumable.

The flavorant reservoir may further comprise one or more porous wicks 134j extending across at least part of fluid flow passage 106j, so as to be presented to a fluid flow therethrough (e.g., transverse to a longitudinal axis of the passage 106j), as illustrated exemplarily in FIG. 38, where the reservoir is denoted as 133j-1. In embodiments where the flavorant reservoir 133j-1 surrounds a portion of the passage 106j, the porous wick 134j may extend substantially completely across the passage 106j, such that both opposing ends of the wick extend into the flavorant reservoir 133j-1. Alternatively, in embodiments where the flavorant reservoir 133j-1 does not surround the passage 106j, only one end of each porous wick 134j may extend into the flavorant reservoir 133j-1, with the other end being located within the passage 106j. The end or ends of the porous wicks 134j that extend into the flavorant reservoir 133j-1 are arranged to be submerged in the flavorant 132j. In this way, flavorant 132j contained in the flavorant reservoir 133j-1 is conveyed from the end or ends of the porous wick 134j that extend into the flavorant reservoir 133j-1 to a portion of the porous wick 134j located within the passage 106 so as to be exposed to the airflow therein (i.e., caused by a user inhaling).

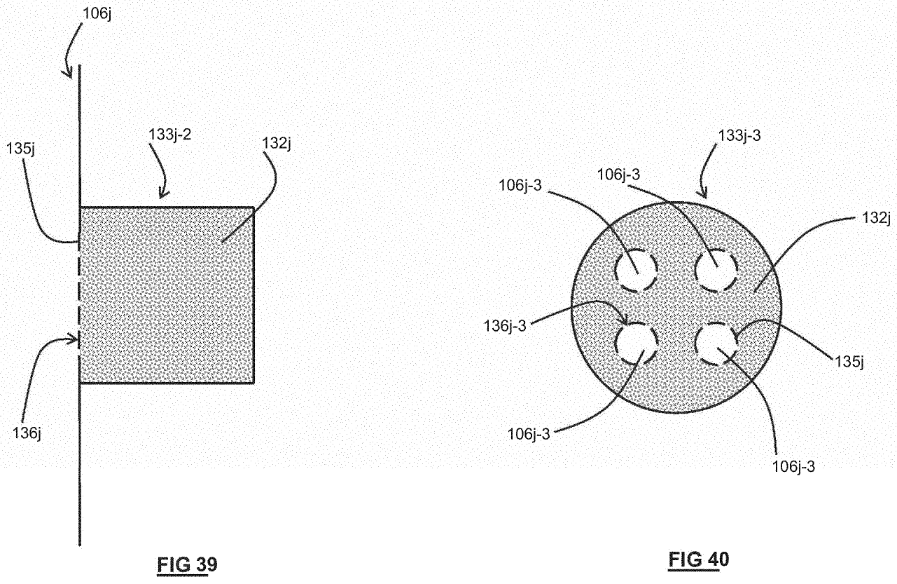

Alternatively, or additionally, at least a part of a barrier separating the passage 106j from the flavorant reservoir 133j-2 may be permeable to flavorant 132j as illustrated exemplarily in FIG. 39. FIG. 39 illustrates a perforated barrier 135j separating the passage 106j from the flavorant reservoir 133j-2. The barrier may thus comprise one or more small apertures 136j to permit the passage of flavorant therethrough, and may be formed as part of the sidewall of the flow passage 106j. In this type of arrangement, flavorant 132j may permeate through the barrier from the flavorant reservoir 133j-2 to the passage 106j, for example by capillary action, so as to be exposed to the airflow in the passage 106j (i.e., caused by a user inhaling).

In an alternative arrangement illustrated in FIG. 40, the flavorant reservoir 133j-3 is housed within the flow passage 106j so as to extend substantially completely across the flow passage 106j. As will be observed, in this arrangement, the reservoir 133j-3 is provided with a plurality of relatively small diameter bores 106j-1, each of which extends fully through the reservoir from its upstream end to its downstream end. Each bore 106j-1 may be defined by a similar permeable barrier 135j-3 (each of which may thus be perforated with small apertures 136j-3) in a similar manner to the barrier arrangement described above with reference to FIG. 40. As will be appreciated, fluid flow through the flow passage 106j will thus become split into separate flow streams through the bores 106j-1 of the reservoir, the flow streams then rejoining and mixing with one another downstream of the reservoir. This type of multi-bore configuration of the reservoir 133j-3 can be effective to increase the effective surface area through which the flavorant can permeate, when compared to the single barrier arrangement of FIG. 39, which can be effective to increase the concentration of flavorant which may be entrained in the airflow through the passage 106j.

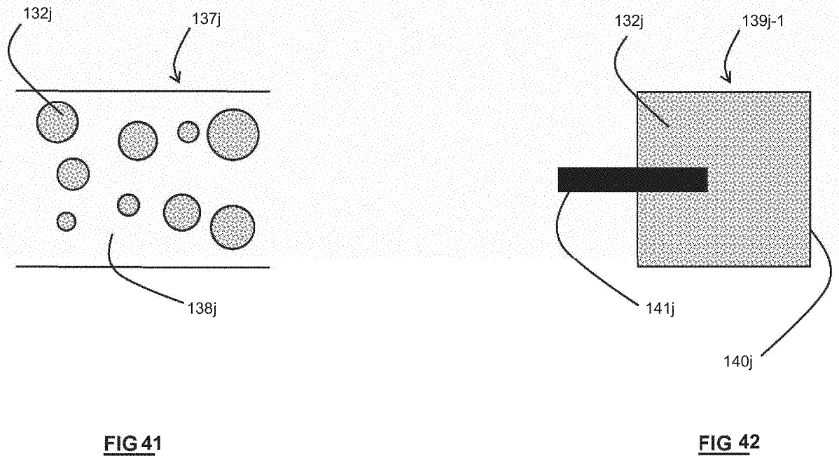

Alternatively, with reference to FIG. 41, in embodiments where the additional heater 130j is provided within the main body 102j of the system, it is envisaged that the source of flavorant 132j may be a flavored article 137j, comprising a substrate 138j carrying the flavorant 132j. Such a flavored article 137j may be received within the fluid flow passage 106j, which may be shaped to provide a recess or receptacle 1000 within which the flavored article 137j may be located. At least part of the substrate 138j may be formed from a polymeric material (e.g., silicone). Further, at least part of the substrate 138j may be formed from a porous material, foam or foamed material as illustrated exemplarily in FIG. 41. Still further, at least a part of the substrate 138j may be formed from an air permeable material. The flavorant 132j may be one or both of comprised within or coated on a surface of the substrate 138j. The flavorant 132j may be introduced into the flavored article 137j by coating (e.g., spray coating). Alternatively, or additionally, the flavorant 132j may be introduced or impregnated into the flavored article 137j by, for example, immersion of the substrate 138j in a liquid comprising flavorant 132j. This process of introducing flavorant 132j could either be carried out during manufacture of the flavored article 137j, or by an end-user.

Alternatively, having regard to FIG. 42, the source of flavorant 132j may be a “flavor pod” 139j comprising a container 140j at least partially filled with the flavorant 132j. Similar to the flavored article 137j illustrated in FIG. 41, the flavor pod 139j may be received within the passage 106j, which may be shaped to provide a recess or receptacle 1000 into which the flavor pod 139j may be located. When in place, a surface of the flavor pod 139j-2 may define part of a peripheral wall of the passage 106j. The flavor pod 139j-1 may furthermore comprise one or more porous wicks 141j extending at least partially across the passage 106j (e.g., transverse to a longitudinal axis of the passage 106j), so as to be presented to a fluid flow therethrough. At least one end of each porous wick 141j extends into the flavor pod container 140j, so as to be submerged in the flavorant 132j. In this way, the flavorant 132j contained in the flavor pod container 140j is conveyed from the end or ends of the porous wick 141j extending into the flavor pod container 140j to a portion of the porous wick 141j located within the passage 106j, so as to be exposed to the airflow therein (i.e., caused by a user inhaling).

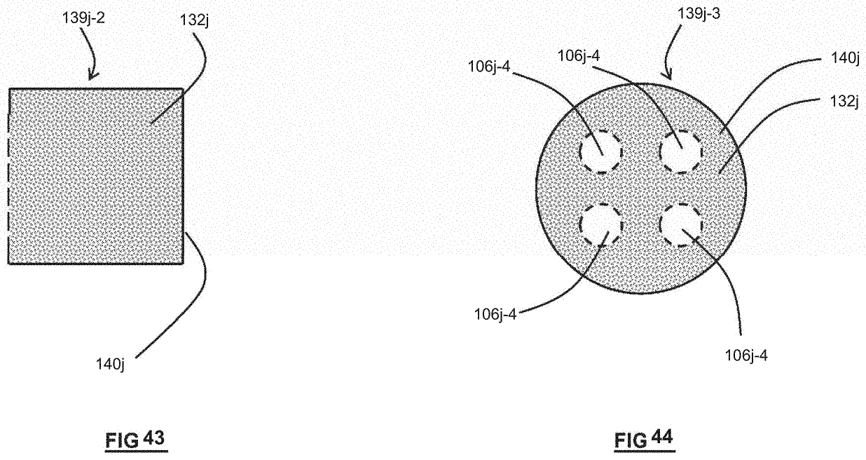

Additionally, or alternatively, at least a part of a surface of the flavor pod 139j located adjacent to, or defining part of passage 106j may be flavorant-permeable as illustrated exemplarily in FIG. 43.

Alternatively, the flavor pod 139j-3 may have a plurality of fluid passages or bores 106j-2 therethrough, at least part of the wall or barrier dividing each bore 106d from the flavorant container 140j being flavorant-permeable as illustrated exemplarily in FIG. 44. As will be appreciated by those of skill in the art, these arrangements of FIGS. 43 and 44 bear similarities with the arrangements of FIGS. 39 and 40. In such arrangements, flavorant 132j may permeate from the flavor pod 139j to the passage 106j for entrainment in the airflow therethrough (i.e., caused by a user inhaling).

The recess or receptacle 1000 in which flavored article 137j or flavor pod 139j is locatable may be provided within the main body 102j, the system 101j, or within the consumable 103j. The flavored article 137j or flavor pod 139j may be releasably attached to the consumable 103j, for example to the end of the consumable which is configured to engage with the main body 102j. Various means of attachment between the source of flavorant 132j and the consumable 103j may be possible, including, but not limited to, an interference fit, a snap fit comprising one or more raised bumps and corresponding recesses on the interfacial surface between the two components, or through physically coupling together by screwing one onto the other, through a bayonet fitting, through a latching mechanism, or through a clip or clasp. If the source of flavorant 130j is attached via a latching mechanism, this may further include a button by which the latch may be released. A clip or clasp may be fixed in position relative to the consumable 103j, or hingedly, rotatably, or slidably attached to the consumable 103j so as to be movable to allow the flavored article 137j or flavor pod 139j to be fitted and released.

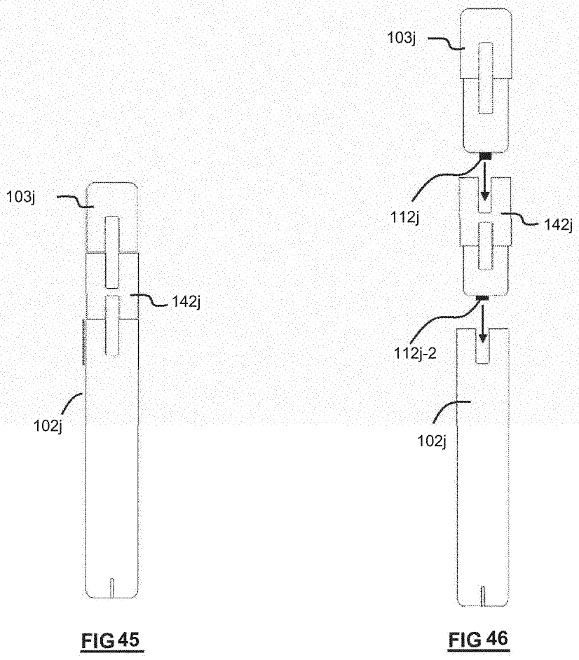

The flavored article 137j or flavor pod 139j may alternatively be comprised within a flavor part 142j of the smoking substitute system 101j, in the manner illustrated schematically in FIGS. 45 and 46. Said flavor part 142j may be located between the consumable 103j and the main body 102j, such that each engages with an opposing end or side of the flavor part 142j, but not directly with each other. The flavor part 142j in such an embodiment may further comprise an electrical contact 112j-2 and connection therethrough, so that power may be conveyed from the contacts of the main body 102j to the contacts 112j of the consumable 103j, via the flavor part 142j. The interface between the consumable 103j and flavor part 142j may be configured to be the same as the interface between the flavor part 142j and main body 102j, such that the user is afforded the option of choosing to use the smoking substitute device 101j either with or without the flavor part 142j.

The flavored article 137j or flavor pod 139j may therefore be provided as a stand-alone consumable, independent of the consumable 103j comprising the aerosol generator. One or more flavored articles 137j or flavor pods 139j may be provided in a retail pack. Each flavored article 137j or flavor pod 139j within a pack may comprise the same flavorant 132j. Alternatively, the pack may comprise flavored articles 137j or flavor pods 139j containing different flavorants 132j, in order to provide a user with a selection of possible flavorants 132j.

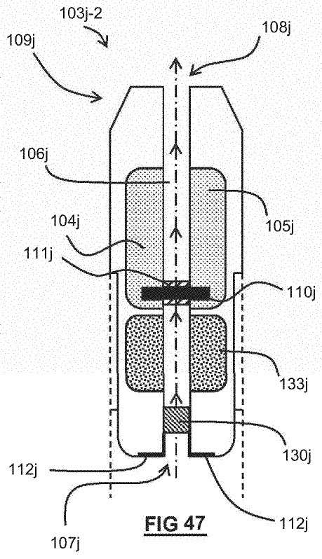

In alternative embodiments, it is envisaged that the additional heater 130j may be provided within a consumable 103j-2, located along the fluid passage 106j-2 between fluid inlet 107j-2 and the aerosol generator of the consumable, as illustrated exemplarily in FIG. 47. In such an embodiment, the source of flavorant 132j could be a flavorant reservoir 133j. In such a configuration, the main body 102j may not comprise a fluid inlet 107j-1, outlet 108j-1 or passage 106j-1.

Eleventh Mode: A Delivery of Flavor to a User by Olfactory Flavor Delivery

Aspects and embodiments of the eleventh mode of the present disclosure will now be discussed with reference to the accompanying figures. Further aspects and embodiments of the eleventh mode will be apparent to those skilled in the art. All documents mentioned in this text are incorporated herein by reference.

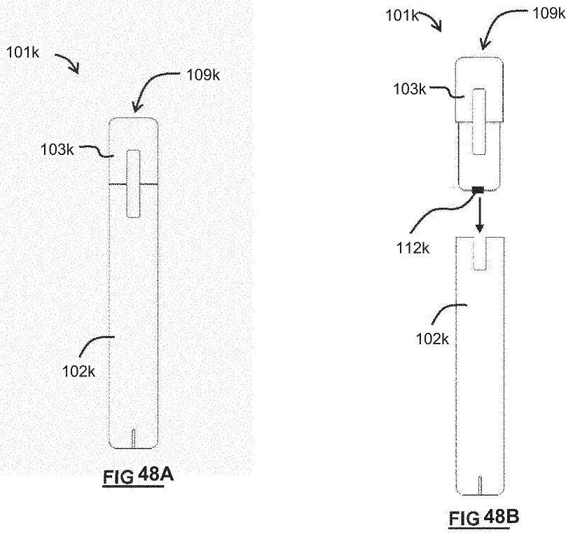

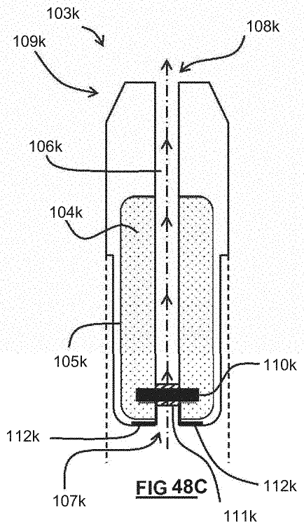

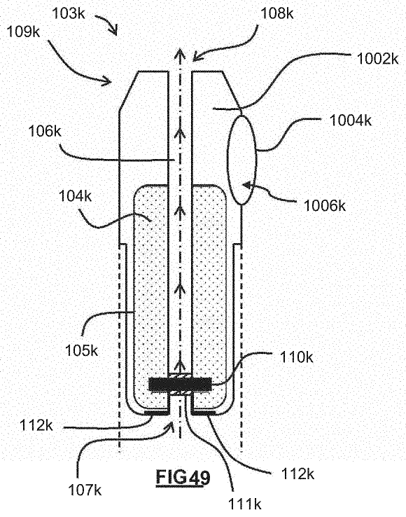

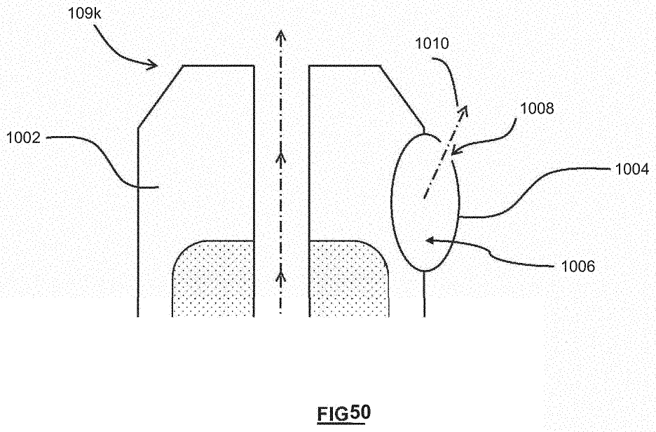

FIGS. 48A and 48B illustrate a smoking substitute system in the form of an e-cigarette system 101k. The system 101k comprises an e-cigarette device defining a main body 102k of the system 101k, and a smoking substitute apparatus in the form of an e-cigarette consumable (or “pod”) 103k. In the illustrated embodiment the consumable 103k (smoking substitute apparatus) is removable from the main body (e-cigarette device), so as to be a replaceable component of the system 101k. In other words, the e-cigarette system 101k is a closed system.

As is apparent from FIGS. 48A and 48B, the consumable 103k is configured to engage the main body 102k. FIG. 48A shows the main body 102k and the consumable 103k in an engaged state, whilst FIG. 48B shows the main body 102k and the consumable 103k in a disengaged state. When engaged, a portion of the consumable 103k is received in a cavity of the main body 102k and is retained in the engaged position by way of a snap-engagement mechanism. In other embodiments, the main body 102k and consumable 103k may be engaged by screwing one into (or onto) the other, through a bayonet fitting, or by way of an interference fit.

The system 101k is configured to vaporize an aerosol-former, which in the illustrated embodiment, is in the form of a nicotine-based e-liquid 104k. The e-liquid 104k comprises nicotine and a base liquid including propylene glycol and/or vegetable glycerin. In the present embodiment, the e-liquid 104k is flavorless (i.e., it does not include any added flavorant). That is, if the e-liquid 104k were to be inhaled (i.e., in aerosol form) by a user, it would not have a particularly perceptible flavor or taste.

As is more apparent from FIG. 48C, this e-liquid 104k is stored within a reservoir in the form of a tank 105k that forms part of the consumable 103k. In the illustrated embodiment, the consumable 103k is a “single-use” consumable 103k. That is, upon exhausting the e-liquid 104k in the tank 105k, the intention is that the user disposes of the entire consumable 103k. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105k surrounds, and thus defines a portion of, a passage 106k that extends between an inlet 107k and an outlet 108k at opposing ends of the consumable 103k. In this respect, the passage comprises an upstream end at the end of the consumable 103k that engages with the main body 102k, and a downstream end at an opposing end of the consumable 103k that comprises a mouthpiece 109k of the system 101k. When the consumable 103k is engaged with the main body 102k, a user can inhale (i.e., take a puff) via the mouthpiece 109k so as to draw air through the passage 106k, and so as to form an airflow (indicated by arrows) in a direction from the inlet 107k to the outlet 108k of the passage 106k. Although not illustrated, the passage 106k may be partially defined by a tube (e.g., a metal tube) extending through the consumable 103k. The passage 106k is in fluid communication with a gap defined between the consumable 103k and the main body 102k (when engaged) such that air outside of the system 101k is drawn into the passage 106k (during an inhale).

The smoking substitute system 101k is configured to vaporize the e-liquid 104k for inhalation by a user. To provide this, the consumable 103k comprises a heater having of a porous wick 110k and a resistive heating element in the form of a heating filament 111k that is helically wound around a portion of the porous wick 110k. The porous wick 110k extends across the passage 106k (i.e., transverse to a longitudinal axis of the passage 106k) and opposing ends of the wick 110k extend into the tank 105k (so as to be submerged in the e-liquid 104k). In this way, e-liquid 104k contained in the tank 105k is conveyed from the opposing ends of the porous wick 110k to a central portion of the porous wick 110k so as to be exposed to the airflow in the passage 106k (i.e., caused by a user inhaling). In other embodiments the heating filament 111k and/or wick 110k may form part of the main body (but may engage the tank 105k during engagement of the main body 102k and the consumable 103k).

The helical filament 111k is wound about this exposed central portion of the porous wick 110k and is electrically connected to an electrical interface in the form of electrical contacts 112k mounted at the end of the consumable that is proximate the main body 102k (when engaged). When the consumable 103k is engaged with the main body 102k, the electrical contacts 112k contact corresponding electrical contacts (not shown) of the main body 102k. The main body electrical contacts are electrically connected to a power source (not shown) of the main body 102k, such that (in the engaged position) the filament 111k is electrically connected to the power source. In this way, power can be supplied by the main body 102k to the filament 111k in order to heat the filament 111k. This heat is transferred from the filament 111k to the porous wick 110k which causes e-liquid 104k conveyed by the porous wick 110k to increase in temperature to a point at which it vaporizes. The vaporized e-liquid becomes entrained in the airflow and, between the vaporization point at the filament 111k and the outlet 108k of the passage 106k, condenses to form an aerosol. This aerosol is then inhaled, via the mouthpiece 109k, by a user of the system 101k.

The power source of the main body 102k may be in the form of a battery (e.g., a rechargeable battery).

The main body 102k may comprise a connector in the form of, e.g., a USB port for recharging this battery. The main body 102k may also comprise a controller that controls the supply of power from the power source to the main body electrical contacts (and thus to the filament 111k). That, is the controller may be configured to control a voltage applied across the main body electrical contacts, and thus the voltage applied across the filament 111k. In this way, the filament 111k may only be heated under certain conditions (e.g., during a puff and/or only when the system is in an active state). In this respect, the main body 102k may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102k and consumable 103k may comprise a further interface which may, for example, be in the form of an RFID reader, a barcode or QR code reader. This interface may be able to identify a characteristic (e.g., a type) of a consumable 103k engaged with the main body 102k. In this respect, the consumable 103k may include any one or more of an RFID chip, a barcode or QR code, or memory within which is an identifier and which can be interrogated via the interface.

FIG. 49 illustrates a consumable 103k for a smoking substitute device such as the electronic cigarette system shown in FIGS. 48A-C. The consumable 103k has an outer casing 1002 in which the tank 105k containing the e-liquid 104k is disposed. The passage 106k is disposed through the outer casing 1002.

A flavor delivery apparatus 1004 is positioned in the outer casing 1002. The flavor delivery apparatus 1004 is a compressible pocket. The compressible pocket 1004 is molded integrally as part of the outer casing 1002 so that at least part of the pocket extends outwardly from an outer surface of the outer casing 1002. In FIG. 49, a portion of the pocket is positioned inside the outer casing 1002 of the consumable 103k, and another portion of the pocket is positioned to extend away and outwardly from the outer casing 1002 in an uncompressed state.

The compressible pocket 1004 is distinct and separate from the tank 105k containing the e-liquid, and the passage 106k through the consumable 103k. The compressible pocket is thus fluidly isolated from the tank 105k and the passage 106k. Specifically, the flow path of the air from the inlet 107k to the outlet 108k of the passage 106k is thus separate and distinct from the pocket 1004 so that the flow of air through the tank 105k does not come into contact with the compressible pocket 1004 or the contents thereof.

The compressible pocket 1004 has an internal volume defining a reservoir which contains a fragranced liquid 1006. The fragranced liquid 1006 is configured to release a fragrance, smell or odor, and may be oil-based and/or sugar-based. The compressible pocket may also contain air.

The pocket 1004 has an outlet mister having a plurality of slits and/or holes. The outlet mister is formed from a silicon valve. The outlet mister is configured to form a mist of fragranced liquid 1006 as the fragranced liquid is urged therethrough, the resulting mist thus being released from the pocket 1004 into the surrounding ambient air (as shown using the dashed arrow 1010 in FIG. 50). The pocket 1004 also has a one-way valve inlet which allows surrounding ambient air into the pocket 1004, but prevents air escaping from the pocket 1004.

In FIG. 50, the outlet mister is integral with the valve inlet, such that they form a single combined valve 1008.

During compression of the pocket 1004 from an uncompressed state (as shown in FIGS. 49 and 50) to a compressed state, the single combined valve 1008 acts as the outlet mister. After the compression of the pocket 1004, the single combined valve 1008 acts as a one-way valve outlet which allows surrounding air into the pocket 1004, but prevents air in the pocket 1004 from escaping from the pocket. Thus, the pocket 1004 can return from the compressed state to the uncompressed state after each compression of the pocket 1004.

A user of the electronic cigarette device can actuate the release of flavor from the pocket 1004 by compressing, or pushing, the outwardly directed portion of the pocket 1004 towards the outer casing 1002 of the consumable. In other words, the user compresses the pocket 1004 from the uncompressed state to the compressed state. The movement of the pocket 1004 from the uncompressed state to the compressed state acts to blow a combination of air and fragranced liquid 1006 in the pocket 1004 through the single combined valve 1008. During compression, the single combined valve 1008 acts as an outlet mister and creates a fine mist of droplets of the fragranced liquid 1006 (hereinafter a fragranced vapor mist) which passes out of the single combined valve 1008 and into the surrounding ambient air. The fragranced vapor mist can then travel towards a user's nose for olfactory flavor delivery.

After compression of the pocket 1004, and when the user has stopped compressing the pocket 1004, the single combined valve 1008 acts as a one-way valve, allowing surrounding outside air into the pocket through the single combined valve 1008, but preventing air already inside the pocket 1004 from escaping from the pocket 1004. As the single combined valve 1008 acts as a one-way valve after compression of the pocket 1004, the pocket 1004 returns from the compressed state to the uncompressed state. The pocket 1004 can then be compressed again by a user to repeatedly release fragranced vapor mist.

In an alternative embodiment, the one-way valve may be separate from the outlet mister.

The pocket 1004 is positioned on an outside of the outer casing 1002 of the consumable, such that a user can easily access the pocket 1004 for compression with, for example, a finger in order to actuate flavor delivery.

The outlet mister (which in the embodiment shown in FIG. 50 is the single combined valve 1008) is positioned on the pocket 1004 near an upper end of the pocket 1004 adjacent to the mouthpiece 109k of the consumable 103k. Therefore, in use (i.e., during vaping), the outlet mister is positioned underneath and/or adjacent to the nose of the user for olfactory flavor delivery.

In some embodiments, it is envisaged that the flavor delivery article may be detachable from the outer casing 1002. For example, the flavor delivery article may be releasably clipped onto the outer casing 1002 of the consumable 103k in order to permit convenient replacement and/or change the flavor delivery article. Therefore, a user can change the type of flavor (e.g., from a fruit flavor to a spiced flavor, for example), or can replace an empty flavor delivery article, in which all the fragranced liquid has been used. Alternatively, the flavor delivery article may be integral with or otherwise permanently attached to the outer casing 1002, such that the flavor of the consumable may only be changed and/or the flavor delivery article replaced along with the replaceable consumable 103k.

Alternatively, and/or additionally, the flavor delivery article may be refillable with fragranced liquid such that the flavor delivery article is reusable.

In an alternative embodiment, the pocket is not attached to the consumable 103k, but is instead attached to the main body 102k of the smoking substitute device.

Twelfth Mode: A Flavor Delivery Article for a Smoking Substitute Device

Aspects and embodiments of the twelfth mode of the present disclosure will now be discussed with reference to the accompanying figures. Further aspects and embodiments of the twelfth mode will be apparent to those skilled in the art. All documents mentioned in this text are incorporated herein by reference.

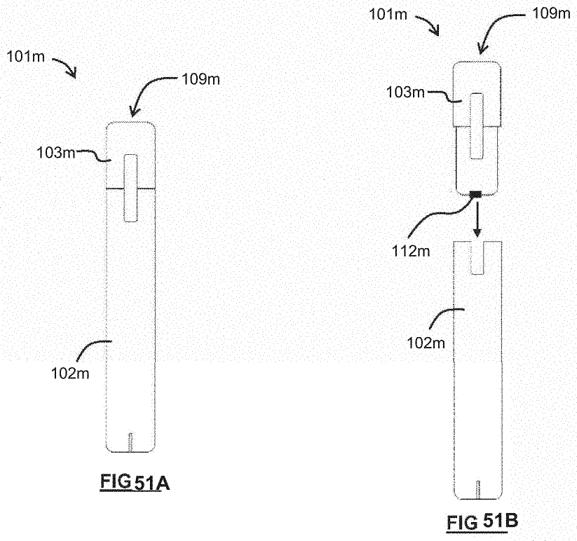

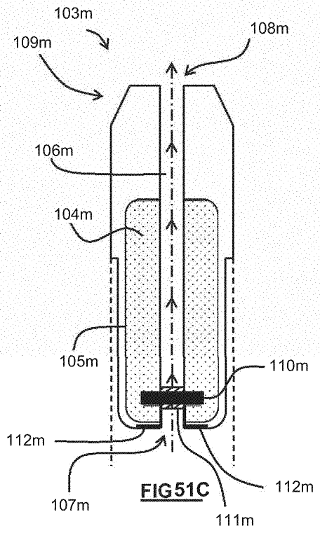

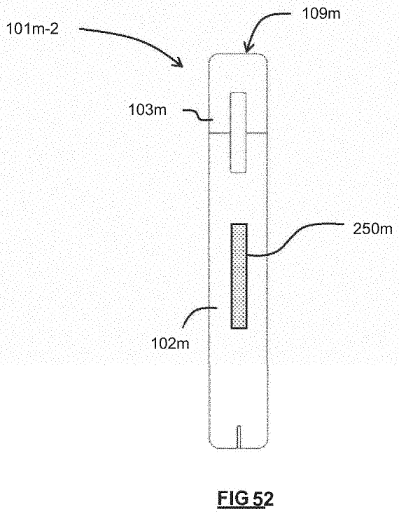

FIGS. 51A and 51B illustrate a smoking substitute system in the form of an e-cigarette system 101m. The system 101m comprises an e-cigarette device defining a main body 102m of the system 101m, and a smoking substitute apparatus in the form of an e-cigarette consumable (or “pod”) 103m. In the illustrated embodiment the consumable 103m (smoking substitute apparatus) is removable from the main body (e-cigarette device), so as to be a replaceable component of the system 101m. In other words, the e-cigarette system 101m is a closed system.

As is apparent from FIGS. 51A and 51B, the consumable 103m is configured to engage the main body 102m. FIG. 51A shows the main body 102m and the consumable 103m in an engaged state, whilst FIG. 51B shows the main body 102m and the consumable 103m in a disengaged state. When engaged, a portion of the consumable 103m is received in a cavity of the main body 102m and is retained in the engaged position by way of a snap-engagement mechanism. In other embodiments, the main body 102m and consumable 103m may be engaged by screwing one into (or onto) the other, through a bayonet fitting, or by way of an interference fit.

The system 101m is configured to vaporize an aerosol-former, which in the illustrated embodiment, is in the form of a nicotine-based e-liquid 104m. The e-liquid 104m comprises nicotine and a base liquid including propylene glycol and/or vegetable glycerin. In the present embodiment, the e-liquid 104m is flavorless (and does not include any added flavorant). That is, if the e-liquid 104m were to be inhaled (i.e., in aerosol form) by a user, it would not have a particularly perceptible flavor or taste.

As is more apparent from FIG. 51C, this e-liquid 104m is stored within a reservoir in the form of a tank 105m that forms part of the consumable 103m. In the illustrated embodiment, the consumable 103m is a “single-use” consumable 103m. That is, upon exhausting the e-liquid 104m in the tank 105m, the intention is that the user disposes of the entire consumable 103m. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105m surrounds, and thus defines a portion of, a passage 106m that extends between an inlet 107m and an outlet 108m at opposing ends of the consumable 103m. In this respect, the passage comprises an upstream end at the end of the consumable 103m that engages with the main body 102m, and a downstream end at an opposing end of the consumable 103m that comprises a mouthpiece 109m of the system 101m. When the consumable 103m is engaged with the main body 102m, a user can inhale (i.e., take a puff) via the mouthpiece 109m so as to draw air through the passage 106m, and so as to form an airflow (indicated by arrows) in a direction from the inlet 107m to the outlet 108m of the passage 106m. Although not illustrated, the passage 106m may be partially defined by a tube (e.g., a metal tube) extending through the consumable 103m. The passage 106m is in fluid communication with a gap defined between the consumable 103m and the main body 102m (when engaged) such that air outside of the system 101m is drawn into the passage 106m (during an inhale).

The smoking substitute system 101m is configured to vaporize the e-liquid 104m for inhalation by a user. To provide this, the consumable 103m comprises a heater having of a porous wick 110m and a resistive heating element in the form of a heating filament 111m that is helically wound around a portion of the porous wick 110m. The porous wick 110m extends across the passage 106m (i.e., transverse to a longitudinal axis of the passage 106m) and opposing ends of the wick 110m extend into the tank 105m (so as to be submerged in the e-liquid 104m). In this way, e-liquid 104m contained in the tank 105m is conveyed from the opposing ends of the porous wick 110m to a central portion of the porous wick 110m so as to be exposed to the airflow in the passage 106m (i.e., caused by a user inhaling). In other embodiments the heating filament 111m and/or wick 110m may form part of the main body (but may engage the tank 105m during engagement of the main body 102m and the consumable 103m).

The helical filament 111m is wound about this exposed central portion of the porous wick 110m and is electrically connected to an electrical interface in the form of electrical contacts 112m mounted at the end of the consumable that is proximate the main body 102m (when engaged). When the consumable 103m is engaged with the main body 102m, the electrical contacts 112m contact corresponding electrical contacts (not shown) of the main body 102m. The main body electrical contacts are electrically connected to a power source (not shown) of the main body 102m, such that (in the engaged position) the filament 111m is electrically connected to the power source. In this way, power can be supplied by the main body 102m to the filament 111m in order to heat the filament 111m. This heat is transferred from the filament 111m to the porous wick 110m which causes e-liquid 104m conveyed by the porous wick 110m to increase in temperature to a point at which it vaporizes. The vaporized e-liquid becomes entrained in the airflow and, between the vaporization point at the filament 111m and the outlet 108m of the passage 106m, condenses to form an aerosol. This aerosol is then inhaled, via the mouthpiece 109m, by a user of the system 101m.

The power source of the main body 102m may be in the form of a battery (e.g., a rechargeable battery).

The main body 102m may comprise a connector in the form of, e.g., a USB port for recharging this battery. The main body 102m may also comprise a controller that controls the supply of power from the power source to the main body electrical contacts (and thus to the filament 111m). That, is the controller may be configured to control a voltage applied across the main body electrical contacts, and thus the voltage applied across the filament 111m. In this way, the filament 111m may only be heated under certain conditions (e.g., during a puff and/or only when the system is in an active state). In this respect, the main body 102m may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102m and consumable 103m may comprise a further interface which may, for example, be in the form of an RFID reader, a barcode or QR code reader. This interface may be able to identify a characteristic (e.g., a type) of a consumable 103m engaged with the main body 102m. In this respect, the consumable 103m may include any one or more of an RFID chip, a barcode or QR code, or memory within which is an identifier and which can be interrogated via the interface.

FIG. 52 illustrates a smoking substitute device 101m-2 similar to that shown in FIGS. 51A-51C. However, smoking substitute device 101m-2 additionally comprises a flavor delivery article 250m for delivering flavor to a user during vaping. The flavor delivery article 250m is attached to an outer surface of the main body 102m of the smoking substitute device 101m-2. The flavor delivery article 250m sits in a recess formed in the main body 102m, and extends away from the main body 102m. In an alternative embodiment, the flavor delivery article 250m is flush with the outer surface of the main body 102m so that it does not extend away from the main body 102m.

Similarly, to the smoking substitute system as described in FIGS. 51A-51C, one end of the consumable 103m of smoking substitute device 101m-2 defines a mouthpiece 109m for delivering vaporized e-liquid 104m to a user during vaping. The flavor delivery article 250m is positioned such that during vaping, the flavor delivery article 250m is adjacent to, or proximate to, the nose of the user. In other words, the flavor delivery article 250m is positioned on the top side of the smoking substitute device 101m-2 to sit close to the nose of a user. Accordingly, when a user inhales during vaping, vaporized e-liquid 104m containing nicotine is inhaled via the user's mouth, and flavor is inhaled via the user's nose by olfactory flavor delivery.

The flavor delivery article 250m is attachable to and detachable from the main body 102m by a clip (although other means of reversible attachment of the flavor delivery article to the main body may be used). In an alternative embodiment, the flavor delivery article 250m may be attachable to the consumable 103m instead of the main body 102m.

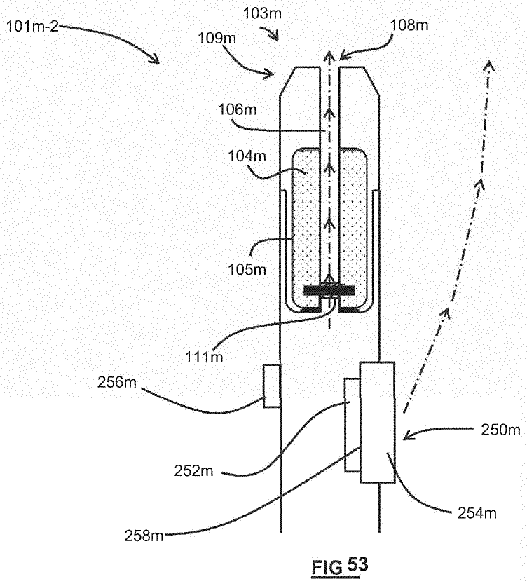

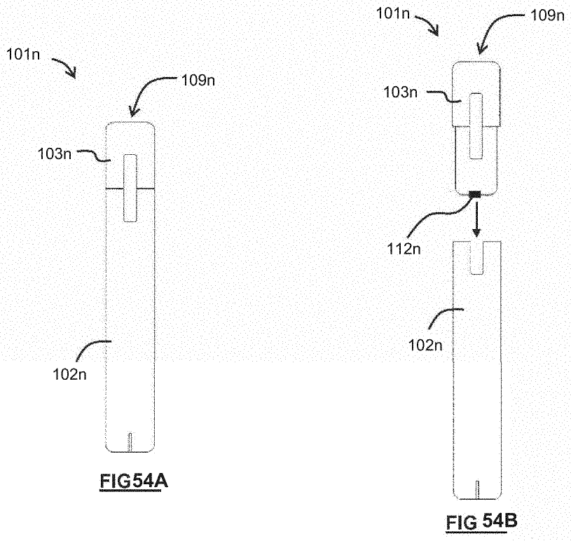

As shown in FIG. 53, the flavor delivery article 250m comprises a piezoelectric motor 252m and a pad of absorbable material 254m. A fragranced liquid (i.e., a scented liquid) is absorbed into the pad 254m. The piezoelectric motor 252m is positioned adjacent to the pad 254m. Specifically, a resonating surface 258m of the piezoelectric motor 252m is positioned adjacent to and in contact with the pad 254m containing the absorbed fragranced liquid.

Although not shown in FIG. 53, both the piezoelectric motor 252m and filament 111m are connectable to a power source of the main body 102m. As described above with reference to FIG. 51C, main body electrical contacts are electrically connected to the power source of the main body 102m, such that (in the engaged position) the filament 111m is electrically connected to the power source. In this way, power can be supplied by the main body 102m to the filament 111m in order to heat the filament 111m and vaporize the e-liquid 104m for inhalation by a user. Similarly, the main body electrical contact also electrically connects the piezoelectric motor 252m to the power source of the main body 102m (in the engaged position) so that power can be supplied from the main body 102m to the piezoelectric motor 252m.

A controller (not shown) in the main body 102m controls the supply of power from the power source to the filament 111m and the piezoelectric motor 252m. The controller is configured to control a voltage across the filament 111m and across the piezoelectric motor 252m.

If the controller instructs the supply of power to the piezoelectric motor 252m, a high frequency signal is supplied to the piezoelectric motor 252m. A piezoelectric element formed from a piezoelectric material in the piezoelectric motor 252m converts this signal to vibrationary motion at the same high frequency and vibrates the resonating surface 258m at this same high frequency. As the resonating surface 258m is positioned adjacent to, and touching, the pad 254m containing the absorbed fragranced liquid, the resonating surface 258m vibrates at the same high frequency against the pad 254m. The force on the pad 254m resulting from the high-frequency oscillation of the resonating surface 258m against the pad 254m, leads to agitation of the fragranced liquid absorbed within the pad 254m. This excitement of the fragranced liquid results in the formation of fine droplets of fragranced liquid which are released from the pad 254m as a mist.

As shown by the arrows from the pad 254m in FIG. 53, the mist of fragranced liquid travels towards the nose of the user (during vaping, when the user has the mouthpiece 109m in their mouth). Accordingly, e-liquid aerosol enters the mouth of the user through the mouthpiece 109m for the delivery of nicotine, and the mist of fragranced liquid enters the nose of the user for the delivery of flavor.

The controller controls the supply of power from the power source to the piezoelectric motor 252m. A user can directly control the supply of power by a dial 256m on the main body 103m. Movement of the dial 256m results in the controller either increasing or decreasing the amount of power, or the frequency of the signal, supplied to the piezoelectric motor 252m. Accordingly, the amplitude and/or frequency of vibration of the resonating surface 258m of the piezoelectric motor 252m can be altered, and the amount of fragranced liquid released from the pad 254m can be altered. The user can therefore control the intensity and/or strength of flavor received from the flavor delivery article 250m.

In an alternative embodiment, a user may be able to control the supply of power to the piezoelectric motor 252m by a button, slider or other means.

The controller may also control the supply of power to the piezoelectric motor 252m on the basis of whether or not a user is puffing on the smoking substitute device 101m-2. The main body 102m includes a puff sensor (not shown) that is configured to detect a puff (i.e., inhalation) on the smoking substitute device 101m-2. The puff sensor is operatively connected to the controller so as to be able to provide a signal, to the controller, which is indicative of a puff state (i.e., puffing or not puffing). The puff sensor may, for example, be in the form of a pressure sensor or an acoustic sensor. The controller also controls power supply to the piezoelectric motor 252m in response to a puff detection by the puff sensor. Specifically, the controller may only supply power to the piezoelectric motor 252m in response to a puff detection signal indicating a user puff, such that flavor is only released when a user is taking a puff (i.e., sucking on) the smoking substitute device.

In this way, the flavor delivery article 250m is only activated to release flavor when a user is taking a puff (i.e., inhaling through) the smoking substitute device 101m-2.

In alternative embodiments, the flavor delivery article 250m may comprise a pocket containing the fragranced fluid, or a crystalized membrane, instead of the absorbable pad 254m. The dial 256m for controlling the power supplied to the piezoelectric motor 252m may be formed on the flavor delivery article 250m, consumable 103m or the main body 102m.

In a further alternative embodiment, the flavor delivery article 250m may be integral with the main body 102m or the consumable 103m, such that the flavor delivery article 250m is not detachable from the main body 102m or consumable 103m.

Thirteenth Mode: A Smoking Substitute Apparatus Capable of Delivering Flavor to a User

Aspects and embodiments of the thirteenth mode of the present disclosure will now be discussed with reference to the accompanying figures. Further aspects and embodiments of the thirteenth mode will be apparent to those skilled in the art. All documents mentioned in this text are incorporated herein by reference.

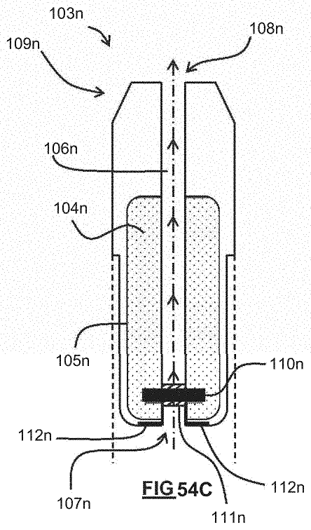

FIGS. 54A and 54B illustrate a smoking substitute system in the form of an e-cigarette system 101n. The system 101n comprises an e-cigarette device defining a main body 102n of the system 101n, and a smoking substitute apparatus in the form of an e-cigarette consumable (or “pod”) 103n. In the illustrated embodiment the consumable 103n (smoking substitute apparatus) is removable from the main body (e-cigarette device), so as to be a replaceable component of the system 101n. In other words, the e-cigarette system 101n is a closed system.

As is apparent from FIGS. 54A and 54B, the consumable 103n is configured to engage the main body 102n. FIG. 54A shows the main body 102n and the consumable 103n in an engaged state, whilst FIG. 54B shows the main body 102n and the consumable 103n in a disengaged state. When engaged, a portion of the consumable 103n is received in a cavity of the main body 102n and is retained in the engaged position by way of a snap-engagement mechanism. In other embodiments, the main body 102n and consumable 103n may be engaged by screwing one into (or onto) the other, through a bayonet fitting, or by way of an interference fit.

The system 101n is configured to vaporize an aerosol-former, which in the illustrated embodiment, is in the form of a nicotine-based e-liquid 104n. The e-liquid 104n comprises nicotine and a base liquid including propylene glycol and/or vegetable glycerin. In the present embodiment, the e-liquid 104n is flavorless (and does not include any added flavorant). That is, if the e-liquid 104n were to be inhaled (i.e., in aerosol form) by a user, it would not have a particularly perceptible flavor or taste.

As is more apparent from FIG. 54C, this e-liquid 104n is stored within a reservoir in the form of a tank 105n that forms part of the consumable 103n. In the illustrated embodiment, the consumable 103n is a “single-use” consumable 103n. That is, upon exhausting the e-liquid 104n in the tank 105n, the intention is that the user disposes of the entire consumable 103n. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer). The tank 105n surrounds, and thus defines a portion of, a passage 106n that extends between an inlet 107n and an outlet 108n at opposing ends of the consumable 103n. In this respect, the passage comprises an upstream end at the end of the consumable 103n that engages with the main body 102n, and a downstream end at an opposing end of the consumable 103n that comprises a mouthpiece 109n of the system 101n. When the consumable 103n is engaged with the main body 102n, a user can inhale (i.e., take a puff) via the mouthpiece 109n so as to draw air through the passage 106n, and so as to form an airflow (indicated by arrows) in a direction from the inlet 107n to the outlet 108n of the passage 106n. Although not illustrated, the passage 106n may be partially defined by a tube (e.g., a metal tube) extending through the consumable 103n. The passage 106n is in fluid communication with a gap defined between the consumable 103n and the main body 102n (when engaged) such that air outside of the system 101n is drawn into the passage 106n (during an inhale).

The smoking substitute system 101n is configured to vaporize the e-liquid 104n for inhalation by a user. To provide this, the consumable 103n comprises a heater having of a porous wick 110n and a resistive heating element in the form of a heating filament 111n that is helically wound around a portion of the porous wick 110n. The porous wick 110n extends across the passage 106n (i.e., transverse to a longitudinal axis of the passage 106n) and opposing ends of the wick 110n extend into the tank 105n (so as to be submerged in the e-liquid 104n). In this way, e-liquid 104n contained in the tank 105n is conveyed from the opposing ends of the porous wick 110n to a central portion of the porous wick 110n so as to be exposed to the airflow in the passage 106n (i.e., caused by a user inhaling). In other embodiments the heating filament 111n and/or wick 110n may form part of the main body (but may engage the tank 105n during engagement of the main body 102n and the consumable 103n).

The helical filament 111n is wound about this exposed central portion of the porous wick 110n and is electrically connected to an electrical interface in the form of electrical contacts 112n mounted at the end of the consumable that is proximate the main body 102n (when engaged). When the consumable 103n is engaged with the main body 102n, the electrical contacts 112n contact corresponding electrical contacts (not shown) of the main body 102n. The main body electrical contacts are electrically connected to a power source (not shown) of the main body 102n, such that (in the engaged position) the filament 111n is electrically connected to the power source. In this way, power can be supplied by the main body 102n to the filament 111n in order to heat the filament 111n. This heat is transferred from the filament 111n to the porous wick 110n which causes e-liquid 104n conveyed by the porous wick 110n to increase in temperature to a point at which it vaporizes. The vaporized e-liquid becomes entrained in the airflow and, between the vaporization point at the filament 111n and the outlet 108n of the passage 106n, condenses to form an aerosol. This aerosol is then inhaled, via the mouthpiece 109n, by a user of the system 101n.

The power source of the main body 102n may be in the form of a battery (e.g., a rechargeable battery).

The main body 102n may comprise a connector in the form of, e.g., a USB port for recharging this battery. The main body 102n may also comprise a controller that controls the supply of power from the power source to the main body electrical contacts (and thus to the filament 111n). That, is the controller may be configured to control a voltage applied across the main body electrical contacts, and thus the voltage applied across the filament 111n. In this way, the filament 111n may only be heated under certain conditions (e.g., during a puff and/or only when the system is in an active state). In this respect, the main body 102n may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102n and consumable 103n may comprise a further interface which may, for example, be in the form of an RFID reader, a barcode or QR code reader. This interface may be able to identify a characteristic (e.g., a type) of a consumable 103n engaged with the main body 102n. In this respect, the consumable 103n may include any one or more of an RFID chip, a barcode or QR code, or memory within which is an identifier and which can be interrogated via the interface.

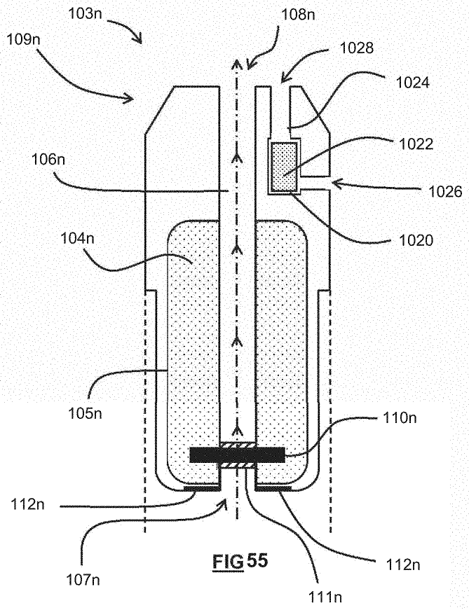

FIG. 55 illustrates a smoking substitute apparatus which is similar to the smoking substitute apparatus of FIGS. 54A-54C, but additionally includes a flavor cavity 1020. The flavor cavity 1020 has a reservoir formed therein which contains a powdered flavorant 1022. The smoking substitute apparatus is thus a hybrid pod which has both a tank 105n containing e-liquid 104n and a flavor cavity 1020 containing a flavored powder 1022.

Similarly, to in FIGS. 54A-54C, a first flow passage 106n extends between a first flow passage inlet 107n and a first flow passage outlet 108n. The first flow passage outlet 108n is formed in the mouthpiece 109n of the consumable 103n. The first flow passage 106n guides aerosol vapor of the e-liquid 104n from the tank 105n to the first flow passage outlet 108n in the mouthpiece 109n, for inhalation by a user.

As shown in FIG. 55, a second flow passage 1024 extends between a second flow passage inlet 1026 and a second flow passage outlet 1028. The second flow passage 1024 may be formed by one or more hollow tubes extending through the interior of the consumable 103n. The second flow passage outlet 1028 is formed in the mouthpiece 109n, and the flavor cavity 1020 containing the flavored powder 1022 is formed in a portion of the second flow passage 1024. Thus, the second flow passage 1024 acts to guide ambient air from an outside of the consumable 103n through the second flow passage inlet 1026 and the flavor cavity 1020, to the second flow passage outlet 1028.

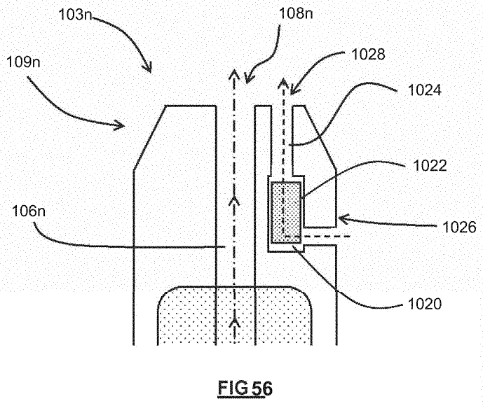

The flow of ambient air through the second flow passage 1024 is shown in FIG. 56 by a dashed arrow. When a user of the smoking substitute system draws on the mouthpiece 109n, ambient air is sucked through the second flow passage 1024, and thus through the flavor cavity 1020. The flavored powder 1022 is entrained in the flow of ambient air through the second flow cavity 1024 and is therefore carried through the second flow passage outlet 1028 by the flow of air and into the mouth of the user for inhalation.

The first flow passage 106n and second flow passage 1024 are fluidly isolated from one another along their entire length to avoid e-liquid 104n mixing with the powder 1022. Thus, the second flow passage 1024 is isolated from the tank 105n and the heating element.

The second flow passage 1024 may be adjustable (i.e., tunable) to alter the amount of powder entrained in the air flow into the mouth of a user. The size and/or shape of the second flow passage air inlet 1026 or second flow passage air outlet 1028 may be adjustable to change the amount of powder entrained through the second flow passage per use (i.e., per puff) of the smoking substitute system. A user may be able to control the size or shape of the second flow passage inlet 1026 or outlet 1028 by the use of a button, or slider. The button or slider may act to partially close the second flow passage inlet 1026 or outlet 1028.

Alternatively (or additionally), the first flow passage 106n may be adjustable to change the relative amount of powder 1022 and aerosol vapor delivered to the mouth of a user through the mouthpiece 109n.

Although not shown in the figures, there may be a flow passage obstructer extendable across the second flow passage 1024. The flow passage obstructer may be formed as a flap, cap, lid or a sliding obstructer, and may be operable to partially, and/or completely block the second flow passage 1024 so that ambient air cannot flow through the second flow passage 1024. The second flow passage obstructer may be formed over the second flow passage inlet 1026 as a cap or plug, or may be formed elsewhere in the second flow passage 1024.

In other embodiments, the second flow passage 1024 may be non-linear and/or tortuous, and follow a winding, meandering path. The second flow passage 1024 may spiral around the first flow passage 106n.

In FIGS. 55 and 56, the second flow passage air inlet 1026 is formed in an outer side wall of the consumable 103n. However, other configurations may be possible. For example, the first flow passage inlet 107n may be formed together with the second flow passage inlet 1026 to form a combined flow inlet upstream of the tank 105n and the heating element. Air may travel through the combined flow inlet into a combined flow passage which branches into the first flow passage 106n and second flow passage 1024 upstream of the heating element and tank 105n. The flow of ambient air entering the consumable 103n may be split into two passages; the first guiding vaporized e-liquid 104n to the mouthpiece 109n, and the second guiding powdered flavorant 1022 to the mouthpiece 109n. Similarly, to the embodiment shown in FIGS. 55 and 3, the flavored powder 1022 does not mix or come into contact with the e-liquid 104n.

Fourteenth Mode: A Mouthpiece for a Smoking Substitute Apparatus Having One or More Electrode

Aspects and embodiments of the fourteenth mode of the present disclosure will now be discussed with reference to the accompanying figures. Further aspects and embodiments of the fourteenth mode will be apparent to those skilled in the art. All documents mentioned in this text are incorporated herein by reference.

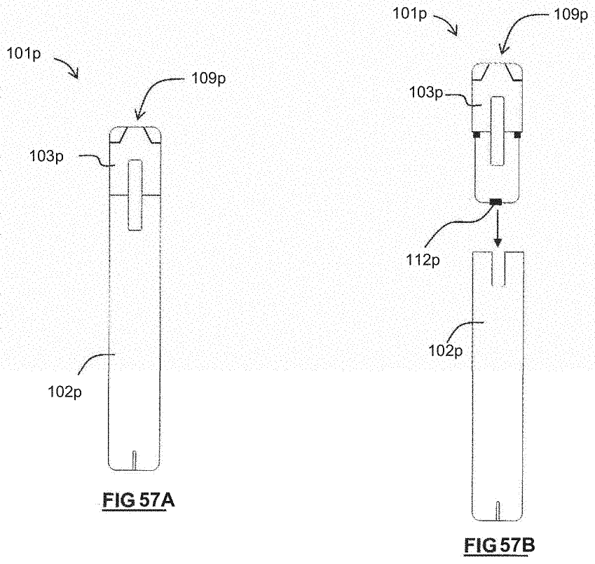

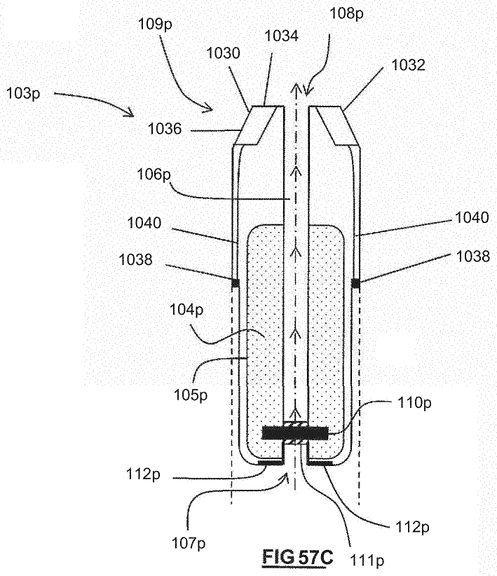

FIGS. 57A and 57B illustrate a smoking substitute apparatus 101p in the form of an e-cigarette. The apparatus 101p comprises an e-cigarette device defining a main body 102p of the apparatus 101p, and a cartridge 103p in the form of an e-cigarette consumable (or “pod”). In the illustrated embodiment the cartridge 103p (or aerosol delivery device) is removable from the main body (e-cigarette device), so as to be a replaceable component of the apparatus 101p. In other words, the e-cigarette 101p is a “closed system” smoking substitute apparatus. An expanded schematic cross-sectional view of the cartridge is shown in FIG. 57C.

As is apparent from FIGS. 57A and 57B, the cartridge 103p is configured to engage the main body 102p. FIG. 57A shows the main body 102p and the cartridge 103p in an engaged state, whilst FIG. 57B shows the main body 102p and the cartridge 103p in a disengaged state. When engaged, a portion of the cartridge 103p is received in a cavity of the main body 102p and is retained in the engaged position by way of a snap-engagement mechanism. In other embodiments, the main body 102p and cartridge 103p may be engaged by screwing one into (or onto) the other, through a bayonet fitting, or by way of an interference fit.

The apparatus 101p is configured to vaporize an aerosol-former, which in the illustrated embodiment, is in the form of a nicotine-based e-liquid 104p. The e-liquid 104p comprises nicotine and a base liquid including propylene glycol and/or vegetable glycerin. In the present embodiment, the e-liquid 104p is flavorless (and does not include any added flavorant). That is, if the e-liquid 104p were to be inhaled (i.e., in aerosol form) by a user, it would not have a particularly perceptible flavor or taste.

As is more apparent from FIG. 57C, this e-liquid 104p is stored within a reservoir (receptacle) in the form of a tank 105p that forms part of the cartridge 103p. In the illustrated embodiment, the cartridge 103p is a “single-use” cartridge 103p. That is, upon exhausting the e-liquid 104p in the tank 105p, the intention is that the user disposes of the entire cartridge 103p. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105p surrounds, and thus defines a portion of, a passage 106p that extends between an inlet 107p and an outlet 108p at opposing ends of the cartridge 103p. In this respect, the passage comprises an upstream end at the end of the cartridge 103p that engages with the main body 102p, and a downstream end at an opposing end of the cartridge 103p that comprises a mouthpiece 109p of the apparatus 101p. When the cartridge 103p is engaged with the main body 102p, a user can inhale (i.e., take a puff) via the mouthpiece 109p so as to draw air through the passage 106p, and so as to form an airflow (indicated by arrows) in a direction from the inlet 107p to the outlet 108p of the passage 106p. Although not illustrated, the passage 106p may be partially defined by a tube (e.g., a metal tube) extending through the cartridge 103p. The passage 106p is in fluid communication with a gap defined between the cartridge 103p and the main body 102p (when engaged) such that air outside of the system 101p is drawn into the passage 106p (during an inhale).

The smoking substitute apparatus 101p is configured to vaporize the e-liquid 104p for inhalation by a user. To provide this, the cartridge 103p comprises a heater having of a porous wick 110p and a resistive heating element in the form of a heating filament 111p that is helically wound around a portion of the porous wick 110p. The porous wick 110p extends across the passage 106p (i.e., transverse to a longitudinal axis of the passage 106p) and opposing ends of the wick 110p extend into the tank 105p (so as to be submerged in the e-liquid 104p). In this way, e-liquid 104p contained in the tank 105p is conveyed from the opposing ends of the porous wick 110p to a central portion of the porous wick 110p so as to be exposed to the airflow in the passage 106p (i.e., caused by a user inhaling). In other embodiments the heating filament 111p and/or wick 110p may form part of the main body (but may engage the tank 105p during engagement of the main body 102p and the cartridge 103p).

The helical filament 111p is wound about this exposed central portion of the porous wick 110p and is electrically connected to an electrical interface in the form of electrical contacts 112p mounted at the end of the cartridge that is proximate the main body 102p (when engaged). When the cartridge 103p is engaged with the main body 102p, the electrical contacts 112p contact corresponding electrical contacts (not shown) of the main body 102p. The main body electrical contacts are electrically connected to a power source (not shown) of the main body 102p, such that (in the engaged position) the filament 111p is electrically connected to the power source. In this way, power can be supplied by the main body 102p to the filament 111p in order to heat the filament 111p. This heat is transferred from the filament 111p to the porous wick 110p which causes e-liquid 104p conveyed by the porous wick 110p to increase in temperature to a point at which it vaporizes. The vaporized e-liquid becomes entrained in the airflow and, between the vaporization point at the filament 111p and the outlet 108p of the passage 106p, condenses to form an aerosol. This aerosol is then inhaled, via the mouthpiece 109p, by a user of the system 101p.

The power source of the main body 102p may be in the form of a battery (e.g., a rechargeable battery).

The main body 102p may comprise a connector in the form of, e.g., a USB port for recharging this battery. The main body 102p also comprises a controller (not shown) that controls the supply of power from the power source to the main body electrical contacts (and thus to the filament 111p). That, is the controller may be configured to control a voltage applied across the main body electrical contacts, and thus the voltage applied across the filament 111p. In this way, the filament 111p may only be heated under certain conditions (e.g., during a puff and/or only when the system is in an active state). In this respect, the main body 102p may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102p and cartridge 103p may comprise a further interface which may, for example, be in the form of an RFID reader, a barcode or QR code reader. This interface may be able to identify a characteristic (e.g., a type) of a cartridge 103p engaged with the main body 102p. In this respect, the cartridge 103p may include any one or more of an RFID chip, a barcode or QR code, or memory within which is an identifier and which can be interrogated via the interface.

The controller may further comprise a communication interface for communicating with an external device. For example, the controller may include a wireless interface for wireless communication (e.g., Bluetooth, Wi-Fi) or a wired interface for wired communication (e.g., USB).

The mouthpiece 109p of the cartridge 103p includes a pair of electrodes 120,122 for stimulating a user's tongue to simulate flavor delivery. The electrodes 1030, 1032 are arranged on either side of the outlet 108p in the mouthpiece 109p. The electrodes 1030, 1032 are exposed on an outer surface of the mouthpiece. Each of the electrodes 1030, 1032 includes a first portion disposed on a forward-facing surface 1034 of the mouthpiece 109p, and a second portion disposed on a later-facing surface 1036 of the mouthpiece 109p. Such a layout of the electrodes 1030, 1032 may serve to increase a contact area between the electrodes 1030, 1032 and the user's tongue, to improve quality of flavor simulation.

The pair of electrodes 1030, 1032 is arranged to electrically stimulate the user's tongue by passing a current through a portion of the user's tongue located between the pair of electrodes 1030, 1032. The electrodes 1030, 1032 are connected to respective electrical contacts 1038 disposed on a side of the cartridge 103p. The electrodes 1030, 1032 are connected to the electrical contacts 1038 via wires 1040 which extend through a portion of the cartridge 103p. When the cartridge 103p is engaged with the main body 102p, the electrical contacts 1038 contact corresponding electrical contacts (not shown) of the main body 102p. The corresponding electrical contacts of the main body 102p are connected to the controller in the main body 102p. In this manner, when the cartridge 103p is engaged with the main body 102p, a stimulation signal generated by the controller may be transmitted to the electrodes 1030, 1032 to stimulate the user's tongue.

The controller is configured to generate and control a stimulation signal that is transmitted to the electrodes 1030, 1032 when the cartridge 103p is engaged with the main body 102p. In this embodiment, the stimulation signal generated by the controller is an electrical current which is to be passed through the user's tongue via the electrodes 1030, 1032 to stimulate the user's tongue. The controller may, for example, generate and control the stimulation signal by controlling a voltage applied across the electrodes by the power supply in the main body. The controller may control (e.g., adjust) the stimulation signal to simulate a desired flavor.

As an example, the stimulation signal may include current pulses which are delivered to the user's tongue via the pair of electrodes 1030, 1032. The controller may adjust parameters of the current pulses including magnitude of the current pulses, pulse duration, and pulse frequency to simulate a desired flavor, as a simulated flavor perceived by the user may depend on these parameters.

Example currents that may be passed through the user's tongue to simulate flavor may include currents having a magnitude between 10 μA and 500 μA (preferably between 20 μA and 200 μA), and pulse frequencies between 50 Hz and 1200 Hz. The current pulses may, for example, be square wave pulses.

The pulses may be generated using pulse-width modulation techniques.

Of course, these parameters may be adapted to a given user's physiology and sensitivity to electrical stimulation via the electrodes.

The controller may be configured to generate and transmit a stimulation signal to the electrodes 1030, 1032 when the puff sensor detects that the user is using the smoking substitute apparatus 101p. In this manner, when a user puffs on the apparatus 101p, the controller may simultaneously activate the heater (filament 111p) to generate an aerosol from the e-liquid 104p, and transmit a simulation signal to the electrodes 1030, 1032 to simulate a flavor for the user. Thus, the user may experience simulated flavor delivery via the electrodes 1030, 1032 whilst they puff on the apparatus 101p.

In the example shown, the electrodes 1030, 1032 are arranged to electrically stimulate the user's tongue. In other examples, one or both of the electrodes 120,122 may be arranged to thermally stimulate the user's tongue (in addition or alternatively to electrical stimulation). For example, a temperature actuator (e.g., heater or Peltier module) may be provided in the mouthpiece 109p and thermally coupled to one of the electrodes, to cause a temperature change (e.g., temperature increase or decrease) of the electrode. The controller may be configured to transmit a stimulation signal (e.g., an electrical current or voltage) to the thermal actuator, to vary the temperature of the thermal actuator and hence thermally stimulate the user's tongue via the electrode.

Thermally stimulating the user's tongue may involve varying the temperature of one or both of the electrodes 1030, 1032 between 20° C. and 35° C.

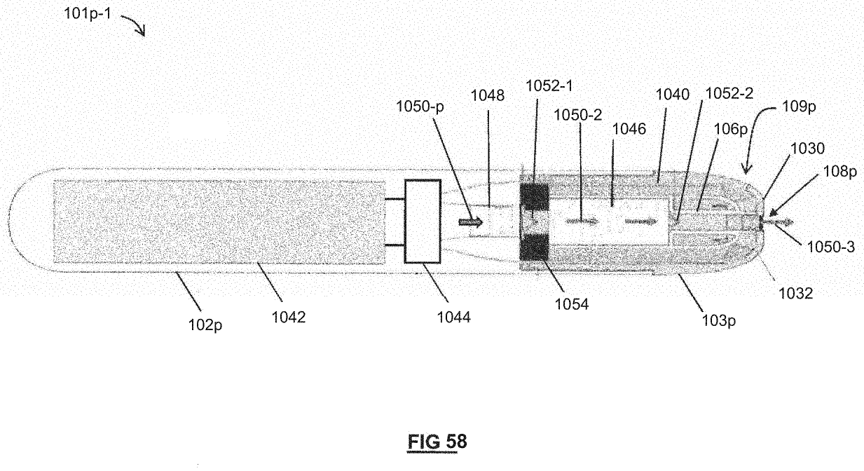

FIG. 58 shows a schematic cross-sectional view of a vaporless smoking substitute apparatus 101p-1 that is another embodiment of the disclosure. The apparatus 101p-1 includes many components which correspond to those described above in relation to apparatus 101p. Components of apparatus 101p-1 which correspond to those already described in relation to apparatus 101p are given the same reference numeral as in FIGS. 57A-57C and are not described again.

As can be seen in FIG. 58, apparatus 101p-1 includes a battery 1042 (e.g., rechargeable battery) and a controller 1044 connected to the battery 1042, both of which are located in its main body 102p.

Instead of an e-liquid, the cartridge 103p of apparatus 101p-1 includes a porous substrate 1046 that is imbued with nicotine liquid. The porous substrate 1046 may be any suitable porous material, e.g., a cloth, mesh, sponge or the like. The porous substrate 1046 is disposed in a receptacle 1048 of the cartridge 103p. The porous substrate 1046 is located so that it is disposed within the passage 106p in the cartridge 103p.

A mesh heater 1048 is disposed in the body 102p of the apparatus 101p-1 and electrically connected to the controller 1044 so that the controller can control power delivered to the mesh heater 1048. The mesh heater 1048 may be made of a resistive mesh material that generates heat when a current is passed through it.

The mesh heater 1048 is disposed upstream of an inlet of the passage 106p in the cartridge 103p. When a user puffs on the mouthpiece 109p of apparatus 101p-1, ambient air may be drawn into the apparatus 101p-1 via an air inlet (not shown) formed in a side of the main body 102p. The ambient air may then be drawn through the mesh heater 1048 (as illustrated by arrow 1050-1) so that it is heated. The heated air is then entrained into the passage 106p in the cartridge 103p and through the porous substrate 1046 (arrow 1050-2). As the heated air passes through the porous substrate 1046, it may pick up nicotine particles from the porous substrate 1046 to for a mixture of heated air and nicotine particles (aerosol). The aerosol comprising the nicotine particles may then be inhaled by the user through the outlet 108p in the mouthpiece 109p (arrow 1050-3). In this manner, apparatus 101p-1 may produce a vaporless aerosol comprising heated air and nicotine particles. In this manner, a user of the apparatus 101p-1 may not emit a cloud of vapor following an inhalation of aerosol from the apparatus 101p-1.

The apparatus 101p-1 includes a first one-way valve 1052-1 located at an inlet of the passage 106p in the cartridge, and a second one-way valve 1052-2 located in the passage 106p downstream of the porous substrate 1046. The first and second one-way valves 1052-1, 1052-2 are arranged to only allow air flow in one direction, from the mesh heater 1048 towards the outlet 108p of the mouthpiece 109p. This may prevent aerosol from being blown back into the mesh heater 1048, which could cause deterioration of the mesh heater 1048 over time. A seal 1054 is also provided at a lower end of the cartridge 103p, around the inlet of the passage 106p, to prevent leakage at an interface between the cartridge 103p and the main body 102p.

The electrodes 1030, 1032 of apparatus 101p-1 are configured in a similar way to those of apparatus 101p. In particular, they are connected to the controller 1044 via wires 1040, so that a stimulation signal may be transmitted to the electrodes 1030, 1032 to stimulate the user's tongue. As can be seen in FIG. 58, different electrical connections are used for connecting the electrodes 1030, 1032 and the mesh heater 1048 to the controller 1044. This may avoid interference between signals generated by the controller to control the electrodes 1030, 1032 and the mesh heater 1048.

Similar to the discussion above in relation to apparatus 101p, the controller may be configured to a) generate a stimulation signal for stimulating the user's tongue in response to a detection that the user is puffing on the mouthpiece 109p, and b) control the mesh heater 1048 to heat ambient air drawn into the apparatus 101p-1 to generate an aerosol. Puff detection may be performed via a puff detector (not shown) that is connected to the controller. In this manner, flavor delivery may be simulated when the user inhales an aerosol from the apparatus 101p-1. As the aerosol is vaporless and substantially odorless, the apparatus 101p-1 may provide an “invisible” experience, as it may not produce any perceptible vapors or odors.

The cartridge 103p may be removable from the main body 102p of apparatus 101p-1, so as to be a replaceable component of the apparatus 101p-1. In this case, a connector (not shown) may be provided in the cartridge 103p for connecting the wires 1040 to the controller 1044. In some cases, the porous substrate 1046 may be removable from the cartridge 103p, so that it can be replaced without having to replace the whole cartridge.

In some embodiments (not shown) the electrodes 1030, 1032 of apparatus 101p and/or apparatus 101p-1 may be provided as part of a removable cap that is mountable over the mouthpiece 109p. In this manner, the rest of the cartridge 103p may be replaced without having to replace the electrodes 1030, 1032. This may reduce the cost of the cartridge 103p, where the cartridge is intended as a consumable component.

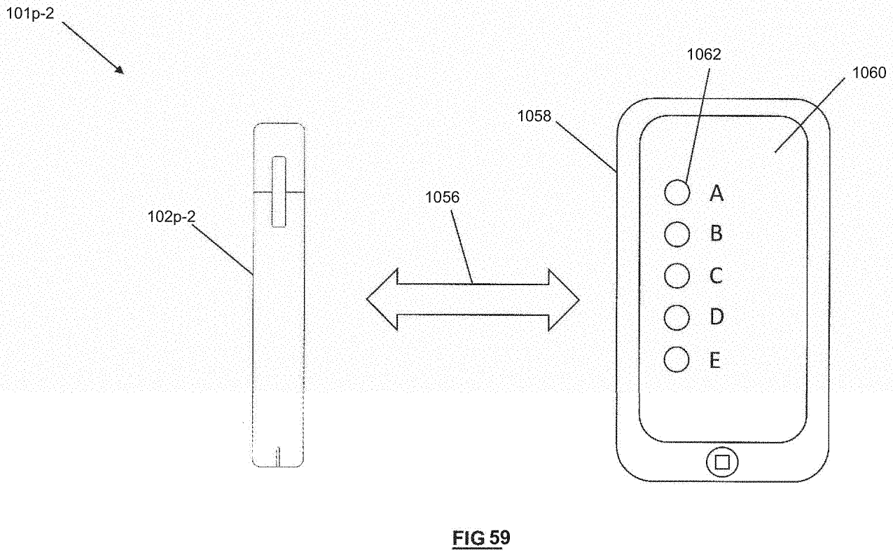

FIG. 59 is a diagram illustrating a smoking substitute system 101p-2 according to an embodiment of the disclosure. The smoking substitute system 101p-2 includes a smoking substitute apparatus 102p-2 and a mobile device 1058. The smoking substitute apparatus 102p-2 may, for example, be apparatus 101p or apparatus 101p-1 described above. The smoking substitute apparatus 102p-2 is communicatively coupled to the mobile device 1058 (as illustrated by arrow 1056), so that data may be exchanged between the apparatus 102p-2 and the mobile device. In particular, a controller in the apparatus 102p-2 may be communicatively coupled to the mobile device 1058. Preferably, they may be communicatively coupled via a Bluetooth connection or other near-field communication method. In the example shown, the mobile device 1058 is a smartphone, however other types of mobile device may be used (e.g., tablet computer, laptop, etc.).

The mobile device 1058 includes software installed thereon for controlling a stimulation signal generated by the controller of apparatus 102p-2 for stimulating a user's tongue via electrodes on a mouthpiece of the apparatus 102p-2 (e.g., electrodes 1030, 1032). The mobile device 1058 may transmit a control signal to the controller of apparatus 102p-2 for controlling the stimulation signal. The controller may then generate and/or control a stimulation signal provided to the electrodes based on the received control signal. The control signal may include an indication of parameters which are to be used for the stimulation signal. In this manner, parameters of the stimulation signal may be controlled by the mobile device 1058 to control flavor simulation by the apparatus 102p-2.

The mobile device 1058 includes software installed thereon for generating a user interface 1060 to enable a user to select a flavor to be simulated by the apparatus 102p-2. In the example shown, the user interface is arranged to present a user with multiple selectable flavor options 1062. In the example shown, the user interface 1060 includes selectable flavor options A, B, C, D and E. When the user selects one of the displayed options, the mobile device transmits a control signal to the mobile device 1058, the control signal including instructions for simulating the selected flavor. In this manner, a user may easily select and control a flavor simulated by the apparatus via the electrodes on the mouthpiece of the apparatus 102p-2.

The mobile device 1058 may include a memory which stores a set of flavors and, for each of the flavors, corresponding parameters for the stimulation signal to simulate that flavor. Thus, when a flavor is selected, the mobile device 1058 may transmit a control signal including an indication of the parameters for the stimulation signal to simulate that flavor to the controller of the apparatus 102p-2. The controller may then generate and control the stimulation signal for the electrodes based on the parameters received from the mobile device 1058.

The mobile device 1058 may be configured to perform a calibration procedure, in order to more accurately simulate flavor for a particular user. The calibration may involve transmission of a test control signal by the mobile device 1058 to the controller of the apparatus 102p-2. The controller may then generate a stimulation signal based on the received test control signal to stimulate the user's tongue. The mobile device may present to the user a user interface including a series of selectable options corresponding to possible flavors perceived by the user as a result of the stimulation. The user may then indicate, via the user interface, which flavor they perceived. The mobile device 1058 may record the user's response.

The mobile device 1058 may be configured to repeat this procedure for multiple different control signals, in order to map out the user's response to different stimulation signals. In this manner, it may be possible to identify which stimulation signals result in which flavors perceived by the user. This information may then be used by the mobile device 1058 to simulate particular flavors for that user. This may enable more accurate flavor simulation for that user.

In some cases, the mobile device 1058 may be configured to adjust the control signal transmitted to the controller of the apparatus based on the indication received from the user. In this manner, the control signal may be adjusted until the user indicates that they perceive a particular flavor. This may enable the flavor simulation to be fine-tuned to the user's sense of taste.

The mobile device 1058 may be connected to a cloud server (not shown), so that user data relating to taste profiles and calibration data may be stored in the cloud. The mobile device 1058 may also access a “flavor library” stored in the cloud, which includes information on how to simulate various flavors (e.g., parameters of the stimulation signal for simulating the various flavors). In this manner, the user may have access to a wide range of flavors stored in the cloud.

Fifteenth Mode: Presenting a Source of Flavorant to Fluid Flow Through a Smoking Substitute Device

Aspects and embodiments of the fifteenth mode of the present disclosure will now be discussed with reference to the accompanying figures. Further aspects and embodiments of the fifteenth mode will be apparent to those skilled in the art. All documents mentioned in this text are incorporated herein by reference.

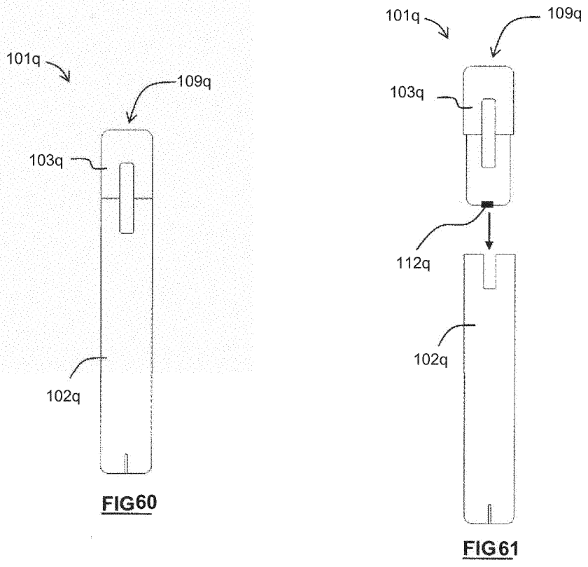

FIGS. 60 and 61 illustrate a smoking substitute system in the form of an e-cigarette system 101q. The system 101q comprises an e-cigarette device defining a reusable main body 102q of the system 101q, and a smoking substitute device in the form of an e-cigarette consumable (or “pod”) 103q, which may also be referred to as a precursor part. In the illustrated embodiment the consumable 103q (smoking substitute device) is removable from the main body (e-cigarette device), so as to be a replaceable component of the system 101q. In other words, the e-cigarette system 101q is a closed system.

As is apparent from FIGS. 60 and 61, the consumable 103q is configured to engage the main body 102q. FIG. 60 shows the main body 102q and the consumable 103q in an engaged state, whilst FIG. 61 shows the main body 102q and the consumable 103q in a disengaged state. When engaged, a portion of the consumable 103q is received in a cavity of the main body 102q and is retained in the engaged position by way of a snap-engagement mechanism. In other embodiments, the main body 102q and consumable 103q may be engaged by screwing one into (or onto) the other, through a bayonet fitting, or by way of an interference fit.

The system 101q is configured to vaporize an aerosol-former or aerosol precursor, which, in the illustrated embodiment, is in the form of a nicotine-based e-liquid 104q. The e-liquid 104q comprises nicotine and a base liquid including propylene glycol and/or vegetable glycerin. In the present embodiment, the e-liquid 104q is flavorless (and does not include any added flavorant). That is, if the e-liquid 104q were to be inhaled (i.e., in aerosol form) by a user, it would not have a particularly perceptible flavor or taste. It is to be appreciated, however, that in other embodiments the e-liquid 104q may comprise an inherent flavorant. In the present specification, the term flavorant may be understood as referring to one or more substances effective to activate at least one of an olfactory receptor in a human nasal cavity; and a taste receptor in a human oral cavity.

As is more apparent from FIG. 62, the e-liquid 104q is stored within a precursor reservoir in the form of a tank 105q that forms part of the consumable 103q. In the illustrated embodiment, the consumable 103q is a “single-use” consumable 103q. That is, upon exhausting the e-liquid 104q in the tank 105q, the intention is that the user disposes of the entire consumable 103q. In other embodiments, the e-liquid (i.e., aerosol precursor 104q) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105q surrounds, and thus defines a portion of, a fluid flow passage 106q-2 that extends between an inlet 107q-2 and an outlet 108q at opposing ends of the consumable 103q. In this respect, the passage 106q-2 comprises an upstream end at the end of the consumable 103q that engages with the main body 102q, and a downstream end at an opposing end of the consumable 103q that comprises a mouthpiece 109q of the system 101q. The fluid flow passage 106q-2 comprises an inlet 107q-2 at its upstream end, and an outlet at its downstream end, the outlet 108q-2 thus forming part of, or being located at, the mouthpiece 109q. When the consumable 103q is engaged with the main body 102q, a user can inhale (i.e., take a puff) via the mouthpiece 109q so as to draw air through the passage 106q-2, and so as to form an airflow (indicated by arrows) in a direction from the inlet 107q-2 to the outlet 108q-2 of the passage 106q-2.

The passage 106q-2 may be partially defined by a tube (e.g., a metal tube or a tube formed from plastic material) extending through the consumable 103q. At its upstream inlet end, the passage 106q-2 may be in fluid communication with a gap defined between the consumable 103q and the main body 102q when the consumable 103q and the main body 102q are interengaged such that when a user draws on the outlet 108q-2 at the mouthpiece 109q, ambient air from outside of the system 101q is drawn into the passage 106q-2 via the inlet 107q-2.

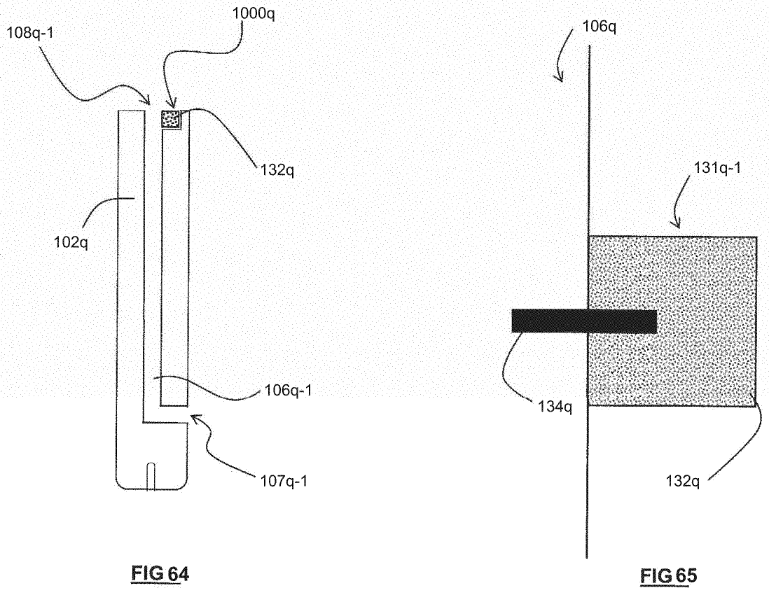

Alternatively, the main body 102q may additionally comprise a fluid passage 106q-1, extending from a main body inlet 107q-1 to a main body outlet 108q-1 as illustrated, for example, in FIG. 64. In such an arrangement, the main body outlet 108q-1 may be arranged such that when the main body 102q and consumable 103q are interengaged, a substantially fluid-tight seal is formed at the connection point between main body outlet 108q-1 and fluid inlet 107q-2, meaning that main body fluid passage 106q-1 and consumable passage 106q-2 become interconnected to form a single passage 106q extending through the smoking substitute system 101q and fluidly connecting main body inlet 107q-1 and consumable outlet 108q-2. Furthermore, the main body fluid passage 106q-1 or the consumable fluid passage 106q-2 may be shaped to provide a recess or receptacle 1000q into which a source of flavorant 132q may be received as will be described in more detail hereinafter.

The configuration of the passage 106q-1 illustrated in FIG. 64 is for illustrative purposes only, and is not intended to be limiting. For example, the fluid inlet 107q-1 might alternatively be located closer to or further away from the end of main body 102q which is configured to engage with the smoking substitute device 103q than illustrated, or may even be located on a different face of the main body 102q. The recess 1000q may be omitted, or may be located at a different position along the fluid passage 106q-1. Other components of the main body 102q (e.g., the power source) are not illustrated in FIG. 64 for clarity, but may be arranged to be adjacent to or surround all or part of the fluid passage 106q-1.

The smoking substitute device 101q is configured to vaporize the e-liquid 104q for inhalation by a user. To provide this function, the consumable 103q is provided with an aerosol generator comprising a porous wick 110q and a resistive heating element in the form of a heating filament 111q that is helically wound around a portion of the porous wick 110q. The aerosol generator is arranged between the inlet 107q-1 and the outlet 108q-2 of the smoking substitute system 101q, and is in fluid communication with both the inlet 107q-1 and the outlet 108q-2. The porous wick 110q extends across the passage 106q (e.g., transverse to a longitudinal axis of the passage 106q) and opposing ends of the wick 110q extend into the tank 105q so as to be submerged in the e-liquid 104q and thus to draw e-liquid from the tank 105q via wicking or capillary action). In this way, e-liquid 104q contained in the tank 105q is conveyed from the opposing ends of the porous wick 110q to a central portion of the porous wick 110q so as to be exposed to the airflow in the passage 106q caused by a user inhaling or drawing on the mouthpiece 109q.

The helical filament 111q is wound about the exposed central portion of the porous wick 110q and is electrically connected to an electrical interface in the form of electrical contacts 112q mounted at the end of the consumable that is proximate the main body 102q (when engaged).

When the consumable 103q is engaged with the main body 102q, the electrical contacts 112q make contact with corresponding electrical contacts (not shown) of the main body 102q. The main body electrical contacts are electrically connected to a power source (not shown) of the main body 102q, such that the filament 111q is electrically connected to the power source when the consumable 103q and the main body 102q are interengaged. In this way, power can be supplied by the main body 102q to the filament 111q in order to heat the filament 111q. This heat is transferred from the filament 111q to the porous wick 110q which causes e-liquid 104q conveyed by the porous wick 110q to increase in temperature to a point at which it vaporizes. The vaporized e-liquid becomes entrained in the airflow past the wick 110q and, between the vaporization point at the filament 111q and the outlet 108q of the passage 106q, condenses to form an aerosol. This aerosol is then inhaled, via the mouthpiece 109q, by a user of the system 101q.

The power source of the main body 102q may be in the form of a battery (e.g., a rechargeable battery).

The main body 102q may comprise a connector in the form of, e.g., a USB port for recharging this battery.

The main body 102q may also comprise a controller that controls the supply of power from the power source to the main body electrical contacts (and thus to the filament 111q). That, is the controller may be configured to control a voltage applied across the main body electrical contacts, and thus the voltage applied across the filament 111q. In this way, the filament 111q may only be heated under certain conditions (e.g., during a puff and/or only when the system is in an active state). In this respect, the main body 102q may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102q and consumable 103q may comprise a further interface which may, for example, be in the form of an RFID reader, a barcode or QR code reader. This interface may be able to identify a characteristic (e.g., a type) of a consumable 103q engaged with the main body 102q. In this respect, the consumable 103q may include any one or more of an RFID chip, a barcode or QR code, or memory within which is an identifier and which can be interrogated via the interface.

In the illustrated smoking substitute system 101q, a source of flavorant 132q is provided along a fluid passage 106q connecting the fluid inlet 107q and the aerosol generator such that the source is presented to a flow of fluid from said inlet 107q towards said aerosol generator, said flow of fluid being effective to release flavorant 132q from the source for entrainment in said fluid upstream of the aerosol generator. The source of flavorant 132q is arranged within the smoking substitute system 101q such that it is substantially thermally isolated from the filament 111q of the aerosol generator, and optionally is also substantially thermally isolated from the power source and/or other potentially heat-generating components of the system, such as electronic circuitry within the main body. To achieve this thermal isolation, embodiments are proposed in which the source of flavorant 132q is thermally insulated. Thermal isolation of the source of flavorant 132q in this manner may, for example, allow for the use of volatile flavorants 132q which would otherwise be too rapidly released into said fluid flow if exposed to elevated temperatures, or which may be susceptible to thermal degradation if subjected to elevated temperatures.

In embodiments in which the e-liquid 104q contains an inherent flavorant, it will be appreciated that the release of flavorant 132q from the source in the manner noted above will serve to supplement, and optionally blend with, the flavorant of the e-liquid 104q in use. For example, the flavorant 132q of the source may have the same flavor as that in the e-liquid 104q in order to supplement the flavor provided within the e-liquid and thereby provide the user with an intensified flavor sensation. Alternatively, the flavorant 132q of the source may be complementary to that in the e-liquid 104q (i.e., the flavors/aromas of the two flavorants, when mixed, provide a pleasing sensory combination to a user). Providing the flavorant 132q separately from the aerosol precursor 104q affords the user an opportunity to select from or change between different flavorants 132q without necessitating a change of e-liquid, for example during a vaping session.

Various arrangements within the smoking substitute device 101q, and specific configurations of the source of flavorant 132q are possible, some examples of which are described below.

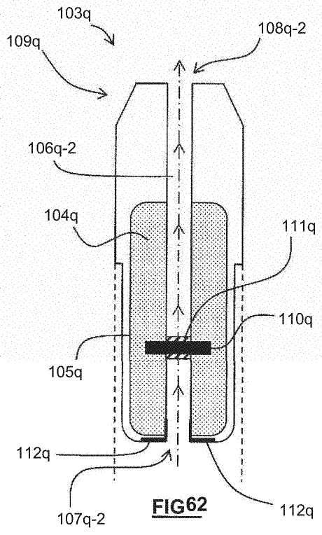

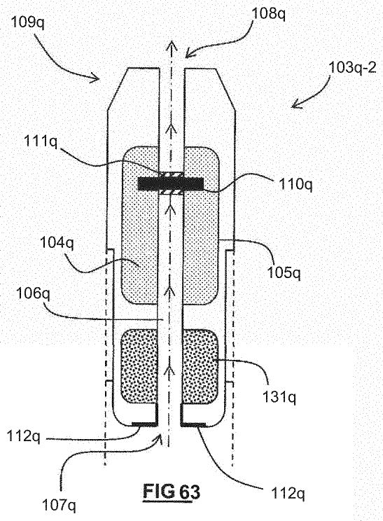

For example, the source of flavorant 132q may be located in a consumable 103q-2 as illustrated exemplarily in FIG. 63, the source of flavorant 132q being arranged so as to be thermally isolated from the aerosol generator smoking substitute device 101q, and preferably also from the power source and any other potentially heat-generating components of the system such as electronic circuitry. As illustrated in FIG. 63, the source of flavorant is thus spaced from the aerosol generator (along the flow passage 106q in the arrangement illustrated) and may be separated therefrom by thermally insulating material such as a suitable plastic material, and the tank 105q.

Alternatively, the source of flavorant 132q may be located in a main body 102q having a fluid passage 106q-1 therethrough. Such a fluid passage is illustrated exemplarily in FIG. 64. The source of flavorant 132q may be located at any position along said fluid passage 106q-1, provided that the source of flavorant 132q remains substantially thermally isolated from the aerosol generator of the smoking substitute system 101q, and preferably also from the power source and any other potentially heat-generating components of the system such as electronic circuitry. Part of passage 106q-1 may be shaped to form a recess, receptacle or chamber 120 into which a source of flavorant 132q may be received.

The source of flavorant 132q may be a flavorant reservoir 131q located either in the main body 102q, or as part of a consumable smoking substitute device 103q. The flavorant reservoir 131q may surround, and thus define, at least part of the passage 106q. Alternatively, the flavorant reservoir 131q may be located adjacent to, but not surrounding the passage 106q. The flavorant 132q comprised in such a reservoir 131q may be provided, for example, as a flavored liquid. A flavorant reservoir 131q that is provided within the main body 102q may be user-refillable, and may thus be located proximate to the interface between main body 102q and smoking substitute device 103q to allow access for refilling. Meanwhile, a flavorant reservoir comprised within the smoking substitute device may be at least partially filled during manufacture of the smoking substitute device 103q. In the latter configuration, the main body 102q may not comprise a fluid inlet 107q-1, outlet 108q-1 or passage 106q-1.

The flavorant reservoir may further comprise one or more porous wicks 134q extending across at least part of the fluid flow passage 106q so as to be presented to a fluid flow therethrough (e.g., transverse to a longitudinal axis of the passage 106q), as illustrated exemplarily in FIG. 65, where the flavorant reservoir is denoted as 131q-1. In embodiments where the flavorant reservoir surrounds a portion of passage 106q, the porous wick 134q may extend substantially completely across the passage 106q, such that both opposing ends of the wick extend into flavorant reservoir. Alternatively, in embodiments where the flavorant reservoir 131q-1 does not surround the passage 106q, only one end of each porous wick 134q may extend into the flavorant reservoir, with the other end being located within the passage 106q. The end or ends of the porous wicks 134q that extend into the flavorant reservoir 131q-1 are arranged to be submerged in the flavorant 132q. In this way, flavorant 132q contained in the flavorant reservoir 131q-1 is conveyed from the end or ends of the porous wick 134q that extend into the flavorant reservoir 131q-1 to a portion of the porous wick 134q located within the passage 106q so as to be exposed to the airflow therein (i.e., caused by a user inhaling).

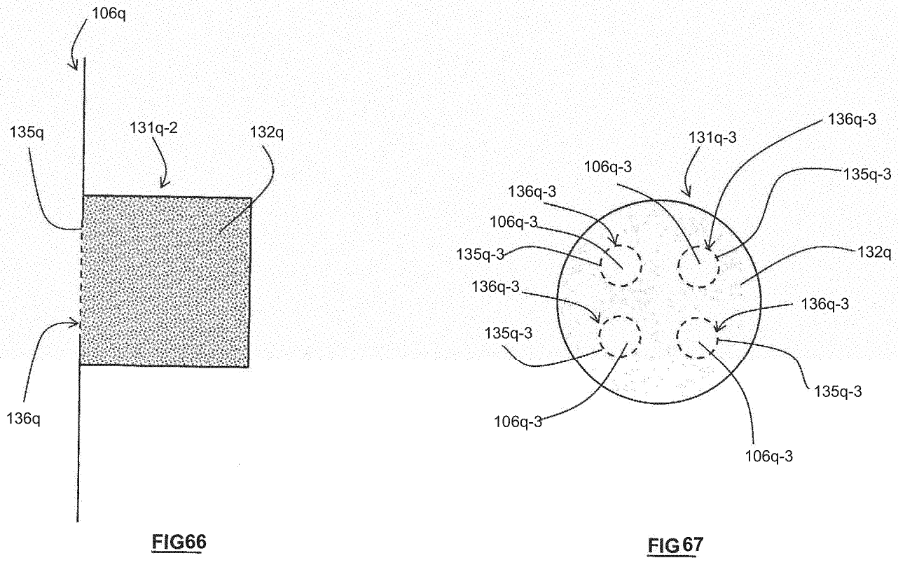

Alternatively, or additionally, at least a part of a barrier separating the passage 106q from the flavorant reservoir 131q-2 may be permeable to flavorant 132q as illustrated exemplarily in FIG. 66. FIG. 66 illustrates a perforated barrier 135q separating the passage 106q from the flavorant reservoir 131q-2. The barrier may thus comprise one or more small apertures 136q to permit the passage of flavorant therethrough, and may be formed as part of the sidewall of the flow passage 106q. In this type of arrangement, flavorant 132q may then permeate through the barrier 135q from the flavorant reservoir 131q-2 to the passage 106q, for example by capillary action, so as to be exposed to the airflow in the passage 106q (i.e., caused by a user inhaling).

In an alternative arrangement illustrated in FIG. 67, the flavorant reservoir 131q-2 is housed within the flow passage 106q so as to extend substantially completely across the flow passage 106q. As will be observed, in this arrangement, the flavorant reservoir 131q-2 is provided with a plurality of relatively small diameter bores 106q-3, each of which extends fully through the reservoir from its upstream end to its downstream end. Each bore 106q-3 may be defined by a similar permeable barrier 135q-3 (each of which may thus be perforated with small apertures 136q-3) in a similar manner to the barrier arrangement described above with reference to FIG. 65. As will be appreciated, fluid flow through the flow passage 106q will thus become split into separate flow streams through the bores 106q-3 of the reservoir, the flow streams then rejoining and mixing with one another downstream of the reservoir. This type of multi-bore configuration of the reservoir 131q-3 can be effective to increase the effective surface area through which the flavorant can permeate, when compared to the single barrier arrangement of FIG. 65, which can be effective to increase the concentration of flavorant which may be entrained in the airflow through the passage 106q.

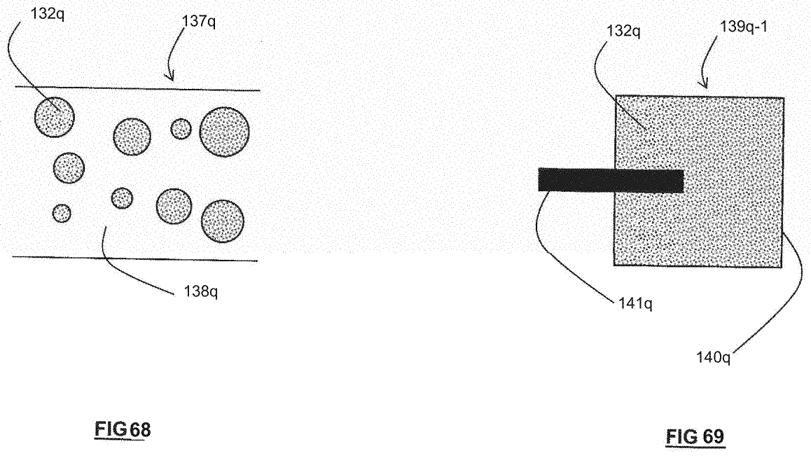

Alternatively, with reference to FIG. 68, it is envisaged that the source of flavorant 132q may be a flavored article 137q, comprising a substrate 138q carrying the flavorant 132q. Such a flavored article 137q may be received within the fluid flow passage 106q, which may be shaped to provide a recess, receptacle, or chamber 1000q within which the flavored article 137q may be located. At least part of the substrate 138q may be formed from a polymeric material (e.g., silicone). Further, at least part of the substrate 138q may be formed from a porous material, foam or foamed material as illustrated exemplarily in FIG. 68. Still further, at least a part of the substrate 138q may be formed from an air permeable material. The flavorant 132q may be one or both of comprised within or coated on a surface of the substrate 138q. The flavorant 132q may be introduced into the flavored article 137q by coating (e.g., spray coating). Alternatively, or additionally, the flavorant 132q may be introduced or impregnated into the flavored article 137q by, for example, immersion of the substrate 138q in a liquid comprising flavorant 132q. This process of introducing flavorant 132q could either be carried out during manufacture of the flavored article 137q, or by an end-user.

Alternatively, having regard to FIG. 69, the source of flavorant 132q may be a “flavor pod” 139q comprising a container 140q at least partially filled with the flavorant 132q. A flavor pod 139q may be received within the passage 106q, which may be shaped to provide a recess or receptacle into which the flavor pod 139q may be located. When in place, a surface of the flavor pod 139q may define part of a peripheral wall of the passage 106q. The flavor pod, denoted as 139q-1 in FIG. 69, may furthermore comprise one or more porous wicks 141q extending at least partially across the passage 106q (e.g., transverse to a longitudinal axis of the passage 106q) so as to be presented to a fluid flow therethrough. At least one end of each porous wick 141q extends into the flavor pod container 140q, so as to be submerged in the flavorant 132q. In this way, the flavorant 132q contained in the flavor pod container 140q is conveyed from the end or ends of the porous wick 141q extending into the flavor pod container 140q to a portion of the porous wick 141q located within the passage 106q so as to be exposed to the airflow therein (i.e., caused by a user inhaling).

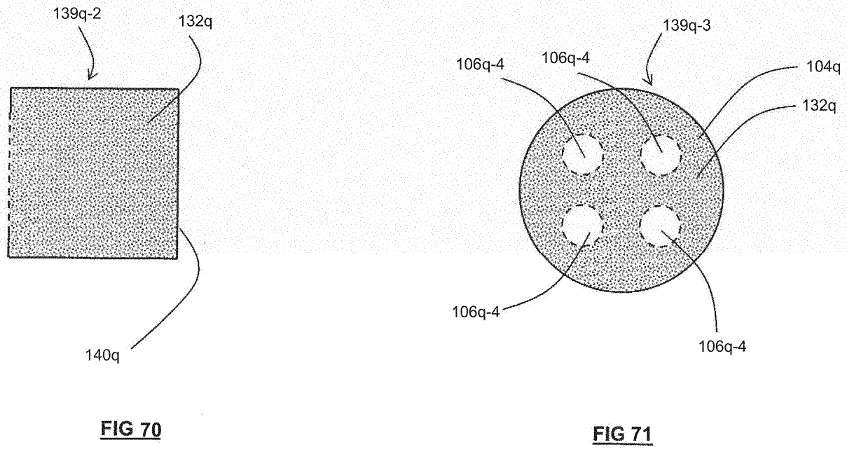

Additionally, or alternatively, at least a part of a surface of the flavor pod 139q located adjacent to, or defining part of passage 106q may be flavorant permeable as illustrated exemplarily in FIG. 70, where the flavor pod is denoted as 139q-2. Alternatively, the flavor pod 139q-2 may have a plurality of fluid passages or bores 106q-4 therethrough, at least part of the wall or barrier dividing each bore 106q-4 from the flavorant container 138q being flavorant-permeable as illustrated exemplarily in FIG. 71. As will be appreciated by those of skill in the art, these arrangements of FIGS. 70 and 71 bear similarities with the arrangements of FIGS. 66 and 67. In such arrangements, flavorant 132q may permeate from the flavor pod 139q to the passage 106q for entrainment in the airflow therethrough (i.e., caused by a user inhaling).

The recess or receptacle 1000q in which flavored article 137q or flavor pod 139q is locatable may be provided within the main body 102q, the system 101q, or within the consumable 103q. The flavored article 137q or flavor pod 139q may be a removable, replaceable, and/or consumable flavor part 142q of the smoking substitute system 101q. The flavored article 137q or flavor pod 139q may be releasably attached to the consumable 103q, for example to the end of the consumable which is configured to engage with the main body 102q. Various means of attachment between the source of flavorant 132q and the consumable 103q may be possible, including, but not limited to, an interference fit, a snap fit comprising one or more raised bumps and corresponding recesses on the interfacial surface between the two components, or through physically coupling together by screwing one onto the other, through a bayonet fitting, through a latching mechanism, or through a clip or clasp. If the source of flavorant 132q is attached via a latching mechanism, this may further include a button by which the latch may be released. A clip or clasp may be fixed in position relative to the consumable 103q, or hingedly, rotatably, or slidably attached to the consumable so as to be movable to allow the flavored article 137q or flavor pod 137 to be fitted and released.

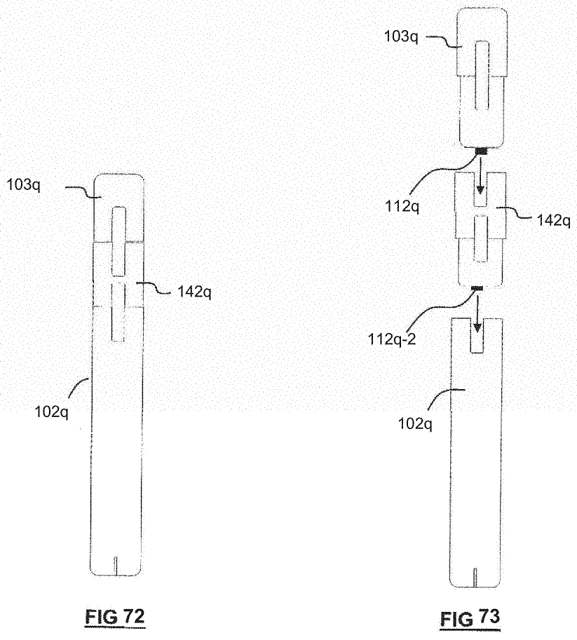

The flavored article 137q or flavor pod 139q may alternatively be comprised within a flavor part 142q of the smoking system, in the manner illustrated schematically in FIGS. 72 and 73. Said flavor part 142q may be located between the consumable 103q and main body 102q, such that each engages with an opposing end or side of the flavor part 142q, but not directly with each other. The flavor part 142q in such an embodiment may further comprise an electrical contact 112q-2 and connection therethrough, so that power may be conveyed from the contacts of the main body 102q to the contacts 112q of the consumable 103q via the flavor part 142q. The interface between the consumable 103q and flavor part 142q may be configured to be the same as the interface between the flavor part 142q and main body 102q, such that the user is afforded the option of choosing to use the smoking substitute device 101q either with or without the flavor part 142q.

A flavored article 137q or flavor pod 139q may therefore be provided as a stand-alone consumable, independent of the consumable 103q comprising the aerosol generator. One or more flavored articles 137q or flavor pods 139q may be provided in a retail pack. Each flavored article 137q or flavor pod 139q within a pack may comprise the same flavorant 132q. Alternatively, the pack may comprise flavored articles 137q or flavor pods 139q containing different flavorants 132q, in order to provide a user with a selection of possible flavorants 132q.

Alternatively, in embodiments where the source of flavorant 132q is comprised within a consumable 103q-2, one or more consumables 103q-2 may be provided in a retail pack. Each consumable 103q-2 within a pack may comprise the same flavorant 132q. Alternatively, the pack may comprise consumables 103q-2 containing different flavorants 132q in order to provide a user with a selection of possible flavorants 132q.

Sixteenth Mode: A Flavor Delivery Article that Releases a Flavorant in the Presence of an Aerosol Generated from a Smoking Substitution Apparatus

Aspects and embodiments of the sixteenth mode of the present disclosure will now be discussed with reference to the accompanying figures. Further aspects and embodiments of the sixteenth mode will be apparent to those skilled in the art. All documents mentioned in this text are incorporated herein by reference.

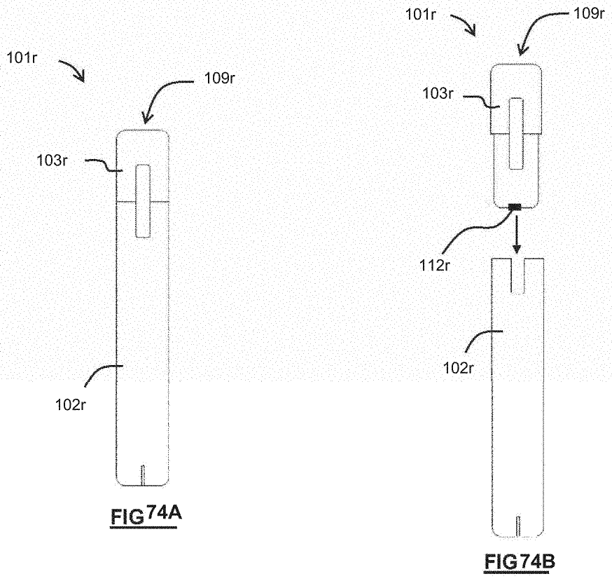

FIGS. 74A and 74B illustrate a smoking substitute system in the form of an e-cigarette system 101r. The system 101r comprises an e-cigarette device defining a main body 102r of the system 101r, and a smoking substitute apparatus in the form of an e-cigarette consumable (or “pod”) 103r. The smoking substitute apparatus is a smoking substitute apparatus. In the illustrated embodiment the consumable 103r (smoking substitute apparatus) is removable from the main body (e-cigarette device), so as to be a replaceable component of the system 101r. In other words, the e-cigarette system 101r is a closed system.

As is apparent from FIGS. 74A and 74B, the consumable 103r is configured to engage the main body 102r. FIG. 74A shows the main body 102r and the consumable 103r in an engaged state, whilst FIG. 74B shows the main body 102r and the consumable 103r in a disengaged state. When engaged, a portion of the consumable 103r is received in a cavity of the main body 102r and is retained in the engaged position by way of a snap-engagement mechanism. In other embodiments, the main body 102r and consumable 103r may be engaged by screwing one into (or onto) the other, through a bayonet fitting, or by way of an interference fit.

The system 101r is configured to vaporize an aerosol-former, which in the illustrated embodiment, is in the form of a nicotine-based e-liquid 104r. The e-liquid 104r comprises nicotine and a base liquid including propylene glycol and/or vegetable glycerin. In the present embodiment, the e-liquid 104r is flavorless (and does not include any added flavorant). That is, if the e-liquid 104r were to be inhaled (i.e., in aerosol form) by a user, it would not have a particularly perceptible flavor or taste.

As is more apparent from FIG. 74C, this e-liquid 104r is stored within a reservoir in the form of a tank 105r that forms part of the consumable 103r. In the illustrated embodiment, the consumable 103r is a “single-use” consumable 103r. That is, upon exhausting the e-liquid 104r in the tank 105r, the intention is that the user disposes of the entire consumable 103r. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105r surrounds, and thus defines a portion of, a passage 106r that extends between an inlet 107r and an outlet 108r at opposing ends of the consumable 103r. In this respect, the passage comprises an upstream end at the end of the consumable 103r that engages with the main body 102r, and a downstream end at an opposing end of the consumable 103r that comprises a mouthpiece 109r of the system 101r. When the consumable 103r is engaged with the main body 102r, a user can inhale (i.e., take a puff) via the mouthpiece 109r so as to draw air through the passage 106r, and so as to form an airflow (indicated by arrows) in a direction from the inlet 107r to the outlet 108r of the passage 106r. Although not illustrated, the passage 106r may be partially defined by a tube (e.g., a metal tube) extending through the consumable 103r. The passage 106r is in fluid communication with a gap defined between the consumable 103r and the main body 102r (when engaged) such that air outside of the system 101r is drawn into the passage 106r (during an inhale).

The smoking substitute system 101r is configured to vaporize the e-liquid 104r for inhalation by a user. To provide this, the consumable 103r comprises a heater having of a porous wick 110r and a resistive heating element in the form of a heating filament 111r that is helically wound around a portion of the porous wick 110r. The porous wick 110r extends across the passage 106r (i.e., transverse to a longitudinal axis of the passage 106r) and opposing ends of the wick 110r extend into the tank 105r (so as to be submerged in the e-liquid 104r). In this way, e-liquid 104r contained in the tank 105r is conveyed from the opposing ends of the porous wick 110r to a central portion of the porous wick 110r so as to be exposed to the airflow in the passage 106r (i.e., caused by a user inhaling).

The helical filament 111r is wound about this exposed central portion of the porous wick 110r and is electrically connected to an electrical interface in the form of electrical contacts 112r mounted at the end of the consumable that is proximate the main body 102r (when engaged). When the consumable 103r is engaged with the main body 102r, the electrical contacts 112r contact corresponding electrical contacts (not shown) of the main body 102r. The main body electrical contacts are electrically connected to a power source (not shown) of the main body 102r, such that (in the engaged position) the filament 111r is electrically connected to the power source. In this way, power can be supplied by the main body 102r to the filament 111r in order to heat the filament 111r. This heat is transferred from the filament 111r to the porous wick 110r which causes e-liquid 104r conveyed by the porous wick 110r to increase in temperature to a point at which it vaporizes. The vaporized e-liquid becomes entrained in the airflow and, between the vaporization point at the filament 111r and the outlet 108r of the passage 106r, condenses to form an aerosol. This aerosol is then inhaled, via the mouthpiece 109r, by a user of the system 101r.

The power source of the main body 102r may be in the form of a battery (e.g., a rechargeable battery).

The main body 102r may comprise a connector in the form of, e.g., a USB port for recharging this battery. The main body 102r may also comprise a controller that controls the supply of power from the power source to the main body electrical contacts (and thus to the filament 111r). That, is the controller may be configured to control a voltage applied across the main body electrical contacts, and thus the voltage applied across the filament 111r. In this way, the filament 111r may only be heated under certain conditions (e.g., during a puff and/or only when the system is in an active state). In this respect, the main body 102r may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102r and consumable 103r may comprise a further interface which may, for example, be in the form of an RFID reader, a barcode or QR code reader. This interface may be able to identify a characteristic (e.g., a type) of a consumable 103r engaged with the main body 102r. In this respect, the consumable 103r may include any one or more of an RFID chip, a barcode or QR code, or memory within which is an identifier and which can be interrogated via the interface.

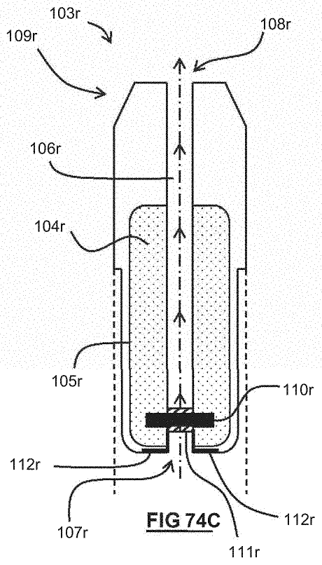

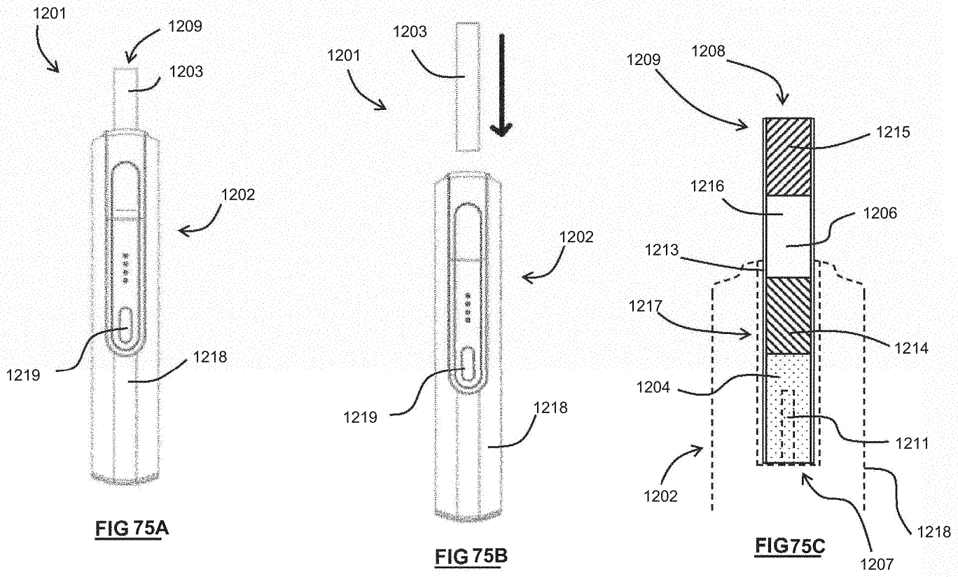

FIGS. 75A and 75B illustrate a smoking substitute system the form of a heated tobacco (HT) system 1201. The system 1201 comprises an HT device 1202 and an aerosol-forming article in the form of a HT consumable 1203.

The consumable 1203 is configured to engage the device 1202 by way of an interference fit. FIG. 75A shows the device 1202 and the consumable 1203 in an engaged state, and FIG. 75B shows the device 1202 and the consumable 1203 in a disengaged state.

Returning now to the device 201, FIG. 75C illustrates a detailed view of the end of the device 1201 that is configured to engage with the consumable 1203. The cap of the device 1202 includes an opening to an internal cavity defined by the cap. The opening and the cavity are formed so as to receive at least a portion of the consumable 1203. During engagement of the consumable 1203 with the device 1202, a portion of the consumable 1203 is received through the opening and into the cavity. After engagement, the downstream end of the consumable 1203 protrudes from the opening and thus also protrudes from the device 1202. The opening includes laterally disposed notches. When a consumable 1203 is received in the opening, these notches remain open and could, for example, be used for retaining a cover in order to cover the end of the device 1202.

The consumable 1203 generally resembles a cigarette. In this respect, the consumable 1203 has a generally cylindrical form with a diameter of approximately 7 mm and an axial length of approximately 70 mm. The consumable 1203 comprises an outer wrapping layer 1213 defining a passage 1206 extending between openings at opposing ends of the consumable 1203. Thus, the passage comprises an upstream end (and opening) 1207 at the end of the consumable 1203 that engages with the device 1202, and a downstream end (and opening) 1208 at an opposing end of the consumable 1203 that projects from the device 1202 (when engaged) and defines a mouth end 1209 of the system 1201. When the consumable 1203 is engaged with the device 1202, a user can inhale (i.e., take a puff) via the mouth end 1209 so as to draw air through the passage 1206, and so as to form an airstream in a direction from the upstream end 1207 to the downstream end 1208 of the passage 1206.

The consumable 1203 comprises an aerosol former in the form of an aerosol-forming substrate 1204 (see FIG. 75C) that is disposed at the upstream end 1207 of the passage 1206. The consumable 1203 further comprises an upstream filter 1214 adjacent the aerosol-forming substrate 1204, a terminal filter 1215 at the downstream end 1208, and a spacer 1216 interposed between the terminal 1215 and upstream 1214 filters.

The aerosol forming substrate 1204 comprises tobacco material that may, for example, include any suitable parts of the tobacco plant (e.g., leaves, stems, roots, bark, seeds and flowers). In order to generate an aerosol, the aerosol forming substrate 1204 comprises at least one volatile compound that is intended to be vaporized/aerosolized and that may provide the user with a recreational and/or medicinal effect when inhaled. The aerosol-forming substrate 1204 may further comprise one or more additives. For example, such additives may be in the form of humectants (e.g., propylene glycol and/or vegetable glycerin), flavorants, fillers, aqueous/non-aqueous solvents and/or binders.

The device comprises a heating element 1211 that projects into the aerosol-forming substrate 1204 when the consumable 1203 is engaged with the device 1202. This heating element 1211 is electrically connected to a power supply (not shown) of the device 1202 and, when activated, heats the aerosol-forming substrate 1204 such that vapor is released from the aerosol-forming substrate 1204. When a user inhales via the mouth end 1209, air is drawn through the heated aerosol-forming substrate 1204 and the vapor becomes entrained in the resultant airflow. As the vapor flows from the aerosol-forming substrate 1204 to the downstream end 1208 of the passage 1206 (through the filters 1214, 1215 and the spacer 1216), it condenses into an aerosol and the aerosol is inhaled by the user.

As is apparent from FIG. 75C, when engaged, the consumable 1203 is received in a cavity 1217 formed in a housing 1218 of the device 1202. Whilst not shown in the figures, the housing 1218 accommodates (in addition to the abovementioned power supply) a controller for controlling power supply to the heating element 1211. Control of the heating element 1211 may be performed in response to a user input (e.g., via a button 1219 disposed on the housing 1218) and/or a signal received from a puff sensor (configured to indicate a puff state).

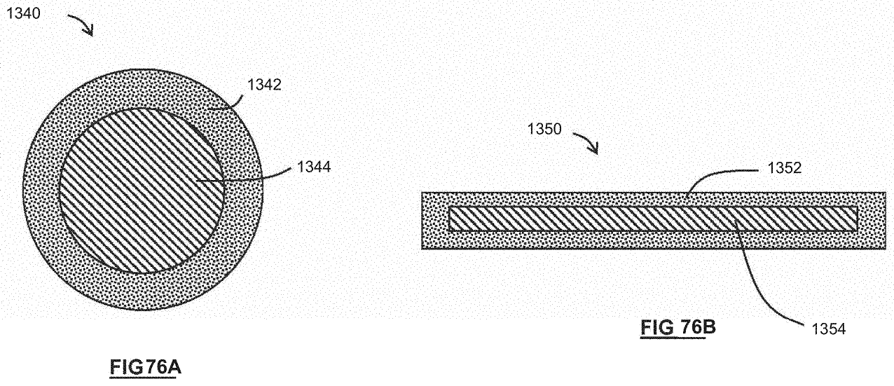

FIG. 76A is a cross sectional view of a flavor delivery article according to an embodiment of the present disclosure. The flavor delivery article as shown in FIG. 76A is a capsule, or a microcapsule 1340. The capsule 1340 comprises a barrier or shell 1342 for encapsulating a flavorant 1344. In the illustrated embodiment, the flavorant 1344 is a freeze dried flavorant. The freeze dried flavorant 1344 having hydrophilic properties and therefore it readily forms a reconstituted flavorant 1344 upon contacting water, e.g., saliva in a user's oral cavity. The flavorant 1344 is shielded from the environment by the barrier 1342. More specifically, the barrier 1342 forms an impermeable barrier to stop moisture ingress, as well as the release of flavorant 1344. That is, the barrier 1342 is insoluble in water or saliva. Thus, the capsule 1340 remains stable once it is received in the user's oral cavity.

The capsule 1340 is spherical and may have a particle size ranging from 1 mm to 20 mm. In this example, a single capsule 1340 with a 5 mm diameter is sufficient to provide the required flavorant release over a vaping session. In some other embodiments, a plurality of microcapsules 1340, having a particle size of or less than 1 mm, may be required to provide the same flavorant release. For example, the plurality of microcapsules 1340 may resemble a free-flowing powder, and the user may control the flavor release by varying the number of microcapsules to apply to his/her oral cavity.

In some embodiments, a single application of the capsule 1340 to a user's oral cavity is sufficient to provide flavorant release over a vaping session. In some other embodiments, a plurality of applications of capsule 1340 to a user's oral cavity may be required throughout a vaping session. For example, the user may require a plurality of doses of capsule 1340 through the vaping session.

In the illustrated embodiment, the barrier 1342 is formed of a hydrogel composite comprising chitosan. The chitosan contained in the hydrogel composite causes the barrier to disintegrate, leak or break down, when it is exposed to an alkaline environment. For example, the barrier containing chitosan may gradually disintegrate when the pH value of the oral cavity exceeds 7.5. Furthermore, said chitosan component may cause the barrier 1342 to disintegrate more rapidly upon exposing to highly alkaline environment. For example, when puffing on the smoking substitute apparatus, the aerosol containing free base nicotine increases the alkalinity of the oral cavity to a level at or above pH 7.8. At such alkaline environment, the chitosan in the barrier 1342 disintegrates or breaks down rapidly, and thereby exposing and releasing the flavorant to the saliva in the oral cavity.

The chitosan contained in the hydrogel may be provided with a degree of deacetylation (DDT %) ranging from 75% to 99%. In this example, the hydrogel comprises chitosan with a degree of deacetylation (DDT %) of 85%.

Furthermore, the hydrogel may comprise chitosan at a concentration between 0.1% w/w and 20% w/w. In this example, the hydrogel comprises chitosan at a concentration of 10% w/w.

For example, when the barrier 1342 is breached as it contacts the aerosol, the saliva may ingress or leach into the barrier 1342, and thereby dissolves the freeze dried flavorant 1344 stored therein. The dissolved flavorant 1344 may then be released from the capsule 1340 and to the user's taste receptors in the oral cavity.

Therefore, the presence of an aerosol enables the rapid release of flavorant 1344 from the capsule 1340. On the other hand, in the absence of an aerosol the capsule 1340 may remain stable, or at least the capsule 1340 does not rapidly disintegrate, when it is received in the user's oral cavity.

The capsule 1340 in this example is produced by first preparing the flavorant before freeze drying the flavorant in a mold. This produces a solid spherical flavorant 1344 block.

The barrier 1342, on the other hand, may be produced by mixing chitosan, along with other excipients, such as xanthan gum, water, and optionally propylene glycol and/or vegetable glycerin. The mixture is dried to produce a barrier gel, or a hydrogel.

Subsequently, the spherical flavorant 1344 is inserted into barrier gel 1342 in a mold, and thereby encapsulates the flavorant in the barrier. For example, the method includes: i) layering the barrier gel 1342 in a mold; ii) inserting the spherical flavorant 1344; and (iii) rolling the barrier gel 1342 to form a capsule 1340.

FIG. 76B is a cross sectional view of a flavor delivery article according to another embodiment of the present disclosure. The flavor delivery article as shown in FIG. 76B is a composite sheet 1350. Similar to the capsule 1340 as shown in FIG. 76A, the composite sheet 1350 comprises a barrier 1352 for encapsulating a planar shaped flavorant 1354. In the illustrated embodiment, the flavorant 1354 is a freeze dried flavorant. The freeze dried flavorant 1354 having hydrophilic properties and therefore it readily forms a reconstituted flavorant 1354 upon contacting water, e.g., saliva in a user's oral cavity. The flavorant 1354 is shielded from the environment by the barrier 1352. More specifically, the barrier 1352 forms an impermeable barrier to stop moisture ingress, as well as the release of flavorant. That is, the barrier 1352 is insoluble in water or saliva. The use of composite sheet 1350 is beneficial because of its large surface area. For example, a single composite sheet 1350 covers a significant portion of a user's tongue and therefore allows flavorant to uniformly released across the many taste receptors thereat.

The composite sheet 1350 may be formed in a similar manner to the capsule 1340. In this case, the flavorant is freeze-dried in a planar mold, which forms a planar shaped flavorant 1354. The flavorant 1354 is then sandwiched in between two layers of barrier gel 1352 to form the composite sheet 1350.

Using the capsule 1340 as an example, the flavor delivery article can be applied directly to a user's oral cavity. For example, the user may manually place one or more of the capsules 1340 into his/her oral cavity, or the user may use a suitable dispenser (not shown) for dispensing a predetermined number or volume of capsules. The use of dispenser allows a precise quantity of capsules 1340 to be dispensed, thereby allowing a repeatable vaping experience. The dispenser may be an adjustable dispenser which allows the user to vary the quantity of capsules to dispense.

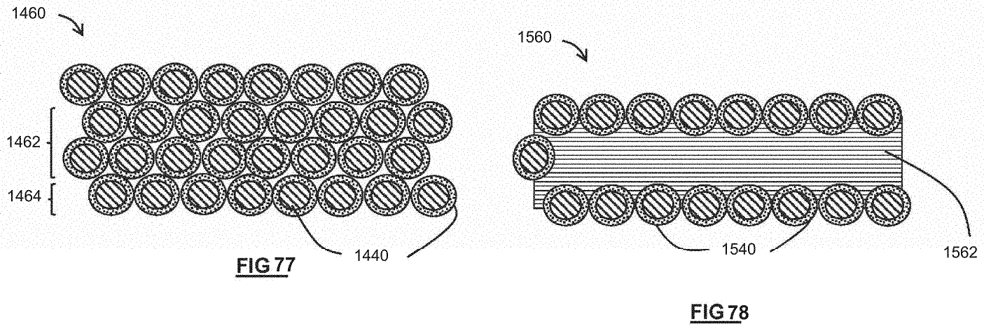

FIG. 77 is a cross sectional view of a flavor delivery article according to another embodiment of the present disclosure. In the illustrated embodiment, the flavor delivery article may take the form of a tablet 1460. Said tablet 1460 may be formed by compressing a plurality of capsules 1440, or adhering them together. Each of the tablets contains a predetermined quantity of capsules 1440 and therefore the amount of flavorant contained in each of the tablet can be precisely controlled. The use of tablet allows flavorant to be released in a gradual manner. That is, the flavorant contained at the core of tablet 1462 would not be released to the oral cavity until the capsules 1440 at the peripheral layer 1464 of the tablet 1460 has disintegrated or break away from the tablet 1460.

FIG. 78 is a cross sectional view of a flavor delivery article 1560 according to another embodiment of the present disclosure. In the illustrated embodiment, the capsules 1540 are coated, or adhered, onto a substrate 1562. The substrate 1562 may be a cardboard or a blotting paper that is insoluble in saliva. In use, the substrate 1562 may be placed in the user's oral cavity and thereby allowing the capsules 1540 to contact the aerosol and release the flavorant stored therein. The substrate 1562 may be removed from the user's oral cavity after use.

Alternatively, the substrate 1562 may be soluble in saliva and thereby gradually releases the capsules 1540 to the user's oral cavity as the substrate 1562 dissolves. The soluble substrate 1560 may be a candy, such as hard-boiled sweets and lollipops, having the capsules 1540 coated onto the surface or within the substrate. The soluble substrate 1562 may comprise an additional flavorant different to the flavorant contained in the capsules 1540, such that the additional flavorant in the substrate 1562 may be released as the substrate 1562 dissolves. This may allow sequential release of different flavoring, since the additional flavorant may be readily released as the substrate 1562 dissolves in the saliva, wherein the capsules 1540 may not activate until it is in contact with the aerosol.

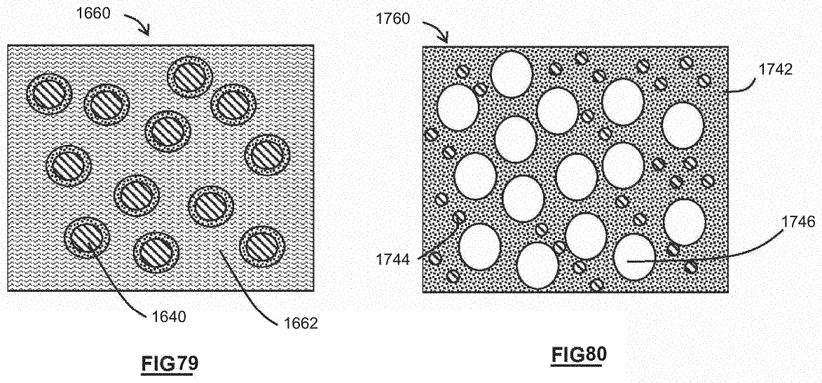

FIG. 79 is a cross sectional view of a flavor delivery article 1660 according to another embodiment of the present disclosure. In the illustrated embodiment, the capsule 1640 may be suspended in a carrier liquid 1662, e.g., water, to form a solid-liquid suspension 1660. That is, with the aid of the carrier liquid 1662, the flavor delivery article 1660 may be able to flow and therefore such arrangement allows said flavor delivery article 1660 to be dispensed more easily. For example, the flavor delivery article suspension may be applied to a user's oral cavity using a spray pump (not shown), wherein each pump stroke delivers a predetermined quantity of capsules 1640. Alternatively, the flavor delivery article may be applied to a user's oral cavity using a dropper or a syringe (not shown), wherein the user may specify the quantity of capsules 1640 by varying the dose of flavor delivery article suspension.

In some other embodiments, the carrier liquid 1662 applied in the solid-liquid suspension 1660 may be a viscous fluid and the suspension may take the form of a paste. In use, the user may apply the paste 1660 directly in the oral cavity, e.g., on the tongue or the teeth. The paste may allow the particle to temporarily adhere to the user's oral cavity, and thereby prolonging the duration of flavorant release.

FIG. 80 is a cross sectional view of a flavor delivery article 1760 according to another embodiment of the present disclosure. In the illustrated embodiment, the flavor delivery article is in the form of a porous structure formed from a barrier material 1742, e.g., a chitosan hydrogel. In this embodiment, the flavorant 1744 is embedded in the barrier material 1742 and is releasable from said barrier material 1742 when the flavor delivery article 1760 comes into contact with a nicotine containing aerosol. That is, the barrier material 1742 is configured to disintegrate or erode away when it contacts the aerosol and thereby releasing the flavorant 1744. In this embodiment, the porous structure comprises a plurality of voids 1746 in the barrier material, wherein the majority of voids 1746 are in fluid communication with the atmosphere. This allows aerosol to flow to or to penetrate into the core portion of the flavor delivery article 1760 and thereby increases the contacting area between the barrier material 1742 and the aerosol, thus allowing a more efficient release of the flavorant 1744.

The porous flavor delivery article 1760 may be formed by mixing or blending the flavorant 1744 with a barrier material 1742, such as chitosan cross-linked with glutaraldehyde. The mixture is then freeze-dried to from the porous flavor delivery article 1760. Since the porous flavor delivery article 1760 is formed by blending flavorant 1744 with the barrier material 1742, a small portion, or insignificant portion of flavorant may be disposed onto the surface of the porous flavor delivery article 1760.

Alternatively, the barrier 1342, 1352, 1742 in any one of the flavor delivery articles 1340, 1350, 1460, 1560, 1660 and 1760 shown in FIGS. 76 to 80 is configured to disintegrate in response to a change in temperature induced by the aerosol. For example, the barrier 1342, 1352, 1742 may disintegrate at a temperature above 40° C. That is, the flavor delivery article may remain stable, e.g., not disintegrating instantaneously, in a user's oral cavity until it is heated by a stream of aerosol having a temperature exceeding 40° C. For example, the aerosol generated by a heated tobacco apparatus, e.g., a heat not burn apparatus, may be exhausted at a temperature above 40° C., and therefore sufficient to cause the barrier 1342, 1352, 1742 to disintegrate rapidly. The barrier 1342, 1352, 1742 in these cases may comprise any one or more of poloxamers, cellulose and xyloglucan.

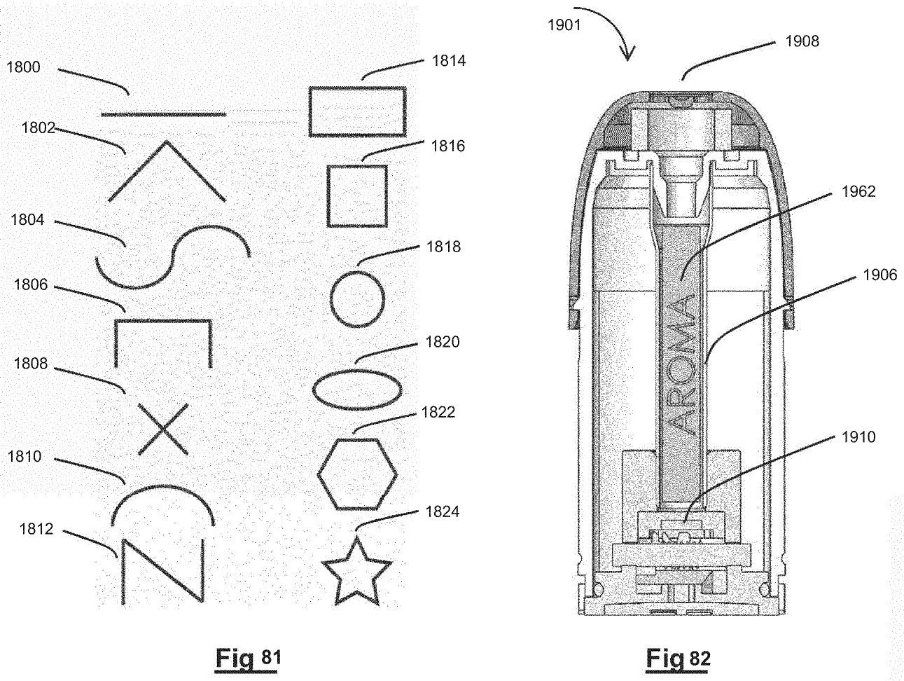

FIG. 81 shows cross-sectional views of possible flavorant carrying substrate shape profiles. Shown on the left-hand side are open profile options and shown on the right-hand side are solid or hollow profile options. The left-hand side from top to bottom shows flat 1800, bent 1802, S-curved 1804, U-shaped 1806, X-shaped 1808, semicircle shaped 1810 and Z-shaped 1812 cross-section profiles. The right-hand side from top to bottom shows rectangular 1814, square 1816, circle 1818, oval 1820, hexagonal 1822 and star-shaped 1824 cross-section profiles.

FIG. 82 is sectional view of a smoking substitute apparatus 1901 having a thin, flat substrate 1962 (e.g., 1 mm thick) that is situated in the passage 1906 above the aerosol generator. The flat substrate is positioned along the length of the cylindrical passage between the aerosol generator and the outlet. In this manner, the aerosol vapor that is generate will pass along both the largest sides of the flat substrate to maximize release and entrainment of the flavorant from the substrate.

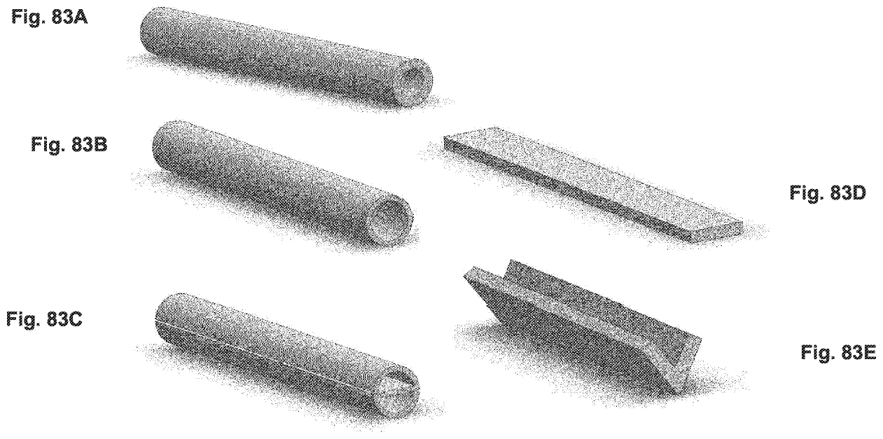

FIGS. 83A to 83E show perspective views of five flavor delivery article carrying substrate shapes. FIG. 83A shows a cylindrical tube with a 2 mm internal diameter, 0.7 mm wall thickness, 32 mm length and 201 mm² internal surface area. FIG. 83B shows a cylindrical tube with a 2.7 mm internal diameter, 0.35 mm wall thickness, 32 mm length and 271 mm² internal surface area. FIG. 83C shows an e-shaped tube with a 0.35 mm wall thickness, 32 mm length and an internal surface area of 436 mm².FIG. 83D shows a flat card that is 3.4 mm wide, 0.7 mm thick, 32 mm long with a surface area of 218 mm². FIG. 83E shows a V-shaped substrate with 2.9 mm long outside length of the legs, 0.7 mm wall thickness, 32 mm length and 298 mm² surface area.

FIG. 84 is a side view of a smoking substitute apparatus 2101 having a substrate 2162 comprising a flavor delivery article 2160, wherein the substrate 2162 protrudes from the passage 2106 of the smoking substitute apparatus (i.e., it is not fully contained within the smoking substitute apparatus) and through the outlet 2108. The substrate 2162 may therefore directly contact the user's oral cavity and/or tongue to enhance the flavor experience.

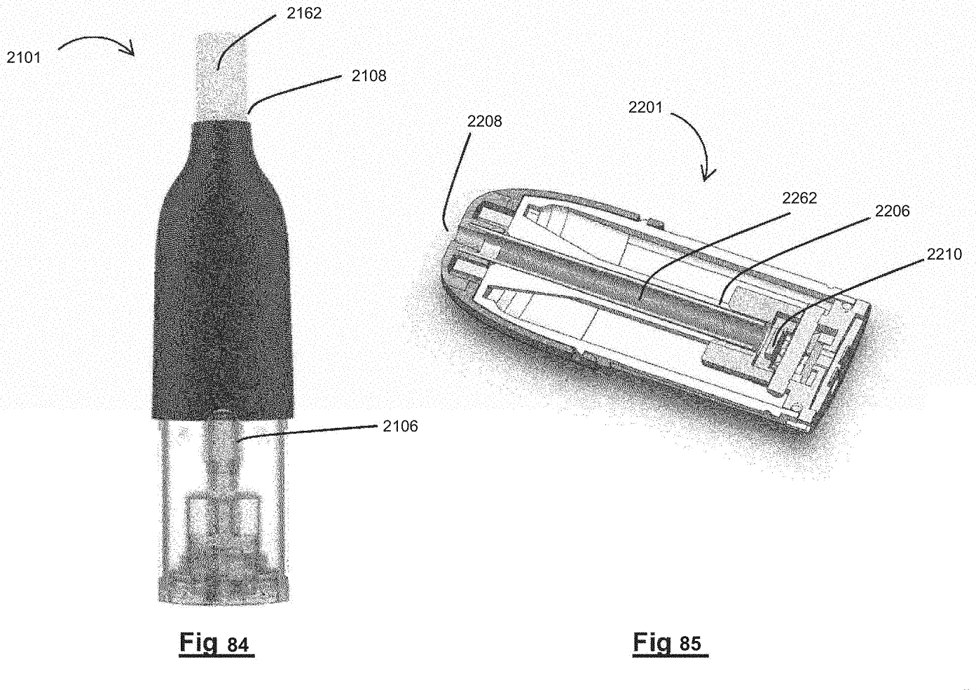

FIG. 85 is a sectional view of a smoking substitute apparatus 2201 having a flavor delivery article 2260 on a cylindrical substrate 2262 that is positioned along the length of the substantially cylindrical passage 2206 between the aerosol generator 2210 and the outlet 2208. The cylindrical substrate 2262 is sized such that it contacts the surface of the passage 2206 and so aerosol passes only across the internal surface of the substrate 2262.

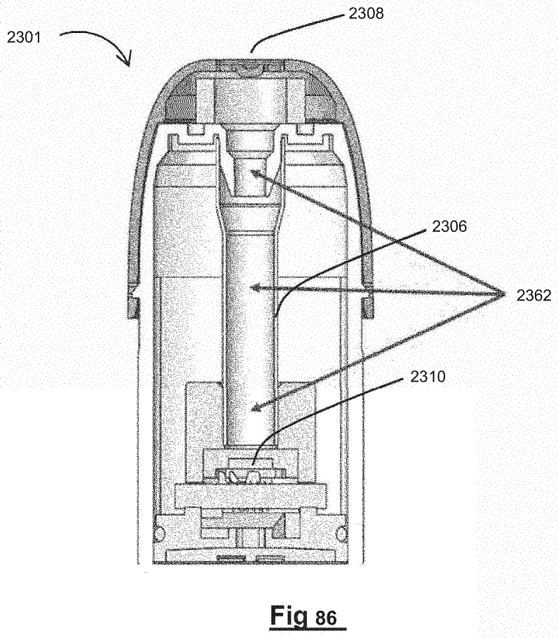

FIG. 86 is a sectional view of a smoking substitute apparatus 2301 having a flavor delivery article 2362 applied directly to the surface of the passage 2306, such as at the positions indicated by the arrows and therebetween.

Seventeenth Mode: A Flavored Article which can be Placed Over at Least Part of the Fluid Inlet

Aspects and embodiments of the seventeenth mode of the present disclosure will now be discussed with reference to the accompanying figures. Further aspects and embodiments of the seventeenth mode will be apparent to those skilled in the art. All documents mentioned in this text are incorporated herein by reference.

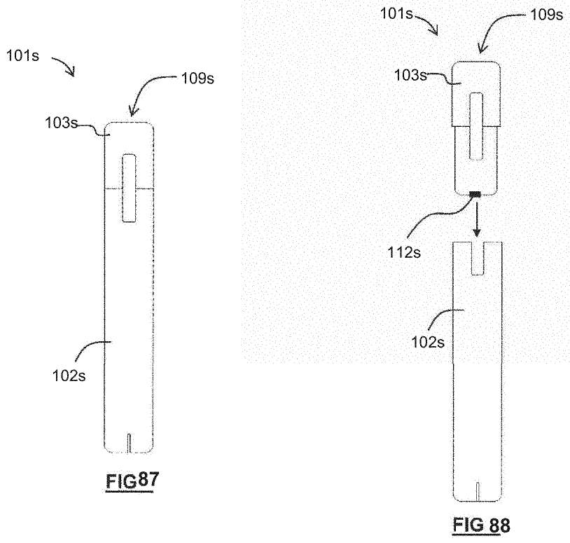

FIGS. 87 and 88 illustrate a smoking substitute system in the form of an e-cigarette system 101s. The system 101s comprises an e-cigarette device defining a main body 102s of the system 101s, and a smoking substitute device in the form of an e-cigarette consumable (or “pod”) 103s. In the illustrated embodiment the consumable 103s (smoking substitute device) is removable from the main body (e-cigarette device), so as to be a replaceable component of the system 101s. In other words, the e-cigarette system 101s is a closed system.

As is apparent from FIGS. 87 and 88, the consumable 103s is configured to engage the main body 102s. FIG. 87 shows the main body 102s and the consumable 103s in an engaged state, whilst FIG. 88 shows the main body 102s and the consumable 103s in a disengaged state. When engaged, a portion of the consumable 103s is received in a cavity of the main body 102s and is retained in the engaged position by way of a snap-engagement mechanism. In other embodiments, the main body 102s and consumable 103s may be engaged by screwing one into (or onto) the other, through a bayonet fitting, or by way of an interference fit.

The system 101s is configured to vaporize an aerosol former or aerosol precursor, which, in the illustrated embodiment, is in the form of a nicotine-based e-liquid 104s. The e-liquid 104s comprises nicotine and a base liquid including propylene glycol and/or vegetable glycerin. In the present embodiment, the e-liquid 104s is flavorless and so does not include any added flavorant. That is, if the e-liquid 104s were to be inhaled (i.e., in aerosol form) by a user, it would not have a particularly perceptible flavor or taste. It is to be appreciated, however, that in other embodiments the e-liquid 104s may comprise an inherent flavorant. In the present specification, the term flavorant may be understood as referring to one or more substances effective to activate at least one of an olfactory receptor in a human nasal cavity; and a taste receptor in a human oral cavity.

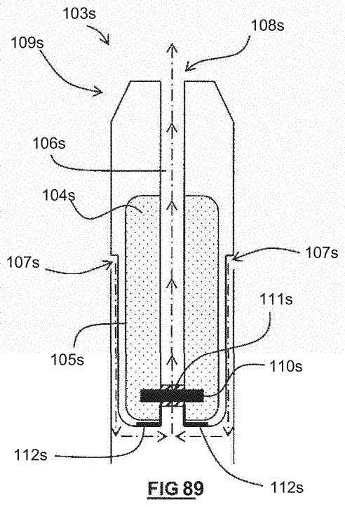

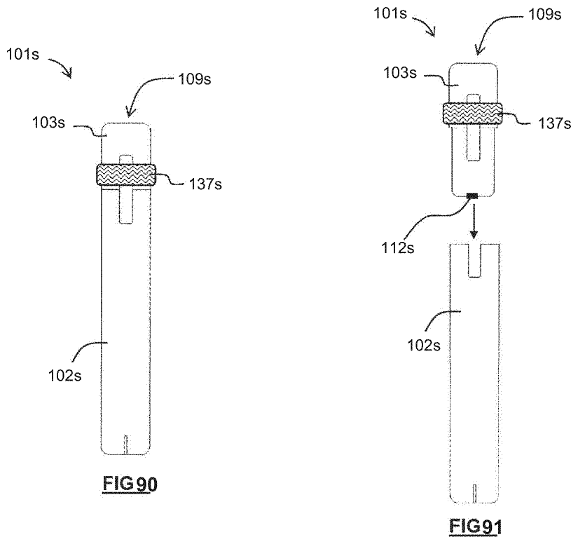

As is more apparent from FIG. 89, the e-liquid 104s is stored within a reservoir in the form of a tank 105s that forms part of the consumable 103s. In the illustrated embodiment, the consumable 103s is a “single-use” consumable 103s. That is, upon exhausting the e-liquid 104s in the tank 105s, the intention is that the user disposes of the entire consumable 103s. In other embodiments, the e-liquid (i.e., aerosol precursor) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105s surrounds, and thus defines a portion of, a flow passage 106s that extends between an inlet 107s and an outlet 108s at opposing ends of the consumable 103s. In this respect, the flow passage 106s comprises an upstream end at the end of the consumable 103s that engages with the main body 102s, and a downstream end at an opposing end of the consumable 103s that comprises a mouthpiece 109s of the system 101s. When the consumable 103s is engaged with the main body 102s, a user can inhale (i.e., take a puff) via the mouthpiece 109s so as to draw air through the passage 106s, and so as to form an airflow (indicated by arrows) in a direction from the inlet 107s to the outlet 108s of the passage 106s. Although not illustrated, the passage 106s may be partially defined by a tube (e.g., a metal tube) extending through the consumable 103s. The passage 106s is in fluid communication with a gap defined between the consumable 103s and the main body 102s (when engaged) such that air outside of the system 101s is drawn into the passage 106s (during an inhale).

The smoking substitute system 101s is configured to vaporize the e-liquid 104s for inhalation by a user. To provide this, the consumable 103s comprises a heater having of a porous wick 110s and a resistive heating element in the form of a heating filament 111s that is helically wound around a portion of the porous wick 110s. The porous wick 110s and heating filament 111s may be referred to collectively as an aerosol generator. The aerosol generator is arranged between the inlet 107s and outlet 108s of the smoking substitute system 101s, and is in fluid communication with both the inlet 107s and outlet 108s. The porous wick 110s extends across the passage 106s (i.e., transverse to a longitudinal axis of the passage 106s) and opposing ends of the wick 110s extend into the tank 105s (so as to be submerged in the e-liquid 104s). In this way, e-liquid 104s contained in the tank 105s is conveyed from the opposing ends of the porous wick 110s to a central portion of the porous wick 110s so as to be exposed to the airflow in the passage 106s (i.e., caused by a user inhaling).

The helical filament 111s is wound about the exposed central portion of the porous wick 110s and is electrically connected to an electrical interface in the form of electrical contacts 112s mounted at the end of the consumable that is proximate the main body 102s (when engaged). When the consumable 103s is engaged with the main body 102s, the electrical contacts 112s contact corresponding electrical contacts (not shown) of the main body 102s. The main body electrical contacts are electrically connected to a power source (not shown) of the main body 102s, such that (in the engaged position) the filament 111s is electrically connected to the power source. In this way, power can be supplied by the main body 102s to the filament 111s in order to heat the filament 111s. This heat is transferred from the filament 111s to the porous wick 110s which causes e-liquid 104s conveyed by the porous wick 110s to increase in temperature to a point at which it vaporizes. The vaporized e-liquid becomes entrained in the airflow. Between the vaporization point at the filament 111s and the outlet 108s of the passage 106s, the vaporized e-liquid condenses to form an aerosol. This aerosol is then inhaled via the mouthpiece 109s by a user of the system 101s.

The power source of the main body 102s may be in the form of a battery (e.g., a rechargeable battery).

The main body 102s may comprise a connector in the form of, e.g., a USB port for recharging this battery. The main body 102s may also comprise a controller that controls the supply of power from the power source to the main body electrical contacts (and thus to the filament 111s). That, is the controller may be configured to control a voltage applied across the main body electrical contacts, and thus the voltage applied across the filament 111s. In this way, the filament 111s may only be heated under certain conditions (e.g., during a puff and/or only when the system is in an active state). In this respect, the main body 102s may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102s and consumable 103s may comprise a further interface which may, for example, be in the form of an RFID reader, a barcode or QR code reader. This interface may be able to identify a characteristic (e.g., a type) of a consumable 103s engaged with the main body 102s. In this respect, the consumable 103s may include any one or more of an RFID chip, a barcode or QR code, or memory within which is an identifier and which can be interrogated via the interface. A similar interface may also be comprised in the flavored article 137s and smoking substitute device 103s.

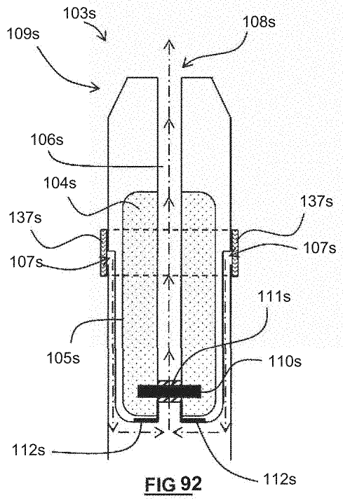

As is illustrated in FIGS. 90, 91, and 92, a flavored article 137s is arranged external of the smoking substitute system such that it covers at least part of the inlet 107s of the smoking substitute system 101s. Air drawn through the passage 106s by a user drawing via the outlet 108s or mouthpiece 109s therefore passes through the flavored article 137s. The flavored article 137s includes a substrate 138s which carries a flavorant 132s. Air drawn through the flavored article 137s is effective to release flavorant 132s from the flavored article 137s for entrainment in the airflow through the smoking substitute system 101s.

For example, the flavorant 132s of the flavored article 137s may have the same flavor as that in the e-liquid 104s in order to supplement the flavor provided within the e-liquid 104s and thereby provide the user with an intensified flavor sensation. Alternatively, the flavorant 132s of the flavored article 137s may be complementary to that in the e-liquid 104s (i.e., the flavors/aromas of the two flavorants, when mixed, provide a pleasing sensory combination to a user). Providing the flavorant 132s separately from the aerosol precursor 104s affords the user an opportunity to select from or change between different flavorants 132 without necessitating a change of e-liquid, for example during a vaping session.

The flavored article 137s may be arranged such that it extends towards the mouthpiece 109s from the inlet, along the exterior of the consumable pod 103s, such that at least part of the flavored article is arranged proximate to the mouthpiece 109s and will thus be proximate to the nose of a user during usage. Such an arrangement may allow an aroma from the flavored article 137s to directly enter the nasal cavity of a user in addition to the flavorant entrained in the airflow passing through the smoking substitute device 103s.

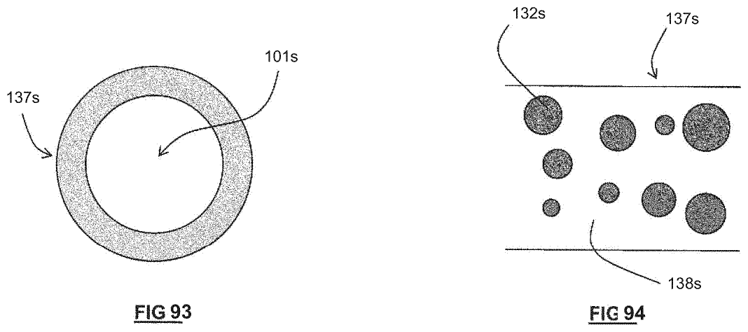

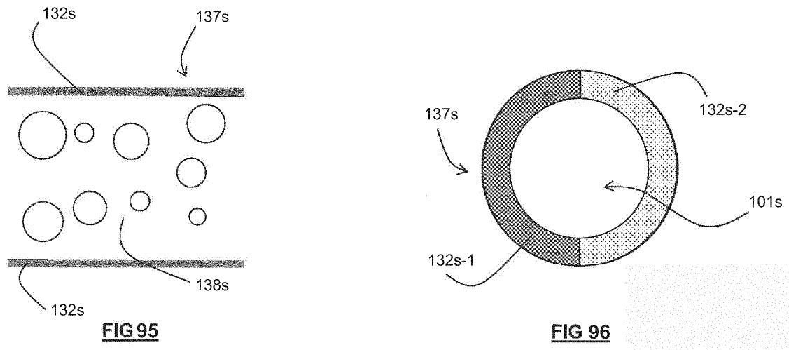

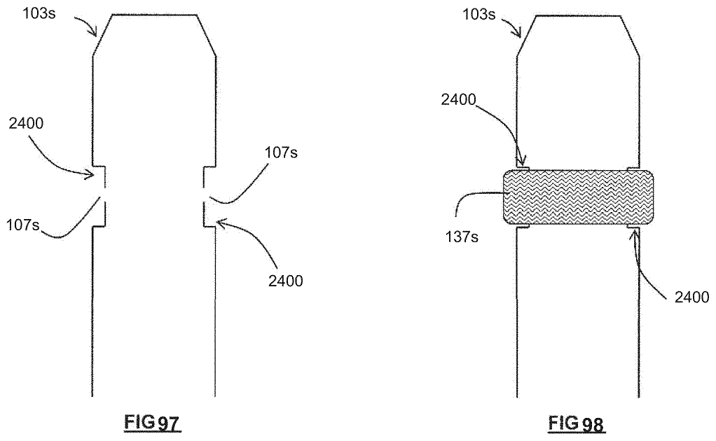

FIG. 93 shows an exemplary flavored article 137s according to the present disclosure. The flavored article 137s is substantially annular in shape, such that it can extend around part of the smoking substitute device 101s. While the flavored article 137s is illustrated as substantially circular, it may be a different shape (e.g., elliptical, oval, rectangular) to correspond, at least approximately, with the shape of the exterior of smoking substitute device 103s proximate to its inlet 106s. The flavored article 137s may be removable from the smoking substitute device 103s. A user may thereby select a flavored article 137s which carries a different flavorant 132s, allowing them to readily change flavor during a vaping session.

At least a part of the flavored article 137s may be deformable or stretchable for ease of fitting and removal from the smoking substitute device 103s. For example, at least a part the substrate 138s of the flavored article may be formed from a deformable or stretchable material. It is to be appreciated that the natural shape of the flavored article 137s (i.e., the shape of the flavored article 137s when not fitted to the smoking substitute device 103s) may be different to the external shape of the smoking substitute device 103s. Such a flavored article would be deformed by fitting to the smoking substitute device 103s such that it conforms to the external shape of said smoking substitute device 103s.

At least a part of the flavored article 137s may be resiliently deformable, such that it returns to or towards its original shape when an applied deforming force is removed. At least a part of the substrate 138s of the flavored article may, for example, be formed from a resiliently deformable material. The resiliently deformable material from which said part of the substrate 138s is formed may, for example, be viscoelastic or elastomeric.

Alternatively, or additionally, at least a part of the flavored article 137s may be substantially rigid. For example, at least a part of the substrate 138s may be formed from a substantially rigid material.

At least part of the substrate 138s may be formed from a polymeric material (e.g., silicone). Further, at least part of the substrate 138s may be formed from a foam or foamed material. Still further, at least a part of the substrate 138s may be formed from an air permeable material.

As illustrated in FIG. 94, at least part of the substrate 138s may have a porous structure, and the pores may carry the flavorant 132s. As illustrated in FIG. 95, at least part of the substrate 138s may have a porous structure or be otherwise air permeable, but with flavorant 132s provided as a coating on the surface of the flavored article 137s. At least part of the flavored article 137s may comprise the flavorant 132s both within the substrate 138s, and as a coating on the surface of the substrate 138s (not illustrated).

Flavorant 132s may be introduced into the flavored article 137s by coating (e.g., spray coating). Alternatively, or additionally, flavorant 132s may be introduced or impregnated into the flavored article 137s by, for example, immersion of the substrate 138s in a liquid comprising flavorant 132s.

The flavored article 137s may be provided with a visual indication of the flavorant 132s therein. For example, at least part of the flavored article 137s may be colored according to the flavorant. Alternatively, or additionally, the flavored article 137s may be labelled with a textual or symbolic representation of the flavorant 132s. Such a representation may be molded in to the shape of the substrate 138s, or printed on to the substrate 138s. If a region of the flavored article 137s is colored, then at least part of the colored region may be configured to “fade” (i.e., the color becoming less intense) or otherwise change color during use, to provide a visual indicator to the user of the amount of flavorant 132s remaining.

The flavored article 137s may comprise a plurality of flavorants 132s. FIG. 96 illustrates an exemplary flavored article comprising two flavorants 132s-1, 132s-2. For example, if the flavored article 137s is annular, then regions comprising different flavorants 132s may form respective sectors of the annular shape (i.e., the regions may be arranged circumferentially, with each region being part of the circumference of the annular flavored article 137s). In such an embodiment, a user is able to rotate the flavored article about the smoking substitute device 103s to place a different region over the inlet 107s, and thereby select a different flavor. The parts of the flavored article 137s with different flavorant 132s may be provided with a visual indication of the flavorant 132s therein (e.g., color). The flavorants 132s comprised within adjacent regions may be selected to have complementary flavors/aromas. A user may thereby arrange the flavored article 137s such that air drawn into the inlet 107s passes through both of two adjacent flavor regions to provide a mixture of flavors. Alternatively (not illustrated) a region substantially without flavorant may separate each of the regions comprising flavorant 132s, ensuring that a user can receive a single desired flavorant 132s, even from a flavored article 137s comprising a plurality of flavorants 132s-1, 132s-2.

The flavored article 137s may be attached to or fitted to the smoking substitute device 103s during manufacture of the smoking substitute device 103s or smoking substitute system 101s. If the smoking substitute device 103s is in the form of a consumable, then the flavored article 137s may be permanently attached to the smoking substitute device 103s, such that the smoking substitute device 103s and flavored article 137s collectively form a single consumable 103s.

Alternatively, the flavored article 137s may be removable from the smoking substitute device 103s or smoking substitute system 101s, and may therefore itself be a consumable. In this case, the smoking substitute device may be shaped to ensure that the flavored article 137s can be correctly positioned relative to the inlet 107s.

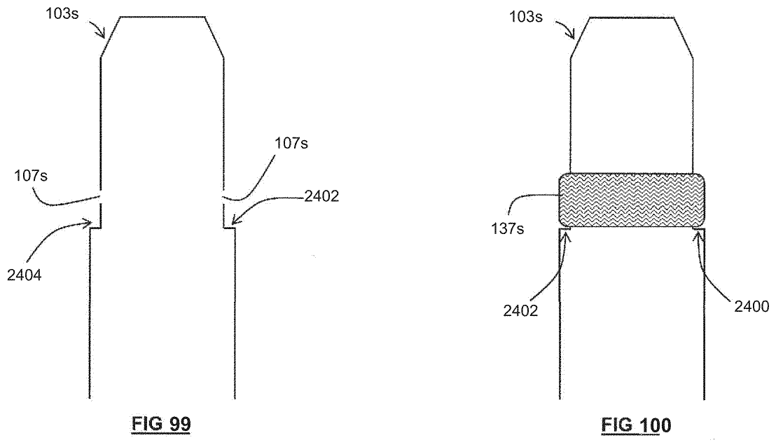

For example, as illustrated in FIGS. 97 and 98, the smoking substitute device 103s may comprise a peripheral groove, slot or recess 2400 into which the flavored article 137s can be fitted. As illustrated in FIGS. 99 and 100, the smoking substitute device 103s may have an outwardly projecting lip or shoulder 2402 against which the flavored article can be positioned. The slot 2400 or lip 2402 may be located at or in the region of the interface between the main body 102s and smoking substitute device 103s. In this way, a user can fit the flavored article 137s without having to move or slide it along the full length of the smoking substitute device 103s, as they can instead disengage the main body 102s and 103s prior to fitting the flavored article 137s.

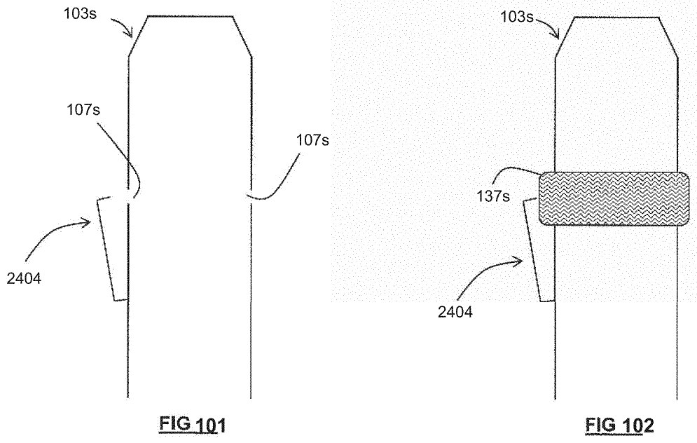

Alternatively, or additionally, as illustrated in FIGS. 101 and 102, the smoking substitute system 101s may comprise a means of fastening (e.g., a clip or clasp) 2404 by which the flavored article 137s may be secured in place. Such clip 2402 may be fixed in position, allowing a flavored article 137s to be fitted between it and the smoking substitute device 103s. For example, the clip 2404 may be bendable to allow the flavored article 137s to be fitted between it and the smoking substitute device 103s. Alternatively, clip 2404 may be hinged, for example at an attachment point between the clip 2404 and the smoking substitute device 103s, such that it can be angled towards or away from the smoking substitute device 103s to allow the flavored article 137s to be fitted. Alternatively, or additionally, the clip 2404 may be attached to be rotatable about an axis perpendicular to the surface of the smoking substitute device 103s. For example, the clip 2404 may be attached to the smoking substitute device 103s by a bearing or a ball and socket joint. Alternatively, the clip 2404 may be slidable along the length of the smoking substitute device 103s, or around the exterior surface of the smoking substitute device 103s. Any of the movable clips 2404 described herein may further be spring-loaded to hold it in position. Further, any of the clips described herein may instead form part of the main body 102s, and extend from there across the fitting position of the flavored article 137s.

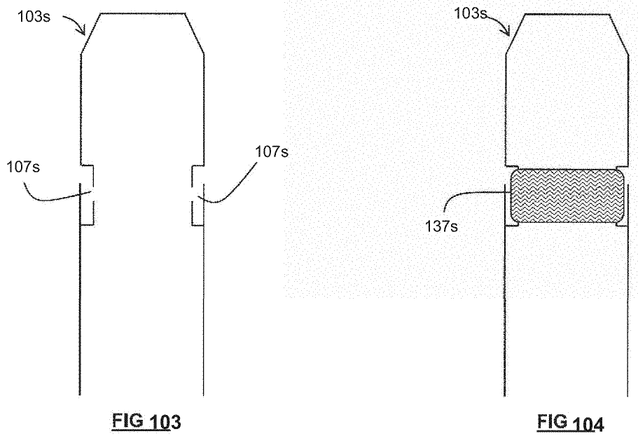

The flavored article 137s may be fully located externally to the outer surface of the smoking substitute device 103s, such as illustrated in the above-described embodiments. Alternatively, as illustrated in FIGS. 103 and 104, a part of the outer casing of the smoking substitute device 103s may be configured to cover at least a part of the flavored article 137s. This may, for example, further ensure that the flavored article cannot be inadvertently moved once in place. The part of the exterior surface that covers the flavored article 137s may, for example, be part of the main body 102s which is configured to overlap with a corresponding peripheral groove, slot, or recess in the smoking substitute device 103s when the main body 102s and smoking substitute device 103s are engaged. Alternatively, a part of the exterior surface of the smoking substitute device 103s may be configured as a slidable portion, which can then be moved to cover at least part of the flavored article 137s. Such a slidable portion may be spring-loaded to hold it in position.

Alternatively, or additionally (not shown), the smoking substitute device 103s may include a marking (e.g., a line, arrow, or colored region) to indicate correct positioning of the flavored article 137s.

A flavored article 137s, or a substitute smoking device 103s including a flavored article 137s may be provided in a retail pack comprising one or more consumables. The pack may comprise consumables, each being or comprising a flavored article 137s having the same flavorant 132s. Alternatively, the pack may comprise consumables, each being or comprising a flavored article 137s having a different flavorant 132s to provide a selection of possible flavorants to the user.

Eighteenth Mode: A Smoking Substitute Apparatus with a Flavorant on an Outside Surface

Aspects and embodiments of the eighteenth mode of the present disclosure will now be discussed with reference to the accompanying figures. Further aspects and embodiments of the eighteenth mode will be apparent to those skilled in the art. All documents mentioned in this text are incorporated herein by reference.

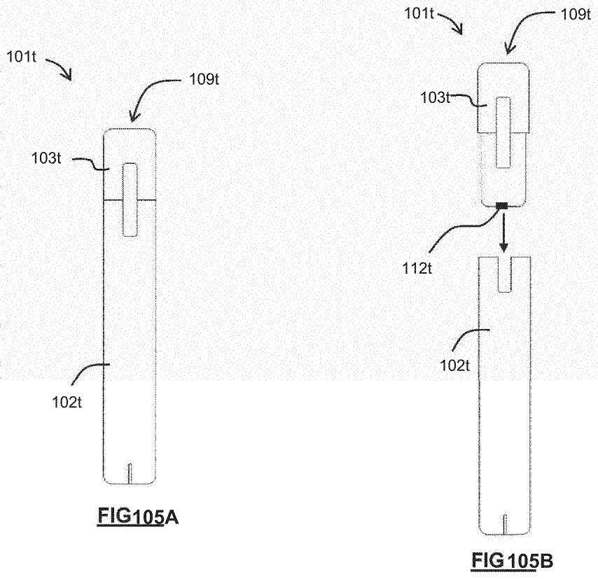

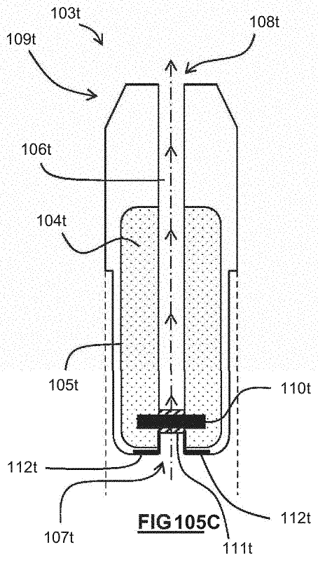

FIGS. 105A and 1056 illustrate a smoking substitute system in the form of an e-cigarette system 101t. The system 101t comprises an e-cigarette device defining a main body 102t of the system 101t, and a smoking substitute apparatus in the form of an e cigarette consumable (or “pod”) 103t. In the illustrated embodiment the consumable 103t (smoking substitute apparatus) is removable from the main body (e-cigarette device), so as to be a replaceable component of the system 101t. In other words, the e-cigarette system 101t is a closed system.

As is apparent from FIGS. 105A and 1056, the consumable 103t is configured to engage the main body 102t. FIG. 105A shows the main body 102t and the consumable 103t in an engaged state, whilst FIG. 1056 shows the main body 102t and the consumable 103t in a disengaged state. When engaged, a portion of the consumable 103t is received in a cavity of the main body 102t and is retained in the engaged position by way of a snap-engagement mechanism. In other embodiments, the main body 102t and consumable 103t may be engaged by screwing one into (or onto) the other, through a bayonet fitting, or by way of an interference fit.

The system 101t is configured to vaporize an aerosol-former, which in the illustrated embodiment, is in the form of a nicotine-based e-liquid 104t. The e-liquid 104t comprises nicotine and a base liquid including propylene glycol and/or vegetable glycerin. In the present embodiment, the e-liquid 104t is flavorless (and does not include any added flavorant). That is, if the e-liquid 104t were to be inhaled (i.e., in aerosol form) by a user, it would not have a particularly perceptible flavor or taste.

As is more apparent from FIG. 105C, this e-liquid 104t is stored within a reservoir in the form of a tank 105t that forms part of the consumable 103t. In the illustrated embodiment, the consumable 103t is a “single-use” consumable 103t. That is, upon exhausting the e-liquid 104t in the tank 105t, the intention is that the user disposes of the entire consumable 103t. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105t surrounds, and thus defines a portion of, a passage 106t that extends between an inlet 107t and an outlet 108t at opposing ends of the consumable 103t. In this respect, the passage comprises an upstream end at the end of the consumable 103t that engages with the main body 102t, and a downstream end at an opposing end of the consumable 103t that comprises a mouthpiece 109t of the system 101t. When the consumable 103t is engaged with the main body 102t, a user can inhale (i.e., take a puff) via the mouthpiece 109t so as to draw air through the passage 106t, and so as to form an airflow (indicated by arrows) in a direction from the inlet 107t to the outlet 108t of the passage 106t. Although not illustrated, the passage 106t may be partially defined by a tube (e.g., a metal tube) extending through the consumable 103t. The passage 106t is in fluid communication with a gap defined between the consumable 103t and the main body 102t (when engaged) such that air outside of the system 101t is drawn into the passage 106t (during an inhale).

The smoking substitute system 101t is configured to vaporize the e-liquid 104t for inhalation by a user. To provide this, the consumable 103t comprises a heater having of a porous wick 110t and a resistive heating element in the form of a heating filament 111t that is helically wound around a portion of the porous wick 110t. The porous wick 110t extends across the passage 106t (i.e., transverse to a longitudinal axis of the passage 106t) and opposing ends of the wick 110t extend into the tank 105t (so as to be submerged in the e liquid 104t). In this way, e-liquid 104t contained in the tank 105t is conveyed from the opposing ends of the porous wick 110t to a central portion of the porous wick 110t so as to be exposed to the airflow in the passage 106t (i.e., caused by a user inhaling).

The helical filament 111t is wound about this exposed central portion of the porous wick 110t and is electrically connected to an electrical interface in the form of electrical contacts 112t mounted at the end of the consumable that is proximate the main body 102t (when engaged). When the consumable 103t is engaged with the main body 102t, the electrical contacts 112t contact corresponding electrical contacts (not shown) of the main body 102t. The main body electrical contacts are electrically connected to a power source (not shown) of the main body 102t, such that (in the engaged position) the filament 111t is electrically connected to the power source. In this way, power can be supplied by the main body 102t to the filament 111t in order to heat the filament 111t. This heat is transferred from the filament 111t to the porous wick 110t which causes e-liquid 104t conveyed by the porous wick 110t to increase in temperature to a point at which it vaporizes. The vaporized e-liquid becomes entrained in the airflow and, between the vaporization point at the filament 111t and the outlet 108t of the passage 106t, condenses to form an aerosol. This aerosol is then inhaled, via the mouthpiece 109t, by a user of the system 101t.

The power source of the main body 102t may be in the form of a battery (e.g., a rechargeable battery). The main body 102t may comprise a connector in the form of, e.g., a USB port for recharging this battery. The main body 102t may also comprise a controller that controls the supply of power from the power source to the main body electrical contacts (and thus to the filament 111t). That, is the controller may be configured to control a voltage applied across the main body electrical contacts, and thus the voltage applied across the filament 111t. In this way, the filament 111t may only be heated under certain conditions (e.g., during a puff and/or only when the system is in an active state). In this respect, the main body 102t may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102t and consumable 103t may comprise a further interface which may, for example, be in the form of an RFID reader, a barcode or QR code reader. This interface may be able to identify a characteristic (e.g., a type) of a consumable 103t engaged with the main body 102t. In this respect, the consumable 103t may include any one or more of an RFID chip, a barcode or QR code, or memory within which is an identifier and which can be interrogated via the interface.

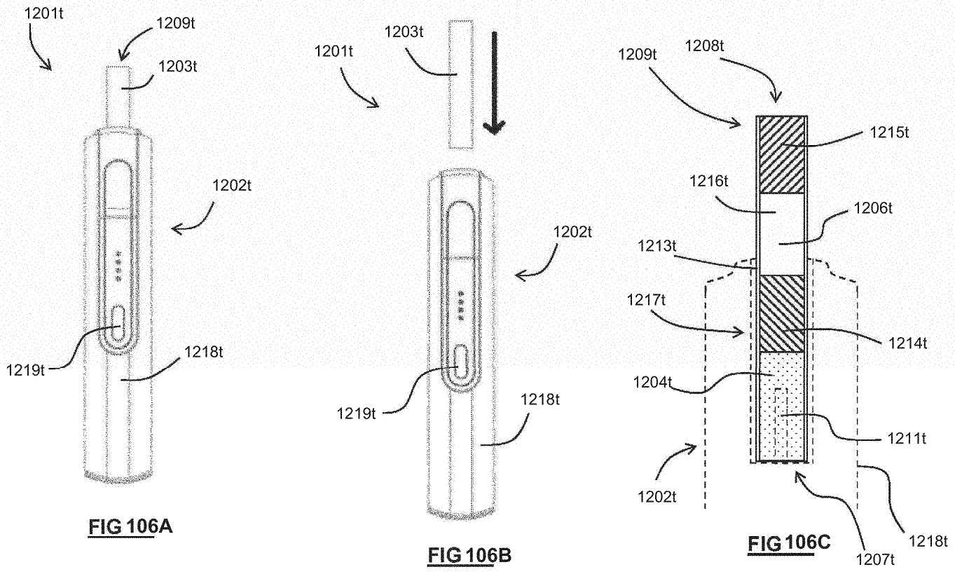

FIGS. 106A and 106B illustrate a smoking substitute system the form of a heated tobacco (HT) system 1201t. The system 1201t comprises an HT device 1202t and an aerosol-forming article in the form of a HT consumable 1203t.

The consumable 1203t is configured to engage the device 1202t by way of an interference fit. FIG. 106A shows the device 1202t and the consumable 1203t in an engaged state, and FIG. 106B shows the device 1202t and the consumable 1203t in a disengaged state.

The consumable 1203t generally resembles a cigarette. In this respect, the consumable 1202t has a generally cylindrical form with a diameter of approximately 7 mm and an axial length of approximately 70 mm. The consumable 1203t comprises an outer wrapping layer 1213t defining a passage 1206t extending between openings at opposing ends of the consumable 1203t.

Thus, the passage comprises an upstream end (and opening) 1207t at the end of the consumable 1203t that engages with the device 1202t, and a downstream end (and opening) 1208t at an opposing end of the consumable 1203t that projects from the device 1202t (when engaged) and defines a mouth end 1209t of the system 1201t. When the consumable 1203t is engaged with the device 1202t, a user can inhale (i.e., take a puff) via the mouth end 1209t so as to draw air through the passage 1206t, and so as to form an airstream in a direction from the upstream end 1207t to the downstream end 1208t of the passage 1206t.

The consumable 1203t comprises an aerosol former in the form of an aerosol-forming substrate 1204t that is disposed at the upstream end 1207t of the passage 1206t. The consumable 1203t further comprises an upstream filter 1214t adjacent the aerosol-forming substrate 1204t, a terminal filter 1215t at the downstream end 1208t, and a spacer 1216t interposed between the terminal 1215t and upstream 1214t filters.

The aerosol forming substrate 1204t comprises tobacco material that may, for example, include any suitable parts of the tobacco plant (e.g., leaves, stems, roots, bark, seeds and flowers). In order to generate an aerosol, the aerosol forming substrate 1204t comprises at least one volatile compound that is intended to be vaporized/aerosolized and that may provide the user with a recreational and/or medicinal effect when inhaled. The aerosol forming substrate 1204t may further comprise one or more additives. For example, such additives may be in the form of humectants (e.g., propylene glycol and/or vegetable glycerin), flavorants, fillers, aqueous/non-aqueous solvents and/or binders.

The device comprises a heating element 1211t that projects into the aerosol-forming substrate 1204t when the consumable 1203t is engaged with the device 1202t. This heating element 1211t is electrically connected to a power supply (not shown) of the device 1202t and, when activated, heats the aerosol-forming substrate 1204t such that vapor is released from the aerosol forming substrate 1204t. When a user inhales via the mouth end 1209t, air is drawn through the heated aerosol-forming substrate 1204t and the vapor becomes entrained in the resultant airflow. As the vapor flows from the aerosol-forming substrate 1204t to the downstream end 1208t of the passage 1206t (through the filters 1214t, 1215t and the spacer 1216t), it condenses into an aerosol and the aerosol is inhaled by the user.

As is apparent from FIG. 106C, when engaged, the consumable 1203t is received in a cavity 1217t formed in a housing 1218t of the device 1202t. Whilst not shown in the figures, the housing 1218t accommodates (in addition to the abovementioned power supply) a controller for controlling power supply to the heating element 1211t. Control of the heating element 1211t may be performed in response to a user input (e.g., via a button 1219t disposed on the housing 1218t) and/or a signal received from a puff sensor (configured to indicate a puff state).

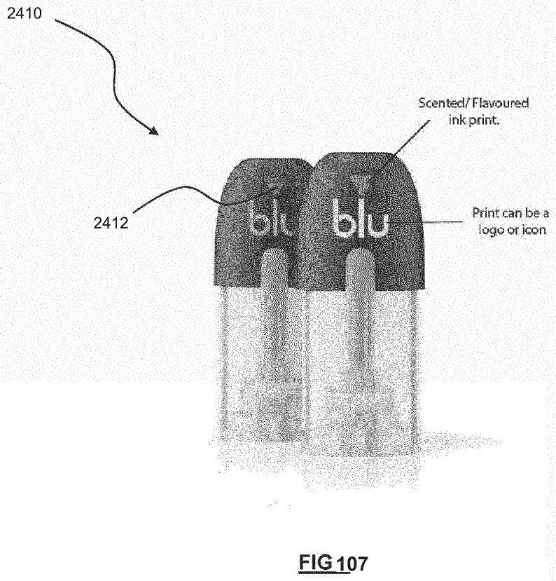

FIG. 107 shows an example of a consumable 2410, which on which is printed a visual graphic 2412, in this case a logo. The visual graphic 2412 is printed using flavored or scented ink, in line with either the first or second aspects of the present disclosure.

Nineteenth Mode: A Smoking Substitute Apparatuses Configured in Some Way to Receive a Flavorant

Aspects and embodiments of the nineteenth mode of the present disclosure will now be discussed with reference to the accompanying figures. Further aspects and embodiments of the nineteenth mode will be apparent to those skilled in the art. All documents mentioned in this text are incorporated herein by reference.

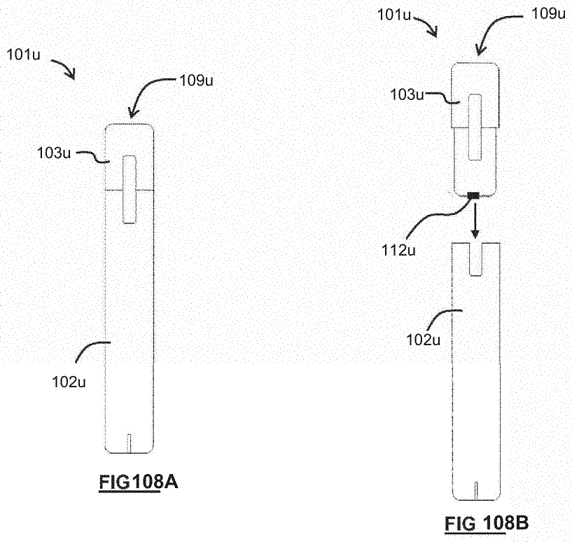

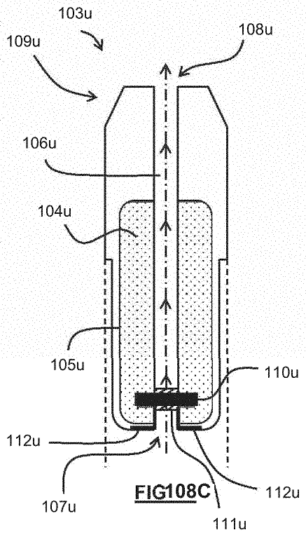

FIGS. 108A and 108B illustrate a smoking substitute system in the form of an e-cigarette system 101u. The system 101u comprises an e-cigarette device defining a main body 102u of the system 101u, and a smoking substitute apparatus in the form of an e cigarette consumable (or “pod”) 103u. In the illustrated embodiment the consumable 103u (smoking substitute apparatus) is removable from the main body (e-cigarette device), so as to be a replaceable component of the system 101u. In other words, the e-cigarette system 101u is a closed system.

As is apparent from FIGS. 108A and 108B, the consumable 103u is configured to engage the main body 102u. FIG. 108A shows the main body 102u and the consumable 103u in an engaged state, whilst FIG. 108B shows the main body 102u and the consumable 103u in a disengaged state. When engaged, a portion of the consumable 103u is received in a cavity of the main body 102u and is retained in the engaged position by way of a snap-engagement mechanism. In other embodiments, the main body 102u and consumable 103u may be engaged by screwing one into (or onto) the other, through a bayonet fitting, or by way of an interference fit.

The system 101u is configured to vaporize an aerosol-former, which in the illustrated embodiment, is in the form of a nicotine-based e-liquid 104u. The e-liquid 104u comprises nicotine and a base liquid including propylene glycol and/or vegetable glycerin. In the present embodiment, the e-liquid 104u is flavorless (and does not include any added flavorant). That is, if the e-liquid 104u were to be inhaled (i.e., in aerosol form) by a user, it would not have a particularly perceptible flavor or taste.

As is more apparent from FIG. 108C, this e-liquid 104u is stored within a reservoir in the form of a tank 105u that forms part of the consumable 103u. In the illustrated embodiment, the consumable 103u is a “single-use” consumable 103u. That is, upon exhausting the e-liquid 104u in the tank 105u, the intention is that the user disposes of the entire consumable 103u. In other embodiments, the e-liquid (i.e., aerosol former) may be the only part of the system that is truly “single-use”. That is, the tank may be refillable with e-liquid or the e-liquid may be stored in a non-consumable component of the system. For example, the e-liquid may be stored in a tank located in the main body or stored in another component that is itself not single-use (e.g., a refillable cartomizer).

The tank 105u surrounds, and thus defines a portion of, a passage 106u that extends between an inlet 107u and an outlet 108u at opposing ends of the consumable 103u. In this respect, the passage comprises an upstream end at the end of the consumable 103u that engages with the main body 102u, and a downstream end at an opposing end of the consumable 103u that comprises a mouthpiece 109u of the system 101u. When the consumable 103u is engaged with the main body 102u, a user can inhale (i.e., take a puff) via the mouthpiece 109u so as to draw air through the passage 106u, and so as to form an airflow (indicated by arrows) in a direction from the inlet 107u to the outlet 108u of the passage 106u. Although not illustrated, the passage 106u may be partially defined by a tube (e.g., a metal tube) extending through the consumable 103u. The passage 106u is in fluid communication with a gap defined between the consumable 103u and the main body 102u (when engaged) such that air outside of the system 101u is drawn into the passage 106u (during an inhale).

The smoking substitute system 101u is configured to vaporize the e-liquid 104u for inhalation by a user. To provide this, the consumable 103u comprises a heater having of a porous wick 110u and a resistive heating element in the form of a heating filament 111u that is helically wound around a portion of the porous wick 110u. The porous wick 110u extends across the passage 106u (i.e., transverse to a longitudinal axis of the passage 106u) and opposing ends of the wick 110u extend into the tank 105u (so as to be submerged in the e liquid 104u). In this way, e-liquid 104u contained in the tank 105u is conveyed from the opposing ends of the porous wick 110u to a central portion of the porous wick 110u so as to be exposed to the airflow in the passage 106u (i.e., caused by a user inhaling).

The helical filament 111u is wound about this exposed central portion of the porous wick 110u and is electrically connected to an electrical interface in the form of electrical contacts 112u mounted at the end of the consumable that is proximate the main body 102u (when engaged). When the consumable 103u is engaged with the main body 102u, the electrical contacts 112u contact corresponding electrical contacts (not shown) of the main body 102u. The main body electrical contacts are electrically connected to a power source (not shown) of the main body 102u, such that (in the engaged position) the filament 111u is electrically connected to the power source. In this way, power can be supplied by the main body 102u to the filament 111u in order to heat the filament 111u. This heat is transferred from the filament 111u to the porous wick 110u which causes e-liquid 104u conveyed by the porous wick 110u to increase in temperature to a point at which it vaporizes. The vaporized e-liquid becomes entrained in the airflow and, between the vaporization point at the filament 111u and the outlet 108u of the passage 106u, condenses to form an aerosol. This aerosol is then inhaled, via the mouthpiece 109u, by a user of the system 101u.

The power source of the main body 102u may be in the form of a battery (e.g., a rechargeable battery).

The main body 102u may comprise a connector in the form of, e.g., a USB port for recharging this battery. The main body 102u may also comprise a controller that controls the supply of power from the power source to the main body electrical contacts (and thus to the filament 111u). That, is the controller may be configured to control a voltage applied across the main body electrical contacts, and thus the voltage applied across the filament 111u. In this way, the filament 111u may only be heated under certain conditions (e.g., during a puff and/or only when the system is in an active state). In this respect, the main body 102u may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body 102u and consumable 103u may comprise a further interface which may, for example, be in the form of an RFID reader, a barcode or QR code reader. This interface may be able to identify a characteristic (e.g., a type) of a consumable 103u engaged with the main body 102u. In this respect, the consumable 103u may include any one or more of an RFID chip, a barcode or QR code, or memory within which is an identifier and which can be interrogated via the interface.

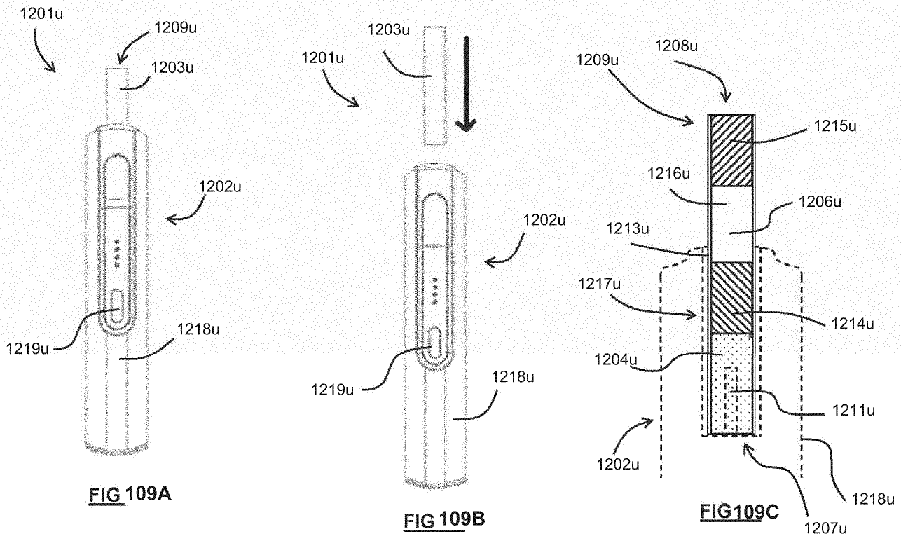

FIGS. 109A and 109B illustrate a smoking substitute system the form of a heated tobacco (HT) system 1201u. The system 1201u comprises an HT device 1202u and an aerosol-forming article in the form of a HT consumable 1203u.

The consumable 1203u is configured to engage the device 1202u by way of an interference fit. FIG. 109A shows the device 1202u and the consumable 1203u in an engaged state, and FIG. 2B shows the device 1202u and the consumable 1203u in a disengaged state.

The consumable 1203u generally resembles a cigarette. In this respect, the consumable 1203u has a generally cylindrical form with a diameter of approximately 7 mm and an axial length of approximately 70 mm. The consumable 1203u comprises an outer wrapping layer 1213u defining a passage 1206u extending between openings at opposing ends of the consumable 1203u.

Thus, the passage comprises an upstream end (and opening) 207 at the end of the consumable 1203u that engages with the device 1202u, and a downstream end (and opening) 1208u at an opposing end of the consumable 1203u that projects from the device 1202u (when engaged) and defines a mouth end 1209u of the system 1201u. When the consumable 1203u is engaged with the device 1202u, a user can inhale (i.e., take a puff) via the mouth end 1209u so as to draw air through the passage 1206u, and so as to form an airstream in a direction from the upstream end 1207u to the downstream end 1208u of the passage 1206u.

The consumable 1203u comprises an aerosol former in the form of an aerosol-forming substrate 1204u that is disposed at the upstream end 1207u of the passage 1206u. The consumable 1203u further comprises an upstream filter 1214u adjacent the aerosol-forming substrate 1204u, a terminal filter 1215u at the downstream end 1208u, and a spacer 1216u interposed between the terminal filter 1215u and upstream filter 1214u.

The aerosol forming substrate 1204u comprises tobacco material that may, for example, include any suitable parts of the tobacco plant (e.g., leaves, stems, roots, bark, seeds and flowers). In order to generate an aerosol, the aerosol forming substrate 1204u comprises at least one volatile compound that is intended to be vaporized/aerosolized and that may provide the user with a recreational and/or medicinal effect when inhaled. The aerosol-forming substrate 1204u may further comprise one or more additives. For example, such additives may be in the form of humectants (e.g., propylene glycol and/or vegetable glycerin), flavorants, fillers, aqueous/non-aqueous solvents and/or binders.

The device comprises a heating element 1211u that projects into the aerosol-forming substrate 1204u when the consumable 1203u is engaged with the device 1202u. This heating element 1211u is electrically connected to a power supply (not shown) of the device 1202u and, when activated, heats the aerosol-forming substrate 1204u such that vapor is released from the aerosol-forming substrate 1204u. When a user inhales via the mouth end 1209u, air is drawn through the heated aerosol-forming substrate 1204u and the vapor becomes entrained in the resultant airflow. As the vapor flows from the aerosol-forming substrate 1204u to the downstream end 1208u of the passage 1206u (through the filters 1214u, 1215u and the spacer 1216u), it condenses into an aerosol and the aerosol is inhaled by the user.

As is apparent from FIG. 109C, when engaged, the consumable 1203u is received in a cavity 1217u formed in a housing 1218u of the device 1202u. Whilst not shown in the figures, the housing 1218u accommodates (in addition to the abovementioned power supply) a controller for controlling power supply to the heating element 1211u. Control of the heating element 1211u may be performed in response to a user input (e.g., via a button 1219u disposed on the housing 1218u) and/or a signal received from a puff sensor (configured to indicate a puff state).

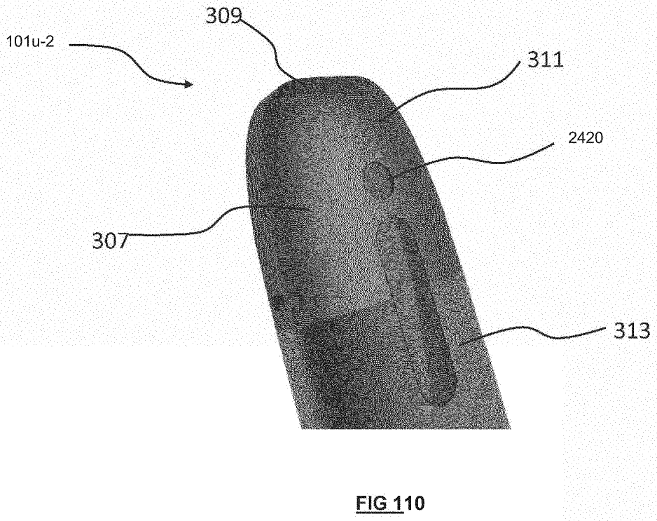

FIG. 110 shows an end of an e-cigarette system 101u-2, which may be as shown in FIGS. 108A to 108C. E-cigarette system 101u-2 includes a recess 2420 which is located on the top surface 307 of the mouthpiece end 309 of the portion of the consumable 311 which is visible when the consumable is engaged with the main body 313. In the embodiment shown in FIG. 110, the recess 2420 which is shaped as a shallow cylinder, and is configured to retain liquid flavorant which is stored therein against gravity, e.g., by exploiting the surface tension of the liquid flavorant (not shown). In this embodiment, and others, the geometry of the recess 2420 may be such that it is small enough so as only to hold enough liquid flavorant for a limited amount of vaping, e.g., 3 three-second inhalations, before the liquid flavorant is completely volatilized. By having such a small volume recess 2420, a user is able to switch flavorants at leisure, because each individual portion supplied to the recess 2420 does not last long before fully evaporating.

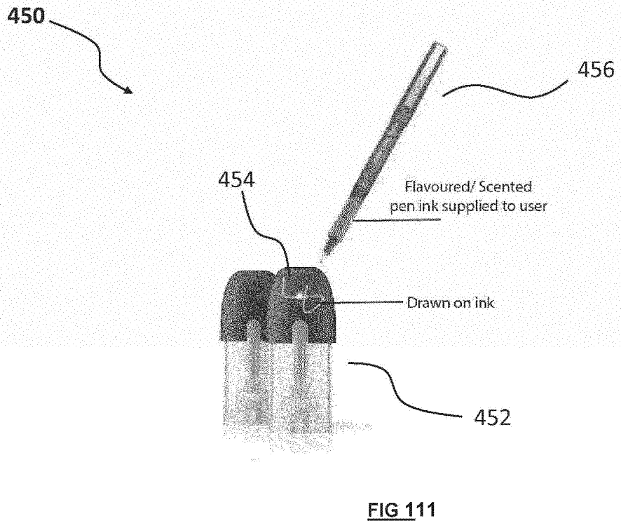

FIG. 111 shows an example of a smoking substitute kit 450 according to the fourth aspect of the disclosure. The smoking substitute kit 450 includes a consumable 452 having a flavorant receiving portion 454 at its upper end, and a flavorant applicator in the form of pen 456. As discussed, and as is shown in FIG. 111, a user is able to apply liquid flavorant in the form of flavored or scented ink by using the pen 456. In order to achieve this, the flavorant receiving portion 454 is adapted so that the ink from the pen 456 is able to stick to the surface of the consumable 452.

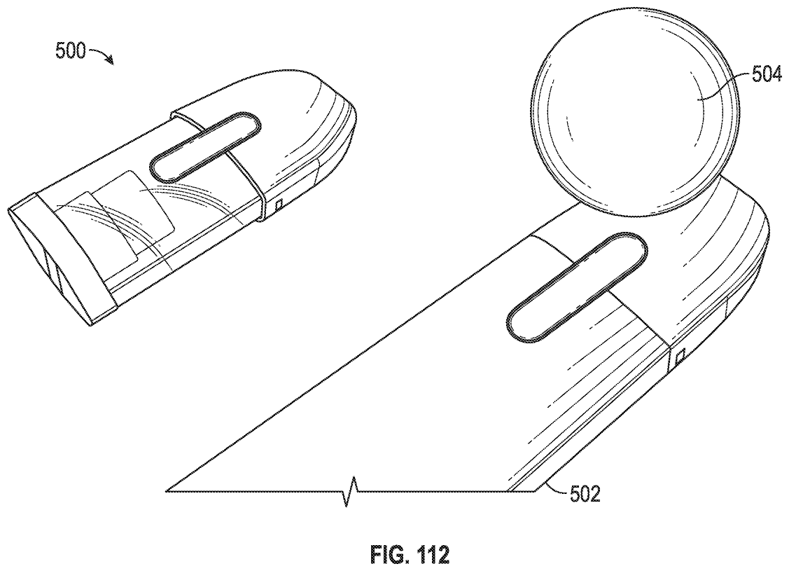

FIG. 112 shows another example of a smoking substitute kit 500 according to the fourth aspect of the disclosure. The smoking substitute kit 500 includes smoking substitute apparatus 502 and flavorant applicator 506. The flavorant applicator 506 in FIG. 112 is spherical and includes liquid flavorant which is encapsulated in microcapsules which are configured to rupture and deposit the flavorant onto the surface of the smoking substitute apparatus 502 when the flavorant applicator 506 is rubbed or scratched, or under the application of heat.

This works using the same mechanism as “scratch and sniff”.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the disclosure in diverse forms thereof.

While the disclosure has been described in conjunction with the exemplary embodiments described above, many equivalent modifications and variations will be apparent to those skilled in the art when given this disclosure. Accordingly, the exemplary embodiments of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the disclosure that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. A smoking substitute apparatus, comprising:
a substrate for a reservoir having an aerosol former stored therein, the substrate comprising:
a property-modifying agent disposed on the substrate;
wherein the substrate is configured to be received in the reservoir to contact the aerosol former, so as to facilitate a release of the property-modifying agent from the substrate to the aerosol former; and
wherein the substrate is insoluble in the aerosol former.

2. The smoking substitute apparatus of claim 1, wherein the property-modifying agent is configured to be deposited on a surface of the substrate.

3. The smoking substitute apparatus of claim 1, wherein the substrate is coated in a soluble coating containing the property-modifying agent.

4. The smoking substitute apparatus of claim 1, wherein the substrate comprises an absorbent paper.

5. The smoking substitute apparatus of claim 1, wherein the substrate is configured to be removably received in the reservoir.

6. The smoking substitute apparatus of claim 1, wherein an amount of property-modifying agent releasable to the aerosol former corresponds to a predetermined period of time the substrate is received in the reservoir.

7. The smoking substitute apparatus substrata of claim 1, wherein the substrate is configured to be received in a reservoir of a smoking substitute apparatus.

8. The smoking substitute apparatus of claim 1, wherein the property-modifying agent comprises flavorant and/or colorant.

9. The smoking substitute apparatus of claim 1, further comprising:
a reservoir for receiving the substrate, the reservoir configured to store an aerosol former therein, the reservoir comprising an opening for providing access to the substrate as it extends into the reservoir to contact the aerosol former.

10. The smoking substitute apparatus of claim 9, wherein the reservoir comprises a closure for closing the opening, wherein the closure is configured to open to provide access for the substrate.

11. The smoking substitute apparatus of claim 10, wherein the closure comprises a valve having a resilient member for forming a peripheral seal with the substrate as it extends through the opening.

12. (canceled)

13. A smoking substitute kit comprising:
a substrate for a reservoir having an aerosol former stored therein, the substrate comprising: a property-modifying agent disposed on the substrate; wherein the substrate is configured to be received in the reservoir to contact the aerosol former, so as to facilitate a release of the property-modifying agent from the substrate to the aerosol former, and wherein the substrate is insoluble in the aerosol former; and
the reservoir, wherein the reservoir is configured to store an aerosol former therein, the reservoir comprising an opening for providing access to the substrate as it extends into the reservoir to contact the aerosol former.

14. A method comprising:
inserting a substrate into an opening of a reservoir so that the substrate extends into the reservoir and contacts an aerosol former, wherein the aerosol former is a liquid aerosol former, and wherein the substrate has an aerosol former stored therein, the substrate comprising:
a property-modifying agent disposed on the substrate; wherein the substrate is configured to be received in the reservoir to contact the aerosol former, so as to facilitate a release of the property-modifying agent from the substrate to the aerosol former, and wherein the substrate is insoluble in the aerosol former.

15. The smoking substitute kit of claim 13,
wherein the aerosol former is a liquid aerosol former.

16.-312. (canceled)

313. The method of claim 14, wherein the reservoir is configured to store an aerosol former therein, the reservoir comprising an opening for providing access to the substrate as it extends into the reservoir to contact the aerosol former.

314. The method of claim 313, wherein the reservoir comprises a closure for closing the opening, wherein the closure is configured to open to provide access for the substrate.

315. The method of claim 314, wherein the closure comprises a valve having a resilient member for forming a peripheral seal with the substrate as it extends through the opening.
